(19) United States
(12) Patent Application Publication
(10) Pub. No.: US 2003/0148295 A1
(43) Pub. Date: Aug. 7, 2003

(54) Expression profiles and methods of use

(76) Inventors: Jackson Shek-Lam Wan, San Diego, CA (US); Yixin Wang, San Diego, CA (US)

Correspondence Address: PRESTON GATES ELLIS & ROUVELAS MEEDS LLP, 1735 NEW YORK AVENUE, NW, SUITE 500, WASHINGTON, DC, 20006, US

(21) Appl. No.: 10/101,510

(22) Filed: Mar. 20, 2002

Related U.S. Application Data
(60) Provisional application No. 60/276,947, filed on Mar. 20, 2001.

(51) Int. Cl. C12Q 1/68; C07H 21/04; C12N 9/00; C12P 21/02; C12N 5/06

(52) U.S. Cl. 435/6; 435/69.1; 435/183; 435/320.1; 435/325; 536/23.2

(57) ABSTRACT

The present invention relates to gene expression profiles, algorithms to generate gene expression profiles, microarrays comprising nucleic acid sequences representing gene expression profiles, methods of using gene expression profiles and microarrays, and business methods directed to the use of gene expression profiles, microarrays, and algorithms. The present invention further relates to protein expression profiles, algorithms to generate protein expression profiles, microarrays comprising protein-capture agents that bind proteins comprising protein expression profiles, methods of using protein expression profiles and microarrays, and business methods directed to the use of protein expression profiles, microarrays, and algorithms.

### CROSS REFERENCE TO RELATED APPLICATIONS

The present application is related to and claims, under 35 U.S.C. §119(e), the benefit of U.S. Provisional Patent Application Serial No. 60/276,947, filed Mar. 20, 2001, which is incorporated herein by reference.

### FIELD OF THE INVENTION

The present invention relates to gene expression profiles, algorithms to generate gene expression profiles, microarrays comprising nucleic acid sequences representing gene expression profiles, methods of using gene expression profiles and microarrays, and business methods directed to the use of gene expression profiles, microarrays, and algorithms.

The present invention further relates to protein expression profiles, algorithms to generate protein expression profiles, microarrays comprising protein-capture agents that bind proteins comprising protein expression profiles, methods of using protein expression profiles and microarrays, and business methods directed to the use of protein expression profiles, microarrays, and algorithms.

### BACKGROUND OF THE INVENTION

The identification and analysis of a particular gene or protein generally has been accomplished by experiments directed specifically towards that gene or protein. With the recent advances, however, in the sequencing of the human genome, the challenge is to decipher the expression, function, and regulation of thousands of genes, which cannot be realistically accomplished by analyzing one gene or protein at a time. To address this situation, DNA microarray technology has proven to be a valuable tool. By taking advantage of the sequence information obtained from DNA microarrays, the expression and functional relationship of thousands of genes may be resolved.

The expression profiles of thousands of genes have been examined en masse via cDNA and oligonucleotide microarrays. See, e.g., Lockhart et al., NUCLEIC ACIDS SYMP. SER. 11-12 (1998); Shalon et al., 46 PATHOL. BIOL. 107-109 (1998); Schena et al., 16 TRENDS BIOTECHNOL. 301-306 (1998). Several studies have analyzed gene expression profiles in yeast, mammalian cell lines, and disease tissues. See, e.g., Welford et al., 26 NUCLEIC ACIDS RES. 3059-3065 (1998); Cho et al., 2 MOL. CELL 65-73 (1997); Heller et al., 94 PROC. NATL. ACAD. SCI. USA 2150-2155 (1997); Schena et al., 93 PROC. NATL. ACAD. SCI. USA 10614-10619 (1996).

Microarray technology provides the means to decipher the function of a particular gene based on its expression profile and alterations in its expression levels. In addition, this technology may be used to define the components of cellular pathways as well as the regulation of these cellular components. High-density oligonucleotide microarrays may be used to simultaneously monitor thousands of genes or possibly entire genomes (e.g., Saccharomyces cerevisiae).

Microarrays may also be used for genetic and physical mapping of genomes, DNA sequencing, genetic diagnosis, and genotyping of organisms. Microarrays may be used to determine a medical diagnosis. For example, the identity of a pathogenic microorganism may be established unambiguously by hybridizing a patient sample to a microarray containing the genes from many types of known pathogenic DNA. A similar technique may also be used for genotyping an organism. For genetic diagnostics, a microarray may contain multiple forms of a mutated gene or multiple genes associated with a particular disease. The microarray may then be probed with DNA or RNA, isolated from a patient sample (e.g., blood sample), which may hybridize to one of the mutated or disease genes.

Microarrays containing molecular expression markers or predictor genes may be used to confirm tissue or cell identifications. In addition, disease progression may be monitored by analyzing the expression patterns of the predictor genes in disease tissues. An alteration in gene expression may be used to define the specific disease state and stage of the disease. Monitoring the efficacy of certain drug regimens may also be accomplished by analyzing the expression patterns of the predictor genes. For example, decreases or increases in gene expression may be indicative of the efficacy of a particular drug.

Generally, oligonucleotide probes are used to detect complementary nucleic acid sequences in a particular tissue or cell type. The oligonucleotide probes may be covalently attached to a support, and arrays of oligonucleotide probes immobilized on solid supports are used to detect specific nucleic acid sequences. To assess gene expression in a given tissue or cell sample, DNA or RNA is isolated from the tissue or cell, labeled with a fluorescent dye, and then hybridized to the DNA microarray. The microarray may contain hundreds to thousands of DNA sequences selected from cDNA libraries, genomic DNA, or expressed sequence tags (ESTs). These DNA sequences may be spotted or synthesized onto the support and then crosslinked to the support by ultraviolet radiation. Following hybridization, the fluorescence intensities of the microarray are analyzed, and these measurements are then used to determine the presence or relative quantity of a particular gene within the sample. This hybridization pattern is used to generate a gene expression profile of the target tissue or cell type.

Thus, differences in gene expression profiles may be used to identify the pathology of many diseases involving alterations of gene expression. The types of genes and their expression levels may distinguish normal tissue and diseased tissue. For example, cancer cells evolve from normal cells into highly invasive, metastatic malignancies, which frequently are induced by activation of oncogenes, or inactivation of tumor suppressor genes. Differentially expressed sequences can serve as markers or predictors of the transformed state and are, therefore, of potential value in the diagnosis and classification of tumors. The assessment of expression profiles may provide meaningful information with respect to tumor type and stage, treatment methods, and prognosis.

### SUMMARY OF THE INVENTION

The present invention relates to gene expression profiles, algorithms to generate gene expression profiles, microarrays comprising nucleic acid sequences representing gene expression profiles, methods of using gene expression profiles and microarrays, and business methods directed to the use of gene expression profiles, microarrays, and algorithms.

In a specific embodiment of the present invention, the gene expression profile may be an endothelial cell gene expression profile comprising one or more nucleic acid sequences substantially homologous to a nucleic acid sequence or complementary sequence thereof selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 48; SEQ ID NO: 63; SEQ ID NO: 70; SEQ ID NO: 82; SEQ ID NO: 94; and SEQ ID NO: 144. With regard to this gene expression profile, the present invention provides a microarray comprising one or more protein-capture agents that specifically bind to all or a portion of one or more of the proteins encoded by the genes comprising the gene expression profile.

In another embodiment of the present invention, the gene expression profile may be a muscle cell gene expression profile comprising one or more nucleic acid sequences substantially homologous to a nucleic acid sequence or complementary sequence thereof selected from the group consisting of SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 39; SEQ ID NO: 40; SEQ ID NO: 41; SEQ ID NO: 42; SEQ ID NO: 54; SEQ ID NO: 55; and SEQ ID NO: 69. With regard to this gene expression profile, the present invention provides a microarray comprising one or more protein-capture agents that specifically bind to all or a portion of one or more of the proteins encoded by the genes comprising the gene expression profile.

In an alternative embodiment of the present invention, the gene expression profile may be a primary cell gene expression profile comprising one or more nucleic acid sequences or complementary sequences thereof, or portions of said nucleic acid sequences or complementary sequences thereof, selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 1; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186.

With regard to this gene expression profile, the present invention provides a microarray comprising one or more protein-capture agents that specifically bind to all or a portion of one or more of the proteins encoded by the genes comprising the gene expression profile.

In a further aspect of the present invention, the gene expression profile may be an epithelial cell gene expression profile comprising one or more nucleic acid sequences or complementary sequences thereof, or portions of said nucleic acid sequences or complementary sequences thereof, selected from the group consisting of SEQ ID NO: 47; SEQ ID NO: 60; SEQ ID NO: 67; SEQ ID NO: 73; SEQ ID NO: 75; SEQ ID NO: 76; SEQ ID NO: 77; SEQ ID NO: 78; SEQ ID NO: 80; SEQ ID NO: 96; SEQ ID NO: 98; SEQ ID NO: 99; SEQ ID NO: 111; SEQ ID NO: 112; SEQ ID NO: 123; SEQ ID NO: 127; SEQ ID NO: 131; SEQ ID NO: 150; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186. With regard to this gene expression profile, the present invention provides a microarray comprising one or more protein-capture agents that specifically bind to all or a portion of one or more of the proteins encoded by the genes comprising the gene expression profile.

In yet another embodiment, a keratinocyte epithelial cell gene expression profile may comprise one or more nucleic acid sequences or complementary sequences thereof, or portions of said nucleic acid sequences or complementary sequences thereof, selected from the group consisting of SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; and SEQ ID NO: 211. With regard to this gene expression profile, the present invention provides a microarray comprising one or more protein-capture agents that specifically bind to all or a portion of one or more of the proteins encoded by the genes comprising the gene expression profile.

The present invention also provides a mammary epithelial cell gene expression profile comprising one or more nucleic acid sequences or complementary sequences thereof, or portions of said nucleic acid sequences or complementary sequences thereof, selected from the group consisting of SEQ ID NO: 78; SEQ ID NO: 212; SEQ ID NO: 213; SEQ ID NO: 216; SEQ ID NO: 225; SEQ ID NO: 226; SEQ ID NO: 227; SEQ ID NO: 239; SEQ ID NO: 271; SEQ ID NO: 285; and SEQ ID NO: 289. With regard to this gene expression profile, the present invention provides a microarray comprising one or more protein-capture agents that specifically bind to all or a portion of one or more of the proteins encoded by the genes comprising the gene expression profile.

In an alternative embodiment, a bronchial epithelial cell gene expression profile may comprise one or more nucleic acid sequences or complementary sequences thereof, or portions of said nucleic acid sequences or complementary sequences thereof, selected from the group consisting of SEQ ID NO: 27; SEQ ID NO: 131; SEQ ID NO: 150; SEQ ID NO: 169; SEQ ID NO: 214; SEQ ID NO: 215; SEQ ID NO: 223; SEQ ID NO: 224; SEQ ID NO: 241; SEQ ID NO: 243; SEQ ID NO: 244; SEQ ID NO: 255; SEQ ID NO: 256; SEQ ID NO: 261; and SEQ ID NO: 314. With regard to this gene expression profile, the present invention provides a microarray comprising one or more protein-capture agents that specifically bind to all or a portion of one or more of the proteins encoded by the genes comprising the gene expression profile.

The present invention also provides a prostate epithelial cell gene expression profile, which may comprise one or more nucleic acid sequences or complementary sequences thereof, or portions of said nucleic acid sequences or complementary sequences thereof, selected from the group consisting of SEQ ID NO: 64; SEQ ID NO: 217; SEQ ID NO: 218; SEQ ID NO: 259; SEQ ID NO: 293; SEQ ID NO: 302; and SEQ ID NO: 320. With regard to this gene expression profile, the present invention provides a microarray comprising one or more protein-capture agents that specifically bind to all or a portion of one or more of the proteins encoded by the genes comprising the gene expression profile.

In yet another embodiment, a renal cortical epithelial cell gene expression profile may comprise one or more nucleic acid sequences or complementary sequences thereof, or portions of said nucleic acid sequences or complementary sequences thereof, selected from the group consisting of SEQ ID NO: 49; SEQ ID NO: 57; SEQ ID NO: 104; SEQ ID NO: 123; SEQ ID NO: 160; SEQ ID NO: 165; SEQ ID NO: 166; SEQ ID NO: 219; SEQ ID NO: 267; SEQ ID NO: 270; SEQ ID NO: 279; SEQ ID NO: 280; SEQ ID NO: 283; SEQ ID NO: 291; SEQ ID NO: 305; SEQ ID NO: 307; SEQ ID NO: 310; SEQ ID NO: 313; SEQ ID NO: 325; SEQ ID NO: 326; and SEQ ID NO: 327. With regard to this gene expression profile, the present invention provides a microarray comprising one or more protein-capture agents that specifically bind to all or a portion of one or more of the proteins encoded by the genes comprising the gene expression profile.

The present invention further provides a renal proximal tubule epithelial cell gene expression profile comprising one or more nucleic acid sequences or complementary sequences thereof, or portions of said nucleic acid sequences or complementary sequences thereof, selected from the group consisting of SEQ ID NO: 106; SEQ ID NO: 138; SEQ ID NO: 158; SEQ ID NO: 228; SEQ ID NO: 236; SEQ ID NO: 242; SEQ ID NO: 250; SEQ ID NO: 258; SEQ ID NO: 260; SEQ ID NO: 262; SEQ ID NO: 266; SEQ ID NO: 272; SEQ ID NO: 273; SEQ ID NO: 274; SEQ ID NO: 275; SEQ ID NO: 276; SEQ ID NO: 278; SEQ ID NO: 284; SEQ ID NO: 288; SEQ ID NO: 295; SEQ ID NO: 296; SEQ ID NO: 297; SEQ ID NO: 299; SEQ ID NO: 300; SEQ ID NO: 301; SEQ ID NO: 306; SEQ ID NO: 308; SEQ ID NO: 309; SEQ ID NO: 311; SEQ ID NO: 316; SEQ ID NO: 318; SEQ ID NO: 321; SEQ ID NO: 322; SEQ ID NO: 328; and SEQ ID NO: 329. With regard to this gene expression profile, the present invention provides a microarray comprising one or more protein-capture agents that specifically bind to all or a portion of one or more of the proteins encoded by the genes comprising the gene expression profile.

In a specific embodiment, a small airway epithelial cell gene expression profile may comprise one or more nucleic acid sequences or complementary sequences thereof, or portions of said nucleic acid sequences or complementary sequences thereof, selected from the group consisting of SEQ ID NO: 173; SEQ ID NO: 174; SEQ ID NO: 183; SEQ ID NO: 220; SEQ ID NO: 221; SEQ ID NO: 222; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 237; SEQ ID NO: 238; SEQ ID NO: 240; SEQ ID NO: 245; SEQ ID NO: 246; SEQ ID NO: 247; SEQ ID NO: 248; SEQ ID NO: 249; SEQ ID NO: 251; SEQ ID NO: 252; SEQ ID NO: 254; SEQ ID NO: 257; SEQ ID NO: 263; SEQ ID NO: 264; SEQ ID NO: 265; SEQ ID NO: 268; SEQ ID NO: 269; SEQ ID NO: 270; SEQ ID NO: 277; SEQ ID NO: 281; SEQ ID NO: 282; SEQ ID NO: 286; SEQ ID NO: 287; SEQ ID NO: 290; SEQ ID NO: 294; SEQ ID NO: 298; SEQ ID NO: 303; SEQ ID NO: 312; SEQ ID NO: 315; SEQ ID NO: 317; and SEQ ID NO: 319. With regard to this gene expression profile, the present invention provides a microarray comprising one or more protein-capture agents that specifically bind to all or a portion of one or more of the proteins encoded by the genes comprising the gene expression profile.

The present invention also provides a renal epithelial cell gene expression profile comprising one or more nucleic acid sequences or complementary sequences thereof, or portions of said nucleic acid sequences or complementary sequences thereof, selected from the group consisting of SEQ ID NO: 37; SEQ ID NO: 253; SEQ ID NO: 304; SEQ ID NO: 323; and SEQ ID NO: 324. With regard to this gene expression profile, the present invention provides a microarray comprising one or more protein-capture agents that specifically bind to all or a portion of one or more of the proteins encoded by the genes comprising the gene expression profile.

In yet another embodiment of the present invention, the gene expression profiles may comprise one or more genes, wherein said gene expression profile is generated from a cell type selected from the group comprising coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesangial cells, coronary artery smooth muscle, bronchial smooth muscle, uterine smooth muscle, lung fibroblast, osteoblasts, and prostate stromal cells.

In another embodiment of the present invention, the microarray may be a microarray comprising an endothelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 48; SEQ ID NO: 63; SEQ ID NO: 70; SEQ ID NO: 82; SEQ ID NO: 94; and SEQ ID NO: 144.

The microarrays of the present invention may also comprise a microarray comprising a muscle cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 39; SEQ ID NO: 40; SEQ ID NO: 41; SEQ ID NO: 42; SEQ ID NO: 54; SEQ ID NO: 55; and SEQ ID NO: 69.

Also within the scope of the present invention are microarrays comprising a primary cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 1; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; SEQ ID NO: 102; SEQ ID NO: 103; SEQ ID NO: 104; SEQ ID NO: 105; SEQ ID NO:. 106; SEQ ID NO: 107; SEQ ID NO: 108; SEQ ID NO: 109; SEQ ID NO: 110; SEQ ID NO: 11; SEQ ID NO: 112; SEQ ID NO: 113; SEQ ID NO: 114; SEQ ID NO: 115; SEQ ID NO: 116; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186.

In a further embodiment, the microarray may be a microarray comprising an epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 47; SEQ ID NO: 60; SEQ ID NO: 67; SEQ ID NO: 73; SEQ ID NO: 75; SEQ ID NO: 76; SEQ ID NO: 77; SEQ ID NO: 78; SEQ ID NO: 80; SEQ ID NO: 96; SEQ ID NO: 98; SEQ ID NO: 99; SEQ ID NO: 11; SEQ ID NO: 112; SEQ ID NO: 123; SEQ ID NO: 127; SEQ ID NO: 131; SEQ ID NO: 150; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186.

In yet another embodiment, a microarray may comprise a keratinocyte epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; and SEQ ID NO: 211.

The present invention also provides a microarray comprising a mammary epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 78; SEQ ID NO: 212; SEQ ID NO: 213; SEQ ID NO: 216; SEQ ID NO: 225; SEQ ID NO: 226; SEQ ID NO: 227; SEQ ID NO: 239; SEQ ID NO: 271; SEQ ID NO: 285; and SEQ ID NO: 289.

In an alternative embodiment, a microarray may comprise a bronchial epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 27; SEQ ID NO: 131; SEQ ID NO: 150; SEQ ID NO: 169; SEQ ID NO: 214; SEQ ID NO: 215; SEQ ID NO: 223; SEQ ID NO: 224; SEQ ID NO: 241; SEQ ID NO: 243; SEQ ID NO: 244; SEQ ID NO: 255; SEQ ID NO: 256; SEQ ID NO: 261; and SEQ ID NO: 314.

The present invention also provides a microarray comprising a prostate epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 64; SEQ ID NO: 217; SEQ ID NO: 218; SEQ ID NO: 259; SEQ ID NO: 293; SEQ ID NO: 302; and SEQ ID NO: 320.

In yet another embodiment, a microarray comprises a renal cortical epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 49; SEQ ID NO: 57; SEQ ID NO: 104; SEQ ID NO: 123; SEQ ID NO: 160; SEQ ID NO: 165; SEQ ID NO: 166; SEQ ID NO: 219; SEQ ID NO: 267; SEQ ID NO: 270; SEQ ID NO: 279; SEQ ID NO: 280; SEQ ID NO: 283; SEQ ID NO: 291; SEQ ID NO: 305; SEQ ID NO: 307; SEQ ID NO: 310; SEQ ID NO: 313; SEQ ID NO: 325; SEQ ID NO: 326; and SEQ ID NO: 327.

The present invention further provides a microarray comprising a renal proximal tubule epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 106; SEQ ID NO: 138; SEQ ID NO: 158; SEQ ID NO: 228; SEQ ID NO: 236; SEQ ID NO: 242; SEQ ID NO: 250; SEQ ID NO: 258; SEQ ID NO: 260; SEQ ID NO: 262; SEQ ID NO: 266; SEQ ID NO: 272; SEQ ID NO: 273; SEQ ID NO: 274; SEQ ID NO: 275; SEQ ID NO: 276; SEQ ID NO: 278; SEQ ID NO: 284; SEQ ID NO: 288; SEQ ID NO: 295; SEQ ID NO: 296; SEQ ID NO: 297; SEQ ID NO: 299; SEQ ID NO: 300; SEQ ID NO: 301; SEQ ID NO: 306; SEQ ID NO: 308; SEQ ID NO: 309; SEQ ID NO: 311; SEQ ID NO: 316; SEQ ID NO: 318; SEQ ID NO: 321; SEQ ID NO: 322; SEQ ID NO: 328; and SEQ ID NO: 329.

In a specific embodiment, a microarray may comprise a small airway epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 173; SEQ ID NO: 174; SEQ ID NO: 183; SEQ ID NO: 220; SEQ ID NO: 221; SEQ ID NO: 222; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 237; SEQ ID NO: 238; SEQ ID NO: 240; SEQ ID NO: 245; SEQ ID NO: 246; SEQ ID NO: 247; SEQ ID NO: 248; SEQ ID NO: 249; SEQ ID NO: 251; SEQ ID NO: 252; SEQ ID NO: 254; SEQ ID NO: 257; SEQ ID NO: 263; SEQ ID NO: 264; SEQ ID NO: 265; SEQ ID NO: 268; SEQ ID NO: 269; SEQ ID NO: 270; SEQ ID NO: 277; SEQ ID NO: 281; SEQ ID NO: 282; SEQ ID NO: 286; SEQ ID NO: 287; SEQ ID NO: 290; SEQ ID NO: 294; SEQ ID NO: 298; SEQ ID NO: 303; SEQ ID NO: 312; SEQ ID NO: 315; SEQ ID NO: 317; and SEQ ID NO: 319.

The present invention also provides a microarray comprising a renal epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 37; SEQ ID NO: 253; SEQ ID NO: 304; SEQ ID NO: 323; and SEQ ID NO: 324.

In yet another embodiment, a microarray may comprise one or more nucleic acid sequences substantially homologous to a nucleic acid sequence or complementary sequence thereof, or portions of said nucleic acid sequence or complementary sequence thereof, selected from the group consisting of SEQ ID NO: 27; SEQ ID NO: 37; SEQ ID NO: 49; SEQ ID NO: 57; SEQ ID NO: 64; SEQ ID NO: 70; SEQ ID NO: 78; SEQ ID NO: 104; SEQ ID NO: 106; SEQ ID NO: 123; SEQ ID NO: 131; SEQ ID NO: 138; SEQ ID NO: 150; SEQ ID NO: 158; SEQ ID NO: 160; SEQ ID NO: 165; SEQ ID NO: 166; SEQ ID NO: 169; SEQ ID NO: 173; SEQ ID NO: 174; SEQ ID NO: 183; SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; SEQ ID NO: 211; SEQ ID NO: 212; SEQ ID NO: 213; SEQ ID NO: 214; SEQ ID NO: 215; SEQ ID NO: 216; SEQ ID NO: 217; SEQ ID NO: 218; SEQ ID NO: 219; SEQ ID NO: 220; SEQ ID NO: 221; SEQ ID NO: 222; SEQ ID NO: 223; SEQ ID NO: 224; SEQ ID NO: 225; SEQ ID NO: 226; SEQ ID NO: 227; SEQ ID NO: 228; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 236; SEQ ID NO: 237; SEQ ID NO: 238; SEQ ID NO: 239; SEQ ID NO: 240; SEQ ID NO: 241; SEQ ID NO: 242; SEQ ID NO: 243; SEQ ID NO: 244; SEQ ID NO: 245; SEQ ID NO: 246; SEQ ID NO: 247; SEQ ID NO: 248; SEQ ID NO: 249; SEQ ID NO: 250; SEQ ID NO: 251; SEQ ID NO: 252; SEQ ID NO: 253; SEQ ID NO: 254; SEQ ID NO: 255; SEQ ID NO: 256; SEQ ID NO: 257; SEQ ID NO: 258; SEQ ID NO: 259; SEQ ID NO: 260; SEQ ID NO: 261; SEQ ID NO: 262; SEQ ID NO: 263; SEQ ID NO: 264; SEQ ID NO: 265; SEQ ID NO: 266; SEQ ID NO: 267; SEQ ID NO: 268; SEQ ID NO: 269; SEQ ID NO: 270; SEQ ID NO: 271; SEQ ID NO: 272; SEQ ID NO: 273; SEQ ID NO: 274; SEQ ID NO: 275; SEQ ID NO: 276; SEQ ID NO: 277; SEQ ID NO: 278; SEQ ID NO: 279; SEQ ID NO: 280; SEQ ID NO: 281; SEQ ID NO: 282; SEQ ID NO: 283; SEQ ID NO: 284; SEQ ID NO: 285; SEQ ID NO: 286; SEQ ID NO: 287; SEQ ID NO: 288; SEQ ID NO: 289; SEQ ID NO: 290; SEQ ID NO: 291; SEQ ID NO: 293; SEQ ID NO: 294; SEQ ID NO: 295; SEQ ID NO: 296; SEQ ID NO: 297; SEQ ID NO: 298; SEQ ID NO: 299; SEQ ID NO: 300; SEQ ID NO: 301; SEQ ID NO: 302; SEQ ID NO: 303; SEQ ID NO: 304; SEQ ID NO: 305; SEQ ID NO: 306; SEQ ID NO: 307; SEQ ID NO: 308; SEQ ID NO: 309; SEQ ID NO: 310; SEQ ID NO: 311; SEQ ID NO: 312; SEQ ID NO: 313; SEQ ID NO: 314; SEQ ID NO: 315; SEQ ID NO: 316; SEQ ID NO: 317; SEQ ID NO: 318; SEQ ID NO: 320; SEQ ID NO: 321; SEQ ID NO: 322; SEQ ID NO: 323; SEQ ID NO: 324; SEQ ID NO: 325; SEQ ID NO: 326; SEQ ID NO: 327; SEQ ID NO: 328; and SEQ ID NO: 329.

In another embodiment, the present invention provides a microarray comprising a gene expression profile comprising one or more genes or oligonucleotide probes obtained therefrom, wherein said gene expression profile is generated from a cell type selected from the group comprising coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesangial cells, coronary artery smooth muscle, bronchial smooth muscle, uterine smooth muscle, lung fibroblast, osteoblasts, and prostate stromal cells.

This invention also relates to methods of doing business comprising the steps of determining the level of RNA expression for an RNA sample, wherein the RNA sample is amplified, fluorescently labeled, and hybridized to a microarray containing a plurality of nucleic acid sequences, and wherein the microarray is scanned for fluorescence; normalizing the expression levels using an algorithm, and scoring the RNA sample against a gene expression profile database. In one embodiment, the RNA sample is obtained from a patient and the patient sample includes, but is not limited to, blood, amniotic fluid, plasma, semen, bone marrow, and tissue biopsy.

In another aspect of this method, the algorithm is either the MaxCor algorithm or the Mean Log Ratio algorithm. The invention described herein further provides algorithms useful for generating gene expression profiles. Specifically, the present invention provides for either the MaxCor algorithm or the Mean Log Ratio algorithm to generate a gene expression profile.

The present invention also relates to a method of constructing a gene expression profile comprising the steps of hybridizing prepared RNA samples to a microarray containing a plurality of known nucleic acid sequences representing genes of a particular organism; obtaining an expression level for each gene on a microarray; and normalizing the expression level for each gene on a microarray to control standards.

In a further aspect, the method of constructing a gene expression profile comprises the steps applying an algorithm to each of the normalized gene expression levels; performing a correlation analysis for all normalized gene expression microarrays within a group of samples; establishing a gene expression profile using a signature extraction algorithm; and validating the gene expression profile.

In one embodiment, the algorithm of the profile construction method is the MaxCor algorithm. Specifically, the MaxCor algorithm is used to generate a numeric value that is assigned to each gene based upon the expression level contained on the microarray. In one embodiment, the numeric value is between the range of (−1,+1). In particular, a negative numeric value represents a gene with relatively lower expression; a zero numeric value represents no relative gene expression difference; and a positive numeric value represents a gene with relatively higher expression.

In one embodiment, the numeric value is between the range of (−2,+2). In particular, a negative numeric value represents a gene with relatively lower expression; a zero numeric value represents no relative gene expression difference; and a positive numeric value represents a gene with relatively higher expression.

In another embodiment, the algorithm of the profile construction method is the Mean Log Ratio algorithm. Specifically, the Mean Log Ratio algorithm is used to generate a numeric value that is assigned to each gene based upon the expression level contained on the microarray. In one embodiment, the numeric value is between the range of (−1,+1). In particular, a negative numeric value represents a gene with relatively lower expression; a zero numeric value represents no relative gene expression difference; and a positive numeric value represents a gene with relatively higher expression.

In one embodiment, the numeric value is between the range of (−2,+2). In particular, a negative numeric value represents a gene with relatively lower expression; a zero numeric value represents no relative gene expression difference; and a positive numeric value represents a gene with relatively higher expression.

The present invention further provides a method, in a computer system, for constructing and analyzing a gene expression profile comprising the steps of inputting gene expression data for each of a plurality of genes; normalizing expression data by transforming said data into log ratio values; filtering weak differential values; applying an algorithm to each of said normalized gene expression values; performing a classification analysis for all normalized gene expression values; establishing a gene expression profile; and validating the gene expression profile. The algorithm may be the MaxCor algorithm or the Mean Log Ratio algorithm.

This invention is also related to computer programs for constructing and analyzing a gene expression signature. These computer programs may comprise computer code that receives as input gene expression data for a plurality of genes; computer code that normalizes expression data by transforming the data into log ratio values; computer code that applies an algorithm to each of the normalized gene expression values; computer code that performs a correlation analysis for the normalized gene expression values; computer code that establishes and validates the gene expression profile; and computer readable medium that stores computer code. The computer program may utilize the MaxCor algorithm or the Mean Log Ratio algorithm for gene expression profile analysis.

The present invention also provides methods for identifyng the phenotype of an unknown cell. This method comprises applying an algorithm to extract a gene expression profile from gene expression data generated from the cell; and matching the gene expression profile to a gene expression profile generated from a cell of known phenotype. In one embodiment, the algorithm is the MaxCor algorithm. In an alternative embodiment, the algorithm is the Mean Log Ratio algorithm.

In a particular embodiment, the application of an algorithm to extract a gene expression profile comprises setting a cutoff value for expression relative to normalized values, wherein said cutoff value is at least about two-fold induction above the normalized values. Moreover, the matching step may be performed using a database comprising one or more gene expression profiles generated from cells of known phenotype.

The present invention further provides methods for distinguishing cell types comprising using an algorithm to generate a gene expression profile from a biological sample; and matching said generated gene expression profile to a gene expression profile of a specific cell type. In one embodiment, the algorithm is the MaxCor algorithm. In an alternative embodiment, the algorithm is the Mean Log Ratio algorithm.

In a further embodiment, the specific cell type is selected from the group consisting of coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesangial cells, coronary artery smooth muscle, bronchial smooth muscle, uterine smooth muscle, lung fibroblast, osteoblasts, and prostate stromal cells.

In a specific embodiment, the present invention provides a method for determining the phenotype of a cell comprising the steps of applying an algorithm to extract a protein expression profile from protein expression data generated from the cell and matching the protein expression profile to a protein expression profile generated from a cell of known phenotype.

In one embodiment, the algorithm is the MaxCor algorithm. In an alternative embodiment, the algorithm is the Mean Log Ratio algorithm. In yet another embodiment, the applying step comprises setting a cutoff value for expression relative to normalized values, wherein said cutoff value is at least about two-fold induction above the normalized values. In yet another embodiment, the matching step is performed using a database comprising one or more protein expression profiles generated from cells of known phenotype.

The present invention provides a method for distinguishing cell types comprising the step of matching a protein expression profile generated from a biological sample using an algorithm to a known protein expression profile of a specific cell type. In one embodiment, the algorithm is the MaxCor algorithm. In an alternative embodiment, the algorithm is the Mean Log Ratio algorithm.

In a further embodiment, the specific cell type is selected from the group consisting of coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesangial cells, coronary artery smooth muscle, bronchial smooth muscle, uterine smooth muscle, lung fibroblast, osteoblasts, and prostate stromal cells.

### BRIEF DESCRIPTION OF THE DRAWINGS

FIG. 1. Laser capture microdissection (LCM) of 10 μm Nissl-stained sections of adult rat large and small dorsal root ganglion (DRG) neurons. The arrows indicate DRG neurons to be captured (top panel). The middle and bottom panels show successful capture and film transfer respectively.

FIGS. 2a-2b. Microarray of cDNA expression patterns of small (S) and large (L) neurons. FIG. 2a is an example of the cDNA microarray data obtained. Boxed in white is an identical region of the microarray for L1 and S1 samples that is enlarged (shown directly below). In FIG. 2b, scatter plots are shown that demonstrate the correlation between independent amplifications of S1 vs. S2, S1 vs. S3, L1 vs. L2, and L (L1 and L2) vs. S(S1, S2, and S3).

FIG. 3. Preferentially expressed mRNAs identified in small DRG neurons. The ratio value describes the mean fluorescence intensity ratio of the small DRG neurons as compared to the large DRG neurons.

FIG. 4. Preferentially expressed mRNAs identified in large DRG neurons. The ratio value describes the mean fluorescence intensity ratio of the large DRG neurons as compared to the small DRG neurons.

FIG. 5. Representative fields of in situ hybridization of rat DRG with selected cDNAs. The sections were Nissl-counterstained. The left panel shows results with radiolabeled probes encoding neurofilament-high (NF-H), neurofilament-low (NF-L) and β-1 subunit of the voltage-gated sodium channel (SCNβ-1). Arrows in the left panel denote identifiable small neurons. The right panel shows representative fields from radiolabeled probes encoding calcitonin gene-related product (CGRP), voltage-gated sodium channel (NaN), and phospholipase C delta-4 (PLC). Arrows in the right panel denote identifiable large neurons. The large arrowhead denotes a large neuron which is also labeled.

FIG. 6. In situ hybridization of selected cDNAs identified in small DRG neurons and large DRG neurons. Based on quantitative measurements comparing the overall intensity of signal in small and large neurons and the percentage of cells labeled within the total population of either small or large neurons, the preferential expression of these mRNAs was demonstrated.

FIG. 7. Profile extraction analysis of several primary cell types. Clustering analysis of the gene expression profiles of the primary cell samples confirmed that these cell types could be classified into three groups: endothelial, epithelial, and muscle cell.

FIG. 8. Cluster analysis of the 30 gene expression vectors using the hclust algorithm in the S-plus statistical package (MathSoft, Inc., Cambridge, Mass.). The hclust algorithm groups together primary cells with similar gene expression patterns. The three sample groups (endothelial, epithelial, and muscle cells) were easily separated.

FIGS. 9a-9t. The gene expression profile of human primary cells. The profile represents 459 genes identified from 30 primary cell types. The sequence source (Seq. Source) is the gene database (GB: GenBank; INCYTE: Incyte Genomes) from which the sequence was selected. The endothelial, epithelial, and muscle profile values are the numeric representation of the specific profile. The p-value is based on the Kruskal-Wallis rank test in which smaller p-values represent clones with higher discriminate power for classifying samples. The source description identifies the particular gene.

FIGS. 10a-10c. The gene expression profile of endothelial cells. The sequence source (Seq. Source) is the gene database (GB: GenBank; INCYTE: Incyte Genomes) from which the sequence was selected. The endothelial, epithelial, and muscle profile values are the numeric representation of the specific profile. The p-value is based on the Kruskal-Wallis rank test in which smaller p-values represent clones with higher discriminate power for classifying samples. The source description identifies the particular gene.

FIGS. 11a-11c. The gene expression profile of epithelial cells. The sequence source (Seq. Source) is the gene database (GB: GenBank; INCYTE: Incyte Genomes) from which the sequence was selected. The endothelial, epithelial, and muscle profile values are the numeric representation of the specific profile. The p-value is based on the Kruskal-Wallis rank test in which smaller p-values represent clones with higher discriminate power for classifying samples. The source description identifies the particular gene.

FIGS. 12a-12b. The gene expression profile of muscle cells. The sequence source (Seq. Source) is the gene database (GB: GenBank; INCYTE: Incyte Genomes) from which the sequence was selected. The endothelial, epithelial, and muscle profile values are the numeric representation of the specific profile. The p-value is based on the Kruskal-Wallis rank test in which smaller p-values represent clones with higher discriminate power for classifying samples. The source description identifies the particular gene.

FIG. 13. The profile vectors (endothelial, epithelial, and muscle) generated by using the Mean Log Ratio and MaxCor algorithms are plotted graphically. The numbers are plotted according to the color bar. Numbers in the middle are plotted with colors in between as indicated.

FIG. 14. Self-validation analysis using the Mean Log Ratio algorithm. Each of the 30 samples was scored against the three expression profiles generated by using all 30 samples. The scores are plotted on the bar chart (white—endothelial, black—epithelial, hatched—muscle). The order of the primary cells is listed in FIG. 7.

FIG. 15. Omit-one analysis using the Mean Log Ratio algorithm. Each of the 30 samples was scored against the three expression profiles generated by using all but the sample omitted. The scores are plotted on the bar chart (white—endothelial, black—epithelial, hatched—muscle). The order of the primary cells is listed on FIG. 7.

FIG. 16. Self-validation analysis using the MaxCor algorithm. Each of the 30 samples were scored against the three expression profiles generated by using all 30 samples. The scores are plotted on the bar chart (white—endothelial, black—epithelial, hatched—muscle). The order of the primary cells is listed on FIG. 7.

FIG. 17. Omit-one analysis using the MaxCor algorithm. Each of the 30 samples was scored against the three expression profiles generated by using all but the sample omitted. The scores are plotted on the bar chart (white—endothelial, black—epithelial, hatched—muscle). The order of the primary cells is listed on FIG. 7.

FIGS. 18a-18f. Gene expression profiles of epithelial cell lines derived from keratinocyte epithelium, mammary epithelium, bronchial epithelium, prostate epithelium, renal cortical epithelium, renal proximal tubule epithelium, small airway epithelium, and renal epithelium. The data is sorted from highest relative expression to lowest relative expression for keratinocyte epithelial cells.

### DETAILED DESCRIPTION OF THE INVENTION

It is to be understood that this invention is not limited to the particular methodology, protocols, cell lines, animal species or genera, constructs, or reagents described and as such may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, reference to “a protein” is a reference to one or more proteins and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are hereby incorporated by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### Definitions

For convenience, the meaning of certain terms and phrases employed in the specification, examples, and appended claims are provided below. The definitions are not meant to be limiting in nature and serve to provide a clearer understanding of certain aspects of the present invention.

The term “genome” is intended to include the entire DNA complement of an organism, including the nuclear DNA component, chromosomal or extrachromosomal DNA, as well as the cytoplasmic domain (e.g., mitochondrial DNA).

The term “gene” refers to a nucleic acid sequence that comprises control and coding sequences necessary for producing a polypeptide or precursor. The polypeptide may be encoded by a full length coding sequence or by any portion of the coding sequence. The gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions.

The term “gene expression” refers to the process by which a nucleic acid sequence undergoes successful transcription and translation such that detectable levels of the nucleotide sequence are expressed.

The terms “gene expression profile” or “gene expression signature” refer to a group of genes representing a particular cell or tissue type (e.g., neuron, coronary artery endothelium, or disease tissue).

The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include, but are not limited to, aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like. Furthermore, the term “nucleic acid sequences” contemplates the complementary sequence and specifically includes any nucleic acid sequence that is substantially homologous to the both the nucleic acid sequence and its complement.

The term “homology”, as used herein, refers to a degree of complementarity. There may be partial homology or complete homology (i.e., identity). A partially complementary sequence is one that at least partially inhibits an identical sequence from hybridizing to a target nucleic acid; it is referred to using the functional term “substantially homologous.”The inhibition of hybridization of the completely complementary sequence to the target sequence may be examined using a hybridization assay (Southern or northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding, the probe will not hybridize to the second non-complementary target sequence.

The term “oligonucleotide” as used herein refers to a nucleic acid molecule comprising, for example, from about 10 to about 1000 nucleotides. Oligonucleotides for use in the present invention are preferably from about 15 to about 150 nucleotides, more preferably from about 150 to about 1000 in length. The oligonucleotide may be a naturally occurring oligonucleotide or a synthetic oligonucleotide. Oligonucleotides may be prepared by the phosphoramidite method (Beaucage and Carruthers, 22 TETRAHEDRON LETT. 1859-62 (1981)), or by the triester method (Matteucci et al., 103 J. AM. CHEM. SOC. 3185 (1981)), or by other chemical methods known in the art.

The terms “modified oligonucleotide” and “modified polynucleotide” as used herein refer to oligonucleotides or polynucleotides with one or more chemical modifications at the molecular level of the natural molecular structures of all or any of the bases, sugar moieties, internucleoside phosphate linkages, as well as to molecules having added substitutions or a combination of modifications at these sites. The internucleoside phosphate linkages may be phosphodiester, phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone internucleotide linkages, or 3′-3′, 5′-3′, or 5′-5′ linkages, and combinations of such similar linkages. The phosphodiester linkage may be replaced with a substitute linkage, such as phosphorothioate, methylamino, methylphosphonate, phosphoramidate, and guanidine, and the ribose subunit of the nucleic acids may also be substituted (e.g., hexose phosphodiester; peptide nucleic acids). The modifications may be internal (single or repeated) or at the end(s) of the oligonucleotide molecule, and may include additions to the molecule of the internucleoside phosphate linkages, such as deoxyribose and phosphate modifications which cleave or crosslink to the opposite chains or to associated enzymes or other proteins. The terms “modified oligonucleotides” and “modified polynucleotides” also include oligonucleotides or polynucleotides comprising modifications to the sugar moieties (e.g., 3′-substituted ribonucleotides or deoxyribonucleotide monomers), any of which are bound together via 5′ to 3′ linkages.

“Biomolecular sequence,” as used herein, is a term that refers to all or a portion of a gene or nucleic acid sequence. A biomolecular sequence may also refer to all or a portion of an amino acid sequence.

The terms “array” and “microarray” refer to the type of genes or proteins represented on an array by oligonucleotides or protein-capture agents, and where the type of genes or proteins represented on the array is dependent on the intended purpose of the array (e.g., to monitor expression of human genes or proteins). The oligonucleotides or protein-capture agents on a given array may correspond to the same type, category, or group of genes or proteins. Genes or proteins may be considered to be of the same type if they share some common characteristics such as species of origin (e.g., human, mouse, rat); disease state (e.g., cancer); functions (e.g., protein kinases;, tumor suppressors); same biological process (e.g., apoptosis, signal transduction, cell cycle regulation, proliferation, differentiation). For example, one array type may be a “cancer array” in which each of the array oligonucleotides or protein-capture agents correspond to a gene or protein associated with a cancer. An “epithelial array” may be an array of oligonucleotides or protein-capture agents corresponding to unique epithelial genes or proteins. Similarly, a “cell cycle array” may be an array type in which the oligonucleotides or protein-capture agents correspond to unique genes or proteins associated with the cell cycle.

The term “cell type” refers to a cell from a given source (e.g., a tissue, organ) or a cell in a given state of differentiation, or a cell associated with a given pathology or genetic makeup.

The term “activation” as used herein refers to any alteration of a signaling pathway or biological response including, for example, increases above basal levels, restoration to basal levels from an inhibited state, and stimulation of the pathway above basal levels.

The term “differential expression” refers to both quantitative as well as qualitative differences in the temporal and tissue expression patterns of a gene or a protein. For example, a differentially expressed gene may have its expression activated or completely inactivated in normal versus disease conditions. Such a qualitatively regulated gene may exhibit an expression pattern within a given tissue or cell type that is detectable in either control or disease conditions, but is not detectable in both. Differentially expressed genes may represent “high information density genes,” “profile genes,” or “target genes.”

Similarly, a differentially expressed protein may have its expression activated or completely inactivated in normal versus disease conditions. Such a qualitatively regulated protein may exhibit an expression pattern within a given tissue or cell type that is detectable in either control or disease conditions, but is not detectable in both. Morever, differntialy expressed genes may represent “high information density proteins,” “profile proteins,” or “target proteins.”

The term “detectable” refers to an RNA expression pattern which is detectable via the standard techniques of polymerase chain reaction (PCR), reverse transcriptase-(RT) PCR, differential display, and Northern analyses, which are well known to those of skill in the art. Similarly, protein expression patterns may be “detected” via standard techniques such as Western blots.

The term “high information density” refers to a gene or protein whose expression pattern may be used as a predictor or diagnostic, may be used in methods for identifying therapeutic compounds, drug or toxicity screening, or identifying cellular signal pathways or co-regulated genes. Identification of high information density genes or proteins is accomplished by assessing the information content of one or more genes or proteins comprising one or more gene or protein expression profiles. Genes or proteins providing the highest amount of information content comprise high information density genes or proteins. High information density genes may also be referred to as “predictor genes.” Similarly, high information density proteins may be referred to as “predictor proteins.”

The term “information content” refers to the value assigned to a particular gene or protein based on quantitative and qualitative expression under selected conditions. Information content may be derived by measuring one or more parameters of gene or protein expression including, but not limited to, the cell type in which the gene or protein is expressed, the magnitude of response over time, and response to chemical or physical stimuli. Algorithms may be used in assessing the information content provided by particular genes or proteins.

A “target gene” refers to a nucleic acid, often derived from a biological sample, to which an oligonucleotide probe is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding probe directed to the target. The target nucleic acid may also refer to the specific subsequence of a larger nucleic acid to which the probe is directed or to the overall sequence (e.g., gene or mRNA) whose expression level it is desired to detect.

A “target protein” refers to an amino acid or protein, often derived from a biological sample, to which a protein-capture agent specifically hybridizes or binds. It is either the presence or absence of the target protein that is to be detected, or the amount of the target protein that is to be quantified. The target protein has a structure that is recognized by the corresponding protein-capture agent directed to the target. The target protein or amino acid may also refer to the specific substructure of a larger protein to which the protein-capture agent is directed or to the overall structure (e.g., gene or mRNA) whose expression level it is desired to detect.

The term “complementary” refers to the topological compatibility or matching together of the interacting surfaces of a probe molecule and its target. The target and its probe can be described as complementary, and furthermore, the contact surface characteristics are complementary to each other. Hybridization or base pairing between nucleotides or nucleic acids, such as, for example, between the two strands of a double-stranded DNA molecule or between an oligonucleotide probe and a target are complementary.

The term “hybridization” refers to the binding, duplexing, or hybridizing of a nucleic acid molecule to a particular nucleic acid sequence under stringent conditions. Hybridization may also refer to the binding of a protein-capture agent to a target protein under certain conditions, such as normal physiological conditions.

The term “stringent conditions” refers to conditions under which a probe may hybridize to its target nucleic acid sequence, but to no other sequences. Stringent conditions are sequence-dependent (e.g., longer sequences hybridize specifically at higher temperatures). Generally, stringent conditions are selected to be about 5° C. lower than the thermal melting point (Tₘ) for the specific sequence at a defined ionic strength and pH. The Tₘ is the temperature (under defined ionic strength, pH, and nucleic acid concentration) at which 50% of the probes complementary to the target sequence hybridize to the target sequence at equilibrium. Typically, stringent conditions will be those in which the salt concentration is at least about 0.01 to about 1.0 M sodium ion concentration (or other salts) at about pH 7.0 to about pH 8.3 and the temperature is at least about 30° C. for short probes (e.g., 10 to 50 nucleotides). Stringent conditions may also be achieved with the addition of destabilizing agents such as formamide.

The term “label” refers to agents that are capable of providing a detectable signal, either directly or through interaction with one or more additional members of a signal producing system. Labels that are directly detectable and may find use in the present invention include: fluorescent labels, where the wavelength of light absorbed by the fluorophore may generally range from about 300 to about 900 nm, usually from about 400 to about 800 nm, and where the absorbance maximum may typically occur at a wavelength ranging from about 500 to about 800 nm. Specific fluorophores for use in singly labeled primers include: fluorescein, rhodamine, BODIPY, cyanine dyes and the like. Radioactive isotopes, such as ³⁵S, ³²P, ³H, and the like may also be utilized as labels. Examples of labels that provide a detectable signal through interaction with one or more additional members of a signal producing system include capture moieties that specifically bind to complementary binding pair members, where the complementary binding pair members comprise a directly detectable label moiety, such as a fluorescent moiety as described above. The label should be such that it does not provide a variable signal, but instead provides a constant and reproducible signal over a given period of time. Capture moieties of interest include ligands (e.g., biotin) where the other member of the signal producing system could be fluorescently labeled streptavidin, and the like. The target molecules may be end-labeled, i.e., the label moiety is present at a region at least proximal to, and preferably at, the 5′ terminus of the target.

The term “oligonucleotide probe” refers to a surface-immobilized oligonucleotide that may be recognized by a particular target. Depending on context, the term “oligonucleotide probes” refers both to individual oligonucleotide molecules and to the collection of oligonucleotide molecules immobilized at a discrete location. Generally, the probe is capable of binding to a target nucleic acid of complementary sequence through one or more types of chemical bonds, usually through complementary base pairing via hydrogen bond formation. As used herein, an oligonucleotide probe may include natural (e.g., A, G, C, or T) or modified bases (e.g., 7-deazaguanosine, inosine). In addition, the bases in an oligonucleotide probe may be joined by a linkage other than a phosphodiester bond, so long as it does not interfere with hybridization. Thus, oligonucleotide probes may be peptide nucleic acids in which the constituent bases are joined by peptide bonds rather than phosphodiester linkages.

The term “protecting group” as used herein, refers to any of the groups which are designed to block one reactive site in a molecule while a chemical reaction is carried out at another reactive site. The proper selection of protecting groups for a particular synthesis may be governed by the overall methods employed in the synthesis. For example, in photolithography synthesis, discussed below, the protecting groups are photolabile protecting groups such as NVOC and MeNPOC. In other methods, protecting groups may be removed by chemical methods and include groups such as FMOC, DMT, and others known to those of skill in the art.

The term “support” or “substrate” refers to material having a rigid or semi-rigid surface. Such materials may take the form of plates or slides, small beads, pellets, disks or other convenient forms, although other forms may be used. In some embodiments, at least one surface of the substrate will be substantially flat. In other embodiments, a roughly spherical shape may be preferred. In the microarrays of the present invention, the oligonucleotide probes or protein-capture agents (defined below) may be stably associated with the surface of a rigid support, i.e., the probes maintain their position relative to the rigid support under hybridization and washing conditions. As such, the oligonucleotide probes or protein-capture agents may be non-covalently or covalently associated with the support surface. Examples of non-covalent association include non-specific adsorption, specific binding through a specific binding pair member covalently attached to the support surface, and entrapment in a support material (e.g., a hydrated or dried separation medium) which presents the oligonucleotide probe or protein-capture agent in a manner sufficient for hybridization to occur. Examples of covalent binding include covalent bonds formed between the oligonucleotide probe or protein-capture agent and a functional group present on the surface of the rigid support (e.g., —OH) where the functional group may be naturally occurring or present as a member of an introduced linking group.

As mentioned above, the microarray may be present on a rigid substrate. By rigid, the support is solid and preferably does not readily bend. As such, the rigid substrates of the microarrays are sufficient to provide physical support and structure to the oligonucleotide probes or protein-capture agents present thereon under the assay conditions in which the microarray is utilized, particularly under high-throughput handling conditions.

The term “spatially directed oligonucleotide synthesis” refers to any method of directing the synthesis of an oligonucleotide to a specific location on a substrate.

The term “background” refers to hybridization signals resulting from non-specific binding, or other interactions, between the labeled target nucleic acids and components of the oligonucleotide microarray (e.g., the oligonucleotide probes, control probes, the array substrate) or between target proteins and the protein-capture agents of a protein microarray. Background signals may also be produced by intrinsic fluorescence of the microarray components themselves. A single background signal may be calculated for the entire array, or a different background signal may be calculated for each target nucleic acid or target protein. The background may be calculated as the average hybridization signal intensity, or where a different background signal is calculated for each target gene or target protein. Alternatively, background may be calculated as the average hybridization signal intensity produced by hybridization to probes that are not complementary to any sequence found in the sample (e.g., probes directed to nucleic acids of the opposite sense or to genes not found in the sample such as bacterial genes where the sample is mammalian nucleic acids). The background can also be calculated as the average signal intensity produced by regions of the array which lack any probes or protein-capture agents at all.

The term “cluster” refers to a group of nucleic acid sequences or amino acid sequences related to one another by sequence homology. In one example, clusters are formed based upon a specified degree of homology and/or overlap (e.g., stringency). “Clustering” may be performed with the nucleic acid or amino acid sequence data. For instance, a sequence thought to be associated with a particular molecular or biological function in one tissue might be compared against another library or database of sequences. This type of search is useful to look for homologous, and presumably functionally related, sequences in other tissues or samples, and may be used to streamline the methods of the present invention in that clustering may be used within one or more of the databases to cluster biomolecular sequences prior to performing methods of the invention. The sequences showing sufficient homology with the representative sequence are considered part of a “cluster.” Such “sufficient” homology may vary within the needs of one skilled in the art.

The term “linker” refers to a moiety, molecule, or group of molecules attached to a solid support, and spacing an oligonucleotide or other nucleic acid fragment from the solid support.

The term “bead” refers to solid supports for use with the present invention. Such beads may have a wide variety of forms, including microparticles, beads, and membranes, slides, plates, micromachined chips, and the like. Likewise, solid supports of the invention may comprise a wide variety of compositions, including glass, plastic, silicon, alkanethiolate-derivatized gold, cellulose, low crosslinked and high crosslinked polystyrene, silica gel, polyamide, and the like. Other materials and shapes may be used, including pellets, disks, capillaries, hollow fibers, needles, solid fibers, cellulose beads, pore-glass beads, silica gels, polystyrene beads optionally crosslinked with divinylbenzene, grafted co-poly beads, poly-acrylamide beads, latex beads, dimethylacrylamide beads optionally crosslinked with N,N-bis-acryloyl ethylene diamine, and glass particles coated with a hydrophobic polymer.

The term “biological sample” refers to a sample obtained from an organism (e.g., patient) or from components (e.g., cells) of an organism. The sample may be of any biological tissue or fluid. The sample may be a “clinical sample” which is a sample derived from a patient. Such samples include, but are not limited to, sputum, blood, blood cells (e.g., white cells), amniotic fluid, plasma, semen, bone marrow, and tissue or fine needle biopsy samples, urine, peritoneal fluid, and pleural fluid, or cells therefrom. Biological samples may also include sections of tissues such as frozen sections taken for histological purposes. A biological sample may also be referred to as a “patient sample.”

“Proteomics” is the study of or the characterization of either the proteome or some fraction of the proteome. The “proteome” is the total collection of the intracellular proteins of a cell or population of cells and the proteins secreted by the cell or population of cells. This characterization includes measurements of the presence, and usually quantity, of the proteins that have been expressed by a cell. The function, structural characteristics (such as post-translational modification), and location within the cell of the proteins may also be studied. “Functional proteomics” refers to the study of the functional characteristics, activity level, and structural characteristics of the protein expression products of a cell or population of cells.

A “protein” means a polymer of amino acid residues linked together by peptide bonds. The term, as used herein, refers to proteins, polypeptides, and peptides of any size, structure, or function. Typically, however, a protein will be at least six amino acids long. If the protein is a short peptide, it will be at least about 10 amino acid residues long. A protein may be naturally occurring, recombinant, or synthetic, or any combination of these. A protein may also comprise a fragment of a naturally occurring protein or peptide. A protein may be a single molecule or may be a multi-molecular complex. The term protein may also apply to amino acid polymers in which one or more amino acid residues is an artificial chemical analogue of a corresponding naturally occurring amino acid.

A “fragment of a protein,” as used herein, refers to a protein that is a portion of another protein. For example, fragments of proteins may comprise polypeptides obtained by digesting full-length protein isolated from cultured cells. In one embodiment, a protein fragment comprises at least about six amino acids. In another embodiment, the fragment comprises at least about ten amino acids. In yet another embodiment, the protein fragment comprises at least about 16 amino acids.

As used herein, an “expression product” is a biomolecule, such as a protein, which is produced when a gene in an organism is expressed. An expression product may comprise post-translational modifications.

The term “protein expression” refers to the process by which a nucleic acid sequence undergoes successful transcription and translation such that detectable levels of the amino acid sequence or protein are expressed.

The terms “protein expression profile” or “protein expression signature” refer to a group of proteins representing a particular cell or tissue type (e.g., neuron, coronary artery endothelium, or disease tissue).

The term “protein-capture agent,” as used herein, refers to a molecule or a multimolecular complex that can bind a protein to itself. In one embodiment, protein-capture agents bind their binding partners in a substantially specific manner. In one embodiment, protein-capture agents may exhibit a dissociation constant (K_{D}) of less than about 10⁻⁶. The protein-capture agent may comprise a biomolecule such as a protein or a polynucleotide. The biomolecule may further comprise a naturally occurring, recombinant, or synthetic biomolecule. Examples of protein-capture agents include antibodies, antigens, receptors, or other proteins, or portions or fragments thereof. Furthermore, protein-capture agents are understood not to be limited to agents that only interact with their binding partners through noncovalent interactions. Rather, protein-capture agents may also become covalently attached to the proteins with which they bind. For example, the protein-capture agent may be photocrosslinked to its binding partner following binding.

A “region of protein-capture agents” is a term that refers to a discrete area of immobilized protein-capture agents on the surface of a substrate. The regions may be of any geometric shape or may be irregularly shaped.

As used herein, the term “binding partner” refers to a protein that may bind to a particular protein-capture agent. In one embodiment, the binding partner binds a protein-capture agent in a substantially specific manner. In some cases, the protein-capture agent may be a cellular or extracellular protein and the binding partner may be the entity normally bound in vivo. In other embodiments, however, the binding partner may be the protein or peptide on which the protein-capture agent was selected (through in vitro or in vivo selection) or raised (as in the case of antibodies). A binding partner may be shared by more than one protein-capture agent. For example, a binding partner that is bound by a variety of polyclonal antibodies may bear a number of different epitopes. One protein-capture agent may also bind to a multitude of binding partners, for example, if the binding partners share the same epitope.

A “population of cells in an organism” means a collection of more than one cell in a single organism or more than one cell originally derived from a single organism. The cells in the collection are preferably all of the same type. They may all be from the same tissue in an organism, for example. Most preferably, gene expression in all of the cells in the population is identical or nearly identical.

“Conditions suitable for protein binding” means those conditions (in terms of salt concentration, pH, detergent, protein concentration, temperature, etc.) that allow for binding to occur between an immobilized protein-capture agent and its binding partner in solution. Preferably, the conditions are not so lenient that a significant amount of nonspecific protein binding occurs.

A “small molecule” comprises a compound or molecular complex, either synthetic, naturally derived, or partially synthetic, composed of carbon, hydrogen, oxygen, and nitrogen, which may also contain other elements, and which may have a molecular weight of less than about 5,000, and in a specific embodiment between about 100 and about 1,500.

The term “antibody” means an immunoglobulin, whether natural or partially or wholly synthetically produced. All derivatives thereof that maintain specific binding ability are also included in the term. The term also covers any protein having a binding domain that is homologous or largely homologous to an immunoglobulin binding domain. An antibody may be monoclonal or polyclonal. The antibody may be a member of any immunoglobulin class, including any of the human classes: IgG, IgM, IgA, IgD, and IgE.

The term “antibody fragment” refers to any derivative of an antibody that is less than full-length. In one aspect, the antibody fragment retains at least a significant portion of the full-length antibody's specific binding ability, specifically, as a binding partner. Examples of antibody fragments include, but are not limited to, Fab, Fab′, F(ab′)₂, scFv, Fv, dsFv diabody, and Fd fragments. The antibody fragment may be produced by any means. For example, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may comprise a single chain antibody fragment. In another embodiment, the fragment may comprise multiple chains that are linked together, for example, by disulfide linkages. The fragment may also comprise a multimolecular complex. A functional antibody fragment may typically comprise at least about 50 amino acids and more typically will comprise at least about 200 amino acids.

As used herein, single-chain Fvs (scFvs) refer to recombinant antibody fragments, consisting of the variable light chain (V_{L}) and variable heavy chain (V_{H}) covalently connected to one another by a polypeptide linker. Either V_{L} or V_{H} may be the NH₂-terminal domain. The polypeptide linker may be of variable length and composition so long as the two variable domains are bridged without serious steric interference. Typically, the linkers are comprised primarily of stretches of glycine and serine residues with some glutamic acid or lysine residues interspersed for solubility.

“Diabodies” refer to dimeric scFvs. The components of diabodies generally have shorter peptide linkers than most scFvs and they show a preference for associating as dimers.

An “Fv” fragment consists of one V_{H} and one V_{L} domain held together by noncovalent interactions. The term “dsFv” is used herein to refer to an Fv with an engineered intermolecular disulfide bond to stabilize the V_{H}-V_{L} pair.

The term “F(ab′)₂” fragment refers to an antibody fragment essentially equivalent to that obtained from immunoglobulins by digestion with an enzyme pepsin at pH 4.0-4.5. The fragment may be recombinantly produced.

A “Fab” fragment is an antibody fragment essentially equivalent to that obtained by reduction of the disulfide bridge or bridges joining the two heavy chain pieces in the F(ab′)₂ fragment. The Fab′ fragment may be recombinantly produced.

A “Fab” fragment is an antibody fragment essentially equivalent to that obtained by digestion of immunoglobulins with the enzyme papain. The Fab fragment may be recombinantly produced. The heavy chain segment of the Fab fragment is the Fd piece.

The term “coating” means a layer that is either naturally or synthetically formed on or applied to the surface of the substrate. For example, the exposure of a substrate, such as silicon, to air results in oxidation of the exposed surface. In the case of a substrate made of silicon, a silicon oxide coating is formed on the surface upon exposure to air. In other instances, the coating is not derived from the substrate and may be placed upon the surface via mechanical, physical, electrical, or chemical means. An example of this type of coating would be a metal coating that is applied to a silicon or polymeric substrate or a silicon nitride coating that is applied to a silicon substrate. Although a coating may be of any thickness, typically the coating has a thickness smaller than that of the substrate.

An “interlayer” or “adhesion layer” refers to an additional coating or layer that is positioned between the first coating and the substrate. Multiple interlayers may be used together. The primary purpose of a typical interlayer is to facilitate adhesion between the first coating and the substrate. One such example is the use of a titanium or chromium interlayer to help adhere a gold coating to a silicon or glass surface. However, other possible functions of an interlayer are also contemplated. For example, some interlayers may perform a role in the detection system of the microarray, such as a semiconductor or metal layer between a nonconductive substrate and a nonconductive coating.

An “organic thinfilm” is a thin layer of organic molecules that has been applied to a substrate or to a coating on a substrate if present. An organic thinfilm may be less than about 20 nm thick. Alternatively, an organic thinfilm may be less than about 10 nm thick. An organic thinfilm may be disordered or ordered. For example, an organic thinfilm can be amorphous (such as a chemisorbed or spin-coated polymer) or highly organized (such as a Langmuir-Blodgett film or self-assembled monolayer). An organic thinfilm may be heterogeneous or homogeneous. In one embodiment, the organic thinfilm is a monolayer. In another embodiment, the organic thinfilm comprises a lipid bilayer. In other embodiments, the organic thinfilm may comprise a combination of more than one form of organic thinfilm. For example, an organic thinfilm may comprise a lipid bilayer on top of a self-assembled monolayer. A hydrogel may also compose an organic thinfilm. The organic thinfilm may have functionalities exposed on its surface that serve to enhance the surface conditions of a substrate or the coating on a substrate in any of a number of ways. For example, exposed functionalities of the organic thinfilm may be useful in the binding or covalent immobilization of the protein-capture agents to the regions of the protein microarray. Alternatively, the organic thinfilm may bear functional groups, such as polyethylene glycol (PEG), which reduce the non-specific binding of molecules to the surface. Other exposed functionalities serve to tether the thinfilm to the surface of the substrate or the coating. Particular functionalities of the organic thinfilm may also be designed to enable certain detection techniques to be used with the surface. Alternatively, the organic thinfilm may serve the purpose of preventing inactivation of a protein-capture agent or the protein binding partner to be bound by a protein-capture agent from occurring upon contact with the surface of a substrate or a coating on the surface of a substrate.

A “monolayer” is a single-molecule thick organic thinfilm. A monolayer may be disordered or ordered. A monolayer may be a polymeric compound, such as a polynonionic polymer, a polyionic polymer, or a block-copolymer. For example, the monolayer may comprise a poly amino acid such as polylysine. In another embodiment, the monolayer may be a self-assembled monolayer. One face of the self-assembled monolayer may comprise chemical functionalities on the termini of the organic molecules that are chemisorbed or physisorbed onto the surface of the substrate or, if present, the coating on the substrate. Examples of suitable functionalities of monolayers include the positively charged amino groups of poly-L-lysine for use on negatively charged surfaces and thiols for use on gold surfaces. Generally, the other face of the self-assembled monolayer is exposed and may bear any number of chemical functionalities or end groups.

A “self-assembled monolayer” is a monolayer that is created by the spontaneous assembly of molecules. The self-assembled monolayer may be ordered, disordered, or exhibit short- to long-range order.

An “affinity tag” is a functional moiety capable of directly or indirectly immobilizing a protein-capture agent onto a substrate surface or an exposed functionality of an organic thinfilm covering the substrate surface. In one embodiment, the affinity tag enables the site-specific immobilization and thus enhances orientation of the protein-capture agent onto the organic thinfilm. In some cases, the affinity tag may be a simple chemical functional group. Other possibilities include amino acids, poly amino acids tags, or full-length proteins. Still other possibilities include carbohydrates and nucleic acids. For example, the affinity tag may be a polynucleotide that hybridizes to another polynucleotide serving as a functional group on the organic thinfilm or another polynucleotide serving as an adaptor. The affinity tag may also be a synthetic chemical moiety. If the organic thinfilm of each of the regions of protein-capture agents comprises a lipid bilayer or monolayer, then a membrane anchor is a suitable affinity tag. The affinity tag may be covalently or noncovalently attached to the protein-capture agent. For example, if the affinity tag is covalently attached to the protein-capture agent it may be attached via chemical conjugation or as a fusion protein. The affinity tag may also be attached to the protein-capture agent via a cleavable linkage. Alternatively, the affinity tag may not be directly in contact with the protein-capture agent. Rather, the affinity tag may be separated from the protein-capture agent by an adaptor. The affinity tag may immobilize the protein-capture agent to the organic thinfilm either through noncovalent interactions or through a covalent linkage.

An “adaptor,” for purposes of this invention, is any entity that links an affinity tag to the protein-capture agent. The adaptor may be, but is not limited to, a discrete molecule that is noncovalently attached to both the affinity tag and the protein-capture agent. The adaptor may be covalently attached to the affinity tag or the protein-capture agent or both, via chemical conjugation or as a fusion protein. Full-length proteins, polypeptides, or peptides may base used as adaptors. Other possible adaptors include carbohydrates or nucleic acids.

The term “fusion protein” refers to a protein composed of two or more polypeptides that, although typically not joined in their native state, are joined by their respective amino and carboxyl termini through a peptide linkage to form a single continuous polypeptide. It is understood that the two or more polypeptide components can either be directly joined or indirectly joined through a peptide linker/spacer.

The term “normal physiological conditions” means conditions that are typical inside a living organism or a cell. Although some organs or organisms provide extreme conditions, the intra-organismal and intra-cellular environment normally varies around pH 7 (i.e., from pH 6.5 to pH 7.5), contains water as the predominant solvent, and exists at a temperature above 0° C. and below 50° C. The concentration of various salts depends on the organ, organism, cell, or cellular compartment used as a reference.

I. Nucleic Acid Microarrays

Microarray technology provides the opportunity to analyze a large number of nucleic acid sequences. This technology may also be utilized for comparative gene expression analysis, drug discovery, and characterization of molecular interactions. With respect to expression analysis, the expression pattern of a particular gene may be used to characterize the function of that gene. In addition, microarrays may be utilized to analyze both the static expression of a gene (e.g., expression in a specific tissue) as well as, dynamic expression of a particular gene (e.g., expression of one gene relative to the expression of other genes) (Duggan et al., 21 NATURE GENET. 10-14 (1999)).

An advantage of the microarray technology is the use of an impermeable, rigid support as compared to the porous membranes used in the traditional blotting methods (e.g., Northern and Southern analyses). Hybridization buffers do not penetrate the support resulting in greater access to the oligonucleotide probes, enhanced rates of hybridization, and improved reproducibility. In addition, the microarray technology provides better image acquisition and image processing (Southern et al., 21 NATURE GENET. 5-9 (1999)). For microarray analysis, nucleic acids (e.g., RNA) may be isolated from a biological sample. Nucleic acid samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

A. Methods for Producing Nucleic Acid Microarrays

The microarrays may be produced through spatially directed oligonucleotide synthesis. Methods for spatially directed oligonucleotide synthesis include, without limitation, light-directed oligonucleotide synthesis, microlithography, application by ink jet, microchannel deposition to specific locations and sequestration with physical barriers. In general, these methods involve generating active sites, usually by removing protective groups, and coupling to the active site a nucleotide that, itself, optionally has a protected active site if further nucleotide coupling is desired.

A microarray may be configured, for example, by in situ synthesis or by direct deposition (“spotting” or “printing”) of synthesized oligonucleotide probes onto the support. The oligonucleotide probes are used to detect complementary nucleic acid sequences in a target sample of interest. In situ synthesis has several advantages over direct placement such as higher yields, consistency, efficiency, cost, and potential use of combinatorial strategies (Southern et al. (1999)). However, for longer nucleic acid sequences such as PCR products, deposition may be the preferred method. Generation of microarrays by in situ synthesis may be accomplished by a number of methods including photochemical deprotection, ink-jet delivery, and flooding channels (Lipshutz et al., 21 NATURE GENET. 20-24 (1999); Blanchard et al., 11 BIOSENSORS AND BIOELECTRONICS, 687-90 (1996); Maskos et al., 21 NUCLEIC ACIDS RES. 4663-69 (1993)).

The present invention relates to the construction of microarrays by the in situ synthesis method using solid-phase DNA synthesis and photolithography (Lipshutz et al. (1999)). Linkers with photolabile protecting groups may be covalently or non-covalently attached to a support (e.g., glass). Light is then directed through a photolithographic screen to specific areas on the support resulting in localized photodeprotection and yielding reactive hydroxyl groups in the illuminated regions. A 3′-O-phosphoramidite-activated deoxynucleoside (protected at the 5′-hydroxyl with a photolabile group) is then incubated with the support and coupling occurs at deprotected sites that were exposed to light. Following the optional capping of unreacted active sites and oxidation, the substrate is rinsed and the surface is illuminated through a second screen, to expose additional hydroxyl groups for coupling to the linker. A second 5′-protected, 3′-O-phosphoramidite-activated deoxynucleoside is presented to the support. The selective photodeprotection and coupling cycles are repeated until the desired products are obtained. Photolabile groups may then be removed and the sequence may be capped. Side chain protective groups may also be removed. Because photolithography is used, the process may be miniaturized to generate high-density microarrays of oligonucleotide probes. Thus, thousands to hundreds of thousands of arbitrary oligonucleotide probes may be generated on a single microarray support using this technology.

To produce a microarray by the spotting method, oligonucleotide probes are prepared, generally by PCR, for printing onto the microarray support. As described for the in situ technique, the probes may be selected from a number of sources including nucleic acid databases such as GenBank, Unigen, HomoloGene, RefSeq, dbEST, and dbSNP (Wheeler et al., 29 NUCLEIC ACIDS RES. 11-16 (2001)). In addition, oligonucleotide probes may be randomly selected from cDNA libraries reflecting, for example, a tissue type (e.g., cardiac or neuronal tissue), or a genomic library representing a species of interest (e.g., Drosophilia melanogaster). If PCR is used to generate the probes, for example, approximately 100-500 pg of the purified PCR product (about 0.6-2.4 kb) may be spotted onto the support (Duggan et al., 1999). The spotting (or printing) may be performed by a robotic arrayer (see, e.g., U.S. Pat. Nos. 6,150,147; 5,968,740; 5,856,101; 5,474,796; and 5,445,934;).

A number of different microarray configurations and methods for their production are known to those of skill in the art and are disclosed in U.S. Pat. Nos. 6,156,501; 6,077,674; 6,022,963; 5,919,523; 5,885,837; 5,874,219; 5,856,101; 5,837,832; 5,770,722; 5,770,456; 5,744,305; 5,700,637; 5,624,711; 5,593,839; 5,571,639; 5,556,752; 5,561,071; 5,554,501; 5,545,531; 5,529,756; 5,527,681; 5,472,672; 5,445,934; 5,436,327; 5,429,807; 5,424,186; 5,412,087; 5,405,783; 5,384,261; 5,242,974; and the disclosures of which are herein incorporated by reference. Patents describing methods of using arrays in various applications include: U.S. Pat. Nos. 5,874,219; 5,848,659; 5,661,028; 5,580,732; 5,547,839; 5,525,464; 5,510,270; 5,503,980; 5,492,806; 5,470,710; 5,432,049; 5,324,633; 5,288,644; 5,143,854; and the disclosures of which are incorporated herein by reference.

B. Microarray Supports

A microarray support may comprise a flexible or rigid substrate. A flexible substrate is capable of being bent, folded, or similarly manipulated without breakage. Examples of solid materials that are flexible solid supports with respect to the present invention include membranes, such as nylon and flexible plastic films. The rigid supports of microarrays are sufficient to provide physical support and structure to the associated oligonucleotides under the appropriate assay conditions.

The support may be biological, nonbiological, organic, inorganic, or a combination of any of these, existing as particles, strands, precipitates, gels, sheets, tubing, spheres, containers, capillaries, pads, slices, films, plates, or slides. In addition, the support may have any convenient shape, such as a disc, square, sphere, or circle. In one embodiment, the support is flat but may take on a variety of alternative surface configurations. For example, the support may contain raised or depressed regions on which the synthesis takes place. The support and its surface may form a rigid support on which the reactions described herein may be carried out. The support and its surface may also be chosen to provide appropriate light-absorbing characteristics. For example, the support may be a polymerized Langmuir Blodgett film, functionalized glass, Si, Ge, GaAs, GaP, SiO₂, SIN₄, modified silicon, or any one of a wide variety of gels or polymers such as (poly)tetrafluoroethylene, (poly)vinylidenedifluoride, polystyrene, polycarbonate, or combinations thereof. The surface of the support may also contain reactive groups, such as carboxyl, amino, hydroxyl, and thiol groups. The surface may be transparent and contain SiOH functional groups, such as found on silica surfaces.

The support may be composed of a number of materials including glass. There are several advantages for utilizing glass supports in constructing a microarray. For example, microarrays prepared using a glass support, generally utilize microscope slides due to the low inherent fluorescence, thus, minimizing background noise. Moreover, hundreds to thousands of oligonucleotide probes may be attached to slide. The glass slides may be coated with polylysine, amino silanes, or amino-reactive silanes that enhance the hydrophobicity of the slide and improve the adherence of the oligonucleotides (Duggan et al. (1999)). Ultraviolet irradiation is used to crosslink the oligonucleotide probes to the glass support. Following irradiation, the support may be treated with succinic anhydride to reduce the positive charge of the amines. For double-stranded oligonucleotides, the support may be subjected to heat (e.g., 95° C.) or alkali treatment to generate single-stranded probes. An additional advantage to using glass is its nonporous nature, thus, requiring a minimal volume of hybridization buffer resulting in enhanced binding of target samples to probes.

In another embodiment, the support may be flat glass or single-crystal silicon with surface relief features of less than about 10 angstroms. The surface of the support may be etched using well-known techniques to provide desired surface features. For example, trenches, v-grooves, or mesa structures allow the synthesis regions to be more closely placed within the focus point of impinging light.

The present invention also relates to nucleic acid microarray supports comprising beads. These beads may have a wide variety of shapes and may be composed of numerous materials. Generally, the beads used as supports may have a homogenous size between about 1 and about 100 microns, and may include microparticles made of controlled pore glass (CPG), highly crosslinked polystyrene, acrylic copolymers, cellulose, nylon, dextran, latex, and polyacrolein. See e.g., U.S. Pat. Nos. 6,060,240; 4,678,814; and 4,413,070.

Several factors may be considered when selecting a bead for a support including material, porosity, size, shape, and linking moiety. Other important factors to be considered in selecting the appropriate support include uniformity, efficiency as a synthesis support, surface area, and optical properties (e.g., autofluoresence). Typically, a population of uniform oligonucleotide or nucleic acid fragment may be employed. However, beads with spatially discrete regions each containing a uniform population of the same oligonucleotide or nucleic acid fragment (and no other), may also be employed. In one embodiment, such regions are spatially discrete so that signals generated by fluorescent emissions at adjacent regions can be resolved by the detection system being employed.

In general, the support beads may be composed of glass (silica), plastic (synthetic organic polymer), or carbohydrate (sugar polymer). A variety of materials and shapes may be used, including beads, pellets, disks, capillaries, cellulose beads, pore-glass beads, silica gels, polystyrene beads optionally crosslinked with divinylbenzene, grafted co-poly beads, polyacrylamide beads, latex beads, dimethylacrylamide beads optionally cross-linked with N,N-1-bis-acryloyl ethylene diamine, and glass particles coated with a hydrophobic polymer (e.g., a material having a rigid or semirigid surface). The beads may also be chemically derivatized so that they support the initial attachment and extension of nucleotides on their surface.

Oligonucleotide probes may be synthesized directly on the bead, or the probes may be separately synthesized and attached to the bead. See e.g., Albretsen et al., 189 ANAL. BIOCHEM. 40-50 (1990); Lund et al., 16 NUCLEIC ACIDS RES. 10861-80 (1988); Ghosh et al., 15 NUCLEIC ACIDS RES. 5353-72 (1987); Wolf et al., 15 NUCLEIC ACIDS RES. 2911-26 (1987). The attachment to the bead may be permanent, or a cleavable linker between the bead and the probe may also be used. The link should not interfere with the probe-target binding during screening. Linking moieties for attaching and synthesizing tags on microparticle surfaces are disclosed in U.S. Pat. No. 4,569,774; Beattie et al., 39 CLIN. CHEM. 719-22 (1993); Maskos and Southern, 20 NUCLEIC ACIDS RES. 1679-84 (1992); Damba et al., 18 NUCLEIC ACIDS RES. 3813-21 (1990); and Pon et al., 6 BIOTECHNIQUES 768-75 (1988). Various links may include polyethyleneoxy, saccharide, polyol, esters, amides, saturated or unsaturated alkyl, aryl, and combinations thereof.

If the oligonucleotide probes are chemically synthesized on the bead, the bead-oligo linkage may be stable during the deprotection step of photolithography. During standard phosphoramidite chemical synthesis of oligonucleotides, a succinyl ester linkage may be used to bridge the 3′ nucleotide to the resin. This linkage may be readily hydrolyzed by NH₃ prior to and during deprotection of the bases. The finished oligonucleotides may be released from the resin in the process of deprotection. The probes may be linked to the beads by a siloxane linkage to Si atoms on the surface of glass beads; a phosphodiester linkage to the phosphate of the 3′-terminal nucleotide via nucleophilic attack by a hydroxyl (typically an alcohol) on the bead surface; or a phosphoramidate linkage between the 3′-terminal nucleotide and a primary amine conjugated to the bead surface.

Numerous functional groups and reactants may be used to detach the oligonucleotide probes. For example, functional groups present on the bead may include hydroxy, carboxy, iminohalide, amino, thio, active halogen (Cl or Br) or pseudohalogen (e.g., CF₃, CN), carbonyl, silyl, tosyl, mesylates, brosylates, and triflates. In some instances, the bead may have protected functional groups that may be partially or wholly deprotected.

1. Microarray Support Surface

The support of the microarrays may comprise at least one surface on which a pattern of oligonucleotide probes is present, where the surface may be smooth or substantially planar, or have irregularities, such as depressions or elevations. The surface on which the probes are located may be modified with one or more different layers of compounds that serve to modulate the properties of the surface. Such modification layers may generally range in thickness from a monomolecular thickness of about 1 mm, preferably from a monomolecular thickness of about 0.1 mm, and most preferred from a monomolecular thickness of about 0.001 mm. Modification layers include, for example, inorganic and organic layers such as metals, metal oxides, polymers, small organic molecules and the like. Polymeric layers include peptides, proteins, polynucleic acids or mimetics thereof (e.g., peptide nucleic acids), polysaccharides, phospholipids, polyurethanes, polyesters, polycarbonates, polyureas, polyamides, polyethyleneamines, polyarylene sulfides, polysiloxanes, polyimides, and polyacetates. The polymers may be hetero- or homopolymeric, and may or may not have separate functional moieties attached.

The oligonucleotide probes of a microarray may be arranged on the surface of the support based on size. With respect to the arrangement according to size, the probes may be arranged in a continuous or discontinuous size format. In a continuous size format, each successive position in the microarray, for example, a successive position in a lane of probes, comprises oligonucleotide probes of the same molecular weight. In a discontinuous size format, each position in the pattern (e.g., band in a lane) represents a fraction of target molecules derived from the original source, where the probes in each fraction will have a molecular weight within a determined range.

The probe pattern may take on a variety of configurations as long as each position in the microarray represents a unique size (e.g., molecular weight or range of molecular weights), depending on whether the array has a continuous or discontinuous format. The microarrays may comprise a single lane or a plurality of lanes on the surface of the support. Where a plurality of lanes are present, the number of lanes will usually be at least about 2 but less than about 200 lanes, preferably more than about 5 but less than about 100 lanes, and most preferred more than about 8 but less than about 80 lanes.

Each microarray may contain oligonucleotide probes isolated from the same source (e.g., the same tissue), or contain probes from different sources (e.g., different tissues, different species, disease and normal tissue). As such, probes isolated from the same source may be represented by one or more lanes; whereas probes from different sources may be represented by individual patterns on the microarray where probes from the same source are similarly located. Therefore, the surface of the support may represent a plurality of patterns of oligonucleotide probes derived from different sources (e.g., tissues), where the probes in each lane are arranged according to size, either continuously or discontinuously.

Surfaces of the support are usually, though not always, composed of the same material as the support. Alternatively, the surface may be composed of any of a wide variety of materials, for example, polymers, plastics, resins, polysaccharides, silica or silica-based materials, carbon, metals, inorganic glasses, membranes, or any of the above-listed substrate materials. The surface may contain reactive groups, such as carboxyl, amino, or hydroxyl groups. The surface may be optically transparent and may have surface SiOH functionalities, such as are found on silica surfaces.

2. Attachment of Oligonucleotide Probes

The surface of the support may possess a layer of linker molecules (or spacers). The linker molecules may be of sufficient length to permit oligonucleotide probes on the support to hybridize to nucleic acid molecules and to interact freely with molecules exposed to the support. The linker molecules may be about 6-50 molecules long to provide sufficient exposure. The linker molecules may also be, for example, aryl acetylene, ethylene glycol oligomers containing about 2-10 monomer units, diamines, diacids, amino acids, or combinations thereof.

The linker molecules may be attached to the support via carbon-carbon bonds using, for example, (poly)trifluorochloroethylene surfaces, or preferably, by siloxane bonds (using, for example, glass or silicon oxide surfaces). Siloxane bonds may be formed via reactions of linker molecules containing trichlorosilyl or trialkoxysilyl groups. The linker molecules may also have a site for attachment of a longer chain portion. For example, groups that are suitable for attachment to a longer chain portion may include amines, hydroxyl, thiol, and carboxyl groups. The surface attaching portions may include aminoalkylsilanes, hydroxyalkylsilanes, bis(2-hydroxyethyl)-aminopropyltriethoxysi lane, 2-hydroxyethylaminopropyltriethoxysilane, aminopropyltriethoxysilane, and hydroxypropyltriethoxysilane. The linker molecules may be attached in an ordered array (e.g., as parts of the head groups in a polymerized Langinuir Blodgett film). Alternatively, the linker molecules may be adsorbed to the surface of the support.

The linker may be a length that is at least the length spanned by, for example, two to four nucleotide monomers. The linking group may be an alkylene group (from about 6 to about 24 carbons in length), a polyethyleneglycol group (from about 2 to about 24 monomers in a linear configuration), a polyalcohol group, a polyamine group (e.g., spermine, spermidine, or polymeric derivatives thereof), a polyester group (e.g., poly(ethylacrylate) from 3 to 15 ethyl acrylate monomers in a linear configuration), a polyphosphodiester group, or a polynucleotide (from about 2 to about 12 nucleic acids). For in situ synthesis, the linking group may be provided with functional groups that can be suitably protected or activated. The linking group may be covalently attached to the oligonucleotide probes by an ether, ester, carbamate, phosphate ester, or amine linkage. In one embodiment, linkages are phosphate ester linkages, which can be formed in the same manner as the oligonucleotide linkages. For example, hexaethyleneglycol may be protected on one terminus with a photolabile protecting group (e.g., NVOC or MeNPOC) and activated on the other terminus with 2-cyanoethyl-N,N-diisopropylamino-chlorophosphite to form a phosphoramidite. This linking group may then be used for construction of oligonucleotide probes in the same manner as the photolabile-protected, phosphoramidite-activated nucleotides.

Furthermore, the linker molecules and oligonucleotide probes may contain a functional group with a bound protective group. In one embodiment, the protective group is on the distal or terminal end of the linker molecule opposite the support. The protective group may be either a negative protective group (e.g., the protective group renders the linker molecules less reactive with a monomer upon exposure) or a positive protective group (e.g., the protective group renders the linker molecules more reactive with a monomer upon exposure). In the case of negative protective groups, an additional reactivation step may be required, for example, through heating. The protective group on the linker molecules may be selected from a wide variety of positive light-reactive groups preferably including nitro aromatic compounds, such as o-nitrobenzyl derivatives or benzylsulfonyl. Other protective groups include 6-nitroveratryloxycarbonyl (NVOC), 2-nitrobenzyloxycarbonyl (NBOC) or α,α-dimethyl-dimethoxybenzyloxycarbonyl (DDZ). Photoremovable protective groups are described in, for example, Patchornik, 92 J. AM. CHEM. SOC. 6333 (1970) and Amit et al., 39 J. ORG. CHEM. 192 (1974).

C. Oligonucleotide Probes

A microarray may contain any number of different oligonucleotide probes. The microarray may have from about 2 to about 100 probes, about 100 to about 10,000 probes, or between about 10,000 and about 1,000,000 probes. In addition, the microarray may have a density of more than 100 oligonucleotide probes at known locations per cm², more than 1,000 probes per cm², or more than 10,000 per cm².

To detect gene expression, oligonucleotide probes may be designed and synthesized based on known sequence information. For example, 20- to 30-mer oligonucleotides that may be derived from known cDNA or EST sequences may be selected to monitor expression (Lipshutz et al. (1999)). The oligonucleotide probes may be selected from a number of sources including nucleic acid databases such as GenBank, Unigen, HomoloGene, RefSeq, dbEST, and dbSNP (Wheeler et al., 29 NUCL. ACIDS RES. 11-16 (2001)). Generally, the probe is complementary to the reference sequence, preferably unique to the tissue or cell type (e.g., skeletal muscle, neuronal tissue) of interest, and preferably hybridizes with high affinity and specificity (Lockhart et al., 14 NATURE BIOTECHNOL. 1675-80 (1996)). In addition, the oligonucleotide probe may represent non-overlapping sequences of the reference sequence that improves probe redundancy resulting in a reduction in false positive rate and an increased accuracy in target quantitation (Lipshutz et al. (1999)).

In one embodiment of the present invention, the oligonucleotide probes are relatively unique, for example, at least about 60-80% of the probes may comprise unique oligonucleotides. In another embodiment, modified oligonucleotides from about 80-300 nucleotides in length, or from about 100-200 nucleotides in length, may be used on the microarrays. These are especially useful in place of cDNAs for determining the presence of mRNA in a sample, as the modified oligonucleotides have the advantage of rapid synthesis and purification and analysis before attachment to the substrate surface. In particular, oligonucleotides with 2′-modified sugar groups demonstrate increased binding affinity with RNA, and these oligonucleotides are particularly advantageous in identifying mRNA in a sample exposed to a microarray.

Generally, the oligonucleotide probes are generated by standard synthesis chemistries such as phosphoramidite chemistry (U.S. Pat. Nos. 4,980,460; 4,973,679; 4,725,677; 4,458,066; and 4,415,732; Beaucage and Iyer, 48 TETRAHEDRON 2223-2311 (1992)). Alternative chemistries that create non-natural backbone groups, such as phosphorothionate and phosphoroamidate may also be employed.

Using the “flow channel” method, oligonucleotide probes are synthesized at selected regions on the support by forming flow channels on the surface of the support through which appropriate reagents flow or in which appropriate reagents are placed. For example, if a monomer is to be bound to the support in a selected region, all or part of the surface of the selected region may be activated for binding by flowing appropriate reagents through all or some of the channels, or by washing the entire support with appropriate reagents. After placing a channel block on the surface of the support, a reagent containing the monomer may flow through or may be placed in all or some of the channels. The channels provide fluid contact to the first selected region, thereby binding the monomer on the support directly or indirectly (via a spacer) in the first selected region.

If a second monomer is coupled to a second selected region, some of which may be included among the first selected region, the second selected region may be in fluid contact with second flow channels through translation, rotation, or replacement of the channel block on the surface of the support; through opening or closing a selected valve; or through deposition. The second region may then be activated. Thereafter, the second monomer may then flow through or may be placed in the second flow channels, binding the second monomer to the second selected region. Thus, the resulting oligonucleotides bound to the support are, for example, A, B, and AB. The process is repeated to form a microarray of oligonucleotide probes of desired length at known locations on the support.

Microarrays may have a plurality of modified oligonucleotides or polynucleotides stably associated with the surface of a support, e.g., covalently attached to the surface with or without a linker molecule. Each oligonucleotide on the array comprises a modified oligonucleotide composition of known identity and usually of known sequence. By stable association, the associated modified oligonucleotides maintain their position relative to the support under hybridization and washing conditions.

The oligonucleotides may be non-covalently or covalently associated with the support surface. Examples of non-covalent association include non-specific adsorption, binding based on electrostatic interactions (e.g., ion pair interactions), hydrophobic interactions, hydrogen bonding interactions, and specific binding through a specific binding pair member covalently attached to the support surface. Examples of covalent binding include covalent bonds formed between the oligonucleotides and a functional group present on the surface of the rigid support (e.g., —OH), where the functional group may be naturally occurring or present as a member of an introduced linking group.

II. Protein Microarrays

Although attempts to evaluate gene activity and to decipher biological processes have traditionally focused on genomics, proteomics offers a promising look at the biological functions of a cell. Proteomics involves the qualitative and quantitative measurement of gene activity by detecting and quantitating expression at the protein level, rather than at the messenger RNA level. Proteomics also involves the study of non-genome encoded events including the post-translational modification of proteins, interactions between proteins, and the location of proteins within the cell.

The study of gene expression at the protein level is important because many of the most important cellular processes are regulated by the protein status of the cell, not by the status of gene expression. In addition, the protein content of a cell is highly relevant to drug discovery efforts because many drugs are designed to be active against protein targets.

Current technologies for the analysis of proteomes are based on a variety of protein separation techniques followed by identification of the separated proteins. The most popular method is based on 2D-gel electrophoresis followed by “in-gel” proteolytic digestion and mass spectroscopy. This 2D-gel technique requires large sample sizes, is time consuming, and is currently limited in its ability to reproducibly resolve a significant fraction of the proteins expressed by a human cell. Techniques involving some large-format 2D-gels can produce gels that separate a larger number of proteins than traditional 2D-gel techniques, but reproducibility is still poor and over 95% of the spots cannot be sequenced due to limitations with respect to sensitivity of the available sequencing techniques. The electrophoretic techniques are also plagued by a bias towards proteins of high abundance.

Standard assays for the presence of an analyte in a solution, such as those commonly used for diagnostics, for example, involve the use of an antibody which has been raised against the targeted antigen. Multianalyte assays known in the art involve the use of multiple antibodies and are directed towards assaying for multiple analytes. However, these multianalyte assays have not been directed towards assaying the total or partial protein content of a cell or cell population. Furthermore, sample sizes required to adapt such standard antibody assay approaches to the analysis of even a fraction of the estimated 100,000 or more different proteins of a human cell and their various modified states are prohibitively large. Automation and/or miniaturization of antibody assays are required if large numbers of proteins are to be assayed simultaneously. Materials, surface coatings, and detection methods used for macroscopic immunoassays and affinity purification are not readily transferable to the formation or fabrication of miniaturized protein arrays.

Miniaturized DNA chip technologies have been developed and are currently being exploited for the screening of gene expression at the mRNA level. See e.g., U.S. Pat. Nos. 5,744,305; 5,412,087; and 5,445,934. These chips may be used to determine which genes are expressed by different types of cells and in response to different conditions. However, DNA biochip technology is not transferable to protein-binding assays such as antibody assays because the chemistries and materials used for DNA biochips are not readily transferable to use with proteins. Nucleic acids such as DNA withstand temperatures up to 100° C., can be dried and re-hydrated without loss of activity, and can be bound physically or chemically directly to organic adhesion layers supported by materials such as glass while maintaining their activity. In contrast, proteins such as antibodies are preferably kept hydrated and at ambient temperatures are sensitive to the physical and chemical properties of the support materials. Therefore, maintaining protein activity at the liquid-solid interface requires entirely different immobilization strategies than those used for nucleic acids. The proper orientation of the antibody or other protein-capture agent at the interface is desirable to ensure accessibility of their active sites with interacting molecules. With miniaturization of the chip and decreased feature sizes, the ratio of accessible to non-accessible and the ratio of active to inactive antibodies or proteins become increasingly relevant and important.

Thus, there is a need for the ability to assay in parallel a multitude of proteins expressed by a cell or a population of cells in an organism, including up to the total set of proteins expressed by the cell or cells.

A. Microarray Supports

The substrate of the microarray may be either organic or inorganic, biological or non-biological, or any combination of these materials. In addition, the substrate may be transparent or translucent. In one embodiment, the portion of the surface of the substrate on which the regions of protein-capture agents reside is flat and firm. In another embodiment, the portion of the surface of the substrate on which the regions of protein-capture agents reside is semi-firm. Of course, the protein microarrays of the present invention need not necessarily be flat nor entirely two-dimensional. Indeed, significant topological features may be present on the surface of the substrate surrounding the regions, between the regions or beneath the regions. For example, walls or other barriers may separate the regions of the microarray.

Numerous materials are suitable for use as a substrate in the microarray embodiment of the invention. The substrate of the invention microarray may comprise a material selected from the group consisting of silicon, silica, quartz, glass, controlled pore glass, carbon, alumina, titania, tantalum oxide, germanium, silicon nitride, zeolites, and gallium arsenide. Many metals such as gold, platinum, aluminum, copper, titanium, and their alloys may be useful as substrates of the microarray. Alternatively, many ceramics and polymers may also be used as substrates. Polymers that may be used as substrates include, but are not limited to polystyrene; poly(tetra)fluoroethylene (PTFE); polyvinylidenedifluoride; polycarbonate; polymethylmethacrylate; polyvinylethylene; polyethyleneimine; poly(etherether)ketone; polyoxymethylene (POM); polyvinylphenol; polylactides; polymethacrylimide (PMI); polyalkenesulfone (PAS); polypropylethylene, polyethylene; polyhydroxyethylmethacrylate (HEMA); polydimethylsiloxane; polyacrylamide; polyimide; and block-copolymers. The substrate on which the regions of protein-capture agents reside may also be a combination of any of the aforementioned substrate materials.

1. Microarray Support Surface

The support surfaces comprises the surface on which each of the protein-capture agents is immobilized. The support surfaces may comprise the substrate surface, an altered substrate surface, a coating applied to or formed on the substrate surface, or an organic thinfilm applied to or formed on the substrate surface or coating surface. Support surfacess comprise materials suitable for immobilization of the protein-capture agents to the microarrays. Suitable support surfacess include membranes, such as nitrocellulose membranes, polyvinylidenedifluoride (PVDF) membranes, and the like. In another emobdiment, the support surfaces may comprise a hydrogel such as dextran. Alternatively, the support surfaces may comprise an organic thinfilm including lipids, charged peptides (e.g., polylysine or poly-arginine), or a neutral amino acid (e.g., polyglycine).

The support surfaces may also comprise a compound that has the ability to interact with both the substrate and the protein-capture agent. For example, functionalities enabling interaction with the substrate may include hydrocarbons having functional groups (e.g. —O—, —CONH—, CONHCO—, —NH—, —CO—, —S—, —SO—), which may interact with functional groups on the substrate. Functionalities enabling interaction with the protein-capture agent comprise antibodies, antigens, receptor ligands, compounds comprising binding sites for affinity tags, and the like.

In another embodiment, the support surfaces may include a coating. The coating may be formed on, or applied to, the support surfaces. The substrate may be modified with a coating by using thinfilm technology based, for example, on physical vapor deposition (PVD), plasma-enhanced chemical vapor deposition (PECVD), or thermal processing.

Alternatively, plasma exposure may be used to directly activate or alter the substrate and create a coating. For example, plasma etch procedures can be used to oxidize a polymeric surface (for example, polystyrene or polyethylene to expose polar functionalities such as hydroxyls, carboxylic acids, aldehydes and the like) which then acts as a coating.

Furthermore, the coating may comprise a component to reduce non-specific binding. For example, a polypropylene substrate may be coated with a compound, such as bovine serum albumin, to reduce non-specific binding. Next, a support surfaces comprising dextran functionally linked to a receptor which recognizes M13 epitopes is added to distinct locations on the coating such that phage expressing recombinant proteins will be bound.

In an alternative embodiment, the coating may comprise an antibody. More particularly, antibodies that recognize epitope tags engineered into the recombinant proteins may be employed. Alternatively, recombinant proteins may comprise a poly-histidine affinity tag. In this case, an anti-histidine antibody chemically linked to the substrate provides a support surfaces for immobilization of the protein-capture agents.

In yet another embodiment, the coating may comprise a metal film. The metal film may range from about 50 nm to about 500 nm in thickness. Alternatively, the metal film may range from about 1 nm to about 1 μm in thickness.

Examples of metal films that may be used as substrate coatings include aluminum, chromium, titanium, tantalum, nickel, stainless steel, zinc, lead, iron, copper, magnesium, manganese, cadmium, tungsten, cobalt, and alloys or oxides thereof. In one embodiment, the metal film is a noble metal film. Noble metals that may be used for a coating include, but are not limited to, gold, platinum, silver, and copper. In another embodiment, the coating comprises gold or a gold alloy. Electron-beam evaporation may be used to provide a thin coating of gold on the surface of the substrate. Additionally, commercial metal-like substances may be employed such as TALON metal affinity resin and the like.

In alternative embodiments, the coating may comprise a composition selected from the group consisting of silicon, silicon oxide, titania, tantalum oxide, silicon nitride, silicon hydride, indium tin oxide, magnesium oxide, alumina, glass, hydroxylated surfaces, and polymers.

It is contemplated that the coatings of the microarrays may require the addition of at least one adhesion layer or interlayer between the coating and the substrate. The adhesion layer may be at least about 6 angstroms thick but may be much thicker. For example, a layer of titanium or chromium may be desirable between a silicon wafer and a gold coating. In an alternative embodiment, an epoxy glue such as Epo-tek 377® or Epo-tek 301-2®, (Epoxy Technology Inc., Billerica, Mass.) may be used to aid adherence of the coating to the substrate. Determinations as to what material should be used for the adhesion layer would be obvious to one skilled in the art once materials are chosen for both the substrate and coating. In other embodiments, additional adhesion mediators or interlayers may be necessary to improve the optical properties of the microarray, for example, waveguides for detection purposes.

In one embodiment of the invention, the surface of the coating is atomically flat. The mean roughness of the surface of the coating may be less than about 5 angstroms for areas of at least about 25 μm². In a specific embodiment, the mean roughness of the surface of the coating is less than about 3 angstroms for areas of at least about 25 μm². In one embodiment, the coating may be a template-stripped surface. See, e.g., Hegner et al., 291 SURFACE SCIENCE 39-46 (1993); Wagner et al., 11 LANGMUIR 3867-3875 (1995).

Several different types of coating may be combined on the surface. The coating may cover the whole surface of the substrate or only parts of it. In one embodiment, the coating covers the substrate surface only at the site of the regions of protein-capture agents. Techniques useful for the formation of coated regions on the surface of the substrate are well known to those of ordinary skill in the art. For example, the regions of coatings on the substrate may be fabricated by photolithography, micromolding (WO 96/29629), wet chemical or dry etching, or any combination of these.

a. Organic Thinfilms

In a particular embodiment, the support surfaces comprises an organic thinfilm layer. The organic thinfilm on which each of the regions of protein-capture agents resides forms a layer either on the substrate itself or on a coating covering the substrate. In one embodiment, the organic thinfilm on which the protein-capture agents of the regions are immobilized is less than about 20 nm thick. In another embodiment, the organic thinfilm of each of the regions is less than about 10 nm thick.

A variety of different organic thinfilms are suitable for use in the present invention. For example, a hydrogel composed of a material such as dextran may serve as a suitable organic thinfilm on the regions of the microarray. In another embodiment, the organic thinfilm is a lipid bilayer.

In yet another embodiment, the organic thinfilm of each of the regions of the microarray is a monolayer. A monolayer of polyarginine or polylysine adsorbed on a negatively charged substrate or coating may comprise the organic thinfilm. Another option is a disordered monolayer of tethered polymer chains. In a particular embodiment, the organic thinfilm is a self-assembled monolayer. Specifically, the self-assembled monolayer may comprise molecules of the formula X—R—Y, wherein R is a spacer, X is a functional group that binds R to the surface, and Y is a functional group for binding protein-capture agents onto the monolayer. In an alternative embodiment, the self-assembled monolayer is comprised of molecules of the formula (X)ₐ R(Y)_{b} where a and b are, independently, integers greater than or equal to 1 and X, R, and Y are as previously defined.

In another embodiment, the organic thinfilm comprises a combination of organic thinfilms such as a combination of a lipid bilayer immobilized on top of a self-assembled monolayer of molecules of the formula X—R—Y. As another example, a monolayer of polylysine may be combined with a self-assembled monolayer of molecules of the formula X-R-Y. See U.S. Pat. No. 5,629,213.

In all cases, the coating, or the substrate itself if no coating is present, must be compatible with the chemical or physical adsorption of the organic thinfilm on its surface. For example, if the microarray comprises a coating between the substrate and a monolayer of molecules of the formula X—R—Y, then it is understood that the coating must be composed of a material for which a suitable functional group X is available. If no such coating is present, then it is understood that the substrate must be composed of a material for which a suitable functional group X is available.

In one embodiment of the invention, the area of the substrate surface, or coating surface, which separates the regions of protein-capture agents are free of organic thinflim. In an alternative embodiment, the organic thinfilm may extend beyond the area of the substrate surface, or coating surface if present, covered by the regions of protein-capture agents. For example, the entire surface of the microarray may be covered by an organic thinfilm on which the plurality of spatially distinct regions of protein-capture agents reside. An organic thinfilm that covers the entire surface of the microarray may be homogenous or may comprise regions of differing exposed functionalities useful in the immobilization of regions of different protein-capture agents.

In yet another embodiment, the areas of the substrate surface or coating surface between the regions of protein-capture agents are covered by an organic thinfilm, but an organic thinfilm of a different type than that of the regions of protein-capture agents. For example, the surfaces between the regions of protein-capture agents may be coated with an organic thinfilm characterized by low non-specific binding properties for proteins and other analytes.

A variety of techniques may be used to generate regions of organic thinfilm on the surface of the substrate or on the surface of a coating on the substrate. These techniques are well known to those skilled in the art and will vary depending upon the nature of the organic thinfilm, the substrate, and the coating, if present. The techniques will also vary depending on the structure of the underlying substrate and the pattern of any coating present on the substrate. For example, regions of a coating that are highly reactive with an organic thinfilm may have already been produced on the substrate surface. Areas of organic thinfilm may be created by microfluidics printing, microstamping (U.S. Pat. Nos. 5,731,152 and 5,512,131), or microcontact printing (WO 96/29629). Subsequent immobilization of protein-capture agents to the reactive monolayer regions result in two-dimensional arrays of the agents. Inkjet printer heads provide another option for patterning monolayer X—R—Y molecules, or components thereof, or other organic thinfilm components to nanometer or micrometer scale sites on the surface of the substrate or coating. See, e.g., Lemmo et al., 69 ANAL CHEM. 543-551 (1997); U.S. Pat. Nos. 5,843,767 and 5,837,860. In some cases, commercially available arrayers based on capillary dispensing may also be of use in directing components of organic thinfilms to spatially distinct regions of the microarray (OmniGrid® from Genemachines, Inc, San Carlos, Calif., and High-Throughput Microarrayer from Intelligent Bio-Instruments, Cambridge, Mass.). Other methods for the formation of organic thinfilms include in situ growth from the surface, deposition by physisorption, spin-coating, chemisorption, self-assembly, or plasma-initiated polymerization from gas phase.

Diffusion boundaries between the regions of protein-capture agents immobilized on organic thinfilms such as self-assembled monolayers may be integrated as topographic patterns (physical barriers) or surface functionalities with orthogonal wetting behavior (chemical barriers). For example, walls of substrate material may be used to separate some of the regions of protein-capture agents from some of the others or all of the regions from each other. Alternatively, non-bioreactive organic thinfilms, such as monolayers, with different wettability may be used to separate regions of protein-capture agents from one another.

B. Protein-Capture Agents

A protein microarray contemplated by the present invention may contain any number of different proteins, amino acid sequences, nucleic acid sequences, or small molecules. In one embodiment, the microarrays may comprise all or a portion of a gene, including functional derivatives, variants, analogs and portions thereof. The present invention also contemplates microarrays comprising one or more antibodies or functional equivalents thereof that bind proteins, ligands, and/or binding partners.

For example, the proteins expressed by the protein protein-capture agents immobilized on the microarray may be members of the same family. Such families include, but are not limited to, families of growth factor receptors, hormone receptors, neurotransmitter receptors, catecholamine receptors, amino acid derivative receptors, cytokine receptors, extracellular matrix receptors, antibodies, lectins, cytokines, serpins, proteinases, kinases, phosphatases, ras-like GTPases, hydrolases, steroid hormone receptors, transcription factors, DNA binding proteins, zinc finger proteins, leucine-zipper proteins, homeodomain proteins, intracellular signal transduction modulators and effectors, apoptosis-related factors, DNA synthesis factors, DNA repair factors, DNA recombination factors, cell-surface antigens, Hepatitis C virus (HCV) proteases, HIC proteases, viral integrases, and proteins from pathogenic bacteria.

A protein-capture agent on the microarray may be any molecule or complex of molecules that has the ability to bind a protein and immobilize it to the site of the protein-capture agent on the microarray. In one aspect, the protein-capture agent binds its binding partner in a substantially specific manner. For example, the protein-capture agent may be a protein whose natural function in a cell is to specifically bind another protein, such as an antibody or a receptor. Alternatively, the protein-capture agent may be a partially or wholly synthetic or recombinant protein that specifically binds a protein.

Moreover, the protein-capture agent may be a protein which has been selected in vitro from a mutagenized, randomized, or completely random and synthetic library by its binding affinity to a specific protein or peptide target. The selection method used may be a display method such as ribosome display or phage display. Alternatively, the protein-capture agent obtained via in vitro selection may be a DNA or RNA aptamer that specifically binds a protein target. See, e.g., Polyrailo et al., 70 ANAL. CHEM. 3419-25 (1998); Cohen, et al., 94 PROC. NATL. ACAD. SCI. USA 14272-7 (1998); Fukuda, et al., 37 NUCLEIC ACIDS SYMP. SER., 237-8 (1997). Alternatively, the in vitro selected protein-capture agent may be a polypeptide. Roberts and Szostak, 94 PROC. NATL. ACAD. SCI. USA 12297-302 (1997). In yet another embodiment, the protein-capture agent may be a small molecule that has been selected from a combinatorial chemistry library or is isolated from an organism.

In a particular embodiment, however, the protein-capture agents are proteins. The protein-capture agents may be antibodies or antibody fragments. Although antibody moieties are exemplified herein, it is understood that the present arrays and methods may be advantageously employed with other protein-capture agents.

The antibodies or antibody fragments of the microarray may be single-chain Fvs, Fab fragments, Fab′ fragments, F(ab′)₂ fragments, Fv fragments, dsFvs diabodies, Fd fragments, full-length, antigen-specific polyclonal antibodies, or full-length monoclonal antibodies. In a specific embodiment, the protein-capture agents of the microarray are monoclonal antibodies, Fab fragments or single-chain Fvs.

The antibodies or antibody fragments may be monoclonal antibodies, even commercially available antibodies, against known, well-characterized proteins. Alternatively, the antibody fragments may be derived by selection from a library using the phage display method. If the antibody fragments are derived individually by selection based on binding affinity to known proteins, then the binding partners of the antibody fragments are known. In an alternative embodiment of the invention, the antibody fragments are derived by a phage display method comprising selection based on binding affinity to the (typically, immobilized) proteins of a cellular extract or a biological sample. In this embodiment, some or many of the antibody fragments of the microarray would bind proteins of unknown identity and/or function.

1. Attachment of Protein-Capture Agents

It is necessary, however, to immobilize proteins-capture agents on a solid support in a way that preserves their folded conformations. Methods of arraying functionally active proteins using microfabricated polyacrylamide gel pads to preserve samples and microelectrophoresis to accelerate diffusion have been described. Arenkov et al., 278 ANAL. BIOCHEM. 123-31 (2000).

The method of attachment will vary with the substrate and protein-capture agent selected. For example, in the case of a phage display library, the method of attachment may involve either the direct attachment of the phage as for example, by anti-M13 antibodies, or by attachment via the recombinant protein as for example via antibodies to an epitope-tag incorporated in the recombinant sequence, or by binding of a histidine-tag (his-tag) incorporated in the recombinant sequence to a metal coating on the support surfaces.

In one embodiment, the protein-immobilizing regions of the microarray comprise an affinity tag that enhances immobilization of the protein-capture agent onto the organic thinfilm. The use of an affinity tag on the protein-capture agent of the microarray provides several advantages. An affinity tag can confer enhanced binding or reaction of the protein-capture agent with the functionalities on the organic thinfilm, such as Y if the organic thinfilm is a an X—R—Y monolayer as previously described. This enhancement effect may be either kinetic or thermodynamic. The affinity tag/organic thinfilm combination used in the regions of protein-capture agents residing on the microarray allows for immobilization of the protein-capture agents in a manner that does not require harsh reaction conditions which are adverse to protein stability or function. In most embodiments, the protein-capture agents are immobilized to the organic thinfilm in aqueous, biological buffers.

An affinity tag also offers immobilization on the organic thinfilm that is specific to a designated site or location on the protein-capture agent (site-specific immobilization). For this to occur, attachment of the affinity tag to the protein-capture agent must be site-specific. Site-specific immobilization helps ensure that the protein-binding site of the agent, such as the antigen-binding site of the antibody moiety, remains accessible to ligands in solution. Another advantage of immobilization through affinity tags is that it allows for a common immobilization strategy to be used with multiple, different protein-capture agents.

The affinity tag may be attached directly, either covalently or noncovalently, to the protein-capture agent. In an alternative embodiment, however, the affinity tag is either covalently or noncovalently attached to an adaptor that is either covalently or noncovalently attached to the protein-capture agent.

In one embodiment, the affinity tag comprises at least one amino acid. The affinity tag may be a polypeptide comprising at least two amino acids which are reactive with the functionalities of the organic thinfilm. Alternatively, the affinity tag may be a single amino acid that is reactive with the organic thinfilm. Examples of possible amino acids that could be reactive with an organic thinfilm include cysteine, lysine, histidine, arginine, tyrosine, aspartic acid, glutamic acid, tryptophan, serine, threonine, and glutamine. A polypeptide or amino acid affinity tag may be expressed as a fusion protein with the protein-capture agent when the protein-capture agent is a protein, such as an antibody or antibody fragment. Amino acid affinity tags provide either a single amino acid or a series of amino acids that may interact with the functionality of the organic thinfilm, such as the Y-functional group of the self-assembled monolayer molecules. Amino acid affinity tags may be readily introduced into recombinant proteins to facilitate oriented immobilization by covalent binding to the Y-functional group of a monolayer or to a functional group on an alternative organic thinfilm.

The affinity tag may comprise a poly-amino acid tag. A poly-amino acid tag is a polypeptide that comprises from about 2 to about 100 residues of a single amino acid, optionally interrupted by residues of other amino acids. For example, the affinity tag may comprise a poly-cysteine, poly-lysine, poly-arginine, or poly-histidine. Amino acid tags may comprise about two to about twenty residues of a single amino acid, such as, for example, histidines, lysines, arginines, cysteines, glutamines, tyrosines, or any combination of these. For example, an amino acid tag of one to twenty amino acids includes at least one to ten cysteines for thioether linkage; or one to ten lysines for amide linkage; or one to ten arginines for coupling to vicinal dicarbonyl groups. One of ordinary skill in the art can readily pair suitable affinity tags with a given functionality on an organic thinfilm.

The position of the amino acid tag may be at an amino-, or carboxy-terminus of the protein-capture agent which is a protein, or anywhere in-between, as long as the protein-binding region of the protein-capture agent, such as the antigen-binding region of an immobilized antibody moiety, remains in a position accessible for protein binding. Affinity tags introduced for protein purification may be located at the C-terminus of the recombinant protein to ensure that only full-length proteins are isolated during protein purification. For example, if intact antibodies are used on the microarrays, then the attachment point of the affinity tag on the antibody may be located at a C-terminus of the effector (Fc) region of the antibody. If scFvs are used on the arrays, then the attachment point of the affinity tag may also be located at the C-terminus of the molecules.

Affinity tags may also contain one or more unnatural amino acids. Unnatural amino acids may be introduced using suppressor tRNAs that recognize stop codons (i.e., amber) See, e.g., Cload et al., 3 CHEM. BIOL. 1033-1038 (1996); Ellman et al., 202 METHODS ENZYM. 301-336 (1991); and Noren et al., 244 SCIENCE 182-188 (1989). The tRNAs are chemically amino-acylated to contain chemically altered (“unnatural”) amino acids for use with specific coupling chemistries (i.e., ketone modifications, photoreactive groups).

In an alternative embodiment, the affinity tag comprises an intact protein, such as, but not limited to, glutathione S-transferase, an antibody, avidin, or streptavidin.

In embodiments where the protein-capture agent is a protein and the affinity tag is a protein, such as a poly-amino acid tag or a single amino acid tag, the affinity tag may be attached to the protein-capture agent by generating a fusion protein. Alternatively, protein synthesis or protein ligation techniques known to those skilled in the art may be used. For example, intein-mediated protein ligation may be used to attach the affinity tag to the protein-capture agent. See, e.g., Mathys, et al., 231 GENE 1-13 (1999); Evans, et al., 7 PROTEIN SCIENCE 2256-2264 (1998).

Other protein conjugation and immobilization techniques known in the art may be adapted for the purpose of attaching affinity tags to the protein-capture agent. For example, the affinity tag may be an organic bioconjugate that is chemically coupled to the protein-capture agent of interest. Biotin or antigens may be chemically cross-linked to the protein. Alternatively, a chemical crosslinker may be used that attaches a simple functional moiety such as a thiol or an amine to the surface of a protein serving as a protein-capture agent on the microarray.

In one embodiment of the present invention, the organic thinfilm of each of the regions comprises, at least in part, a lipid monolayer or bilayer, and the affinity tag comprises a membrane anchor.

In an alternative embodiment, no affinity tag is used to immobilize the protein-capture agents onto the organic thinfilm. An amino acid or other moiety (such as a carbohydrate moiety) inherent to the protein-capture agent itself may instead be used to tether the protein-capture agent to the reactive group of the organic thinfilm. In one embodiment, the immobilization is site-specific with respect to the location of the site of immobilization on the protein-capture agent. For example, the sulfhydryl group on the C-terminal region of the heavy chain portion of a Fab′ fragment generated by pepsin digestion of an antibody, followed by selective reduction of the disulfide bond between monovalent Fab′ fragments, may be used as the affinity tag. Alternatively, a carbohydrate moiety on the Fc portion of an intact antibody may be oxidized under mild conditions to an aldehyde group suitable for immobilizing the antibody on a monolayer via reaction with a hydrazide-activated Y group on the monolayer. See e.g., U.S. Pat. No. 6,329,209; Dammer et al., 70 BIOPHYS J. 2437-2441 (1996).

Because the protein-capture agents of at least some of the different regions on the microarray are different from each other, different solutions, each containing a different protein-capture agent, must be delivered to the individual regions. Solutions of protein-capture agents may be transferred to the appropriate regions via arrayers, which are well-known in the art and even commercially available. For example, microcapillary-based dispensing systems may be used. These dispensing systems may be automated and computer-aided. A description of and building instructions for an example of a microarrayer comprising an automated capillary system can be found on the internet at http://cmgm.stanford.edu/pbrown/microarray.html and http://cmgm.stanford.edu/pbrown/mguide/index.html. The use of other microprinting techniques for transferring solutions containing the protein-capture agents to the agent-reactive regions is also possible. Ink-jet printer heads may also be used for precise delivery of the protein-capture agents to the agent-reactive regions. Representative, non-limiting disclosures of techniques useful for depositing the protein-capture agents on the appropriate regions of the substrate may be found, for example, in U.S. Pat. Nos. 5,843,767 (ink-jet printing technique, Hamilton 2200 robotic pipetting delivery system); 5,837,860 (ink-jet printing technique, Hamilton 2200 robotic pipetting delivery system); 5,807,522 (capillary dispensing device); and 5,731,152 (stamping apparatus). Other methods of arraying functionally active proteins include attaching proteins to the surfaces of chemically derivatized microscope slides. See MacBeath & Schreiber, 289 SCIENCE 1760-63 (2000).

a. Adaptors

Another embodiment of the protein microarrays of the present invention comprises an adaptor that links the affinity tag to the protein-capture agent on the regions of the microarray. The additional spacing of the protein-capture agent from the surface of the substrate (or coating) that is afforded by the use of an adaptor is particularly advantageous if the protein-capture agent is a protein, because proteins are prone to surface inactivation. The adaptor may afford some additional advantages as well. For example, the adaptor may help facilitate the attachment of the protein-capture agent to the affinity tag. In another embodiment, the adaptor may help facilitate the use of a particular detection technique with the microarray. One of ordinary skill in the art will be able to choose an adaptor which is appropriate for a given affinity tag. For example, if the affinity tag is streptavidin, then the adaptor could be biotin that is chemically conjugated to the protein-capture agent which is to be immobilized.

In one embodiment, the adaptor comprises a protein. In another embodiment, the affinity tag, adaptor, and protein-capture agent together compose a fusion protein. Such a fusion protein may be readily expressed using standard recombinant DNA technology. Protein adaptors are especially useful to increase the solubility of the protein-capture agent of interest and to increase the distance between the surface of the substrate or coating and the protein-capture agent. A protein adaptor can also be very useful in facilitating the preparative steps of protein purification by affinity binding prior to immobilization on the microarray. Examples of possible adaptor proteins include glutathione-S-transferase (GST), maltose-binding protein, chitin-binding protein, thioredoxin, and green-fluorescent protein (GFP). GFP may also be used for quantification of surface binding. In an embodiment in which the protein-capture agent is an antibody moiety comprising the Fe region, the adaptor may be a polypeptide, such as protein G, protein A, or recombinant protein A/G (a gene fusion product secreted from a non-pathogenic form of Bacillus which contains four Fc binding domains from protein A and two from protein G).

2. Preparation of the Protein-capture Agents of the Microarray

The protein-capture agents used on the microarray may be produced by any of the variety of means known to those of ordinary skill in the art. The protein-capture agents may comprise proteins, specifically, antibodies or fragments thereof, ligands, receptor proteins, and small molecules.

In preparation for immobilization to the arrays of the present invention, the antibody moiety, or any other protein-capture agent that is a protein or polypeptide, may be expressed from recombinant DNA either in vivo or in vitro. The cDNA encoding the antibody or antibody fragment or other protein-capture agent may be cloned into an expression vector (many examples of which are commercially available) and introduced into cells of the appropriate organism for expression. A broad range of host cells and protein-capture agents may be used to produce the antibodies and antibody fragments, or other proteins, which serve as the protein-capture agents on the microarray. Expression in vivo may be accomplished in bacteria (e.g., Escherichia coli), plants (e.g., Nicotiana tabacum), lower eukaryotes (e.g., Saccharomyces cerevisiae, Saccharomyces pombe, Pichia pastoris), or higher eukaryotes (e.g., bacculovirus-infected insect cells, insect cells, mammalian cells). For in vitro expression, PCR-amplified DNA sequences may be directly used in coupled in vitro transcription/translation systems (e.g., E. coli S30 lysates from T7 RNA polymerase expressing, preferably protease-deficient strains; wheat germ lysates; reticulocyte lysates). The choice of organism for optimal expression depends on the extent of post-translational modifications (i.e., glycosylation, lipid-modifications) desired. The choice of protein-capture agent also depends on other issues, such as whether an intact antibody is to be produced or just a fragment of an antibody (and which fragment), because disulfide bond formation will be affected by the choice of a host cell. One of ordinary skill in the art will be able to readily choose which host cell type is most suitable for the protein-capture agent and application desired.

DNA sequences encoding affinity tags and adaptors may be engineered into the expression vectors such that the protein-capture agent genes of interest can be cloned in frame either 5′ or 3′ of the DNA sequence encoding the affinity tag and adaptor protein. In most aspects, the expressed protein-capture agents may purified by affinity chromatography using commercially available resins.

Production of a plurality of protein-capture agents may involve parallel processing from cloning to protein expression and protein purification. cDNAs encoding the protein-capture agent of interest may be amplified by PCR using cDNA libraries or expressed sequence tag (EST) clones as templates. For in vivo expression of the proteins, cDNAs may be cloned into commercial expression vectors and introduced into an appropriate organism for expression. For in vitro expression PCR-amplified DNA sequences may be directly used in coupled transcription/translation systems.

E. coli-based protein expression is generally the method of choice for soluble proteins that do not require extensive post-translational modifications for activity. Extracellular or intracellular domains of membrane proteins may be fused to protein adaptors for expression and purification.

The entire approach may be performed using 96-well assay plates. PCR reactions may be carried out under standard conditions. Oligonucleotide primers may contain unique restriction sites for facile cloning into the expression vectors. Alternatively, the TA cloning system may be used. The expression vectors may further contain the sequences for affinity tags and the protein adaptors. PCR products may be ligated into the expression vectors (under inducible promoters) and introduced into the appropriate competent E. coli strain by calcium-dependent transformation (strains include: XL-1 blue, BL21, SG13009 (lon-)). Transformed E. coli cells are plated and individual colonies transferred into 96-microarray blocks. Cultures are grown to mid-log phase, induced for expression, and cells collected by centrifugation. Cells are resuspended containing lysozyme and the membranes broken by rapid freeze/thaw cycles, or by sonication. Cell debris is removed by centrifugation and the supernatants transferred to 96-tube arrays. The appropriate affinity matrix is added, the protein-capture agent of interest is bound and nonspecifically bound proteins are removed by repeated washing and other steps using centrifugation devices. Alternatively, magnetic affinity beads and filtration devices may be used. The proteins are eluted and transferred to a new 96-well microarray. Protein concentrations are determined and an aliquot of each protein-capture agent is spotted onto a nitrocellulose filter and verified by Western analysis using an antibody directed against the affinity tag on the protein-capture agent. The purity of each sample is assessed by SDS-PAGE and Silver staining or mass spectrometry. The protein-capture agents are then snap-frozen and stored at −80° C.

S. cerevisiae allows for the production of glycosylated protein-capture agents such as antibodies or antibody fragments. For production in S. cerevisiae, the approach described above for E. coli may be used with slight modifications for transformation and cell lysis. Transformation of S. cerevisiae may be accomplished by lithium-acetate and cell lysis by lyticase digestion of the cell walls followed by freeze-thaw, sonication or glass-bead extraction. Variations of post-translational modifications may be obtained by using different yeast strains (i.e., S. pombe, P. pastoris).

One aspect of the bacculovirus system is the array of post-translational modifications that can be obtained, although antibodies and other proteins produced in bacculovirus contain carbohydrate structures very different from those produced by mammalian cells. The bacculovirus-infected insect cell system requires cloning of viruses, obtaining high titer stocks and infection of liquid insect cell suspensions (cells such as SF9, SF21).

Mammalian cell-based expression requires transfection and cloning of cell lines. Either lymphoid or non-lymphoid cell may be used in the preparation of antibodies and antibody fragments. Soluble proteins such as antibodies are collected from the medium while intracellular or membrane bound proteins require cell lysis (either detergent solubilization or freeze-thaw). The protein-capture agents may then be purified by a procedure analogous to that described for E. coli.

For in vitro translation, the system of choice is E. coli lysates obtained from protease-deficient and T7 RNA polymerase overexpressing strains. E. coli lysates provide efficient protein expression (30-50 μg/ml lysate). The entire process may be carried out in 96-well arrays. Antibody genes or other protein-capture agent genes of interest may be amplified by PCR using oligonucleotides that contain the gene-specific sequences containing a T7 RNA polymerase promoter and binding site and a sequence encoding the affinity tag. Alternatively, an adaptor protein may be fused to the gene of interest by PCR. Amplified DNAs may be directly transcribed and translated in the E. coli lysates without prior cloning for fast analysis. The antibody fragments or other proteins may then be isolated by binding to an affinity matrix and processed as described above.

Alternative in vitro translation systems that may be used include wheat germ extracts and reticulocyte extracts. In vitro synthesis of membrane proteins or post-translationally modified proteins will require reticulocyte lysates in combination with microsomes.

In one embodiment of the invention, the protein-capture agents on the microarray comprise monoclonal antibodies. The production of monoclonal antibodies against specific protein targets is routine using standard hybridoma technology. In fact, numerous monoclonal antibodies are available commercially.

As an alternative to obtaining antibodies or antibody fragments by cell fusion or from continuous cell lines, the antibody moieties may be expressed in bacteriophage. Such antibody phage display technologies are well known to those skilled in the art. The bacteriophage protein-capture agents allow for the random recombination of heavy- and light-chain sequences, thereby creating a library of antibody sequences that may be selected against the desired antigen. The protein-capture agent may be based on bacteriophage lambda or on filamentous phage. The bacteriophage protein-capture agent may be used to express Fab fragments, Fv's with an engineered intermolecular disulfide bond to stabilize the V_{H}-V_{L} pair (dsFv's), scFvs, or diabody fragments.

The antibody genes of the phage display libraries may be derived from pre-immunized donors. For example, the phage display library could be a display library prepared from the spleens of mice previously immunized with a mixture of proteins, such as a lysate of human T-cells. Immunization may be used to bias the library to contain a greater number of recombinant antibodies reactive towards a specific set of proteins, such as proteins found in human T-cells. Alternatively, the library antibodies may be derived from native or synthetic libraries. The native libraries may be constructed from spleens of mice that have not been contacted by external antigen. In a synthetic library, portions of the antibody sequence, typically those regions corresponding to the complementarity determining regions (CDR) loops, have been mutagenized or randomized.

III. Target Samples

Biological samples may be isolated from several sources including, but not limited to, a patient or a cell line. Patient samples may include blood, urine, amniotic fluid, plasma, semen, bone marrow, and tissues. Once isolated, total RNA or protein may be extracted using methods well known in the art. For example, target samples may be generated from total RNA by dT-primed reverse transcription producing cDNA (see e.g., SAMBROOK ET AL., MOLECULAR CLONING: A LABORATORY MANUAL, Cold Spring Harbor Press, New York (1989); AUSUBEL ET AL., CURRENT PROTOCOLS IN MOLECULAR BIOLOGy, John Wiley & Sons, Inc. (1995)). The cDNA may then be transcribed to cRNA by in vitro transcription resulting in a linear amplification of the RNA. The target samples may be labeled with, for example, a fluorescent dye (e.g., Cy3-dUTP) or biotin. The labeled targets may be hybridized to the microarray. Laser excitation of the target samples produces fluorescence emissions, which are captured by a detector. This information may then be used to generate a quantitative two-dimensional fluorescence image of the hybridized targets.

Gene expression profiles of a particular tissue or cell type may be generated from RNA (i.e., total RNA or mRNA). Reverse transcription with an oligo-dT primer may be used to isolate and generate mRNA from cellular RNA. To maximize the amount of sample or signal, labeled total RNA may also be used. The RNA may be fluorescently labeled or labeled with a radioactive isotope. For radioactive detection, a low energy emitter, such as ³³P-dCTP, is preferred due to close proximity of the oligonucleotide probes on the support. The fluorophores, Cy3-dUTP or Cy5-dUTP, may used for fluorescent labeling. These fluorophores demonstrate efficient incorporation with reverse transcriptase and better yields. Furthermore, these fluorophores possess distinguishable excitation and emission spectra. Thus, two samples, each labeled with a different fluorophore, may be simultaneously hybridized to a microarray.

The nucleic acid sample may be amplified prior to hybridization. Amplification methods include, but are not limited to PCR (INNIS ET AL., PCR PROTOCOLS. A GUIDE TO METHODS AND APPLICATION, Academic Press, Inc. San Diego, (1990)), ligase chain reaction (LCR) (Barringer et al., 89 GENE 117 (1990); Wu and Wallace, 4 GENOMES 560 (1989); and Landegren et al., 241 SCIENCE 1077 (1988)), transcription amplification (Kwoh, et al., 86 PROC. NATL. ACAD. SCI. USA 1173 (1989)), and self-sustained sequence replication (Guatelli, et al., 87 PROC. NATL. ACAD. SCI. USA 1874 (1990)).

The target nucleic acids may be labeled at one or more nucleotides during or after amplification. Labels suitable for use with microarray technology include labels detectable by spectroscopic, photochemical, biochemical, immunochemical, electrical, optical, or chemical means. In one embodiment, the detectable label is a luminescent label, such as fluorescent labels, chemiluminescent labels, bioluminescent labels, and colorimetric labels. In a specific embodiment, the label is a fluorescent label such as fluorescein, rhodamine, lissamine, phycoerythrin, polymethine dye derivative, phosphor, or Cy2, Cy3, Cy3.5, Cy5, Cy5.5, Cy7. Commercially available fluorescent labels include fluorescein phosphoramidites such as Fluoreprime (Pharmacia, Piscataway, N.J.), Fluoredite (Millipore, Bedford, Mass.), and FAM (ABI, Foster City, Calif.). Other labels include biotin for staining with labeled streptavidin conjugate, magnetic beads (e.g., Dynabeads), fluorescent dyes (e.g., texas red, rhodamine, green fluorescent protein), radiolabels (e.g., ³H, ¹²⁵I, ³⁵S, ¹⁴C, or ³²P), enzymes (e.g., horseradish peroxidase, alkaline phosphatase), and colorimetric labels such as colloidal gold or colored glass or plastic (e.g., polystyrene, polypropylene, latex) beads (see e.g., U.S. Pat. Nos. 4,366,241; 4,277,437; 4,275,149; 3,996,345; 3,939,350; 3,850,752; and 3,817,837).

The labeled RNA targets are then hybridized to the microarray. A number of buffers may be used for hybridization assays. By way of example, but not limitation, the buffers can be any of the following: 5 M betaine, 1 M NaCl, pH 7.5; 4.5 M betaine, 0.5 M LiCl, pH 8.0; 3 M TMACl, 50 mM Tris-HCl, 1 mM EDTA, 0.1% N-lauroyl-sarkosine (NLS); 2.4 M TEACl, 50 mM Tris-HCl, pH 8.0, 0.1% NLS; 1 M LiCl, 10 mM Tris-HCl, pH 8.0, 10% formamide; 2 M GuSCN, 30 mM NaCitrate, pH 7.5; 1 M LiCl, 10 mM Tris-HCl, pH 8.0, 1 mM CTAB; 0.3 mM spermine, 10 mM Tris-HCl, pH 7.5; 2 M NH₄OAc with 2 volumes absolute ethanol. Addition volumes of ionic detergents (such as N-lauroyl-sarkosine) may be added to the buffer. Hybridization may be performed at about 20-65° C. (see e.g., U.S. Pat. No. 6,045,996). Additional examples of hybridization conditions are disclosed in SAMBROOK ET AL., (1989); Berger and Kimmel, GUIDE TO MOLECULAR CLONING TECHNIQUES, METHODS IN ENZYMOLOGY, (1987), Volume 152, Academic Press, Inc., San Diego, Calif.; Young and Davis, 80 PROC. NATL. ACAD. SCI. U.S.A 1194 (1983).

The hybridization buffer may be a formamide-based buffer or an aqueous buffer containing dextran sulfate or polyethylene glycol (see e.g., Cheung et al., 21 NATURE GENET. 15-19(1999); SAMBROOK ET AL. (1989)). In addition, the hybridization buffer may contain blocking agents such as sheared salmon sperm DNA or Denhardt's reagent to minimize nonspecific binding or background noise. Approximately 50-200 μg labeled total RNA or 2-5 μg labeled mRNA per hybridization is required for a sufficient fluorescent signal and detection. Typically, the amount of oligonucleotide probes attached to the support is in excess of the labeled target RNA.

Following hybridization, the nucleic acids may be analyzed by detecting one or more labels attached to the target nucleic acids. The labels may be incorporated by any of a number of methods well-known in the art. In one embodiment, the label may be simultaneously incorporated during the amplification step in the preparation of the target nucleic acids. For example, a labeled amplification product may be generated by PCR using labeled primers or labeled nucleotides. Transcription amplification using a labeled nucleotide (e.g., fluorescein-labeled UTP or CTP) incorporates a label into the transcribed nucleic acids. Alternatively, a label may be added directly to the original nucleic acid sample or to the amplification product following amplification. Methods for labeling nucleic acids are well-known in the art and include, for example, nick translation or end-labeling.

The hybridized array is then subjected to laser excitation, which produces an emission with a unique spectra. The spectra are scanned, for example, with a scanning confocal laser microscope generating monochrome images of the microarray. These images are digitally processed and normalized based on a threshold value (e.g., background) using mathematical algorithms. For example, a threshold value of 0 may be assigned when no change in the level of fluorescence is observed; an increase in fluorescence may be assigned a value of +1 and a decrease in fluorescence may be assigned a value of −1. Normalization may be based on a designated subgroup of genes where variations in this subgroup are utilized to generate statistics applicable for evaluating the complete gene microarray. Chen et al., 2 J. BIOMED. OPTICS 364-67 (1997).

Use of one of the protein microarrays of the present invention may involve placing the two-dimensional microarray in a flowchamber with approximately 1-10 μl of fluid volume per 25 mm² overall surface area. The cover over the microarray in the flowchamber is preferably transparent or translucent. In one embodiment, the cover may comprise Pyrex or quartz glass. In other embodiments, the cover may be part of a detection system that monitors interaction between the protein-capture agents immobilized on the microarray and protein in a solution such as a cellular extract from a biological sample. The flowchambers should remain filled with appropriate aqueous solutions to preserve protein activity. Salt, temperature, and other conditions are preferably kept similar to those of normal physiological conditions. Proteins in a fluid solution may be flushed into the flow chamber as desired and their interaction with the immobilized protein-capture agents determined. Sufficient time must be given to allow for binding between the protein-capture agent and its binding partner to occur. The amount of time required for this will vary depending upon the nature and tightness of the affinity of the protein-capture agent for its binding partner. No specialized microfluidic pumps, valves, or mixing techniques are required for fluid delivery to the microarray.

Alternatively, protein-containing fluid may be delivered to each of the regions of protein-capture agents individually. For example, in one embodiment, the regions of the substrate surface where the protein-capture agents reside may be microfabricated in such a way as to allow integration of the microarray with a number of fluid delivery channels oriented perpendicular to the microarray surface, each one of the delivery channels terminating at the site of an individual protein-capture agent-coated region.

The sample, which is delivered to the microarray, will typically be a fluid. In a one embodiment, the sample is a cellular extract or a biological sample. The sample to be assayed may comprise a complex mixture of proteins, including a multitude of proteins which are not binding partners of the protein-capture agents of the microarray. If the proteins to be analyzed in the sample are membrane proteins, then those proteins will typically need to be solubilized prior to administration of the sample to the microarray. If the proteins to be assayed in the sample are proteins secreted by a population of cells in an organism, the sample may be a biological sample. If the proteins to be assayed in the sample are intracellular, a sample may be a cellular extract. In another embodiment, the microarray may comprise protein-capture agents that bind fragments of the expression products of a cell or population of cells in an organism. In such a case, the proteins in the sample to be assayed may have been prepared by performing a digest of the protein in a cellular extract or a biological sample. In an alternative application, the proteins from only specific fractions of a cell are collected for analysis in the sample.

In general, delivery of solutions containing proteins to be bound by the protein-capture agents of the microarray may be preceded, followed, or accompanied by delivery of a blocking solution. A blocking solution contains protein or another moiety that will adhere to sites of non-specific binding on the microarray. For example, solutions of bovine serum albumin or milk may be used as blocking solutions.

The binding partners of the plurality of protein-capture agents on the microarray are proteins that are all expression products, or fragments thereof, of a cell or population of cells of a single organism. The expression products may be proteins, including peptides, of any size or function. They may be intracellular proteins or extracellular proteins. The expression products may be from a one-celled or multicellular organism. The organism may be a plant or an animal. In a specific embodiment of the invention, the binding partners are human expression products, or fragments thereof.

In another embodiment of the present invention, the binding partners of the protein-capture agents of the microarray may be a randomly chosen subset of all the proteins, including peptides, which are expressed by a cell or population of cells in a given organism or a subset of all the fragments of those proteins. Thus, the binding partners of the protein-capture agents of the microarray may represent a wide distribution of different proteins from a single organism.

The binding partners of some or all of the protein-capture agents on the microarray need not necessarily be known. Indeed, the binding partner of a protein-capture agent of the microarray may be a protein or peptide of unknown function. For example, the different protein-capture agents of the microarray may together bind a wide range of cellular proteins from a single cell type, many of which are of unknown identity and/or function.

In another embodiment of the present invention, the binding partners of the protein-capture agents on the microarray are related proteins. The different proteins bound by the protein-capture agents may be members of the same protein family. The different binding partners of the protein-capture agents of the microarray may be either functionally related or simply suspected of being functionally related. The different proteins bound by the protein-capture agents of the microarray may also be proteins that share a similarity in structure or sequence or are simply suspected of sharing a similarity in structure or sequence. For example, the binding partners of the protein-capture agents on the microarray may be growth factor receptors, hormone receptors, neurotransmitter receptors, catecholamine receptors, amino acid derivative receptors, cytokine receptors, extracellular matrix receptors, antibodies, lectins, cytokines, serpins, proteases, kinases, phosphatases, ras-like GTPases, hydrolases, steroid hormone receptors, transcription factors, heat-shock transcription factors, DNA-binding proteins, zinc-finger proteins, leucine-zipper proteins, homeodomain proteins, intracellular signal transduction modulators and effectors, apoptosis-related factors, DNA synthesis factors, DNA repair factors, DNA recombination factors, cell-surface antigens, hepatitis C virus (HCV) proteases or HIV proteases and may correspond to all or part of the proteins encoded by the genes of the gene expression profiles of the present invention.

IV. Control Oligonucleotides and Protein-Capture Agents

Control oligonucleotides corresponding to genomic DNA, housekeeping genes, or negative and positive control genes may also be present on the microarray. Similarly, protein-capture agents that bind housekeeping proteins, or negative and positive control proteins, such as beta actin protein, may also be present on the microarray. These controls are used to calibrate background or basal levels of expression, and to provide other useful information.

Normalization controls may be oligonucleotide probes that are perfectly complementary to labeled reference oligonucleotides that are added to the nucleic acid sample. Normalization controls may be protein-capture agents that bind specifically and consistently to a labeled reference protein that is added to the protein sample. For example, a protein-capture agent/normalization control pair may comprise avidin/streptavidin or a well-known antibody/antigen combination with a known binding coefficient. The signals obtained from the normalization controls after hybridization provide a control for variations in hybridization conditions, label intensity, efficiency, and other factors that may cause the hybridization signal to vary between microarrays. To normalize fluorescence intensity measurements, for example, signals from all probes of the microarray may be divided by the signal from the control probes.

Expression level controls are probes or protein-capture agents that hybridize/bind specifically with constitutively expressed genes in the biological sample and are designed to control the overall metabolic activity of a cell. Analysis of the variations in the levels of the expression control as compared to the expression level of the target nucleic acid or target protein indicates whether variations in the expression level of a gene or protein is due specifically to changes in the transcription rate of that gene or to general variations in the health of the cell. Thus, if the expression levels of both the expression control and the target gene decrease or increase, these alterations may be attributed to changes in the metabolic activity of the cell as a whole, not to differential expression of the target gene or protein in question. If only the expression of the target gene or protein varies, however, then the variation in the expression may be attributed to differences in regulation of that gene or protein and not to overall variations in the metabolic activity of the cell. Constitutively expressed genes such as housekeeping genes (e.g., β-actin gene, transferrin receptor gene, GAPDH gene) may serve as expression level controls.

Mismatch controls may also be used for expression level controls or for normalization controls. These probes and protein-capture agents provide a control for non-specific binding or cross-hybridization to a nucleic acid in the sample other than the target to which the probe is directed. Mismatch controls are oligonucleotide probes identical to the corresponding test or control probes except for the presence of one or more mismatched bases. One or more mismatches (e.g., substituting guanine, cytidine, or thymine for adenine) are selected such that under appropriate hybridization conditions (e.g., stringent conditions), the test or control probe would be expected to hybridize with its target sequence, but the mismatch probe would not hybridize or would hybridize to a significantly lesser extent. Similarly, an antibody may be used as a mismatch control protein-capture agent. For example, an antibody may be used that has a base pair mismatch in the binding domain that affects binding as compared to the normal antibody.

V. Detection Methods and Analysis of Hybridization Results

Methods for signal detection of labeled target nucleic acids hybridized to microarray probes are well-known in the art. For example, a radioactive labeled probe may be detected by radiation emission using photographic film or a gamma counter. For fluorescently labeled target nucleic acids, the localization of the label on the probe microarray may be accomplished with fluorescent microscopy. The hybridized microarray is excited with a light source at the excitation wavelength of the particular fluorescent label and the resulting fluorescence is detected. The excitation light source may be a laser appropriate for the excitation of the fluorescent label.

Confocal microscopy may be automated with a computer-controlled stage to automatically scan the entire microarray. Similarly, a microscope may be equipped with a phototransducer (e.g., a photomultiplier) attached to an automated data acquisition system to automatically record the fluorescence signal produced by hybridization to oligonucleotide probes. See e.g., U.S. Pat. No. 5,143,854.

The present invention also relates to methods for evaluating the hybridization results. These methods may vary with the nature of the specific oligonucleotide probes or protein-capture agent used as well as the controls provided. For example, quantification of the fluorescence intensity for each probe may be accomplished by measuring the probe signal strength at each location (representing a different probe) on the microarray (e.g., detection of the amount of florescence intensity produced by a fixed excitation illumination at each location on the array). The fluorescent intensity for each protein-capture agent and binding pair may be accomplished using similar methods. The absolute intensities of the target nucleic acids or proteins hybridized to the microarray may then be compared with the intensities produced by the controls, providing a measure of the relative expression of the nucleic acids or proteins that hybridize to each of the probes or protein-capture agents.

Normalization of the signal derived from the target nucleic acids to the normalization controls may provide a control for variations in hybridization conditions. Typically, normalization may be accomplished by dividing the measured signal from the other probes or protein-capture agents in the array by the average signal produced by the normalization controls. Normalization may also include correction for variations due to sample preparation and amplification. Such normalization may be accomplished by dividing the measured signal by the average signal from the sample preparation/amplification control probes or protein-capture agents. The resulting values may be multiplied by a constant value to scale the results. Other methods for analyzing microarray data are well-known in the art including coupled two-way clustering analysis, clustering algorithms (hierarchical clustering, self-organizing maps), and support vector machines. See e.g., Brown et al., 97 PROC. NATL. ACAD. SCI. USA 262-67 (2000); Getz et al., 97 PROC. NATL. ACAD. SCI. USA 12079-84 (2000); Holter et al., 97 PROC. NATL. ACAD. SCI. USA 8409-14 (2000); Tamayo et al., 96 PROC. NATL. ACAD. SCI. USA 2907-12 (1999); Eisen et al., 95 PROC. NATL. ACAD. SCI. USA 14863-68 (1998); and Ermolaeva et al, 20 NATURE GENET. 19-23 (1998).

Indeed, the methodologies useful in analyzing gene expression profiles and gene expression data are equally applicable in the context of the study of protein expression. In general, for a variety of applications including proteomics and diagnostics, the methods of the present invention involve the delivery of the sample containing the proteins to be analyzed to the microarrays. After the proteins of the sample have been allowed to interact with and become immobilized on the regions comprising protein-capture agents with the appropriate biological specificity, the presence and/or amount of protein bound at each region is then determined. The detection methods, analysis tools, and algorithms described for the nucleic acid micorarrays are equally applicable in the context of protein microarrays.

In addition to the methods described above, a wide range of detection methods are available to analyze the results of protein microarray experiments. Detection may be quantitative and/or qualitative. The protein microarray may be interfaced with optical detection methods such as absorption in the visible or infrared range, chemoluminescence, and fluorescence (including lifetime, polarization, fluorescence correlation spectroscopy (FCS), and fluorescence-resonance energy transfer (FRET)). Other modes of detection such as those based on optical waveguides (WO 96/26432 and U.S. Pat. No. 5,677,196), surface plasmon resonance, surface charge sensors, and surface force sensors are compatible with many embodiments of the present invention. Alternatively, technologies such as those based on Brewster Angle microscopy (BAM) (Schaafet al., 3 LANGMUIR 1131-1135 (1987)) and ellipsometry (U.S. Pat. Nos. 5,141,311 and 5,116,121; Kim, 22 MACROMOLECULES 2682-2685 (1984)) may be utilized. Quartz crystal microbalances and desorption processes provide still other alternative detection means suitable for at least some embodiments of the invention microarray. See, e.g., U.S. Pat. No. 5,719,060. An example of an optical biosensor system compatible both with some arrays of the present invention and a variety of non-label detection principles including surface plasmon resonance, total internal reflection fluorescence (TIRF), Brewster Angle microscopy, optical waveguide lightmode spectroscopy (OWLS), surface charge measurements, and ellipsometry are discussed in U.S. Pat. No. 5,313,264.

Other different types of detection systems suitable to assay the protein expression arrays of the present invention include, but are not limited to, fluorescence, measurement of electronic effects upon exposure to a compound or analyte, luminescence, ultraviolet visible light, and laser induced fluorescence (LIF) detection methods, collision induced dissociation (CID), mass spectroscopy (MS), CCD cameras, electron and three dimensional microscopy. Other techniques are known to those of skill in the art. For example, analyses of combinatorial arrays and biochip formats have been conducted using LIF techniques that are relatively sensitive. See, e.g., Ideue et al., 337 CHEM. PHYSICS LETTERS 79-84 (2000).

One detection system of particular interest is time-of-flight mass spectrometry (TOF-MS). Using parallel sampling techniques, time-of-flight mass spectrometry may be used for the detailed characterization of hundreds of molecules in a sample mixture at each discreet location within the microarray. Time-of-flight mass spectrometry based systems enable extremely rapid analysis (microseconds to milliseconds instead of seconds for scanning MS devises) high levels of selectivity compared to other techniques with good sensitivity (better than one part per million, as opposed to one part per ten thousand for scanning MS), As a mass spectroscopic technique, time-of-flight mass spectrometry provides molecular weight and structural information for identification of unknown samples.

Additional levels of sensitivity are added by coupling time-of-flight mass spectrometry to another separation system. Thus, in an embodiment, the present invention comprises using ion mobility in combination with time-of-flight mass spectrometry for the analysis of microarrays. The combination of ion mobility and time-of-flight mass spectrometry is referred to as multi-dimensional spectroscopy (MDS). Ions are electrosprayed into the front of the MDS device. Electrospray is a method for ionizing relatively large molecules and having them form a gas phase. The solution containing the sample is sprayed at high voltage, forming charged droplets. These droplets evaporate, leaving the sample's ionized molecules in the gas phase. These ions continue into the ion mobility chamber where the ions travel under the influence of a uniform electric field through a buffer gas. The principle underlying ion mobility separation techniques is that compact ions undergo fewer collisions than ions having extended shapes and thus, have increased mobility. As the separated components (comprising ions/molecules of different mobility) exit the drift tube, they are pulsed into a time-of-flight mass spectrometer.

Although non-label detection methods are generally preferred, some of the types of detection methods commonly used for traditional immunoassays that require the use of labels may be applied to the arrays of the present invention. These techniques include noncompetitive immunoassays, competitive immunoassays, and dual label, radiometric immunoassays. These techniques are primarily suitable for use with the arrays of protein-capture agents when the number of different protein-capture agents with different specificity is small (less than about 100). In the competitive method, binding-site occupancy is determined indirectly. In this method, the protein-capture agents of the microarray are exposed to a labeled developing agent, which is typically a labeled version of the analyte or an analyte analog. The developing agent competes for the binding sites on the protein-capture agent with the analyte. The fractional occupancy of the protein-capture agents on different regions can be determined by the binding of the developing agent to the protein-capture agents of the individual regions.

In the noncompetitive method, binding site occupancy is determined directly. In this method, the regions of the microarray are exposed to a labeled developing agent capable of binding to either the bound analyte or the occupied binding sites on the protein-capture agent. For example, the developing agent may be a labeled antibody directed against occupied sites (i.e., a “sandwich assay”). Alternatively, a dual label, radiometric, approach may be taken where the protein-capture agent is labeled with one label and the second, developing agent is labeled with a second label. See Ekins, et al., 194 CLINICA CHIMICA ACTA. 91-114, (1990). Many different labeling methods may be used in the aforementioned techniques, including radioisotopic, enzymatic, chemiluminescent, and fluorescent methods.

VI. Types Of Microarrays

The microarrays of the present invention may be derived from or representative of a specific organism, or cell type, including human microarrays, cancer microarrays, apoptosis microarrays, oncogene and tumor suppressor microarrays, cell-cell interaction microarrays, cytokine and cytokine receptor microarrays, blood microarrays, cell cycle microarrays, neuroarrays, mouse microarrays, and rat microarrays, or combinations thereof.

In further embodiments, the microarrays may represent diseases including cardiovascular diseases, neurological diseases, immunological diseases, various cancers, infectious diseases, endocrine disorders, and genetic diseases.

Alternatively, the microarrays of the present invention may represent a particular tissue type, such as heart, liver, prostate, lung, nerve, muscle, or connective tissue; preferably coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesangial cells, coronary artery smooth muscle, bronchial smooth muscle, uterine smooth muscle, lung fibroblast, osteoblasts, prostate stromal cells, or combinations thereof.

The present invention contemplates microarrays comprising a gene expression profile comprising one or more nucleic acid sequences including complementary and homologous sequences, wherein said gene expression profile is generated from a cell type selected from the group comprising coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesangial cells, coronary artery smooth muscle, bronchial smooth muscle, uterine smooth muscle, lung fibroblast, osteoblasts, and prostate stromal cells.

The present invention contemplates microarrays comprising one or more protein-capture agents, wherein said protein expression profile is generated from a cell type selected from the group comprising coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesangial cells, coronary artery smooth muscle, bronchial smooth muscle, uterine smooth muscle, lung fibroblast, osteoblasts, and prostate stromal cells.

In a specific embodiment, the present invention provides a microarray comprising an endothelial cell gene expression profile comprising one or more nucleic acid sequences substantially homlogous to a nucleic acid sequence or complementary sequence thereof, or portions of said nucleic acid sequence or complementary sequence thereof, selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 48; SEQ ID NO: 63; SEQ ID NO: 70; SEQ ID NO: 82; SEQ ID NO: 94; and SEQ ID NO: 144.

In another embodiment, a microarray of the present invention may comprise a muscle cell gene expression profile comprising one or more nucleic acid sequences substantially homlogous to a nucleic acid sequence or complementary sequence thereof, or portions of said nucleic acid sequence or complementary sequence thereof, selected from the group consisting of SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 39; SEQ ID NO: 40; SEQ ID NO: 41; SEQ ID NO: 42; SEQ ID NO: 54; SEQ ID NO: 55; and SEQ ID NO: 69.

In an alternative embodiment, a microarray comprises a primary cell gene expression profile comprising one or more nucleic acid sequences substantially homlogous to a nucleic acid sequence or complementary sequence thereof, or portions of said nucleic acid sequence or complementary sequence thereof, selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO: 18; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186.

The present invention also provides a microarray comprising an epithelial cell gene expression profile comprising one or more nucleic acid sequences substantially homlogous to a nucleic acid sequence or complementary sequence thereof, or portions of said nucleic acid sequence or complementary sequence thereof, selected from the group consisting of SEQ ID NO: 47; SEQ ID NO: 60; SEQ ID NO: 67; SEQ ID NO: 73; SEQ ID NO: 75; SEQ ID NO: 76; SEQ ID NO: 77; SEQ ID NO: 78; SEQ ID NO: 80; SEQ ID NO: 96; SEQ ID NO: 98; SEQ ID NO: 99; SEQ ID NO: 111; SEQ ID NO: 112; SEQ ID NO: 123; SEQ ID NO: 127; SEQ ID NO: 131; SEQ ID NO: 150; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186.

In yet another embodiment, a microarray may comprise a keratinocyte epithelial cell gene expression profile comprising one or more nucleic acid sequences substantially homlogous to a nucleic acid sequence or complementary sequence thereof, or portions of said nucleic acid sequence or complementary sequence thereof, selected from the group consisting of SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; and SEQ ID NO: 211.

The present invention also provides a microarray comprising a mammary epithelial cell gene expression profile comprising one or more nucleic acid sequences substantially homlogous to a nucleic acid sequence or complementary sequence thereof, or portions of said nucleic acid sequence or complementary sequence thereof, selected from the group consisting of SEQ ID NO: 78; SEQ ID NO: 212; SEQ ID NO: 213; SEQ ID NO: 216; SEQ TD NO: 225; SEQ ID NO: 226; SEQ ID NO: 227; SEQ ID NO: 239; SEQ ID NO: 271; SEQ ID NO: 285; and SEQ ID NO: 289.

In an alternative embodiment, a microarray may comprise a bronchial epithelial cell gene expression profile comprising one or more nucleic acid sequences substantially homlogous to a nucleic acid sequence or complementary sequence thereof, or portions of said nucleic acid sequence or complementary sequence thereof, selected from the group consisting of SEQ ID NO: 27; SEQ ID NO: 131; SEQ ID NO: 150; SEQ ID NO: 169; SEQ ID NO: 214; SEQ ID NO: 215; SEQ ID NO: 223; SEQ ID NO: 224; SEQ ID NO: 241; SEQ ID NO: 243; SEQ ID NO: 244; SEQ ID NO: 255; SEQ ID NO: 256; SEQ ID NO: 261; and SEQ ID NO: 314.

The present invention also provides a microarray comprising a prostate epithelial cell gene expression profile comprising one or more nucleic acid sequences substantially homlogous to a nucleic acid sequence or complementary sequence thereof, or portions of said nucleic acid sequence or complementary sequence thereof, selected from the group consisting of SEQ ID NO: 64; SEQ ID NO: 217; SEQ ID NO: 218; SEQ ID NO: 259; SEQ ID NO: 293; SEQ ID NO: 302; and SEQ ID NO: 320.

In yet another embodiment, a microarray comprises a renal cortical epithelial cell gene expression profile comprising one or more nucleic acid sequences substantially homlogous to a nucleic acid sequence or complementary sequence thereof, or portions of said nucleic acid sequence or complementary sequence thereof, selected from the group consisting of SEQ ID NO: 49; SEQ ID NO: 57; SEQ ID NO: 104; SEQ ID NO: 123; SEQ ID NO: 160; SEQ ID NO: 165; SEQ ID NO: 166; SEQ ID NO: 219; SEQ ID NO: 267; SEQ ID NO: 270; SEQ ID NO: 279; SEQ ID NO: 280; SEQ ID NO: 283; SEQ ID NO: 291; SEQ ID NO: 305; SEQ ID NO: 307; SEQ ID NO: 310; SEQ ID NO: 313; SEQ ID NO: 325; SEQ ID NO: 326; and SEQ ID NO: 327.

The present invention further provides a microarray comprising one or more nucleic acid sequences substantially homlogous to a nucleic acid sequence or complementary sequence thereof, or portions of said nucleic acid sequence or complementary sequence thereof, selected from the group consisting of SEQ ID NO: 106; SEQ ID NO: 138; SEQ ID NO: 158; SEQ ID NO: 228; SEQ ID NO: 236; SEQ ID NO: 242; SEQ ID NO: 250; SEQ ID NO: 258; SEQ ID NO: 260; SEQ ID NO: 262; SEQ ID NO: 266; SEQ ID NO: 272; SEQ ID NO: 273; SEQ ID NO: 274; SEQ ID NO: 275; SEQ ID NO: 276; SEQ ID NO: 278; SEQ ID NO: 284; SEQ ID NO: 288; SEQ ID NO: 295; SEQ ID NO: 296; SEQ ID NO: 297; SEQ ID NO: 299; SEQ ID NO: 300; SEQ ID NO: 301; SEQ ID NO: 306; SEQ ID NO: 308; SEQ ID NO: 309; SEQ ID NO: 311; SEQ ID NO: 316; SEQ ID NO: 318; SEQ ID NO: 321; SEQ ID NO: 322; SEQ ID NO: 328; and SEQ ID NO: 329.

In a specific embodiment, a microarray may comprise a small airway epithelial cell gene expression profile comprising one or more nucleic acid sequences substantially homlogous to a nucleic acid sequence or complementary sequence thereof, or portions of said nucleic acid sequence or complementary sequence thereof, selected from the group consisting of SEQ ID NO: 173; SEQ ID NO: 174; SEQ ID NO: 183; SEQ ID NO: 220; SEQ ID NO: 221; SEQ ID NO: 222; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 237; SEQ ID NO: 238; SEQ ID NO: 240; SEQ ID NO: 245; SEQ ID NO: 246; SEQ ID NO: 247; SEQ ID NO: 248; SEQ ID NO: 249; SEQ ID NO: 251; SEQ ID NO: 252; SEQ ID NO: 254; SEQ ID NO: 257; SEQ ID NO: 263; SEQ ID NO: 264; SEQ ID NO: 265; SEQ ID NO: 268; SEQ ID NO: 269; SEQ ID NO: 270; SEQ ID NO: 277; SEQ ID NO: 281; SEQ ID NO: 282; SEQ ID NO: 286; SEQ ID NO: 287; SEQ ID NO: 290; SEQ ID NO: 294; SEQ ID NO: 298; SEQ ID NO: 303; SEQ ID NO: 312; SEQ ID NO: 315; SEQ ID NO: 317; and SEQ ID NO: 319.

The present invention also provides a microarray comprising one or more nucleic acid sequences substantially homlogous to a nucleic acid sequence or complementary sequence thereof, or portions of said nucleic acid sequence or complementary sequence thereof, selected from the group consisting of SEQ ID NO: 37; SEQ ID NO: 253; SEQ ID NO: 304; SEQ ID NO: 323; and SEQ ID NO: 324.

In yet another embodiment, a microarray may comprise one or more nucleic acid sequences substantially homlogous to a nucleic acid sequence or complementary sequence thereof, or portions of said nucleic acid sequence or complementary sequence thereof, selected from the group consisting of SEQ ID NO: 27; SEQ ID NO: 37; SEQ ID NO: 49; SEQ ID NO: 57; SEQ ID NO: 64; SEQ ID NO: 70; SEQ ID NO: 78; SEQ ID NO: 104; SEQ ID NO: 106; SEQ ID NO: 123; SEQ ID NO: 131; SEQ ID NO: 138; SEQ ID NO: 150; SEQ ID NO: 158; SEQ ID NO: 160; SEQ ID NO: 165; SEQ ID NO: 166; SEQ ID NO: 169; SEQ ID NO: 173; SEQ ID NO: 174; SEQ ID NO: 183; SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; SEQ ID NO: 211; SEQ ID NO: 212; SEQ ID NO: 213; SEQ ID NO: 214; SEQ ID NO: 215; SEQ ID NO: 216; SEQ ID NO: 217; SEQ ID NO: 218; SEQ ID NO: 219; SEQ ID NO: 220; SEQ ID NO: 221; SEQ ID NO: 222; SEQ ID NO: 223; SEQ ID NO: 224; SEQ ID NO: 225; SEQ ID NO: 226; SEQ ID NO: 227; SEQ ID NO: 228; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 236; SEQ ID NO: 237; SEQ ID NO: 238; SEQ ID NO: 239; SEQ ID NO: 240; SEQ ID NO: 241; SEQ ID NO: 242; SEQ ID NO: 243; SEQ ID NO: 244; SEQ ID NO: 245; SEQ ID NO: 246; SEQ ID NO: 247; SEQ ID NO: 248; SEQ ID NO: 249; SEQ ID NO: 250; SEQ ID NO: 251; SEQ ID NO: 252; SEQ ID NO: 253; SEQ ID NO: 254; SEQ ID NO: 255; SEQ ID NO: 256; SEQ ID NO: 257; SEQ ID NO: 258; SEQ ID NO: 259; SEQ ID NO: 260; SEQ ID NO: 261; SEQ ID NO: 262; SEQ ID NO: 263; SEQ ID NO: 264; SEQ ID NO: 265; SEQ ID NO: 266; SEQ ID NO: 267; SEQ ID NO: 268; SEQ ID NO: 269; SEQ ID NO: 270; SEQ ID NO: 271; SEQ ID NO: 272; SEQ ID NO: 273; SEQ ID NO: 274; SEQ ID NO: 275; SEQ ID NO: 276; SEQ ID NO: 277; SEQ ID NO: 278; SEQ ID NO: 279; SEQ ID NO: 280; SEQ ID NO: 281; SEQ ID NO: 282; SEQ ID NO: 283; SEQ ID NO: 284; SEQ ID NO: 285; SEQ ID NO: 286; SEQ ID NO: 287; SEQ ID NO: 288; SEQ ID NO: 289; SEQ ID NO: 290; SEQ ID NO: 291; SEQ ID NO: 293; SEQ ID NO: 294; SEQ ID NO: 295; SEQ ID NO: 296; SEQ ID NO: 297; SEQ ID NO: 298; SEQ ID NO: 299; SEQ ID NO: 300; SEQ ID NO: 301; SEQ ID NO: 302; SEQ ID NO: 303; SEQ ID NO: 304; SEQ ID NO: 305; SEQ ID NO: 306; SEQ ID NO: 307; SEQ ID NO: 308; SEQ ID NO: 309; SEQ ID NO: 310; SEQ ID NO: 311; SEQ ID NO: 312; SEQ ID NO: 313; SEQ ID NO: 314; SEQ ID NO: 315; SEQ ID NO: 316; SEQ ID NO: 317; SEQ ID NO: 318; SEQ ID NO: 320; SEQ ID NO: 321; SEQ ID NO: 322; SEQ ID NO: 323; SEQ ID NO: 324; SEQ ID NO: 325; SEQ ID NO: 326; SEQ ID NO: 327; SEQ ID NO: 328; and SEQ ID NO: 329.

In a specific embodiment, the present invention provides a microarray comprising one or more protein-capture agents that bind one or more amino acid sequences encoded by all or a portion of one or more nucleic acid sequences selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 48; SEQ ID NO: 63; SEQ ID NO: 70; SEQ ID NO: 82; SEQ ID NO: 94; and SEQ ID NO: 144.

In another embodiment, a microarray may comprise one or more protein-capture agents that bind one or more amino acid sequences encoded by all or a portion of one or more nucleic acid sequences selected from the group consisting of SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 39; SEQ ID NO: 40; SEQ ID NO: 41; SEQ ID NO: 42; SEQ ID NO: 54; SEQ ID NO: 55; and SEQ ID NO: 69.

In an alternative embodiment, a microarray comprises one or more protein-capture agents that bind one or more amino acid sequences encoded by all or a portion of one or more nucleic acid sequences selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO:. 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186.

The present invention also provides a microarray comprising one or more protein-capture agents that bind one or more amino acid sequences encoded by all or a portion of one or more nucleic acid sequences selected from the group consisting of SEQ ID NO: 47; SEQ ID NO: 60; SEQ ID NO: 67; SEQ ID NO: 73; SEQ ID NO: 75; SEQ ID NO: 76; SEQ ID NO: 77; SEQ ID NO: 78; SEQ ID NO: 80; SEQ ID NO: 96; SEQ ID NO: 98; SEQ ID NO: 99; SEQ ID NO: 111; SEQ ID NO: 112; SEQ ID NO: 123; SEQ ID NO: 127; SEQ ID NO: 131; SEQ ID NO: 150; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186.

In yet another embodiment, a microarray may comprise one or more protein-capture agents that bind one or more amino acid sequences encoded by all or a portion of one or more nucleic acid sequences selected from the group consisting of SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; and SEQ ID NO: 211.

The present invention also provides a microarray comprising one or more protein-capture agents that bind one or more amino acid sequences encoded by all or a portion of one or more nucleic acid sequences selected from the group consisting of SEQ ID NO: 78; SEQ ID NO: 212; SEQ ID NO: 213; SEQ ID NO: 216; SEQ ID NO: 225; SEQ ID NO: 226; SEQ ID NO: 227; SEQ ID NO: 239; SEQ ID NO: 271; SEQ ID NO: 285; and SEQ ID NO: 289.

In an alternative embodiment, a microarray may comprise one or more protein-capture agents that bind one or more amino acid sequences encoded by all or a portion of one or more nucleic acid sequences selected from the group consisting of SEQ ID NO: 27; SEQ ID NO: 131; SEQ ID NO: 150; SEQ ID NO: 169; SEQ ID NO: 214; SEQ ID NO: 215; SEQ ID NO: 223; SEQ ID NO: 224; SEQ ID NO: 241; SEQ ID NO: 243; SEQ ID NO: 244; SEQ ID NO: 255; SEQ ID NO: 256; SEQ ID NO: 261; and SEQ ID NO: 314.

The present invention also provides a microarray comprising one or more protein-capture agents that bind one or more amino acid sequences encoded by all or a portion of one or more nucleic acid sequences selected from the group consisting of SEQ ID NO: 64; SEQ ID NO: 217; SEQ ID NO: 218; SEQ ID NO: 259; SEQ ID NO: 293; SEQ ID NO: 302; and SEQ ID NO: 320.

In yet another embodiment, a microarray comprises one or more protein-capture agents that bind one or more amino acid sequences encoded by all or a portion of one or more nucleic acid sequences selected from the group consisting of SEQ ID NO: 49; SEQ ID NO: 57; SEQ ID NO: 104; SEQ ID NO: 123; SEQ ID NO: 160; SEQ ID NO: 165; SEQ ID NO: 166; SEQ ID NO: 219; SEQ ID NO: 267; SEQ ID NO: 270; SEQ ID NO: 279; SEQ ID NO: 280; SEQ ID NO: 283; SEQ ID NO: 291; SEQ ID NO: 305; SEQ ID NO: 307; SEQ ID NO: 310; SEQ ID NO: 313; SEQ ID NO: 325; SEQ ID NO: 326; and SEQ ID NO: 327.

The present invention further provides a microarray comprising one or more protein-capture agents that bind one or more amino acid sequences encoded by all or a portion of one or more nucleic acid sequences selected from the group consisting of SEQ ID NO: 106; SEQ ID NO: 138; SEQ ID NO: 158; SEQ ID NO: 228; SEQ ID NO: 236; SEQ ID NO: 242; SEQ ID NO: 250; SEQ ID NO: 258; SEQ ID NO: 260; SEQ ID NO: 262; SEQ ID NO: 266; SEQ ID NO: 272; SEQ ID NO: 273; SEQ ID NO: 274; SEQ ID NO: 275; SEQ ID NO: 276; SEQ ID NO: 278; SEQ ID NO: 284; SEQ ID NO: 288; SEQ ID NO: 295; SEQ ID NO: 296; SEQ ID NO: 297; SEQ ID NO: 299; SEQ ID NO: 300; SEQ ID NO: 301; SEQ ID NO: 306; SEQ ID NO: 308; SEQ ID NO: 309; SEQ ID NO: 311; SEQ ID NO: 316; SEQ ID NO: 318; SEQ ID NO: 321; SEQ ID NO: 322; SEQ ID NO: 328; and SEQ ID NO: 329.

In a specific embodiment, a microarray may comprise one or more protein-capture agents that bind one or more amino acid sequences encoded by all or a portion of one or more nucleic acid sequences selected from the group consisting of SEQ ID NO: 173; SEQ ID NO: 174; SEQ ID NO: 183; SEQ ID NO: 220; SEQ ID NO: 221; SEQ ID NO: 222; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 237; SEQ ID NO: 238; SEQ ID NO: 240; SEQ ID NO: 245; SEQ ID NO: 246; SEQ ID NO: 247; SEQ ID NO: 248; SEQ ID NO: 249; SEQ ID NO: 251; SEQ ID NO: 252; SEQ ID NO: 254; SEQ ID NO: 257; SEQ ID NO: 263; SEQ ID NO: 264; SEQ ID NO: 265; SEQ ID NO: 268; SEQ ID NO: 269; SEQ ID NO: 270; SEQ ID NO: 277; SEQ ID NO: 281; SEQ ID NO: 282; SEQ ID NO: 286; SEQ ID NO: 287; SEQ ID NO: 290; SEQ ID NO: 294; SEQ ID NO: 298; SEQ ID NO: 303; SEQ ID NO: 312; SEQ ID NO: 315; SEQ ID NO: 317; and SEQ ID NO: 319.

The present invention also provides a microarray comprising one or more protein-capture agents that bind one or more amino acid sequences encoded by all or a portion of one or more nucleic acid sequences selected from the group consisting of SEQ ID NO: 37; SEQ ID NO: 253; SEQ ID NO: 304; SEQ ID NO: 323; and SEQ ID NO: 324.

In yet another embodiment, a microarray may comprise one or more protein-capture agents that substantially bind one or more amino acid sequences encoded by all or a portion of one or more nucleic acid sequences selected from the group consisting of SEQ ID NO: 27; SEQ ID NO: 37; SEQ ID NO: 49; SEQ ID NO: 57; SEQ ID NO: 64; SEQ ID NO: 70; SEQ ID NO: 78; SEQ ID NO: 104; SEQ ID NO: 106; SEQ ID NO: 123; SEQ ID NO: 131; SEQ ID NO: 138; SEQ ID NO: 150; SEQ ID NO: 158; SEQ ID NO: 160; SEQ ID NO: 165; SEQ ID NO: 166; SEQ ID NO: 169; SEQ ID NO: 173; SEQ ID NO: 174; SEQ ID NO: 183; SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; SEQ ID NO: 211; SEQ ID NO: 212; SEQ ID NO: 213; SEQ ID NO: 214; SEQ ID NO: 215; SEQ ID NO: 216; SEQ ID NO: 217; SEQ ID NO: 218; SEQ ID NO: 219; SEQ ID NO: 220; SEQ ID NO: 221; SEQ ID NO: 222; SEQ ID NO: 223; SEQ ID NO: 224; SEQ ID NO: 225; SEQ ID NO: 226; SEQ ID NO: 227; SEQ ID NO: 228; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 236; SEQ ID NO: 237; SEQ ID NO: 238; SEQ ID NO: 239; SEQ ID NO: 240; SEQ ID NO: 241; SEQ ID NO: 242; SEQ ID NO: 243; SEQ ID NO: 244; SEQ ID NO: 245; SEQ ID NO: 246; SEQ ID NO: 247; SEQ ID NO: 248; SEQ ID NO: 249; SEQ ID NO: 250; SEQ ID NO: 251; SEQ ID NO: 252; SEQ ID NO: 253; SEQ ID NO: 254; SEQ ID NO: 255; SEQ ID NO: 256; SEQ ID NO: 257; SEQ ID NO: 258; SEQ ID NO: 259; SEQ ID NO: 260; SEQ ID NO: 261; SEQ ID NO: 262; SEQ ID NO: 263; SEQ ID NO: 264; SEQ ID NO: 265; SEQ ID NO: 266; SEQ ID NO: 267; SEQ ID NO: 268; SEQ ID NO: 269; SEQ ID NO: 270; SEQ ID NO: 271; SEQ ID NO: 272; SEQ ID NO: 273; SEQ ID NO: 274; SEQ ID NO: 275; SEQ ID NO: 276; SEQ ID NO: 277; SEQ ID NO: 278; SEQ ID NO: 279; SEQ ID NO: 280; SEQ ID NO: 281; SEQ ID NO: 282; SEQ ID NO: 283; SEQ ID NO: 284; SEQ ID NO: 285; SEQ ID NO: 286; SEQ ID NO: 287; SEQ ID NO: 288; SEQ ID NO: 289; SEQ ID NO: 290; SEQ ID NO: 291; SEQ ID NO: 293; SEQ ID NO: 294; SEQ ID NO: 295; SEQ ID NO: 296; SEQ ID NO: 297; SEQ ID NO: 298; SEQ ID NO: 299; SEQ ID NO: 300; SEQ ID NO: 301; SEQ ID NO: 302; SEQ ID NO: 303; SEQ ID NO: 304; SEQ ID NO: 305; SEQ ID NO: 306; SEQ ID NO: 307; SEQ ID NO: 308; SEQ ID NO: 309; SEQ ID NO: 310; SEQ ID NO: 311; SEQ ID NO: 312; SEQ ID NO: 313; SEQ ID NO: 314; SEQ ID NO: 315; SEQ ID NO: 316; SEQ ID NO: 317; SEQ ID NO: 318; SEQ ID NO: 320; SEQ ID NO: 321; SEQ ID NO: 322; SEQ ID NO: 323; SEQ ID NO: 324; SEQ ID NO: 325; SEQ ID NO: 326; SEQ ID NO: 327; SEQ ID NO: 328; and SEQ ID NO: 329

VII. Expression Profiles and Microarray Methods of Use

In one aspect, the present invention provides methods for the reproducible measurement and assessment of the expression of specific mRNAs or proteins in a specific set of cells. One method combines and utilizes the techniques of laser capture microdissection, T7-based RNA amplification, production of cDNA from amplified RNA, and DNA microarrays containing immobilized DNA molecules for a wide variety of specific genes to produce a profile of gene expression analysis for very small numbers of specific cells. The desired cells are individually identified and attached to a substrate by the laser capture technique, and the captured cells are then separated from the remaining cells. RNA is then extracted from the captured cells and amplified about one million-fold using the T7-based amplification technique, and cDNA may be prepared from the amplified RNA. A wide variety of specific DNA molecules are prepared that hybridize with specific nucleic acids of the microarray, and the DNA molecules are immobilized on a suitable substrate. The cDNA made from the captured cells is applied to the microarray under conditions that allow hybridization of the cDNA to the immobilized DNA on the array. The expression profile of the captured cells is obtained from the analysis of the hybridization results using the amplified RNA or cDNA made from the amplified RNA of the captured cells, and the specific immobilized DNA molecules on the microarray. The hybridization results demonstrate, for example, which genes of those represented on the microarray as probes are hybridized to cDNA from the captured cells, and/or the amount of specific gene expression. The hybridization results represent the gene expression profile of the captured cells. The gene expression profile of the captured cells can be used to compare the gene expression profile of a different set of captured cells. The similarities and differences provide useful information for determining the differences in gene expression between different cell types, and differences between the same cell type under different conditions.

The techniques used for gene expression analysis are likewise applicable in the context of protein expression profiles. Total protein may be isolated from a cell sample and hybridized to a microarray comprising a plurality of protein-capture agents, which may include antibodies, receptor proteins, small molecules, and the like. Using any of several assays known in the art, hybridization may be detected and analyzed as described above. In the case of fluorescent detection, algorithms may be used to extract a protein expression profile representative of the particular cell type.

The present invention further relates to gene expression profiles and protein expression profiles that define a particular cell or tissue, or a particular cell or tissue state, e.g. a normal or diseased state. Such “cell type specific gene expression profiles” comprise genes that are only expressed in a particular cell, i.e., are differentially expressed between cells. Similarly, cell type specific protein expression profiles comprise proteins that are only expressed in a particular cell, i.e., are differentially expressed between cells. A cell type specific expression profile may define a particular cell type including its origin within the body and cellular state. For example, a cell type gene or protein expression profile may define an epithelial cell and more particularly, an epithelial cell located in a specific tissue, an epithelial cell at a specific stage of the cell cycle, an epithelial cell in a specific state of differentiation, an epithelial cell in an activated state, and/or an epithelial cell in a particular diseased state. Thus, the methodologies, microarrays, and algorithms of the present invention may be used to determine the phenotype of an unknown cell sample.

Moreover, all of the cell type specific gene and/or protein expression profiles may be compiled together in a database to be used for a variety of applications. For example, the profiles and the database may be used in methods for approximating cell type and cell number of a mixed population of cells. Armed with a database of cell type specific gene and/or protein expression profiles, a gene or protein expression profile constructed from a mixed population of cells may be compared against the profile database. Using the alogrithms of the present invention, a user may identify the number and type of cells comprising the mixed population.

In addition, the profiles and database may be used in creating cell type specific gene or protein microarrays. A microarray may be produced that comprises genes or protein-capture agents that represent all cell types or a specific set of cell types, for example, normal colon cells and cancerous colon cells at different stages of disease progression.

The gene expression profiles, protein expression profiles, microarrays, and algorithms of the present invention may also be used to differentiate cell types (e.g., neuron v. muscle cell). For example, mRNA isolated from two different cells may be hybridized to a microarray. The mRNA derived from each of the two cell types may be labeled with different fluorophores so that they may be distinguished. See e.g., Hacia et al., 26 NUCLEIC ACID RES. 3865-66, (1998); Schena et al., 270 SCIENCE 467-70 (1995). For example, mRNA from skeletal muscle cells may be synthesized using a fluorescein-12-UTP, and mRNA from neuronal cells, may be synthesized using biotin-16-UTP. The two mRNAs are then mixed and hybridized to the microarray. The mRNA from skeletal muscle cells will, for example, fluoresce green when the fluorophore is stimulated and the mRNA from neuronal cells will, for example, fluoresce red. The relative signal intensity from each mRNA is determined, and an expression profile for each mRNA is generated and used to identify the cell type. An advantage of using mRNA labeled with two different fluorophores is that a direct and internally controlled comparison of the mRNA levels corresponding to each arrayed gene in the two cell types can be made, and variations due to minor differences in experimental conditions (e.g., hybridization conditions) will not affect subsequent analyses.

In one aspect, the present invention provides gene and protein expression profile useful for identifying specific cell types. For example, the present invention contemplates gene and protein expression profiles generated from numerous cell types including, but not limited to, coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesangial cells, coronary artery smooth muscle, bronchial smooth muscle, uterine smooth muscle, lung fibroblast, osteoblasts, and prostate stromal cells.

Furthermore, the expression profiles and microarrays of the present invention may be used to distinguish normal tissue from diseased tissue, and in particular normal tissue from tumorgenic tissue. In addition, the present invention may also be used for patient diagnosis. Specifically, a patient sample may be hybridized to a microarray representing normal and diseased tissues. The resulting expression pattern of the patient sample may then be compared to the expression profile of a normal tissue sample to determine the disease progression status. For example, alterations in the level of expression of the prostrate-specific antigen (PSA) may be indicative of prostrate cancer and variations of the carcino-embryonic antigen (CEA) may be indicative of colon cancer.

The present invention also relates to methods of using the expression profiles and microarrays. For example, the gene expression profiles and protein expression profiles and microarrays may be used for drug and toxicity screening. Drugs often have side effects that are, in part, due to the lack of target specificity. In vitro assays provide limited information on the specificity of a compound. In contrast, a microarray may reveal the spectrum of genes or proteins affected by a particular drug compound. In considering two different compounds both of which demonstrate specificity for a target protein (e.g., a receptor), if one compound affects the expression of ten genes or proteins and a second compound affects the expression of fifty genes or proteins, the first compound is more likely to have fewer side effects. Because the identity of the genes or proteins is known or determinable, information on other affected genes is informative as to the nature of the side effects. A panel of genes or proteins may be used to test derivatives of a lead compound to determine which of the derivatives have greater specificity than the first compound.

Thus, microarray technology may be used to identify drug compounds that regulate gene and/or protein expression or possess similar mechanisms of action. This technology may also be used to create microarrays that model various diseases and in turn, novel drug compounds may be analyzed as potential therapeutics. In addition, microarrays may be generated that comprise the genes or proteins of one or more of a particular pathogen (e.g., bacteria, viruses, fungi). These microarrays may then be utilized to identify promising antibiotics, antiviral, or antifungal agents.

In another embodiment of the invention, a microarray corresponding to a population of genes or proteins isolated from a particular tissue or cell type is used to detect changes in gene transcription or protein expression which result from exposing the selected tissue or cells to a candidate drug. In this embodiment, tissue or cells derived from an organism, or an established cell line, may be exposed to the candidate drug in vivo or ex vivo. Thereafter, the gene transcripts, primarily mRNA, of the tissue or cells are isolated by methods well-known in the art. See, e.g., SAMBROOK ET AL. (1989). The isolated transcripts or cDNAs complementary to the mRNA are then contacted with a microarray, each microarray probe being specific for a different transcript, under conditions where the transcripts hybridize with a corresponding probe to form hybridization pairs. Similarly, protein may be isolated by methods well-known in the art. The isolated protein sample is then hybridized to a microarray comprising a plurality of protein-capture agents. The microarrays may provide, in aggregate, an ensemble of genes or proteins of the tissue or cell type sufficient to model the transcriptional and/or translational responsiveness of a drug candidate. A hybridization signal may then be detected at each hybridization pair to obtain an expression profile. This profile of the drug-stimulated cells may then be compared with anexpression profile of control cells to obtain a specific drug response profile.

Similarly, for toxicity screening, a cell line or animal (e.g., rat) may be treated with a particular toxin (e.g., carcinogen, immunotoxin, cytotoxin, teratogen, pesticide) to determine its effects on gene expression. As described above, RNA or protein may be isolated from the treated cell line or a tissue (e.g., liver) from the treated animal, and hybridized to a microarray containing oligonucleotide probes or protein-capture agents. The resulting expression profiles may be compared to profiles generated from an untreated animal or cell line. An analysis of the expression pattern of the treated samples may reflect the effects of the particular toxin on gene expression, and possibly predict physiological effects.

This data may be used to identify genetic response profiles. Individual gene or protein responses may be sorted to determine the specificity of each gene or protein to a particular stimulus. An expression profile may be established which weighs the signal patterns proportionally to the specificity of the response. Response profiles for an unknown stimulus (e.g., new chemicals, unknown compounds) may be analyzed by comparing the new stimulus response profiles with response profiles to known chemical stimuli. If there is a gene or protein match, then the response profile identifies a stimulus with the same target as one of the known compounds upon which the response profile database is based. For drug screening, if the response profile is a subset of cells in the support stimulated by a known compound, the new compound may be a candidate for a molecule with greater specificity than the reference compound.

Gene and/or protein expression profiles and microarrays may also be used to identify activating or non-activating compounds. Compounds that increase transcription rates or stimulate the activity of a protein are considered activating, and compounds that decrease rates or inhibit the activity of a protein are non-activating. The biological effects of a compound may be reflected in the biological state of a cell. This state is characterized by the cellular constituents. One aspect of the biological state of a cell is its transcriptional state. The transcriptional state of a cell includes the identities and amounts of the constituent RNA species, especially mRNAs, in the cell under a given set of conditions. Thus, the gene expression profiles, microarrays, and algorithms of the present invention may be used to analyze and characterize the transcriptional state of a given cell or tissue following exposure to an activating or non-activating compound.

The gene expression profiles, microarrays, and algorithms of the present invention may also be used to identify the components of cell signaling pathways. A cell signaling pathway is generally understood to be a collection of the cellular constituents (e.g., DNA, RNA, receptors, second messenger proteins, enzymes). The cellular constituents of a particular signaling pathway may be identified, for example, by variations in the transcription or translation rates. Each cellular constituent is typically influenced by at least one other cellular constituent. Thus, a cell may be exposed to a compound that interacts with a specific cellular constituent. For example, the cell may be exposed to varying concentrations of a specific receptor agonist. An analysis of variations in gene and/or protein expression as compared to an unexposed cell may reveal components of that particular receptor-signaling pathway. Thus, the cellular constituents that vary in a correlated pattern as the concentrations of the drug are increased may be identified as a component of the pathway originating at that drug.

The present invention may also be used to identify co-regulated genes. Similar variations in the transcriptional rate of a particular group of genes may reflect that these genes are similarly regulated. Thus, analysis of the transcriptional state of these genes may be accomplished by hybridization to microarrays. The level of hybridization to the microarray reflects the prevalence of the mRNA transcripts in the cell and may be used to determine if particular genes are co-regulated.

In another embodiment, the gene expression profiles and microarrays of the present invention may also be used to identify a class of diseases. For example, gene expression profiles or protein expression profiles may be used to distinguish tumor types (e.g., lymphomas). By monitoring gene or protein expression, it may be possible to distinguish, for example, Hodgkin lymphoma from non-Hodgkin lymphoma. By identifying the lymphoma type, the appropriate clinical course may be implemented.

In addition, new tumor-associated genes or proteins may be identified by systemically comparing the expression of genes in tumor specimens with their expression in control tissue. For example, genes with elevated levels in tumor cells relative to normal cells, are candidates for genes encoding growth-promoting products (e.g., oncogenes). In contrast, genes with reduced expression levels in tumors, are candidates for genes encoding growth-inhibiting products (e.g., tumor suppressor genes or genes encoding apoptosis-inducing products). Thus, the expression profiles may point to the physiological function or malfunction of the gene product in the organism and shed light on possible treatments.

In a specific embodiment, the present invention provides endothelial cell gene expression profiles comprising one or more nucleic acid sequences substantially homologous to a nucleic acid sequence or complementary sequence thereof selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 48; SEQ ID NO: 63; SEQ ID NO: 70; SEQ ID NO: 82; SEQ ID NO: 94; and SEQ ID NO: 144.

In another embodiment, a muscle cell gene expression profile may comprise one or more nucleic acid sequences substantially homologous to a nucleic acid sequence or complementary sequence thereof selected from the group consisting of SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 39; SEQ ID NO: 40; SEQ ID NO: 41; SEQ ID NO: 42; SEQ ID NO: 54; SEQ ID NO: 55; and SEQ ID NO: 69.

In an alternative embodiment, a primary cell gene expression profile comprises one or more nucleic acid sequences substantially homologous to a nucleic acid sequence or complementary sequence thereof selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 1; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 101; SEQ ID NO: 112; SEQ ID NO: 113; SEQ ID NO: 114; SEQ ID NO: 115; SEQ ID NO: 116; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186.

The present invention also provides an epithelial cell gene expression profile comprising one or more nucleic acid sequences substantially homologous to a nucleic acid sequence or complementary sequence thereof selected from the group consisting of SEQ ID NO: 47; SEQ ID NO: 60; SEQ ID NO: 67; SEQ ID NO: 73; SEQ ID NO: 75; SEQ ID NO: 76; SEQ ID NO: 77; SEQ ID NO: 78; SEQ ID NO: 80; SEQ ID NO: 96; SEQ ID NO: 98; SEQ ID NO: 99; SEQ ID NO: 111; SEQ ID NO: 112; SEQ ID NO: 123; SEQ ID NO: 127; SEQ ID NO: 131; SEQ ID NO: 150; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186.

In yet another embodiment, a keratinocyte epithelial cell gene expression profile may comprise one or more nucleic acid sequences substantially homologous to a nucleic acid sequence or complementary sequence thereof selected from the group consisting of SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; and SEQ ID NO: 211.

The present invention also provides a mammary epithelial cell gene expression profile comprising one or more nucleic acid sequences substantially homologous to a nucleic acid sequence or complementary sequence thereof selected from the group consisting of SEQ ID NO: 78; SEQ ID NO: 212; SEQ ID NO: 213; SEQ ID NO: 216; SEQ ID NO: 225; SEQ ID NO: 226; SEQ ID NO: 227; SEQ ID NO: 239; SEQ ID NO: 271; SEQ ID NO: 285; and SEQ ID NO: 289.

In an alternative embodiment, a bronchial epithelial cell gene expression profile may comprise one or more nucleic acid sequences substantially homologous to a nucleic acid sequence or complementary sequence thereof selected from the group consisting of SEQ ID NO: 27; SEQ ID NO: 131; SEQ ID NO: 150; SEQ ID NO: 169; SEQ ID NO: 214; SEQ ID NO: 215; SEQ ID NO: 223; SEQ ID NO: 224; SEQ ID NO: 241; SEQ ID NO: 243; SEQ ID NO: 244; SEQ ID NO: 255; SEQ ID NO: 256; SEQ ID NO: 261; and SEQ ID NO: 314.

The present invention also provides a prostate epithelial cell gene expression profile, which may comprise one or more nucleic acid sequences substantially homologous to a nucleic acid sequence or complementary sequence thereof selected from the group consisting of SEQ ID NO: 64; SEQ ID NO: 217; SEQ ID NO: 218; SEQ ID NO: 259; SEQ ID NO: 293; SEQ ID NO: 302; and SEQ ID NO: 320.

In yet another embodiment, a renal cortical epithelial cell gene expression profile may comprise one or more nucleic acid sequences substantially homologous to a nucleic acid sequence or complementary sequence thereof selected from the group consisting of SEQ ID NO: 49; SEQ ID NO: 57; SEQ ID NO: 104; SEQ ID NO: 123; SEQ ID NO: 160; SEQ ID NO: 165; SEQ ID NO: 166; SEQ ID NO: 219; SEQ ID NO: 267; SEQ ID NO: 270; SEQ ID NO: 279; SEQ ID NO: 280; SEQ ID NO: 283; SEQ ID NO: 291; SEQ ID NO: 305; SEQ ID NO: 307; SEQ ID NO: 310; SEQ ID NO: 313; SEQ ID NO: 325; SEQ ID NO: 326; and SEQ ID NO: 327.

The present invention further provides renal proximal tubule epithelial cell gene expression profiles comprising one or more nucleic acid sequences substantially homologous to a nucleic acid sequence or complementary sequence thereof selected from the group consisting of SEQ ID NO: 106; SEQ ID NO: 138; SEQ ID NO: 158; SEQ ID NO: 228; SEQ ID NO: 236; SEQ ID NO: 242; SEQ ID NO: 250; SEQ ID NO: 258; SEQ ID NO: 260; SEQ ID NO: 262; SEQ ID NO: 266; SEQ ID NO: 272; SEQ ID NO: 273; SEQ ID NO: 274; SEQ ID NO: 275; SEQ ID NO: 276; SEQ ID NO: 278; SEQ ID NO: 284; SEQ ID NO: 288; SEQ ID NO: 295; SEQ ID NO: 296; SEQ ID NO: 297; SEQ ID NO: 299; SEQ ID NO: 300; SEQ ID NO: 301; SEQ ID NO: 306; SEQ ID NO: 308; SEQ ID NO: 309; SEQ ID NO: 311; SEQ ID NO: 316; SEQ ID NO: 318; SEQ ID NO: 321; SEQ ID NO: 322; SEQ ID NO: 328; and SEQ ID NO: 329.

In a specific embodiment, a small airway epithelial cell gene expression profile may comprise one or more nucleic acid sequences substantially homologous to a nucleic acid sequence or complementary sequence thereof selected from the group consisting of SEQ ID NO: 173; SEQ ID NO: 174; SEQ ID NO: 183; SEQ ID NO: 220; SEQ ID NO: 221; SEQ ID NO: 222; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 237; SEQ ID NO: 238; SEQ ID NO: 240; SEQ ID NO: 245; SEQ ID NO: 246; SEQ ID NO: 247; SEQ ID NO: 248; SEQ ID NO: 249; SEQ ID NO: 251; SEQ ID NO: 252; SEQ ID NO: 254; SEQ ID NO: 257; SEQ ID NO: 263; SEQ ID NO: 264; SEQ ID NO: 265; SEQ ID NO: 268; SEQ ID NO: 269; SEQ ID NO: 270; SEQ ID NO: 277; SEQ ID NO: 281; SEQ ID NO: 282; SEQ ID NO: 286; SEQ ID NO: 287; SEQ ID NO: 290; SEQ ID NO: 294; SEQ ID NO: 298; SEQ ID NO: 303; SEQ ID NO: 312; SEQ ID NO: 315; SEQ ID NO: 317; and SEQ ID NO: 319.

The present invention also provides a renal epithelial cell gene expression profile comprising one or more nucleic acid sequences substantially homologous to a nucleic acid sequence or complementary sequence thereof selected from the group consisting of SEQ ID NO: 37; SEQ ID NO: 253; SEQ ID NO: 304; SEQ ID NO: 323; and SEQ ID NO: 324.

In a specific embodiment, the present invention provides an endothelial cell protein expression profile comprising one or more amino acid sequences encoded by all or a portion of one or more nucleic acid sequences selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 48; SEQ ID NO: 63; SEQ ID NO: 70; SEQ ID NO: 82; SEQ ID NO: 94; and SEQ ID NO: 144.

The present invention also provides a muscle cell protein expression profile comprising one or more amino acid sequences encoded by all or a portion of one or more nucleic acid sequences selected from the group consisting of SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 39; SEQ ID NO: 40; SEQ ID NO: 41; SEQ ID NO: 42; SEQ ID NO: 54; SEQ ID NO: 55; and SEQ ID NO: 69.

In another embodiment, a primary cell protein expression profile may comprise one or more amino acid sequences encoded by all or a portion of one or more nucleic acid sequences selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186.

In yet another embodiment, an epithelial cell protein expression profile may comprise one or more amino acid sequences encoded by all or a portion of one or more nucleic acid sequences selected from the group consisting of SEQ ID NO: 47; SEQ ID NO: 60; SEQ ID NO: 67; SEQ ID NO: 73; SEQ ID NO: 75; SEQ ID NO: 76; SEQ ID NO: 77; SEQ ID NO: 78; SEQ ID NO: 80; SEQ ID NO: 96; SEQ ID NO: 98; SEQ ID NO: 99; SEQ ID NO: 111; SEQ ID NO: 112; SEQ ID NO: 123; SEQ ID NO: 127; SEQ ID NO: 131; SEQ ID NO: 150; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186.

The present invention further provides a keratinocyte epithelial cell protein expression profile comprising one or more amino acid sequences encoded by all or a portion of one or more nucleic acid sequences selected from the group consisting of SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; and SEQ ID NO: 211.

In another embodiment, a mammary epithelial cell protein expression profile may comprise one or more amino acid sequences encoded by all or a portion of one or more nucleic acid sequences selected from the group consisting of SEQ ID NO: 78; SEQ ID NO: 212; SEQ ID NO: 213; SEQ ID NO: 216; SEQ ID NO: 225; SEQ ID NO: 226; SEQ ID NO: 227; SEQ ID NO: 239; SEQ ID NO: 271; SEQ ID NO: 285; and SEQ ID NO: 289.

Still further, the present invention provides a bronchial epithelial cell protein expression profile comprising one or more amino acid sequences encoded by all or a portion of one or more nucleic acid sequences selected from the group consisting of SEQ ID NO: 27; SEQ ID NO: 131; SEQ ID NO: 150; SEQ ID NO: 169; SEQ ID NO: 214; SEQ ID NO: 215; SEQ ID NO: 223; SEQ ID NO: 224; SEQ ID NO: 241; SEQ ID NO: 243; SEQ ID NO: 244; SEQ ID NO: 255; SEQ ID NO: 256; SEQ ID NO: 261; and SEQ ID NO: 314.

In yet another embodiment, a prostate epithelial cell protein expression profile comprises one or more amino acid sequences encoded by all or a portion of one or more nucleic acid sequences selected from the group consisting of SEQ ID NO: 64; SEQ ID NO: 217; SEQ ID NO: 218; SEQ ID NO: 259; SEQ ID NO: 293; SEQ ID NO: 302; and SEQ ID NO: 320.

The present invention also provides a renal cortical epithelial cell protein expression profile comprising one or more amino acid sequences encoded by all or a portion of one or more nucleic acid sequences selected from the group consisting of SEQ ID NO: 49; SEQ ID NO: 57; SEQ ID NO: 104; SEQ ID NO: 123; SEQ ID NO: 160; SEQ ID NO: 165; SEQ ID NO: 166; SEQ ID NO: 219; SEQ ID NO: 267; SEQ ID NO: 270; SEQ ID NO: 279; SEQ ID NO: 280; SEQ ID NO: 283; SEQ ID NO: 291; SEQ ID NO: 305; SEQ ID NO: 307; SEQ ID NO: 310; SEQ ID NO: 313; SEQ ID NO: 325; SEQ ID NO: 326; and SEQ ID NO: 327.

In an alternative embodiment, a renal proximal tubule epithelial cell protein expression profile may comprise one or more amino acid sequences encoded by all or a portion of one or more nucleic acid sequences selected from the group consisting of SEQ ID NO: 106; SEQ ID NO: 138; SEQ ID NO: 158; SEQ ID NO: 228; SEQ ID NO: 236; SEQ ID NO: 242; SEQ ID NO: 250; SEQ ID NO: 258; SEQ ID NO: 260; SEQ ID NO: 262; SEQ ID NO: 266; SEQ ID NO: 272; SEQ ID NO: 273; SEQ ID NO: 274; SEQ ID NO: 275; SEQ ID NO: 276; SEQ ID NO: 278; SEQ ID NO: 284; SEQ ID NO: 288; SEQ ID NO: 295; SEQ ID NO: 296; SEQ ID NO: 297; SEQ ID NO: 299; SEQ ID NO: 300; SEQ ID NO: 301; SEQ ID NO: 306; SEQ ID NO: 308; SEQ ID NO: 309; SEQ ID NO: 311; SEQ ID NO: 316; SEQ ID NO: 318; SEQ ID NO: 321; SEQ ID NO: 322; SEQ ID NO: 328; and SEQ ID NO: 329.

The present invention also provides a small airway epithelial cell protein expression profile comprising one or more amino acid sequences encoded by all or a portion of one or more nucleic acid sequences selected from the group consisting of SEQ ID NO: 173; SEQ ID NO: 174; SEQ ID NO: 183; SEQ ID NO: 220; SEQ ID NO: 221; SEQ ID NO: 222; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 237; SEQ ID NO: 238; SEQ ID NO: 240; SEQ ID NO: 245; SEQ ID NO: 246; SEQ ID NO: 247; SEQ ID NO: 248; SEQ ID NO: 249; SEQ ID NO: 251; SEQ ID NO: 252; SEQ ID NO: 254; SEQ ID NO: 257; SEQ ID NO: 263; SEQ ID NO: 264; SEQ ID NO: 265; SEQ ID NO: 268; SEQ ID NO: 269; SEQ ID NO: 270; SEQ ID NO: 277; SEQ ID NO: 281; SEQ ID NO: 282; SEQ ID NO: 286; SEQ ID NO: 287; SEQ ID NO: 290; SEQ ID NO: 294; SEQ ID NO: 298; SEQ ID NO: 303; SEQ ID NO: 312; SEQ ID NO: 315; SEQ ID NO: 317; and SEQ ID NO: 319.

In a further embodiment, a renal epithelial cell protein expression profile comprises one or more amino acid sequences encoded by all or a portion of one or more nucleic acid sequences selected from the group consisting of SEQ ID NO: 37; SEQ ID NO: 253; SEQ ID NO: 304; SEQ ID NO: 323; and SEQ ID NO: 324.

In addition, the protein expression profiles may be used to create a database and to create specific protein microarrays. Furthermore, the protein microarrays, protein expression profiles, and protein expression profile databases may be useful for epitope mapping, the study of protein-protein interaction, binding of drug candidates to a plurality of proteins, drug-drug interaction (e.g., competition binding studies of two drug candidates), binding of a plurality of drug candidates to a single or several proteins, diagnostics, or antigen mapping.

VIII. High Information Density Genes and Proteins

Although it is possible to analyze the expression of all genes expressed in a cell, a significant number of genes are expressed so infrequently and thus are of limited value in generating gene expression profiles. On the other hand, a number of genes are sufficiently expressed in a cell or differentially expressed between cells to make them useful in analyzing gene expression data. Accordingly, the present invention further provides methods for identifying the subset of genes or proteins that provides the most utility in analyzing gene and protein expression. This subset is termed “high information density genes” and “high information density proteins” and may be used to build microarrays useful for analyzing gene and protein expression and generating gene expression profiles and protein expression profiles.

Indeed, the construction of microarrays comprising nucleic acid sequences or protein-capture agents that represent high information density genes or proteins provides a means for efficiently analyzing gene or protein expression. For example, such microarrays may be universally useful for diagnosing one or many diseases. The high information density gene or protein microarrays of the present invention may comprise the least number of genes or protein-capture agents that are the most useful to researchers and healthcare providers. The microarray may include the least number of genes or protein-capture agents that produce the most specific results with the highest accuracy, specificity, and sensitivity.

More particularly, high information density genes or proteins may be identified by assessing the information content of one or more genes comprising one or more gene expression profiles or one or more proteins comprising one or more protein expression profiles. Genes or proteins providing the highest amount of information content comprise high information density genes or proteins. A high information density gene or protein provides more “information” about a particular tissue type and/or tissue state, as opposed to a gene or protein that is expressed infrequently and, therefore, is of limited value in expression analyses.

Information content may be based upon, but not limited to, the magnitude of response of a gene or protein relative to a reference state or a separate reference gene or protein. For example, the reference state may be baseline expression at a certain time point, such as prior to treatment, or may refer to a physiological state, such as being healthy or status prior to treatment. Another basis for assessing information content is the frequency of detected expression across categories of tissue, diseases, or patients compared to a reference category such as unstimulated or uninfected patients. Information content may also refer to changes in expression levels relative to categories of cells, tissues, organs, or patients.

Methods for identifying high information density genes or proteins that may be used to generate the high information density expression profiles, via the use of microarrays comprising nucleic acids or protein-capture agents representing such genes or proteins, involve algorithms that generate the high information density expression profiles. Using algorithms, genes or proteins may be ranked against each other to determine the relative information content of each gene or protein analyzed. For example, the basis for ranking genes for information content may be an algorithm adding together the number of times the gene or protein is expressed among all categories and time-points, then dividing that number by the sample set size. Furthermore, information content may be subcategorized using an algorithm that ranks the average change in expression level in all instances in which the gene or protein was expressed by the average number of times expressed.

High information density genes or proteins may be selected using an algorithm that ranks expression levels across all tissues, stimuli, and times with weighing in favor of expression that may be greatly increased or decreased among the sets. For example, high information density genes or proteins may be selected using an algorithm that correlates about 90% gene or protein expression in all cell lines or tissues with greater than about a 50% increase or decrease in expression occurring through time or after treatment with all stimuli.

High information density genes or proteins may also be selected using an algorithm that correlates a unique expression profile observed in a single cell line or tissue to a specific disease state for diagnosis or correlates to a treatment modality that may predict a positive or negative outcome. An algorithm that correlates a change in the expression profile in a single cell line or tissue to a specific disease state for diagnosis or a treatment modality that may predict a positive or negative outcome may be used as well. Further, an algorithm that correlates a change in a combination of expression profiles in a single cell line or tissue to a specific disease state for diagnosis, or a treatment modality that may predict a positive or negative outcome, may be used to select high information density genes or proteins.

High information density genes or proteins may be selected from categories that are based on patient characteristics including, for example, gender, age, disease-state, and treatment regime. Another basis for selecting high information density genes or proteins is the time of gene expression. This may include, for example, different times in a disease course, different times after stimuli exposure, different times in organismal development, or different times in the cell cycle. Another selection basis may be an increase or decrease in gene or protein expression in response to a stimulus. For example, the stimulus may include environmental alteration, viral or bacterial infection, drug exposure, protein activation, protein deactivation, chemical exposure, and cell isolation procedure.

Of the various stimuli, environmental alterations may include alterations such as changes in temperature, gas pressure, gas concentration, osmolarity, humidity, and pH. Viral stimuli may include, for example, infection with different viruses such as papilloma viruses, lentiviruses, retroviruses, hepadnaviruses, alphaviruses, flaviviruses, rhabdoviruses, herpesvirues, adenoviruses, picornaviruses, reoviruses, coronaviruses, pox viruses, paramyxoviruses, togaviruses, and arenaviruses. Bacterial stimuli may include, but may not be limited to, lipopolysacharride, formylmethionine, bacterial heat shock proteins and lipoteichoic acid.

Drug exposure stimuli may include, for example, metabolic regulators, calcium ionophores, G protein regulators, translation regulators, and transcription regulators. Protein stimuli may include proteins such as cytokines, matrix proteins, cell surface ligands, acute phase proteins, clotting factors, vasoactive proteins, and mismatched Major Histocompatibility antigens among others. Examples of chemical stimuli include organic compounds, inorganic compounds, metals, and other chemical elements. Examples of cell isolation-procedures stimuli include density gradient purification, chemical digestion, mechanical disaggregation, and centrifugation.

Once identified, the high information density genes may be used to create high information density gene microarrays. Similarly, high information density proteins may be used to create high information density protein microarays. The high information density microarrays may represent a particular tissue type, such as heart, liver, prostate, lung, nerve, muscle, or connective tissue; coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesangial cells, coronary artery smooth muscle, bronchial smooth muscle, uterine smooth muscle, lung fibroblast, osteoblasts, and prostate stromal cells.

The high information density microarrays may be used in the applications described in the present application. For example, the high information density microarrays may be used to diagnose a patient and predict treatment effectiveness. The microarray may comprise the fewest genes or protein-capture agents necessary to produce the most accurate, reproducible, and specific results that correlate to a positive outcome. Once a treatment course begins, the microarray may be used to generate a gene expression profile or a protein expression profile that correlates to a particular outcome. The clinician may then use this information to adjust or change therapy accordingly. The microarray itself may contain genes or protein-capture agents that provide the highest amount of information on at least one type but possibly all therapies, for at least one but possibly all diseases.

Used in diagnostic applications, the high-information density microarray may be compared to standard diagnostic pathologies. Specificity, sensitivity, accuracy, predictive value, and standard error of the microarray may be assessed, as well as confidence intervals and prevalence of a disease in a population using standard techniques. Such diagnostic microarrays may be validated based on at least one of the following parameters or combinations thereof described below, wherein “a” represents the number of true positives, “b” represents the number of false positives, “c” represents the number of false negatives, and “d” represents the number of true negatives.

For example, sensitivity may be defined as a/a+c×100 and indicates the percentage of individuals with the disease that have positive test results. Specificity may be defined as d/b+d and indicates the percentage of individuals who do not have the particular disease and have negative test results. Accuracy (efficiency) may be defined as a+d/a+b+c+d×100 and may be the percentage of true positive and true negative test results that are correctly identified by the test. Prevalence may be defined as a+c/a+b+c+d×100 and may be the frequency of disease in the population at a given time based on the incidence of disease per year per 100,000 people.

Positive predictive value may be defined as a/a+b×100 and may be the percentage of true positive test results based on the prevalence of disease in the population. Negative predictive value may be defined as d/c+d×100 and may be the percentage of true negative test results based on the prevalence of disease in the population.

The standard error (SE) of the diagnostic microarrays may be calculated using the following formula: SE=((p)×((1−p)/n))^{1/2}, where p=sensitivity of the test and n=sample size. The 95% confidence interval may be calculated by the formula: p−(1.96×SE) to p+(1.96×SE), where p=sensitivity of the test and “1.96” may be derived from statistical tables. The high information density microarray may have a gene or combination of genes or a protein-capture agent or a combination of protein-capture agents that yield the highest sensitivity, specificity and accuracy over the widest range of standards, and also offers the best positive and negative predictive value for the most applications.

In another embodiment, a high information-density microarray may comprise the genes or protein-capture agents that best diagnose leukemia in the most patients with the highest accuracy. Such diagnostic genes may be 100% sensitive, 100% specific and 100% accurate. A microarray may also include a combination of genes or protein-capture agents that together, rather than individually, yield high sensitivity, specificity, and accuracy, thus diagnosing leukemia with 100% sensitivity, specificity and accuracy. For example, any two separate genes or protein-capture agents may only offer 50% or less sensitivity, specificity, or accuracy for diagnosis leukemia individually, but if combined on the same microarray the specificity may reach 100% because these genes or proteins are only found together when the patient has leukemia. Hence, the gene or combination of genes or protein or combination of proteins that yield the highest information content on leukemia diagnosis may be included on the microarray.

For predicting treatment efficiency, the microarray may contain the genes or protein-capture agents that best predict treatment outcome for leukemia in patients. An expression profile specific for either positive or negative treatment outcome may be 100% sensitive, 100% specific and 100% accurate. A microarray may also include a combination of genes or protein-capture agents that together, rather than individually, predict outcomes of treatments with 100% sensitivity, specificity, and accuracy. For example, any two separate genes or protein-capture agents may only offer 50% or less sensitivity, specificity, or accuracy for outcomes of various treatment modalities for leukemia individually, but when they are combined the microarray may indicate the outcome of a specific patient treatment with sufficient, preferably 100%, accuracy. Thus, the combinations that yield the highest information content on leukemia treatment modality may be included on the microarray.

The high information-density microarrays may be used for indicating when, for example, erythropoeitin (EPO) treatment would be appropriate for a patient or for monitoring drug effectiveness during such treatment. The expression profiles used on the microarray may be one gene or protein-capture agent that may be 100% specific, 100% sensitive, and 100% accurate for indicating when EPO may be provided as a treatment or determining EPO treatment effectiveness or a combination of genes or protein-capture agents that provides the same accuracy. Accordingly, the microarray can provide valuable information on when EPO is appropriate as a course of treatment and when EPO is effective in that treatment. In like manner, a microarray may be used for indicating when cytokine treatment, such as Interleukin 5, Granulocyte Stimulating Factor, Interleukin 2, and Interleukin 12, would be appropriate for a patient during or after chemotherapy or radiation therapy, or for monitoring drug effectiveness during such treatment.

Cancer treatment is an important field in which these types of microarrays may efficiently be used to indicate when a patient has cancer, the type of cancer the patient has, as well as the best treatment modality and prognosis of the patient. The microarray may also be used to monitor drug effectiveness during cancer treatment by measuring whether cancer is present and to what extent. As an example, and without limitation, the microarray may be used for indicating when a patient has Human Immunodeficiency Virus (HIV), the best treatment modality for that patient, and the prognosis of the patient. By measuring whether HIV is present and to what extent, a microarray containing expression profiles from either the host or pathogen may be used as well to monitor drug effectiveness during HIV treatment.

The nucleic acid and protein microarrays of the present invention may be useful as a diagnostic tool in assessing the effects of treatment with a compound on relative gene and protein expression. In one embodiment of the present invention, the methods described herein may be used to assess the pharmacological effects of one or more of the following growth factors, proteins, cytokines or peptides. The genes and protein-capture agents of the present invention may be specific to such growth factors, proteins, cytokines, and peptides or relate to their expression levels.

Briefly, growth factors are hormones or cytokine proteins that bind to receptors on the cell surface, with the primary result of activating cellular proliferation and/or differentiation. Many growth factors are quite versatile, stimulating cellular division in numerous different cell types, while others are specific to a particular cell-type. The following Table 1 presents several factors, but is not intended to be comprehensive or complete, yet introduces some of the more commonly known factors and their principal activities.

TABLE 1
Growth Factors

| Factor | Principal Source | Primary Activity | Comments |
|---|---|---|---|
| Platelet Derived | Platelets, endothelial | Promotes proliferation of | Dimer required for |
| Growth Factor | cells, placenta. | connective tissue, glial and | receptor binding. |
| (PDGF) | | smooth muscle cells. PDGF | Two different protein |
| | | receptor has intrinsic tyrosine | chains, A and B, form |
| | | kinase activity. | 3 distinct dimer |
| | | | forms. |
| Epidermal | Submaxillary gland, | promotes proliferation of | EGF receptor has |
| Growth Factor | Brunners gland. | mesenchymal, glial and | tyrosine kinase |
| (EGF) | | epithelial cells | activity, activated in |
| | | | response to EGF |
| | | | binding. |
| Fibroblast | Wide range of cells; | Promotes proliferation of | Four distinct |
| Growth Factor | protein is associated with | many cells including skeletal | receptors, all with |
| (FGF) | the ECM; nineteen family | and nervous system; inhibits | tyrosine kinase |
| | members. Receptors | some stem cells; induces | activity. FGF |
| | widely distributed in | mesodermal differentiation. | implicated in mouse |
| | bone, implicated in | Non-proliferative effects | mammary tumors and |
| | several bone-related | include regulation of pituitary | Kaposi's sarcoma. |
| | diseases. | and ovarian cell function. | |
| NGF | | Promotes neurite outgrowth | Several related |
| | | and neural cell survival | proteins first |
| | | | identified as proto- |
| | | | oncogenes; trkA |
| | | | (trackA), trkB, trkC |
| Erythropoietin | Kidney | Promotes proliferation and | Also considered a |
| (Epo) | | differentiation of erythrocytes | ‘blood protein,’ and a |
| | | | colony stimulating |
| | | | factor. |
| Transforming | Common in transformed | Potent keratinocyte growth | Related to EGF. |
| Growth Factor a | cells, found in | factor. | |
| (TGF-α) | macrophages and | | |
| | keratinocytes | | |
| Transforming | Tumor cells, activated | Anti-inflammatory (suppresses | Large family of |
| Growth Factor v | TH₁ cells (T-helper) and | cytokine production and class | proteins including |
| (TGF-β) | natural killer (NK) cells | II MHC expression), | activin, inhibin and |
| | | proliferative effects on many | bone morpho-genetic |
| | | mesenchymal and epithelial | protein. Several |
| | | cell types, may inhibit | classes and |
| | | macrophage and lymphocyte | subclasses of cell- |
| | | proliferation, | surface receptors |
| Insulin-Like | Primarily liver, produced | Promotes proliferation of | Related to IGF-II and |
| Growth Factor-I | in response to GH and | many cell types, autocrine and | proinsulin, also called |
| (IGF-I) | then induces subsequent | paracrine activities in addition | Somatomedin C. |
| | cellular activities, | to the initially observed | IGF-I receptor, like |
| | particularly on bone | endocrine activities on bone. | the insulin receptor, |
| | growth | | has intrinsic tyrosine |
| | | | kinase activity. IGF-I |
| | | | can bind to the |
| | | | insulin receptor. |
| Insulin-Like | Expressed almost | Promotes proliferation of | IGF-II receptor is |
| Growth | exclusively in embryonic | many cell types primarily of | identical to the |
| Factor-II | and neonatal tissues. | fetal origin. Related to IGF-I | mannose-6-phosphate |
| (IGF-II) | | and proinsulin. | receptor that is |
| | | | responsible for the |
| | | | integration of |
| | | | lysosomal enzymes |

Additional growth factors that may be utilized within the methodologies of the present invention include insulin and proinsulin (U.S. Pat. No. 4,431,740); Activin (Vale et al., 321 NATURE 776 (1986); Ling et al., 321 NATURE 779 (1986)); Inhibin (U.S. Pat. Nos. 4,740,587; 4,737,578); and Bone Morphongenic Proteins (BMPs) (U.S. Pat. No. 5,846,931; WOZNEY, CELLULAR & MOLECULAR BIOLOGY OF BONE 131-167 (1993)).

Additional growth factors that may be utilized within the methodologies of the present invention include Activin (Vale et al., 321 NATURE 776 (1986); Ling et al., 321 NATURE 779 (1986)), Inhibin (U.S. Pat. Nos. 4,737,578; 4,740,587), and Bone Morphongenic Proteins (BMPs) (U.S. Pat. No. 5,846,931; WOZNEY, CELLULAR & MOLECULAR BIOLOGY OF BONE 131-67 (1993)).

In another embodiment, the methodologies of the present invention may be used to assess the pharmacological effects a cytokine or cytokine receptor on a patient or cell line. Secreted primarily from leukocytes, cytokines stimulate both the humoral and cellular immune responses, as well as the activation of phagocytic cells. Cytokines that are secreted from lymphocytes are termed lymphokines, whereas those secreted by monocytes or macrophages are termed monokines. A large family of cytokines are produced by various cells of the body. Many of the lymphokines are also known as interleukins (ILs), because they are not only secreted by leukocytes, but are also able to affect the cellular responses of leukocytes. More specifically, interleukins are growth factors targeted to cells of hematopoictic origin. The list of identified interleukins grows continuously. See, e.g., U.S. Pat. No. 6,174,995; U.S. Pat. No. 6,143,289; Sallusto et al., 18 ANNU. REV. IMMUNOL. 593 (2000); Kunkel et al., 59 J. LEUKOCYTE BIOL. 81 (1996).

Additional growth factor/cytokines encompassed in the methodologies of the present invention include pituitary hormones such as CEA, FSH, FSH α, FSH β, Human Chorionic Gonadotrophin (HCG), HCG α, HCG β, uFSH (urofollitropin), GH, LH, LH α, LH β, PRL, TSH, TSH α, TSH β, and CA, parathyroid hormones, follicle stimulating hormones, estrogens, progesterones, testosterones, or structural or functional analog thereof. All of these proteins and peptides are known in the art. Many may be obtained commercially from, e.g., Research Diagnostics, Inc. (Flanders, N.J.).

The cytokine family also includes tumor necrosis factors, colony stimulating factors, and interferons. See, e.g., Cosman, 7 BLOOD CELL (1996); Gruss et al., 85 BLOOD 3378 (1995); Beutler et al., 7 ANNU. REV. IMMUNOL. 625 (1989); Aggarwal et al., 260 J. BIOL. CHEM. 2345 (1985); Pennica et al., 312 NATURE 724 (1984); R & D Systems, CYTOKINE MINI-REVIEWS, at http://www.rndsystems.com.

Several cytokines are introduced, briefly, in Table 2 below.

TABLE 2
Cytokines

| Cytokine | Principal Source | Primary Activity |
|---|---|---|
| Interleukins | Primarily macrophages but also | Costimulation of APCs and T cells; |
| | neutrophils, endothelial cells, smooth | stimulates IL-2 receptor production and |
| IL1-α and β | muscle cells, glial cells, astrocytes, B- | expression of interferon-γ; may induce |
| | and T-cells, fibroblasts, and | proliferation in non-lymphoid cells. |
| | keratinocytes. | |
| IL-2 | CD4+ T-helper cells, activated TH₁ | Major interleukin responsible for clonal |
| | cells, NK cells. | T-cell proliferation. IL-2 also exerts |
| | | effects on B-cells, macrophages, and |
| | | natural killer (NK) cells. . IL-2 receptor |
| | | is not expressed on the surface of resting |
| | | T-cells, but expressed constitutively on |
| | | NK cells, that will secrete TNF-α, IFN-g |
| | | and GM-CSF in response to IL-2, which |
| | | in turn activate macrophages. |
| IL-3 | Primarily T-cells | Also known as multi-CSF, as it stimulates |
| | | stem cells to produce all forms of |
| | | hematopoietic cells. |
| IL-4 | TH₂ and mast cells | B cell proliferation, eosinophil and mast |
| | | cell growth and function, IgE and class II |
| | | MHC expression on B cells, inhibition of |
| | | monokine production |
| IL-5 | TH₂ and mast cells | eosinophil growth and function |
| IL-6 | Macrophages, fibroblasts, endothelial | IL-6 acts in synergy with IL-1 and TNF-α |
| | cells and activated T-helper cells. | in many immune responses, including T- |
| | Does not induce cytokine expression. | cell activation; primary inducer of the |
| | | acute-phase response in liver; enhances |
| | | the differentiation of B-cells and their |
| | | consequent production of |
| | | immunoglobulin; enhances |
| | | Glucocorticoid synthesis. |
| IL-7 | thymic and marrow stromal cells | T and B lymphopoiesis |
| IL-8 | Monocytes, neutrophils, macrophages, | Chemoattractant (chemokine) for |
| | and NK cells. | neutrophils, basophils and T-cells; |
| | | activates neutrophils to degranulate. |
| IL-9 | T cells | hematopoietic and thymopoietic effects |
| IL-10 | activated TH₂ cells, CD8⁺ T and B | inhibits cytokine production, promotes B |
| | cells, macrophages | cell proliferation and antibody production, |
| | | suppresses cellular immunity, mast cell |
| | | growth |
| IL-11 | stromal cells | synergisitc hematopoietic and |
| | | thrombopoietic effects |
| IL-12 | B cells, macrophages | proliferation of NK cells, INF-γ |
| | | production, promotes cell-mediated |
| | | immune functions |
| IL-13 | TH₂ cells | IL-4-like activities |
| IL-18 | macrophages/Kupffer cells, | Interferon-gamma-inducing factor with |
| | keratinocytes, glucocorticoid-secreting | potent pro-inflammatory activity |
| | adrenal cortex cells, and osteoblasts | |
| IL-21 | Activated T cells | IL21 has a role in proliferation and |
| | | maturation of natural killer (NK) cell |
| | | populations from bone marrow, in the |
| | | proliferation of mature B-cell populations |
| | | co-stimulated with anti-CD40, and in the |
| | | proliferation of T cells co-stimulated with |
| | | anti-CD3. |
| IL-23 | Activated dendritic cells | A complex of p19 and the p40 subunit of |
| | | IL-12. IL-23 binds to IL-12R beta 1 but |
| | | not IL-12R beta 2; activates Stat4 in PHA |
| | | blast T cells; induces strong proliferation |
| | | of mouse memory T cells; stimulates IFN- |
| | | gamma production and proliferation in |
| | | PHA blast T cells, as well as in CD45RO |
| | | (memory) T cells. |
| Tumor Necrosis | Primarily activated macrophages. | Once called cachectin; induces the |
| Factor | | expression of other autocrine growth |
| TNF-α | | factors, increases cellular responsiveness |
| | | to growth factors; induces signaling |
| | | pathways that lead to proliferation; |
| | | induces expression of a number of nuclear |
| | | proto-oncogenes as well as of several |
| | | interleukins. |
| (TNF-β) | T-lymphocytes, particularly cytotoxic | Also called lymphotoxin; kills a number |
| | T-lymphocytes (CTL cells); induced | of different cell types, induces terminal |
| | by IL-2 and antigen-T-Cell receptor | differentiation in others; inhibits |
| | interactions. | lipoprotein lipase present on the surface |
| | | of vascular endothelial cells. |
| Interferons | macrophages, neutrophils and some | Known as type I interferons; antiviral |
| INF-α and -β | somatic cells | effect; induction of class I MHC on all |
| | | somatic cells; activation of NK cells and |
| | | macrophages. |
| Interferon | Primarily CD8+ T-cells, activated TH₁ | Type II interferon; induces of class I |
| INF-γ | and NK cells | MHC on all somatic cells, induces class II |
| | | MHC on APCs and somatic cells, |
| | | activates macrophages, neutrophils, NK |
| | | cells, promotes cell-mediated immunity, |
| | | enhances ability of cells to present |
| | | antigens to T-cells; antiviral effects. |
| Monocyte | Peripheral blood | Attracts monocytes to sites of vascular |
| Chemoattractant | monocytes/macrophages | endothelial cell injury, implicated in |
| Protein-1 | | atherosclerosis. |
| (MCP1) | | |
| Colony | | Stimulate the proliferation of specific |
| Stimulating | | pluripotent stem cells of the bone marrow |
| Factors (CSFs) | | in adults. |
| Granulocyte- | | Specific for proliferative effects on cells |
| CSF (G-CSF) | | of the granulocyte lineage; proliferative |
| | | effects on both classes of lymphoid cells. |
| Macrophage- | | Specific for cells of the macrophage |
| CSF (M-CSF) | | lineage. |
| Granulocyte- | | Proliferative effects on cells of both the |
| MacrophageCSF | | macrophage and granulocyte lineages. |
| (GM-CSF) | | |

Other cytokines of interest that may be characterized by the invention described herein include adhesion molecules (R & D Systems, ADHESION MOLECULES I (1996), available at http://www.rndsystems.com); angiogenin (U.S. Pat. No. 4,721,672; Moener et al., 226 EUR. J. BIOCHEM. 483 (1994)); annexin V (Cookson et al., 20 GENOMICS 463 (1994); Grundmann et al., 85 PROC. NATL. ACAD. SCI. USA 3708 (1988); U.S. Pat. No. 5,767,247); caspases (U.S. Pat. No. 6,214,858; Thornberry et al., 281 SCIENCE 1312 (1998)); chemokines (U.S. Pat. Nos. 6,174,995; 6,143,289; Sallusto et al., 18 ANNU. REV. IMMUNOL. 593 (2000) Kunkel et al., 59 J. LEUKOCYTE BIOL. 81 (1996)); endothelin (U.S. Pat. Nos. 6,242,485; 5,294,569; 5,231,166); eotaxin (U.S. Pat. No. 6,271,347; Ponath et al., 97(3) J. CLIN. INVEST. 604-612 (1996)); Flt-3 (U.S. Pat. No. 6,190,655); heregulins (U.S. Pat. Nos. 6,284,535; 6,143,740; 6,136,558; 5,859,206; 5,840,525); Leptin (Leroy et al., 271(5) J. BIOL. CHEM. 2365 (1996); Maffei et al., 92 PNAS 6957 (1995); Zhang et al. (1994) NATURE 372: 425-432); Macrophage Stimulating Protein (MSP) (U.S. Pat. Nos. 6,248,560; 6,030,949; 5,315,000); Neurotrophic Factors (U.S. Pat. Nos. 6,005,081; 5,288,622); Pleiotrophin/Midkine (PTN/MK) (Pedraza et al., 117 J. BIOCHEM. 845 (1995); Tamura et al., 3 ENDOCRINE 21 (1995); U.S. Pat. No. 5,210,026; Kadomatsu et al., 151 BIOCHEM. BIOPHYS. RES. COMMUN. 1312 (1988)); STAT proteins (U.S. Pat. Nos. 6,030,808; 6,030,780; Darnell et al., 277 SCIENCE 1630-1635 (1997)); Tumor Necrosis Factor Family (Cosman, 7 BLOOD CELL (1996); Gruss et al., 85 BLOOD 3378 (1995); Beutler et al., 7 ANNU. REV. IMMUNOL. 625 (1989); Aggarwal et al., 260 J. BIOL. CHEM. 2345 (1985); Pennica et al., 312 NATURE 724 (1984)).

Also of interest regarding cytokines are proteins or chemical moieties that interact with cytokines, such as Matrix Metalloproteinases (MMPs) (U.S. Pat. No. 6,307,089; NAGASE, MATRIX METALLOPROTEINASES IN ZINC METALLOPROTEASES IN HEALTH AND DISEASE (11996)), and Nitric Oxide Synthases (NOS) (Fukuto, 34 ADV. PHARM 11(11995); U.S. Pat. No. 5,268,465).

A further embodiment of the present invention applies the methodologies described herein to the characterization of the pharmacological effects of blood proteins. The term “blood protein” is a generic term for a vast group of proteins generally circulating in blood plasma, and important for regulating coagulation and clot dissolution. See, e.g., Haematologic Technologies, Inc., HTI CATALOG, available at www.haemtech.com. Table 3 introduces, in a non-limiting fashion, some of the blood proteins contemplated by the present invention.

TABLE 3
Blood Proteins

| Protein | Principle Activity | Reference |
|---|---|---|
| Factor V | In coagulation, this glycoprotein pro- | Mann et al., 57 ANN. REV. BIOCHEM. |
| | cofactor, is converted to active cofactor, | 915 (1988); see also Nesheim et al., 254 |
| | factor Va, via the serine protease α- | J. BIOL. CHEM. 508 (1979); Tracy et al., |
| | thrombin, and less efficiently by its | 60 BLOOD 59 (1982); Nesheim et al., 80 |
| | serine protease cofactor Xa. The | METHODS ENZYMOL. 249 (1981); Jenny |
| | prothrombinase complex rapidly | et al., 84 PROC. NATL. ACAD. SCI. USA |
| | converts zymogen prothrombin to the | 4846 (1987). |
| | active serine protease, α-thrombin. | |
| | Down regulation of prothrombinase | |
| | complex occurs via inactivation of Va | |
| | by activated protein C. | |
| Factor VII | Single chain glycoprotein zymogen in | See generally, Broze et al., 80 METHODS |
| | its native form. Proteolytic activation | ENZYMOL. 228 (1981); Bajaj et al., 256 |
| | yields enzyme factor VIIa, which binds | J. BIOL. CHEM. 253 (1981); Williams et |
| | to integral membrane protein tissue | al., 264 J. BIOL. CHEM. 7536 (1989); |
| | factor, forming an enzyme complex that | Kisiel et al., 22 THROMBOSIS RES. 375 |
| | proteolytically converts factor X to Xa. | (1981); Seligsohn et al., 64 J. CLIN. |
| | Also known as extrinsic factor Xase | INVEST. 1056 (1979); Lawson et al., 268 |
| | complex. Conversion of VII to VIIa | J. BIOL. CHEM. 767 (1993). |
| | catalyzed by a number of proteases | |
| | including thrombin, factors IXa, Xa, | |
| | XIa, and XIIa. Rapid activation also | |
| | occurs when VII combines with tissue | |
| | factor in the presence of Ca, likely | |
| | initiated by a small amount of pre- | |
| | existing VIIa. Not readily inhibited by | |
| | antithrombin III/heparin alone, but is | |
| | inhibited when tissue factor added. | |
| Factor IX | Zymogen factor IX , a single chain | Thompson, 67 BLOOD, 565 (1986); |
| | vitamin K-dependent glycoprotein, | Hedner et al., HEMOSTASIS AND |
| | made in liver. Binds to negatively | THROMBOSIS 39-47 (R.W. Colman, J. |
| | charged phospholipid surfaces. | Hirsh, V.J. Marder, E.W. Salzman ed., |
| | Activated by factor XIα or the factor | 2^{nd}ed. J.P. Lippincott Co., Philadelphia) |
| | VIIa/tissue factor/phospholipid | 1987; Fujikawa et al., 45 METHODS IN |
| | complex. Cleavage at one site yields the | ENZYMOLOGY 74 (1974). |
| | intermediate IXα, subsequently | |
| | converted to fully active form IXaβ by | |
| | cleavage at another site. Factor IXaβ is | |
| | the catalytic component of the “intrinsic | |
| | factor Xase complex” (factor | |
| | VIIIa/IXa/Ca²⁺/phospholipid) that | |
| | proteolytically activates factor X to | |
| | factor Xa. | |
| Factor X | Vitamin K-dependent protein zymogen, | See Davie et al., 48 ADV. ENZYMOL 277 |
| | made in liver, circulates in plasma as a | (1979); Jackson, 49 ANN. REV. |
| | two chain molecule linked by a disulfide | BIOCHEM. 765 (1980); see also |
| | bond. Factor Xa (activated X) serves as | Fujikawa et al., 11 BIOCHEM. 4882 |
| | the enzyme component of | (1972); Discipio et al., 16 BIOCHEM. |
| | prothrombinase complex, responsible | 698 (1977); Discipio et al., 18 |
| | for rapid conversion of prothrombin to | BIOCHEM. 899 (1979); Jackson et al., 7 |
| | thrombin. | BIOCHEM. 4506 (1968); McMullen et |
| | | al., 22 BIOCHEM. 2875 (1983). |
| Factor XI | Liver-made glycoprotein homodimer | Thompson et al., 60 J. CLIN. INVEST. |
| | circulates, in a non-covalent complex | 1376 (1977); Kurachi et al., 16 |
| | with high molecular weight kininogen, | BIOCHEM. 5831 (1977); Bouma et al., |
| | as a zymogen, requiring proteolytic | 252 J. BIOL. CHEM. 6432 (1977); |
| | activation to acquire serine protease | Wuepper, 31 FED. PROC. 624 (1972); |
| | activity. Conversion of factor XI to | Saito et al., 50 BLOOD 377 (1977); |
| | factor XIa is catalyzed by factor XIIa. | Fujikawa et al., 25 BIOCHEM. 2417 |
| | XIa unique among the serine proteases, | (1986); Kurachi et al., 19 BIOCHEM. |
| | since it contains two active sites per | 1330 (1980); Scott et al., 69 J. CLIN. |
| | molecule. Works in the intrinsic | INVEST. 844 (1982). |
| | coagulation pathway by catalyzing | |
| | conversion of factor IX to factor IXa. | |
| | Complex form, factor XIa/HMWK, | |
| | activates factor XII to factor XIIa and | |
| | prekallikrein to kallikrein. Major | |
| | inhibitor of XIa is a₁-antitrypsin and | |
| | to lesser extent, antithrombin-III. | |
| | Lack of factor XI procoagulant activity | |
| | causes bleeding disorder: plasma | |
| | thromboplastin antecedent deficiency. | |
| Factor XII | Glycoprotein zymogen. Reciprocal | Schmaier et al., 18-38, and Davie, 242- |
| (Hageman | activation of XII to active serine | 267 HEMOSTASIS & THROMBOSIS |
| Factor) | protease factor XIIa by kallikrein is | (Colman et al., eds., J.B. Lippincott Co., |
| | central to start of intrinsic coagulation | Philadelphia, 1987). |
| | pathway. Surface bound α-XIIa activates | |
| | factor XI to XIa. Secondary cleavage of | |
| | α-XIIa by kallikrein yields β-XIIa, and | |
| | catalyzes solution phase activation of | |
| | kallikrein, factor VII and the classical | |
| | complement cascade. | |
| Factor XIII | Zymogenic form of glutaminyl-peptide | See McDonaugh, 340-357 HEMOSTASIS |
| | γ-glutamyl transferase factor XIIIa | & THROMBOSIS (Colman et al., eds., |
| | (fibrinoligase, plasma transglutaminase, | J.B. Lippincott Co., Philadelphia, 1987); |
| | fibrin stabilizing factor). Made in the | Folk et al., 113 METHODS ENZYMOL. |
| | liver, found extracellularly in plasma | 364 (1985); Greenberg et al., 69 BLOOD |
| | and intracellularly in platelets, | 867 (1987). Other proteins known to be |
| | megakaryocytes, monocytes, placenta, | substrates for Factor XIIIa, that may be |
| | uterus, liver and prostrate tissues. | hemostatically important, include |
| | Circulates as a tetramer of 2 pairs of | fibronectin (Iwanaga et al., 312 ANN. |
| | nonidentical subunits (A₂B₂). Full | NY ACAD. SCI. 56 (1978)), a₂- |
| | expression of activity is achieved only | antiplasmin (Sakata et al., 65 J. CLIN. |
| | after the Ca²⁺- and fibrin(ogen)- | INVEST. 290 (1980)), collagen (Mosher |
| | dependent dissociation of B subunit | et al., 64 J. CLIN. INVEST. 781 (1979)), |
| | dimer from A₂’ dimer. Last of the | factor V (Francis et al., 261 J. BIOL. |
| | zymogens to become activated in the | CHEM. 9787 (1986)), von Willebrand |
| | coagulation cascade, the only enzyme in | Factor (Mosher et al., 64 J. CLIN. |
| | this system that is not a serine protease. | INVEST. 781 (1979)) and |
| | XIIIa stabilizes the fibrin clot by | thrombospondin (Bale et al., 260 J. |
| | crosslinking the α and γ-chains of fibrin. | BIOL. CHEM. 7502 (1985); Bohn, 20 |
| | Serves in cell proliferation in wound | MOL. CELL BIOCHEM. 67 (1978)). |
| | healing, tissue remodeling, | |
| | atherosclerosis, and tumor growth. | |
| Fibrinogen | Plasma fibrinogen, a large glycoprotein, | FURLAN, Fibrinogen, IN HUMAN |
| | disulfide linked dimer made of 3 pairs of | PROTEIN DATA, (Haeberli, ed., VCH |
| | non-identical chains (Aa, Bb and g), | Publishers, N.Y., 1995); Doolittle, in |
| | made in liver. Aa has N-terminal peptide | HAEMOSTASIS & THROMBOSIS, 491-513 |
| | (fibrinopeptide A (FPA), factor XIIIa | (3rd ed., Bloom et al., eds., Churchill |
| | crosslinking sites, and 2 phosphorylation | Livingstone, 1994); HANTGAN, et al., in |
| | sites. Bb has fibrinopeptide B (FPB), 1 | HAEMOSTASIS & THROMBOSIS 269-89 |
| | of 3 N-linked carbohydrate moieties, | (2d ed., Forbes et al., eds., Churchill |
| | and an N-terminal pyroglutamic acid. | Livingstone, 1991). |
| | The g chain contains the other N-linked | |
| | glycos. site, and factor XIIIa cross- | |
| | linking sites. Two elongated subunits | |
| | ((AaBbg)₂) align in an antiparallel way | |
| | forming a trinodular arrangement of the | |
| | 6 chains. Nodes formed by disulfide | |
| | rings between the 3 parallel chains. | |
| | Central node (n-disulfide knot, E | |
| | domain) formed by N-termini of all 6 | |
| | chains held together by 11 disulfide | |
| | bonds, contains the 2 IIa-sensitive sites. | |
| | Release of FPA by cleavage generates | |
| | Fbn I, exposing a polymerization site on | |
| | Aa chain. These sites bind to regions on | |
| | the D domain of Fbn to form proto- | |
| | fibrils. Subsequent IIa cleavage of FPB | |
| | from the Bb chain exposes additional | |
| | polymerization sites, promoting lateral | |
| | growth of Fbn network. Each of the 2 | |
| | domains between the central node and | |
| | the C-terminal nodes (domains D and E) | |
| | has parallel a-helical regions of the Aa, | |
| | Bb and g chains having protease- | |
| | (plasmin-) sensitive sites. Another major | |
| | plasmin sensitive site is in hydrophilic | |
| | preturbance of a-chain from C-terminal | |
| | node. Controlled plasmin degradation | |
| | converts Fbg into fragments D and E. | |
| Fibronectin | High molecular weight, adhesive, | Skorstengaard et al., 161 Eur. J. |
| | glycoprotein found in plasma and | BIOCHEM. 441 (1986); Kornblihtt et al., |
| | extracellular matrix in slightly different | 4 EMBO J. 1755 (1985); Odermatt et |
| | forms. Two peptide chains | al., 82 PNAS 6571 (1985); Hynes, R.O., |
| | interconnected by 2 disulfide bonds, has | ANN. REV. CELL BIOL., 1, 67 (1985); |
| | 3 different types of repeating | Mosher 35 ANN. REV. MED. 561 (1984); |
| | homologous sequence units. Mediates | Rouslahti et al., 44 Cell 517 (1986); |
| | cell attachment by interacting with cell | Hynes 48 CELL 549 (1987); Mosher 250 |
| | surface receptors and extracellular | BIOL. CHEM. 6614 (1975). |
| | matrix components. Contains an Arg- | |
| | Gly-Asp-Ser (RGDS) cell attachment- | |
| | promoting sequence, recognized by | |
| | specific cell receptors, such as those on | |
| | platelets. Fibrin-fibronectin complexes | |
| | stabilized by factor XIIIa-catalyzed | |
| | covalent cross-linking of fibronectin to | |
| | the fibrin a chain. | |
| β₂- | Also called β₂I and Apolipoprotein H. | See, e.g., Lozier et al., 81 PNAS 2640- |
| Glycoprotein I | Highly glycosylated single chain protein | 44 (1984); Kato & Enjyoi 30 BIOCHEM. |
| | made in liver. Five repeating mutually | 11687-94 (1997); Wurm, 16 INT'L J. |
| | homologous domains consisting of | BIOCHEM. 511-15 (1984); Bendixen et |
| | approximately 60 amino acids disulfide | al., 31 BIOCHEM. 3611-17 (1992); |
| | bonded to form Short Consensus | Steinkasserer et al., 277 BIOCHEM. J. |
| | Repeats (SCR) or Sushi domains. | 387-91 (1991); Nimpf et al., 884 |
| | Associated with lipoproteins, binds | BIOCHEM. BIOPHYS. ACTA 142-49 |
| | anionic surfaces like anionic vesicles, | (1986); Kroll et. al. 434 BIOCHEM. |
| | platelets, DNA, mitochondria, and | BIOPHYS. Acta 490-501 (1986); Polz et |
| | heparin. Binding can inhibit contact | al., 11 INT'L J. BIOCHEM. 265-73 |
| | activation pathway in blood coagulation. | (1976); McNeil et al., 87 PNAS 4120-24 |
| | Binding to activated platelets inhibits | (1990); Galli et a;. 1 LANCET 1544-47 |
| | platelet associated prothrombinase and | (1990); Matsuuna et al., II LANCET 177- |
| | adenylate cyclase activities. Complexes | 78 (1990); Pengo et al., 73 THROMBOSIS |
| | between b₂I and cardiolipin have been | & HAEMOSTASIS 29-34 (1995). |
| | implicated in the anti-phospholipid | |
| | related immune disorders LAC and SLE. | |
| Osteonectin | Acidic, noncollagenous glycoprotein | Villarreal et al., 28 BIOCHEM. 6483 |
| | (Mr = 29,000) originally isolated from | (1989); Tracy et al., 29 INT'L J. |
| | fetal and adult bovine bone matrix . May | BIOCHEM. 653 (1988); Romberg et al., |
| | regulate bone metabolism by binding | 25 BIOCHEM. 1176 (1986); Sage & |
| | hydroxyapatite to collagen. Identical to | Bornstein 266 J. BIOL. CHEM. 14831 |
| | human placental SPARC. An alpha | (1991); Kelm & Mann 4 J. BONE MIN. |
| | granule component of human platelets | RES. 5245 (1989); Kelm et al., 80 |
| | secreted during activation. A small | BLOOD 3112 (1992). |
| | portion of secreted osteonectin | |
| | expressed on the platelet cell surface in | |
| | an activation-dependent manner | |
| Plasminogen | Single chain glycoprotein zymogen with | See Robbins, 45 METHODS IN |
| | 24 disulfide bridges, no free sulfhydryls, | ENZYMOLOGY 257 (1976); COLLEN, |
| | and 5 regions of internal sequence | 243-258 BLOOD COAG. (Zwaal et al., |
| | homology, “kringles”, each five triple- | eds., New York, Elsevier, 1986); see |
| | looped, three disulfide bridged, and | also Castellino et al., 80 METHODS IN |
| | homologous to kringle domains in t-PA, | ENZYMOLOGY 365 (1981); Wohl et al., |
| | u-PA and prothrombin. Interaction of | 27 THROMB. RES. 523 (1982); Barlow et |
| | plasminogen with fibrin and α2- | al., 23 BIOCHEM. 2384 (1984); |
| | antiplasmin is mediated by lysine | SOTTRUP-JENSEN ET AL., 3 PROGRESS IN |
| | binding sites. Conversion of | CHEM. FIBRINOLYSIS & THROMBOLYSIS |
| | plasminogen to plasmin occurs by | 197-228 (Davidson et al., eds., Raven |
| | variety of mechanisms, including | Press, New York 1975). |
| | urinary type and tissue type | |
| | plasminogen activators, streptokinase, | |
| | staphylokinase, kallikrein, factors IXa | |
| | and XIIa, but all result in hydrolysis at | |
| | Arg560-Val561, yielding two chains | |
| | that remain covalently associated by a | |
| | disulfide bond. | |
| tissue | t-PA, a serine endopeptidase synthesized | See Plasminogen. |
| Plasminogen | by endothelial cells, is the major | |
| Activator | physiologic activator of plasminogen in | |
| | clots, catalyzing conversion of | |
| | plasminogen to plasmin by hydrolising a | |
| | specific arginine-alanine bond. Requires | |
| | fibrin for this activity, unlike the kidney- | |
| | produced version, urokinase-PA. | |
| Plasmin | See Plasminogen. Plasmin, a serine | See Plasminogen. |
| | protease, cleaves fibrin, and activates | |
| | and/or degrades compounds of | |
| | coagulation, kinin generation, and | |
| | complement systems. Inhibited by a | |
| | number of plasma protease inhibitors in | |
| | vitro. Regulation of plasmin in vivo | |
| | occurs mainly through interaction with | |
| | a₂-antiplasmin, and to a lesser extent, a₂- | |
| | macroglobulin. | |
| Platelet Factor-4 | Low molecular weight, heparin-binding | Rucinski et al., 53 BLOOD 47 (1979); |
| | protein secreted from agonist-activated | Kaplan et al., 53 BLOOD 604 (1979); |
| | platelets as a homotetramer in complex | George 76 BLOOD 859 (1990); Busch et |
| | with a high molecular weight, | al., 19 THROMB. RES. 129 (1980); Rao |
| | proteoglycan, carrier protein. Lysine- | et al., 61 BLOOD 1208 (1983); Brindley, |
| | rich, COOH-terminal region interacts | et al., 72 J. CLIN. INVEST. 1218 (1983); |
| | with cell surface expressed heparin-like | Deuel et al., 74 PNAS 2256 (1981); |
| | glycosaminoglycans on endothelial | Osterman et al., 107 BIOCHEM. |
| | cells, PF-4 neutralizes anticoagulant | BIOPHYS. RES. COMMUN. 130 (1982); |
| | activity of heparin exerts procoagulant | Capitanio et al., 839 BIOGHEM. |
| | effect, and stimulates release of | BIOPHYS. ACTA 161 (1985). |
| | histamine from basophils. Chemotactic | |
| | activity toward neutrophils and | |
| | monocytes. Binding sites on the platelet | |
| | surface have been identified and may be | |
| | important for platelet aggregation. | |
| Protein C | Vitamin K-dependent zymogen, protein | See Esmon, 10 PROGRESS IN THROMB. |
| | C, made in liver as a single chain | & HEMOSTS. 25 (1984); Stenflo, 10 |
| | polypeptide then converted to a disulfide | SEMIN. IN THROMB. & HEMOSTAS. 109 |
| | linked heterodimer. Cleaving the heavy | (1984); Griffen et al., 60 BLOOD 261 |
| | chain of human protein C converts the | (1982); Kisiel et al., 80 METHODS |
| | zymogen into the serine protease, | ENZYMOL. 320 (1981); Discipio et al., |
| | activated protein C. Cleavage catalyzed | 18 BIOCHEM. 899 (1979). |
| | by a complex of α-thrombin and | |
| | thrombomodulin. Unlike other vitamin | |
| | K dependent coagulation factors, | |
| | activated protein C is an anticoagulant | |
| | that catalyzes the proteolytic | |
| | inactivation of factors Va and VIIIa, and | |
| | contributes to the fibrinolytic response | |
| | by complex formation with plasminogen | |
| | activator inhibitors. | |
| Protein S | Single chain vitamin K-dependent | Walker, 10 SEMIN. THROMB. |
| | protein functions in coagulation and | HEMOSTAS. 131 (1984); Dahlback et al., |
| | complement cascades. Does not | 10 SEMIN. THROMB. HEMOSTAS., 139 |
| | possess the catalytic triad. Complexes | (1984); Walker 261 J. BIOL. CHEM. |
| | to C4b binding protein (C4BP) and to | 10941 (1986). |
| | negatively charged phospholipids, | |
| | concentratin C4BP at cell surfaces | |
| | following injury. Unbound S serves as | |
| | anticoagulant cofactor protein with | |
| | activated Protein C. A single cleavage | |
| | by thrombin abolishes protein S cofactor | |
| | activity by removing gla domain. | |
| Protein Z | Vitamin K-dependent, single-chain | Sejima et al., 171 BIOCHEM. |
| | protein made in the liver. Direct | BIOPHYSICS RES. COMM. 661 (1990); |
| | requirement for the binding of thrombin | Hogg et al., 266 J. BIOL. CHEM. 10953 |
| | to endothelial phospholipids. Domain | (1991); Hogg et al., 17 BIOCHEM. |
| | structure similar to that of other vitamin | BIOPHYSICS RES. COMM. 801 (1991); |
| | K-dependant zymogens like factors VII, | Han et al., 38 BIOCHEM. 11073 (1999); |
| | IX, X, and protein C. N-terminal region | Kemkes-Matthes et al., 79 THROMB. |
| | contains carboxyglutamic acid domain | RES. 49 (1995). |
| | enabling phospholipid membrane | |
| | binding. C-terminal region lacks | |
| | “typical” serine protease activation site. | |
| | Cofactor for inhibition of coagulation | |
| | factor Xa by serpin called protein Z- | |
| | dependant protease inhibitor. Patients | |
| | diagnosed with protein Z deficiency | |
| | have abnormal bleeding diathesis during | |
| | and after surgical events. | |
| Prothrombin | Vitamin K-dependent, single-chain | Mann et al., 45 METHODS IN |
| | protein made in the liver. Binds to | ENZYMOLOGY 156 (1976); Magnusson |
| | negatively charged phospholipid | et al., PROTEASES IN BIOLOGICAL |
| | membranes. Contains two “kringle” | CONTROL 123-149 (Reich et al., eds. |
| | structures. Mature protein circulates in | Cold Spring Harbor Labs., New York |
| | plasma as a zymogen and, during | 1975); Discipio et al., 18 BIOCHEM. 899 |
| | coagulation, is proteolytically activated | (1979). |
| | to the potent serine protease α-thrombin. | |
| α-Thrombin | See Prothrombin. During coagulation, | 45 METHODS ENZYMOL. 156 (1976). |
| | thrombin cleaves fibrinogen to form | |
| | fibrin, the terminal proteolytic step in | |
| | coagulation, forming the fibrin clot. | |
| | Thrombin also responsible for feedback | |
| | activation of procofactors V and VIII. | |
| | Activates factor XIII and platelets, | |
| | functions as vasoconstrictor protein. | |
| | Procoagulant activity arrested by | |
| | heparin cofactor II or the antithrombin | |
| | III/heparin complex, or complex | |
| | formation with thrombomodulin. | |
| | Formation of thrombin/thrombomodulin | |
| | complex results in inability of thrombin | |
| | to cleave fibrinogen and activate factors | |
| | V and VIII, but increases the efficiency | |
| | of thrombin for activation of the | |
| | anticoagulant, protein C. | |
| β-Thrombo- | Low molecular weight, heparin-binding, | See, e.g., George 76 BLOOD 859 (1990); |
| globulin | platelet-derived tetramer protein, | Holt & Niewiarowski 632 BIOCHIM. |
| | consisting of four identical peptide | BIOPHYS. ACTA 284 (1980); |
| | chains. Lower affinity for heparin than | Niewiarowski et al., 55 BLOOD 453 |
| | PF-4. Chemotactic activity for human | (1980); Varma et al., 701 BIOCHIM. |
| | fibroblasts, other functions unknown. | BIOPHYS. AGTA 7 (1982); Senior et al., |
| | | 96 J. CELL. BIOL. 382 (1983). |
| Thrombopoietin | Human TPO (Thrombopoietin, Mpl- | Horikawa et al., 90 (10) BLOOD 4031-38 |
| | ligand, MGDF) stimulates the | (1997); de Sauvage et al., 369 NATURE |
| | proliferation and maturation of | 533-58 (1995). |
| | megakaryocytes and promotes increased | |
| | circulating levels of platelets in vivo. | |
| | Binds to c-Mpl receptor. | |
| Thrombo- | High-molecular weight, heparin-binding | Dawes et al., 29 THROMB. RES. 569 |
| spondin | glycoprotein constituent of platelets, | (1983); Switalska et al., 106 J. LAB. |
| | consisting of three, identical, disulfide- | CLIN. MED. 690 (1985); Lawler et al. |
| | linked polypeptide chains. Binds to | 260 J. BIOL. CHEM. 3762 (1985); Wolff |
| | surface of resting and activated platelets, | et al., 261 J. BIOL. CHEM. 6840 (1986); |
| | may effect platelet adherence and | Asch et al., 79 J. CLIN. CHEM. 1054 |
| | aggregation. An integral component of | (1987); Jaffe et al., 295 NATURE 246 |
| | basement membrane in different tissues. | (1982); Wright et al., 33 J. HISTOCHEM. |
| | Interacts with a variety of extracellular | CYTOCHEM. 295 (1985); Dixit et al., |
| | macromolecules including heparin, | 259 J. BIOL. CHEM. 10100 (1984); |
| | collagen, fibrinogen and fibronectin, | Mumby et al., 98 J. CELL. BIOL. 646 |
| | plasminogen, plasminogen activator, | (1984); Lahav et al, 145 EUR. J. |
| | and osteonectin. May modulate cell- | BIOCHEM. 151 (1984); Silverstein et al, |
| | matrix interactions. | 260 J. BIOL. CHEM. 10346 (1985); |
| | | Clezardin et al. 175 EUR. J. BIOCHEM. |
| | | 275 (1988); Sage & Bornstein (1991). |
| Von Willebrand | Multimeric plasma glycoprotein made of | Hoyer 58 BLOOD 1 (1981); Ruggeri & |
| Factor | identical subunits held together by | Zimmerman 65 J. CLIN. INVEST. 1318 |
| | disulfide bonds. During normal | (1980); Hoyer & Shainoff 55 BLOOD |
| | hemostasis, larger multimers of vWF | 1056 (1980); Meyer et al., 95 J. LAB. |
| | cause platelet plug formation by forming | CLIN. INVEST. 590 (1980); Santoro 21 |
| | a bridge between platelet glycoprotein | THROMB. RES. 689 (1981); Santoro, & |
| | IB and exposed collagen in the | Cowan 2 COLLAGEN RELAT. RES. 31 |
| | subendothelium. Also binds and | (1982); Morton et al., 32 THROMB. RES. |
| | transports factor VIII (antihemophilic | 545 (1983); Tuddenham et al., 52 BRIT. |
| | factor) in plasma. | J. HAEMATOL. 259 (1982). |

Additional blood proteins contemplated herein include the following human serum proteins, which may also be placed in another category of protein (such as hormone or antigen): Actin, Actinin, Amyloid Serum P, Apolipoprotein E, B2-Microglobulin, C-Reactive Protein (CRP), Cholesterylester transfer protein (CETP), Complement C3B, Ceruplasmin, Creatine Kinase, Cystatin, Cytokeratin 8, Cytokeratin 14, Cytokeratin 18, Cytokeratin 19, Cytokeratin 20, Desmin, Desmocollin 3, FAS (CD95), Fatty Acid Binding Protein, Ferritin, Filamin, Glial Filament Acidic Protein, Glycogen Phosphorylase Isoenzyme BB (GPBB), Haptoglobulin, Human Myoglobin, Myelin Basic Protein, Neurofilament, Placental Lactogen, Human SHBG, Human Thyroid Peroxidase, Receptor Associated Protein, Human Cardiac Troponin C, Human Cardiac Troponin I, Human Cardiac Troponin T, Human Skeletal Troponin I, Human Skeletal Troponin T, Vimentin, Vinculin, Transferrin Receptor, Prealbumin, Albumin, Alpha-1-Acid Glycoprotein, Alpha-1-Antichymotrypsin, Alpha-1-Antitrypsin, Alpha-Fetoprotein, Alpha-1-Microglobulin, Beta-2-microglobulin, C-Reactive Protein, Haptoglobulin, Myoglobulin, Prealbumin, PSA, Prostatic Acid Phosphatase, Retinol Binding Protein, Thyroglobulin, Thyroid Microsomal Antigen, Thyroxine Binding Globulin, Transferrin, Troponin I, Troponin T, Prostatic Acid Phosphatase, Retinol Binding Globulin (RBP). All of these proteins, and sources thereof, are known in the art. Many of these proteins are available commercially from, for example, Research Diagnostics, Inc. (Flanders, N.J.).

Another embodiment applies the methodologies of the present invention to the analysis of the effects of a neurotransmitter or the receptor of a neurotransmitter on a patient or cell sample. Neurotransmitters are chemicals, some of them proteinaceous, made by neurons and used by them to transmit signals to the other neurons or non-neuronal cells (e.g., skeletal muscle, myocardium, pineal glandular cells) that they innervate. Neurotransmitters produce their effects by being released into synapses when their neuron of origin fires (i.e., becomes depolarized) and then attaching to receptors in the membrane of the post-synaptic cells. This causes changes in the fluxes of particular ions across that membrane, making cells more likely to become depolarized, if the neurotransmitter happens to be excitatory, or less likely if it is inhibitory. Neurotransmitters can also produce their effects by modulating the production of other signal-transducing molecules (“second messengers”) in the post-synaptic cells. See generally COOPER, BLOOM & ROTH, THE BIOCHEM. BASIS OF NEUROPHARMACOLOGY (7th Ed. Oxford Univ. Press, NYC, 1996); http://web.indstate.edu/thcme/mwking/nerves. Neurotransmitters contemplated in the present invention include, but are not limited to, Acetylcholine, Serotonin, γ-aminobutyrate (GABA), Glutamate, Aspartate, Glycine, Histamine, Epinephrine, Norepinephrine, Dopamine, Adenosine, ATP, Nitric oxide, and any of the peptide neurotransmitters such as those derived from pre-opiomelanocortin (POMC), as well as antagonists and agonists of any of the foregoing.

Table 4 presents a non-limiting list and description of some pharmacologically active peptides which may be incorporated into the methods contemplated by the present invention.

TABLE 4
Pharmacologically active peptides

| Binding partner/ | | |
|---|---|---|
| Protein of interest | | |
| (form of peptide) | Pharmacological activity | Reference |
| EPO receptor | EPO mimetic | Wrighton et al., 273 SCIENcE 458-63 |
| (intrapeptide | | (1996); U.S. Pat. No. 5,773,569, issued |
| disulfide-bonded) | | Jun. 30, 1998. |
| EPO receptor | EPO mimetic | Livnah et al., 273 SCIENCE 464-71 |
| (C-terminally cross- | | (1996); Wrighton et al., 15 NATURE |
| linked dimer) | | BIOTECHNOLOGY 1261-5 (1997); Int'l |
| | | Patent Application WO 96/40772, |
| | | published Dec. 19, 1996. |
| EPO receptor | EPO mimetic | Naranda et al., 96 PNAS 7569-74 (1999). |
| (linear) | | |
| c-Mpl | TPO-mimetic | Cwirla et al., 276 SCIENCE 1696-9 (1997); |
| (linear) | | U.S. Pat. No. 5,869,451, issued Feb. |
| | | 9, 1999; U.S. Pat. No. 5,932,946, issued |
| | | Aug. 3, 1999. |
| c-Mpl | TPO-mimetic | Cwirla et al., 276 SCIENCE 1696-9 (1997). |
| (C-terminally cross- | | |
| linked dimer) | | |
| (disulfide-linked | stimulation of | Paukovits et al., 364 HOPPE-SEYLERS Z. |
| dimer) | hematopoesis | PHYSIOL. CHEM. 30311 (1984); |
| | (“G-CSF-mimetic”) | Laerurngal., 16 EXP. HEMAT. 274-80 |
| | | (1988). |
| (alkylene-linked dimer) | G-CSF-mimetic | Batnagar et al., 39 J. MED. CHEM. 38149 |
| | | (1996); Cuthbertson et al., 40 J. MED. |
| | | CHEM. 2876-82 (1997); King et al., 19 |
| | | EXP. HEMATOL. 481 (1991); King et al., |
| | | 86 (Suppl. 1) BLOOD 309 (1995). |
| IL-I receptor | inflammatory and | U.S. Pat. No. 5,608,035; U.S. Pat. No. |
| (linear) | autoimmune diseases (“IL-1 | 5,786,331; U.S Pat. No. 5,880,096; |
| | antagonist” or “IL-1 ra- | Yanofsky et al., 93 PNAS 7381-6 (1996); |
| | mimetic”) | Akeson et al., 271 J. BIOL. CHEM. 30517- |
| | | 23 (1996); Wiekzorek et al., 49 POL. J. |
| | | PHARMACOL. 107-17 (1997); Yanofsky, |
| | | 93 PNAS 7381-7386 (1996). |
| Facteur thyrnique | stimulation of lymphocytes | Inagaki-Ohara et al., 171 CELLULAR |
| (linear) | (FTS-mimetic) | IMMUNOL. 30-40 (1996); Yoshida, 6 J. |
| | | IMMUNOPHARMACOL 141-6 (1984). |
| CTLA4 MAb | CTLA4-mimetic | Fukumoto et al., 16 NATURE BIOTECH. |
| (intrapeptide di-sulfide | | 267-70 (1998). |
| bonded) | | |
| TNF-α receptor | TNF-α antagonist | Takasaki et al., 15 NATURE BIOTECH. |
| (exo-cyclic) | | 1266-70 (1997); WO 98/53842, published |
| | | Dec. 3, 1998. |
| TNF-α receptor | TNF-α antagonist | Chirinos-Rojas, J. IMM., 5621-26. |
| (linear) | | |
| C3b | inhibition of complement | Sahu et al., 157 IMMUNOL. 884-91 (1996); |
| (intrapeptide di-sulfide | activation; autoimmune | Morikis et al., 7 PROTEIN SCI. 619-27 |
| bonded) | diseases (C3b antagonist) | (1998). |
| vinculin | cell adhesion processes, cell | Adey et al., 324 BIOCH EM. J. 523-8 |
| (linear) | growth, differentiation | (1997). |
| | wound healing, tumor | |
| | metastasis (“vinculin | |
| | binding”) | |
| C4 binding protein (C413P) | anti-thrombotic | Linse et al. 272 BIOL. CHEM. 14658-65 |
| (linear) | | (1997). |
| urokinase receptor | processes associated with | Goodson et al., 91 PNAS 7129-33 (1994); |
| (linear) | urokinase interaction with its | International patent application WO |
| | receptor (e.g. angiogenesis, | 97/35969, published Oct. 2, 1997. |
| | tumor cell invasion and | |
| | metastasis; (URK antagonist) | |
| Mdm2, Hdm2 | Inhibition of inactivation of | Picksley et al., 9 ONCOGENE 2523-9 |
| (linear) | p53 mediated by Mdm2 or | (1994); Bottger et al. 269 J. MOL. BIOL. |
| | hdm2; anti-tumor | 744-56 (1997); Bottger et al., 13 |
| | (“Mdm/hdm antagonist”) | ONCOGENE 13: 2141-7 (1996). |
| p21^{WAF1} | anti-tumor by mimicking the | Ball et al., 7 CURR. BIOL. 71-80 (1997). |
| (linear) | activity of p21^{WAF1} | |
| farnesyl transferase | anti-cancer by preventing | Gibbs et al., 77 CELL 175-178 (1994). |
| (linear) | activation of ras oncogene | |
| Ras effector domain | anti-cancer by inhibiting | Moodie et at., 10 TRENDS GENEL 44-48 |
| (linear) | biological function of the ras | (1994); Rodriguez et al., 370 NATURE |
| | oncogene | 527-532 (1994). |
| SH2/SH3 domains | anti-cancer by inhibiting | Pawson et al, 3 CURR. BIOL. 434-432 |
| (linear) | tumor growth with activated | (1993); Yu et al., 76 CELL 933-945 |
| | tyrosine kinases | (1994). |
| p16^{INK4} | anti-cancer by mimicking | Fahraeus et al., 6 CURR. BIOL. 84-91 |
| (linear) | activity of p16; e.g., | (1996). |
| | inhibiting cyclin D-Cdk | |
| | complex (“p, 16-mimetic”) | |
| Src, Lyn | inhibition of Mast cell | Stauffer et al., 36 BIOCHEM. 9388-94 |
| (linear) | activation, IgE-related | (1997). |
| | conditions, type I | |
| | hypersensitivity (“Mast cell | |
| | antagonist”). | |
| Mast cell protease | treatment of inflammatory | International patent application WO |
| (linear) | disorders mediated by | 98/338 12, published Aug. 6, 1998. |
| | release of tryptase-6 (“Mast | |
| | cell protease inhibitors”) | |
| SH3 domains | treatment of SH3-mediated | Rickles et al., 13 EMBO J. 5598- |
| (linear) | disease states (“SH3 | 5604 (1994); Sparks et al., 269 J. |
| | antagonist”) | BIOL. CHEM. 238536 (1994); |
| | | Sparks et al., 93 PNAS 1540-44 |
| | | (1996). |
| HBV core antigen (HBcAg) | treatment of HBV viral | Dyson & Muray, PNAS 2194-98 |
| (linear) | antigen (HBcAg) infections | (1995). |
| | (“anti-HBV”) | |
| selectins | neutrophil adhesion | Martens et al., 270 J. BIOL. |
| (linear) | inflammatory diseases | CHEM. 21129-36 (1995); |
| | (“selectin antagonist”) | European Pat. App. EP 0 714 |
| | | 912, published Jun. 5, 1996. |
| calmodulin | calmodulin | Pierce et al., 1 MOLEC. |
| (linear, cyclized) | antagonist | DIVEMILY 25965 (1995); |
| | | Dedman et al., 267 J. BIOL. |
| | | CHEM. 23025-30 (1993); Adey |
| | | & Kay, 169 GENE 133-34 |
| | | (1996). |
| integrins | tumor-homing; treatment for | International patent applications WO |
| (linear, cyclized) | conditions related to | 95/14714, published Jun. 1, 1995; WO |
| | integrin-mediated cellular | 97/08203, published Mar. 6, 1997; WO |
| | events, including platelet | 98/10795, published Mar. 19, 1998; WO |
| | aggregation, thrombosis, | 99/24462, published May 20, 1999; Kraft |
| | wound healing, osteoporosis, | et al., 274 J. BIOL. CHEM. 1979-85 (1999) |
| | tissue repair, angiogenesis | |
| | (e.g., for treatment of cancer) | |
| | and tumor invasion | |
| | (“integrin-binding”) | |
| fibronectin and extracellular | treatment of inflammatory | International patent application WO |
| matrix components of T-cells | and autoimmune conditions | 98/09985, published Mar. 12, 1998. |
| and macrophages | | |
| (cyclic, linear) | | |
| somatostatin and cortistatin | treatment or prevention of | European patent application EP 0 911 |
| (linear) | hormone-producing tumors, | 393, published Apr. 28, 1999. |
| | acromegaly, giantism, | |
| | dementia, gastric ulcer, | |
| | tumor growth, inhibition of | |
| | hormone secretion, | |
| | modulation of sleep or | |
| | neural activity | |
| bacterial lipopoly-saccharide | antibiotic; septic shock; | U.S. Pat. No. 5,877,151, issued Mar. 2, |
| (linear) | disorders modulatable by | 1999. |
| | CAP3 7 | |
| parelaxin, mellitin | antipathogenic | International patent application WO |
| (linear or cyclic) | | 97/31019, published 28 Aug. 1997. |
| VIP | impotence, neuro- | International patent application WO |
| (linear, cyclic) | degenerative disorders | 97/40070, published Oct. 30, 1997. |
| CTLs | cancer | European patent application EP 0 770 |
| (linear) | | 624, published May 2, 1997. |
| THF-gamma2 | | Burnstein, 27 BIOCHEM. 4066-71 (1988). |
| (linear) | | |
| Amylin | | Cooper, 84 PNAS 8628-32 (1987). |
| (linear) | | |
| Adreno-medullin | | Kitamura, 192 BBRC 553-60 (1993). |
| (linear) | | |
| VEGF | anti-angiogenic; cancer, | Fairbrother, 37 BIOCHEM. 17754-64 |
| (cyclic, linear) | rheumatoid arthritis, diabetic | (1998). |
| | retinopathy, psoriasis | |
| | (“VEGF antagonist′”) | |
| MMP | inflammation and | Koivunen, 17 NATURE BIOTECH. 768-74 |
| (cyclic) | autoimmune disorders; | (1999). |
| | tumor growth (“MMP | |
| | inhibitor”) | |
| HGH fragment | | U.S. Pat. No. 5,869,452, issued |
| (linear) | | Feb. 9, 1999. |
| Echistatin | inhibition of platelet | Gan, 263 J. BIOL. 19827-32 (1988). |
| | aggregation | |
| SLE autoantibody | SLE | International patent application WO |
| (linear) | | 96/30057, published Oct. 3, 1996. |
| GD1 alpha | suppression of tumor | Ishikawa Ct al., 1 FEBS LETT. 20-4 |
| | metastasis | (1998). |
| anti-phospholipid β-2 | endothelial cell activation, | Blank Mal., 96 PNAS 5164-8 (1999). |
| glycoprotein-1 (β2GPI) | anti-phospholipid syndrome | |
| | (APS), thromboembolic | |
| antibodies | phenomena, | |
| | thrombocytopenia, and | |
| | recurrent fetal loss | |
| T-CeII Receptor β chain | diabetes | International patent application WO |
| (linear) | | 96/101214, published Apr. 18, 1996. |

IX. Database Creation, Database Access, and Business Methods

The business methods of the present application relate to the commercial and other uses of the methodologies of the present invention. In one aspect, the business methods include the marketing, sale, or licensing of the present methodologies in the context of providing consumers, i.e., patients, medical practitioners, medical service providers, and pharmaceutical distributors and manufacturers, with the gene expression profiles, high information density gene expression profiles, and/or protein expression profiles provided by the present invention.

Furthermore, the present invention also relates to business methods in which gene expression profiles, high information density gene expression profiles, and/or protein expression profiles are used for analyzing test samples (e.g., patient samples). In a specific embodiment, this method may be accomplished using the gene expression profile microarrays of the present invention. For example, a user (e.g., a health practitioner such as a physician) may obtain a sample (e.g., blood, tissue biopsy) from a patient. The sample may be prepared in-house, for example, using hospital facilities or the sample may be sent to a commercial laboratory facility. Briefly, RNA is extracted from the patient sample using methods that are well-known in the art. See e.g., SAMBROOK ET AL. (1989). The RNA is, for example, then amplified by PCR, labeled with a fluorophore, and hybridized to a support representing a particular gene expression profile. The support is scanned for fluorescence and the results of the scan may be sent to a central gene expression profile database for analysis. In another embodiment, the sample itself is sent to a central laboratory facility for scanning analysis. The scanning results may be sent to the central laboratory facility for analysis via a computer terminal and through the Internet or other means. The connection between the user and the computer system is preferably secure.

In practice, the user may input, for example, information relating to the fluorescence scanning results of the support as well as additional information concerning the patient such as the patient's disease state, clinical chemistry (e.g., red blood cell count, electrolytes), and other factors relating to the patient's disease state. The central computer system may then, through the use of resident computer programs, provide an analysis of the patient's sample and generate a gene expression profile reflecting the patient's genetic profile.

Those skilled in the art will appreciate that the methods and apparatus of the present invention apply to any computer system, regardless of whether the computer system is a complicated multi-user computing apparatus or a single user device such as a personal computer or workstation. A computer system suitably comprises a processor, main memory, a memory controller, an auxiliary storage interface, and a terminal interface, all of which are interconnected. Note that various modifications, additions, substitutions, or deletions may be made to the computer system within the scope of the present invention such as the addition of cache memory or other peripheral devices.

The processor performs computation and control functions of the computer system, and comprises a suitable central processing unit (CPU). The processor may comprise a single integrated circuit, such as a microprocessor, or may comprise any suitable number of integrated circuit devices and/or circuit boards working in cooperation to accomplish the functions of a processor. The processor suitably executes the algorithms (e.g., MaxCor, Mean Log Ratio) of the present invention within its main memory.

The main memory of the computer systems of the present invention suitably contains one or more computer programs relating to the algorithms used to generate the gene expression profiles and an operating system. The term “computer program” is used in its broadest sense, and includes any and all forms of computer programs, including source code, intermediate code, machine code, and any other representation of a computer program. The term “memory,” as used herein, refers to any storage location in the virtual memory space of the system. It should be understood that portions of the computer program and operating system may be loaded into an instruction cache for the main processor to execute, while other files may well be stored on magnetic or optical disk storage devices. In addition, it is to be understood that the main memory may comprise disparate memory locations.

The computer systems of the present invention may also comprise a memory controller, through use of a separate processor, which is responsible for moving requested information from the main memory and/or through the auxiliary storage interface to the main processor. While for the purposes of explanation, the memory controller is described as a separate entity, those skilled in the art understand that, in practice, portions of the function provided by the memory controller may actually reside in the circuitry associated with the main processor, main memory, and/or the auxiliary storage interface.

In a preferred embodiment, the auxiliary storage interface allows the computer system to store and retrieve information from auxiliary storage devices, such as magnetic disks (e.g., hard disks or floppy diskettes) or optical storage devices (e.g., CD-ROM). One suitable storage device is a direct access storage device (DASD). A DASD may be a floppy disk drive, which may read programs and data from a floppy disk. It is important to note that while the present invention has been (and will continue to be) described in the context of a fully functional computer system, those skilled in the art will appreciate that the mechanisms of the present invention are capable of being distributed as a program product in a variety of forms, and that the present invention applies equally regardless of the particular type of signal bearing media to actually carry out the distribution. Examples of signal bearing media include: recordable type media such as floppy disks and CD ROMS, and transmission type media such as digital and analog communication links, including wireless communication links.

Furthermore, the computer systems of the present invention may comprise a terminal interface that allows system administrators and computer programmers to communicate with the computer system, normally through programmable workstations. It should be understood that the present invention applies equally to computer systems having multiple processors and multiple system buses. Similarly, although the system bus of the preferred embodiment is a typical hardwired, multidrop bus, any connection means that supports bidirectional communication in a computer-related environment could be used.

The gene expression profile database, high information density gene expression profile database, and/or protein expression profiles may be an internal database designed to include annotation information about the expression profiles generated by the methods of the present invention and through other sources and methods. Such information may include, for example, the databases in which a given nucleic acid or protein amino acid sequence was found, patient information associated with the expression profile, including age, cancer or tumor type or progression, descriptive information about related cDNA associated with the sequence, tissue or cell source, sequence data obtained from external sources, treatment information, diagnostic and prognostic information, information regarding gene expression and/or protein expression in response to various stimuli, expression profiles for a given gene, high information density gene, and/or protein and the related disease state or course of disease, for example whether the expression profile relates to or signifies a cancerous or pre-cancerous state, and preparation methods. The expression profiles may be based on protein and/or nucleic acid microarray data obtained from publicly available or proprietary sources. The database may be divided into two sections: one for storing the sequences and related expression profiles and the other for storing the associated information. This database may be maintained as a private database with a firewall within the central computer facility. However, this invention is not so limited and the expression profile databases may be made available to the public.

The database may be a network system connecting the network server with clients. The network may be any one of a number of conventional network systems, including a local area network (LAN) or a wide area network (WAN), as is known in the art (e.g., Ethernet). The server may include software to access database information for processing user requests, and to provide an interface for serving information to client machines. The server may support the World Wide Web and maintain a website and Web browser for client use. Client/server environments, database servers, and networks are well documented in the technical, trade, and patent literature.

Through a Web browser, clients may construct search requests for retrieving data from a microarray database, a gene expression database, and/or protein expression database. For example, the user may “point and click” to user interface elements such as buttons, pull down menus, and scroll bars. The client requests may be transmitted to a Web application which formats them to produce a query that may be used to gather information from the system database, based, for example, on microarray or expression data obtained by the client, and/or other phenotypic or genotypic information. For example, the client may submit expression data based on microarray expression profiles obtained from a patient and use the system of the present invention to obtain a diagnosis based on a comparison by the system of the client expression data with the expression data contained in the database. By way of example, the system compares the expression profiles submitted by the client with expression profiles contained in the database and then provides the client with diagnostic information based on the best match of the client expression profiles with the database profiles. In addition, the website may provide hypertext links to public databases such as GenBank and associated databases maintained by the National Center for Biotechnology Information (NCBI), part of the National Library of Medicine as well as any links providing relevant information for gene expression analysis, protein expression analysis, genetic disorders, scientific literature, and the like. Information including, but not limited to, identifiers, identifier types, biomolecular sequences, common cluster identifiers (GenBank, Unigene, Incyte template identifiers, and so forth) and species names associated with each gene, is contemplated.

The present invention also provides a system for accessing bioinformation, including gene expression profiles, high information density gene expression profiles, protein expression profiles, and annotative information, which is useful in the context of the methods of the present invention. The present invention contemplates, in one embodiment, the use of a Graphical User Interface (“GUI”) for the access of gene expression profile information stored in a database. In a preferred embodiment, the GUI may be composed of two frames. A first frame may contain a selectable list of databases accessible by the user. When a database is selected in the first frame, a second frame may display information resulting from the pair-wise comparison of the expression profile database with the client-supplied expression profile as described above, along with any other phenotypic or genotypic information.

The second frame of the GUI may contain a listing of biomolecular sequence expression information and profiles contained in the selected database. Furthermore, the second frame may allow the user to select a subset, including all of the biomolecular sequences, and to perform an operation on the list of biomolecular sequences. In a preferred embodiment, the user may select the subset of biomolecular sequences by selecting a selection box associated with each biomolecular sequence. In a preferred embodiment, the operations that may be performed include, but are not limited to, downloading all listed biomolecular sequences to a database spreadsheet with classification information, saving the selected subset of biomolecular sequences to a user file, downloading all listed biomolecular sequences to a database spreadsheet without classification information, and displaying classification information on a selected subset of biomolecular sequences.

If the user chooses to display classification information on a selected subset of biomolecular sequences, a second GUI may be presented to the user. In one embodiment, the second GUI may contain a listing of one or more external databases used to create the high information density gene expression profile databases as described above. Furthermore, for each external database, the GUI may display a list of one or more fields associated with each external database. In another embodiment, the GUI may allow the user to select or deselect each of the one or more fields displayed in the second GUI. In yet another embodiment, the GUI may allow the user to select or deselect each of the one or more external databases.

In another embodiment, the business methods of the present invention include establishing a distribution system for distributing diagnostic of the present invention for sale, and may optionally include establishing a sales group for marketing the diagnostics. Yet another aspect of the present invention provides a method of conducting a target discovery business comprising identifying, by one or more of the above drug discovery methods, a test compound, as described above, which modulates the level of expression of a gene, a high information density gene, the activity of the gene product, or the activity of the high information density gene product; and optionally conducting therapeutic profiling of compounds identified, or further analogs thereof, for efficacy and toxicity in animals; and optionally licensing or selling, the rights for further drug development of said identified compounds.

Another embodiment of the present invention comprises a variety of business methods including methods for screening drug and toxicity effects on tissue or cell samples. A further aspect of the present invention comprises business methods for providing gene expression profiles, high information density gene expression profiles, and/or protein expression profiles for normal and diseased tissues. Also within the scope of this invention are business methods providing diagnostics and predictors for patient samples.

A further aspect of the present invention comprises business methods for the manufacturing and use of gene microarrays, high information density gene microarrays, and protein microarrays. The business methods further relate to providing information generated by using gene microarrays, gene expression profiles, high information density genes, high information density gene microarrays, high information density gene expression profiles, protein microarrays and protein expression microarrays.

The present invention also provides a business method for determining whether a patient has a disease or disorder associated with the overexpression and/or upregulation of a gene, or a pre-disposition to such a disease or disorder. This method comprises the steps of receiving information related to a gene or protein (e.g., sequence information and/or information related thereto), receiving phenotypic and/or genotypic information associated with the patient, and acquiring information from the databases of the present invention related to the gene or protein and/or related to such a gene- or protein-associated disease or disorder, such as cancer and specifically colon cancer. Based on one or more of the phenotypic and/or genotypic information, the gene or protein information, and the acquired information, this method may further comprise the step of determining whether the subject has a disease or disorder associated with a gene or protein, and specifically a gene or protein of the present invention, or a pre-disposition to such a gene-or protein-associated disease or disorder. The method may also comprise the step of recommending a particular treatment for the disease, disorder or pre-disease condition. Similarly, the present invention contemplates business methods as described above using, for example, high information density genes or proteins.

In one embodiment, the present invention contemplates a business method for determining whether a patient has a cellular proliferation, growth, differentiation, and/or migration disorder or a pre-disposition to a cellular proliferation, growth, differentiation, and/or migration disorder and specifically a cancerous or pre-cancerous state. This method comprises the steps of receiving information related to, e.g., sequence information of a gene or protein of the present invention and/or information related thereto, receiving phenotypic information associated with the patient, acquiring information from the network related to, e.g., sequence information of a gene or proteinand/or information related thereto, and/or related to a cellular proliferation, growth, differentiation, and/or migration disorder and specifically a cancerous or pre-cancerous state. Based on one or more of the phenotypic and/or genotypic information, the sequence information and/or information related thereto, and the acquired information this method may further comprise the step of determining whether the patient has a cellular proliferation, growth, differentiation, and/or migration disorder or a pre-disposition to a cellular proliferation, growth, differentiation, and/or migration disorder and specifically a cancerous or pre-cancerous state. The method may also comprise the step of recommending a particular treatment for the disease, disorder or pre-disease condition. Similarly, the present invention contemplates business methods as described above using, for example, high information density genes or proteins.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

### Example 1

### Cell-Specific Gene Expression Analysis

By integrating laser capture microdissection, RNA amplification, and cDNA microarray technology, diverse cell types obtained in situ may be successfully screened and subsequently identified by differential gene expression. To demonstrate this integration of technologies, the differential gene expressions of large and small-sized neurons in the dorsal root ganglia (DRG) were examined. In general, large DRG are myelinated, fast-conducting neurons that transmit mechanosensory information, and small DRG neurons are unmyelinated, slow-conducting, and transmit nociceptive information.

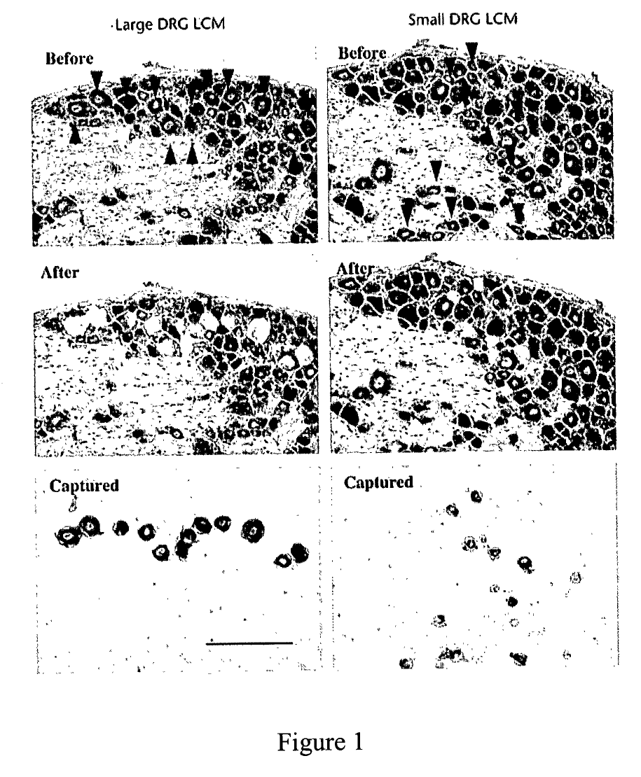

As shown in FIG. 1, large (diameter>40 μm) and small (diameter<25 μm) neurons were cleanly and individually captured via LCM from 10 μm sections of Nissl-stained rat DRGs. For this study, two sets of 1000 large neurons and 3 sets of 1000 small neurons were captured for cDNA microarray analysis.

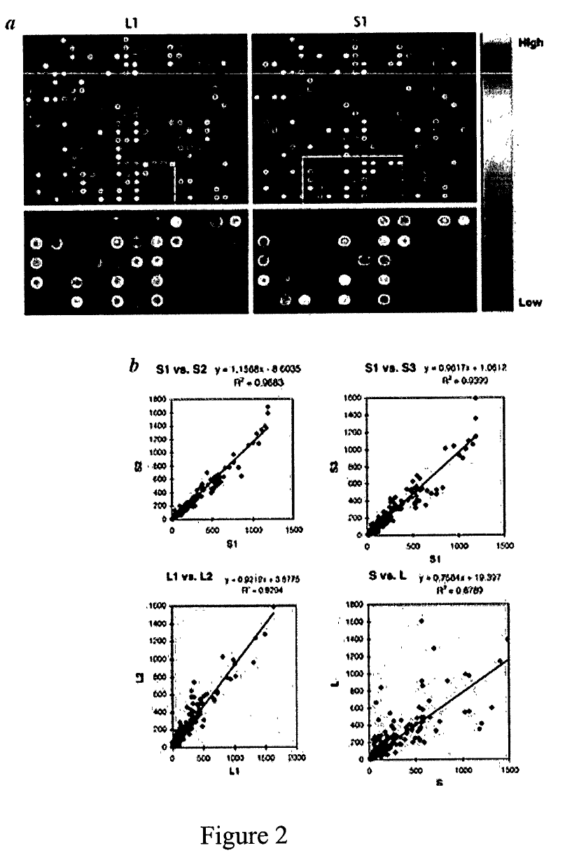

RNA was extracted from each set of neurons and linearly amplified an estimated 106-fold via T7 RNA polymerase. Once amplified, three fluorescently labeled probes were synthesized from an individually amplified RNA (aRNA) and hybridized in triplicate to a microarray (or “chip”) containing 477 cDNAs and 30 cDNAs encoding plant genes (for determination of non-specific nucleic acid hybridization). Expression in each neuronal set (designated as S1, S2, and S3 for small DRG neurons and L1 and L2 for large DRG neurons) was monitored in triplicate, requiring a total of 15 microarrays. The quality of the microarray data is demonstrated in FIG. 2a, which shows pseudocolor arrays, one resulting from hybridization to probes derived from neuronal set SI and the other from neuronal set L2. The enlarged section of the chip displays some differences in fluorescence intensity (i.e., expression levels) for particular cDNAs and demonstrates that regions containing different cDNAs are relatively uniform in size and that the background between these regions is relatively low.

To determine whether a signal corresponding to a particular cDNA is reproducible between different chips, for each neuronal set, the coefficient of variation (CV) was calculated. From these values, the overall average CV for all 477 cDNAs per neuronal set was calculated to be: S1=15.81%, S2=16.93%, S3=17.75%, L1=20.17%, and L2=19.55%.

Independent amplifications (˜10⁶-fold) of different sets of the same neuronal subtype yielded quite similar expression patterns. For example, the correlation of signal intensities between S1 vs. S2 was R²=0.9688, and between S1 vs. S3 was R²=0.9399 (FIG. 2b). Similar results were obtained between the two sets of large neurons: R²=0.929 for L1 vs. L2 (FIG. 2b). Conversely, a comparison between all three small neuronal sets (S1, S2, and S3) versus the two large sets (L1 and L2) yielded a much lower correlation (R²=0.6789), demonstrating as expected that a subgroup of genes are differentially expressed in each of the two neuronal subtypes (FIG. 2b).

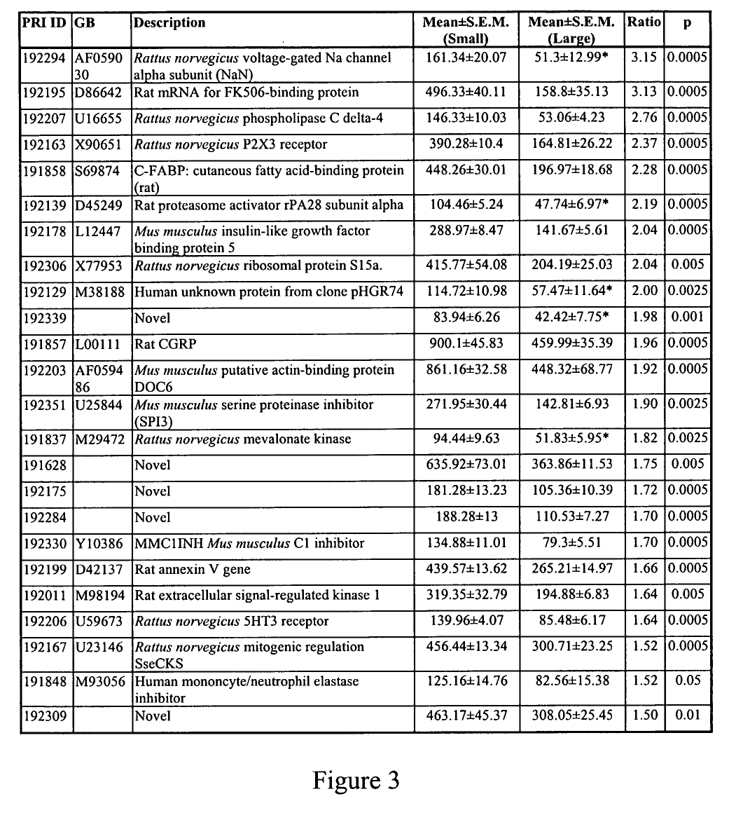

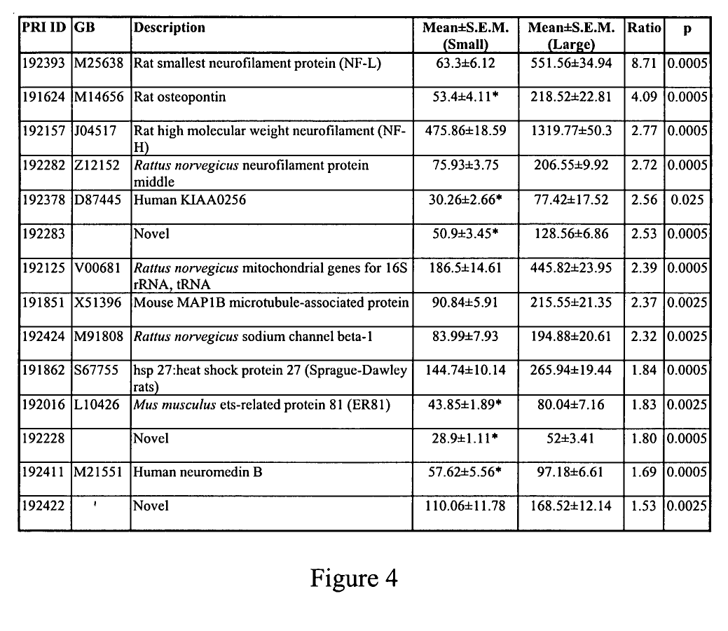

To identify the mRNAs that are differentially expressed in large and small DRG neurons, the 477 cDNAs were examined and those with 1.5-fold or greater differences (at P<0.05) were sequenced. Twenty-seven mRNAs appeared to be preferentially expressed in small DRG neurons and 14 mRNAs were preferentially expressed in large DRG (FIG. 3 and FIG. 4). To confirm the observed differential gene expression, in situ hybridization was performed with a subgroup of these cDNAs.

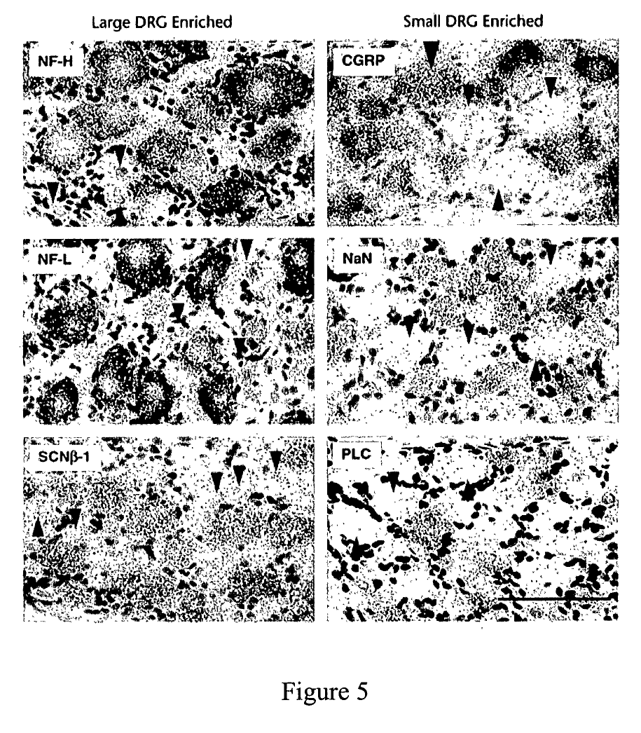

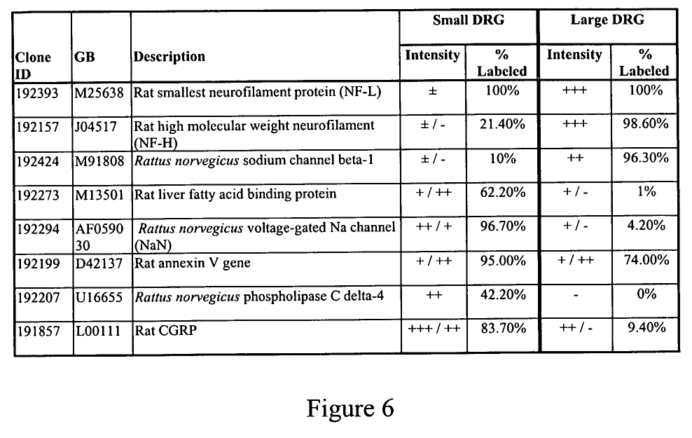

For the small neurons, five mRNAs were examined that encoded the following: fatty acid binding protein, sodium voltage-gated channel (NaN), phospholipase C delta-4, CGRP, and annexin V. For the large DRG neurons, three mRNAs were examined: neurofilament NF-L, neurofilament NF-H, and the beta-1 subunit of voltage-gated sodium channels. Based on quantitative measurements comparing the overall intensity of signal in small and large neurons and the percentage of cells labeled within the total population of either small or large neurons, the preferential expression of these mRNAs was demonstrated in large and small DRG neurons (FIG. 5 and FIG. 6).

Although this study identified preferentially expressed mRNAs within large and small DRG neurons, there is a great deal more heterogeneity within DRG neurons beyond simply small and large. For example, small DRG neurons are unmyelinated, slow-conducting, and transmit nociceptive information; whereas large DRG are myelinated, fast-conducting neurons that transmit mechanosensory information. These structural and functional differences would presumably be reflected in a heterogeneous gene expression. To address this more complicated genetic heterogeneity, immunocytochemistry may be coupled with LCM followed by RNA amplification and cDNA chip analysis as a means to further differentiate cell types within large and small DRG. In addition, chips containing a larger number of cDNAs (i.e., >10,000) can be constructed to more accurately identify the differential gene expression between large and small neurons.

The results shown herein demonstrate that expression profiles generated via these methods may not only be useful for screening cDNAs, but also, more importantly, to produce databases that contain cell type specific gene expression profile. Cell type specificity within a database will give an investigator much greater leverage in understanding the contributions of individual cell types to a particular normal or disease state and thus allow for a much finer hypotheses to be subsequently generated. Furthermore, genes, which are coordinately expressed within a given cell type, can be identified as the database grows to contain numerous gene expression profiles from a variety of cell types (or neuronal subtypes). Coordinate gene expression may also suggest functional coupling between the encoded proteins and therefore aid in determining the function for the vast majority of cDNAs currently cloned.

Laser Capture Microdissection (LCM). Two adult female Sprague Dawley rats were used in this study. Animals were anesthetized with Metofane (Methoxyflurane, Cat#556850, Mallinckrodt Veterinary Inc. Mundelein, Ill.) and sacrificed by decapitation. Using RNase-free conditions, cervical dorsal root ganglia (DRGs) were quickly dissected, placed in cryomolds, covered with frozen-tissue embedding medium OCT (Tissue-Tek, GBI, Inc., Clearwater, Minn.), and frozen in dry ice-cold 2-methylbutane (˜−60° C.). The DRGs were then sectioned at 7-10 μm in a cryostat, mounted on plain (non-coated) clean microscope slides, and immediately frozen on a block of dry ice. The sections were stored at −70° C. until further use.

A quick Nissl (cresyl violet acetate) staining was employed in order to identify the DRG neurons. Slides containing DRG sections were loaded onto a slide holder, immediately fixed in 100% ethanol for 1 minute followed by rehydration via subsequent immersions (5 seconds each) in 95%, 70%, and 50% ethanol diluted in RNase-free deionized water. Next, the slides were stained with 0.5% Nissl/0.1 M sodium acetate buffer for 1 minute, dehydrated in graded ethanol (5 seconds each), and cleared in xylene (1 minute). Once air-dried, the slides were ready for LCM.

The PixCell II LCM™ System from Acturus Engineering Inc. (Mountain View, Calif.) was used for laser-capture. Following manufacture's protocols, 2 sets of large and 3 sets small DRG neurons (1000 cells per set) were laser-captured. The criteria for large and small DRG neurons are as follows: a DRG neuron was classified as small if it had a diameter<25 μm plus an identifiable nucleus whereas a DRG neuron with a diameter>40 μm plus an identifiable nucleus was classified as large.

RNA extraction of LCM samples. Total RNA was extracted from the LCM samples with Micro RNA Isolation Kit (Stratagene, San Diego, Calif.) with some modifications. Briefly, after incubating the LCM samples in 200 μl denaturing buffer and 1.6 μl β-Mercaptoethanol at room temperature for 5 minutes, the LCM samples were extracted with 20 μl of 2 M sodium acetate, 220 μl phenol, and 40 μl chloroform:isoamyl alcohol. The aqueous layer was collected, mixed with 1 μl of 10 mg/ml carrier glycogen, and then precipitated with 200 μl of isopropanol. Following a 70% ethanol wash and air-dry, the pellets were resuspended in 16 μl of RNase-free water, 2 μl 10×DNase I reaction buffer, 1 μl Rnasin, and 1 μl of DNase I, then incubated at 37° C. for 30 minutes to remove any genomic DNA contamination. The phenol-chloroform extraction was repeated. The pellet was resuspend in 11 μl of RNase-free water and used for RT-PCR and RNA amplification.

Reverse transcription (RT) of RNA. First stand synthesis was completed by adding 10 μl of RNA isolated from the LCM samples and 1 μl of 0.5 mg/ml T7-oligo dT primer (5′TCTAGTCGACGGCCAGTGAATTGTAATACGACTCACTATAGGGCGT₂₁-3′). The primer/RNA mix was incubated for 10 minutes at 70° C., followed by a 5-minute incubation at 42° C. Next, 4 μl 5× first strand reaction buffer, 2 μl 0.1 M DTT, 1 μl 10 mM dNTPs, 1 μl RNasin, and 1 μl Superscript II (Invitrogen, Carlsbad, Calif.) were added to the mix and incubated at 42° C. for one hour. Following this incubation, 30 μl second strand synthesis buffer, 3 μl 10 mM dNTPs, 4 μl DNA Polymerase I, 1 μl E. coli RNase H, 1 μl E. coli DNA ligase, and 92 μl RNase-free water were added and samples were incubated at 16° C. for 2 hours. T4 DNA Polymerase (2 μl) was then added to each sample and samples were incubated for 10 minutes at 16° C. The cDNA was then extracted by the phenol-chloroform method and washed 3× with 500 μl water in a Microcon-100 column (Millipore Corp., Bedford, Mass.). After collection from the column, the cDNA was dried to a final volume of 8 μl for in vitro transcription.

RNA amplification. The Ampliscribe T7 Transcription Kit (Epicentre Technologies) was used to amplify RNA. In a microfuge tube, 8 μl double-stranded cDNA; 2 μl of 10× Ampliscribe T7 buffer; 1.5 μl of each 100 mM ATP, CTP, GTP, and UTP; 2 μl 0.1 M DTT; and 2 μl T7 RNA Polymerase was added and then incubated at 42° C. for 3 hours. The amplified RNA (aRNA) was washed 3× in a Microcon-100 column, collected, and dried to a final volume of 10 μl.

Amplified RNA (10 μl) from the first round amplification was mixed with 1 μl random hexamers (1 mg/ml, Pharmacia Corp., Piscataway, N.J.), incubated for 10 minutes at 70° C., chilled on ice, and then equilibrated at room temperature for 10 minutes. For the initial reaction, 4 μl 5× first stand buffer, 2 μl 0.1 M DTT, 1 μl 10 mM dNTPs, 1 μl RNasin, and 1 μl Superscript RT II were added to the aRNA mix, and then incubated at room temperature for 5 minutes followed by a 1-hour incubation at 37° C. Following the 1-hour incubation, 1 μl RNase H was added and the sample was incubated at 37° C. for 20 minutes. For second strand cDNA synthesis, 1 μl T7-oligo dT primer (0.5 mg/ml) was added to the aRNA reaction mix and the sample was incubated at 70° C. for 5 minutes, then for 10 minutes at 42° C. Following this incubation, 30 μl second strand synthesis buffer, 3 μl 10 mM dNTPs, 4 μl DNA Polymerse I, 1 μl E. coli RNase H, 1 μl E. coli DNA ligase, and 90 μl of RNase-free water were added to the sample mix and the sample was then incubated at 37° C. for 2 hours. T4 DNA Polymerase (2 μl) was then added and the sample was incubated for 10 minutes at 16° C. The double-stranded cDNA was extracted with 150 μl phenol/chloroform to remove extraneous protein and purified with Microcon-100 column to remove the unincorporated nucleotides and salts. The cDNA can be used for T7 in vitro transcription and aRNA amplification.

In situ Hybridization. Briefly, cDNAs were subcloned into pBluescript II SK (Stratagene). The cDNA vectors were then linearized and radiolabeled by ³⁵S-UTP incorporation via in vitro transcription with T7 or T3 RNA polymerase. The probes were then purified with Quick Spin™ Columns (Boehringer Mannheim, Indianapolis, Ind.). The radiolabeled probes (10⁷ cpm/probe) were hybridized to rat DRG sections (10 μm, 4% paraformaldehyde-fixed) which were mounted on Superfrost Plus slides (VWR). Following an overnight hybridization at 58° C., the slides were exposed to film. Subsequently, the slides were coated with Kodak liquid emulsion NTB2 and exposed in light-proof boxes for 1-2 weeks at 4° C. The slides were developed in Kodak Developer D-19, fixed in Kodak Fixer, and Nissl stained for expression analysis.

Under light field microscopy, mRNA expression levels of specific cDNAs were semi-quantitatively analyzed. This was accomplished as follows: no expression (−, grains were <5-fold of the background); weak expression (±, grains were 5- to 10-fold of the background); low expression (+, grains were 10- to 20-fold of the background); moderated expression (++, grains were 20- to 30-fold of the background); and strong expression (+++, grains were >30-fold of the background) (FIG. 6). The percentage of small or large neurons expressing a specific mRNA was obtained by counting the number of labeled (above background) and unlabeled cells from four sections (at least 200 cells were counted).

Microarray design. The 477 cDNA clones, obtained from two separate differential display experiments, were printed on silylated slides. The print spots were about 125 μm in diameter and were spaced 300 μm apart from center to center. Plant genes were also printed on the slides to serve as a control for non-specific hybridization.

Microarray probe synthesis. Cy3-labeled cDNA probes were synthesized from aRNA isolated from LCM DRGs with Superscript Choice System for cDNA Synthesis (Invitrogen Corp., Carlsbad, Calif.). In brief, 5 μg aRNA and 3 μg random hexamers were mixed in a total volume of 26 μl (containing RNase-free water), heated to 70° C. for 10 minutes, and then chilled on ice. For the labeling reaction, 10 μl first strand buffer, 5 μl 0.1 M DTT, 1.5 μl Rnasin, 1 μl 25 mM d(GAT)TP, 2 μl 1 mM dCTP, 2 μl Cy3-dCTP, and 2.5 μl Superscript RT II were added to the aRNA mix and incubated at room temperature for 10 minutes, and then for 2 hours at 37° C. To degrade the aRNA template, 6 μl 3N NaOH was added and the sample was incubated at 65° C. for 30 minutes. Following this incubation, 20 μl 1M Tris-HCl (pH 7.4), 12 μl 1N HCl, and 12 μl water were added. The probes were purified with Microcon 30 Columns (Millipore Corp., Bedford, Mass.) and Qiagen Nucleotide Removal Columns (Qiagen Corp., Valencia, Calif.). The probes were vacuum-dried and resuspended in 20 μl of hybridization buffer (5×SSC, 0.2% SDS) containing mouse Cot1 DNA.

Microarray hybridization. Printed glass slides were treated with sodium borohydrate solution (0.066 M NaBH4, 0.06 M NaCl) to ensure amino-linkage of cDNAs to the slides. Then, the slides were boiled in water for 2 minutes to denature the cDNA. Cy3-labeled probes were heated to 99° C. for 5 minutes, cooled to room temperature for 5 minutes, and then applied to the slides. The slides were covered with glass cover slips, sealed with DPX (Fluka) and hybridized at 60° C. for 4-6 hours. At the end of hybridization, the slides were cooled to room temperature. The slides were first washed in 1×SSC and 0.2% SDS at 55° C. for 5 minutes, and then washed in 0.1×SSC and 0.2% SDS for 5 minutes at 55° C. After a quick rinse in 0.1×SSC and 0.2% SDS, the slides were air dried and ready for scanning.

Microarray quantitation. The cDNA microarrays were scanned for Cy3 fluorescence using the ScanArray 3000 (General Scanning, Inc., Watertown, Mass.). ImaGene Software (Biodiscovery, Inc., Marina Del Ray, Calif.) was then subsequently used for quantitation. Briefly, the intensity of each spot (i.e., cDNA) was corrected by subtracting the immediate surrounding background. Next, the corrected intensities were normalized for each cDNA with the following formula:

$$\frac{intensity ( background corrected )}{{75}^{th} \text{-} percentile value of the intensity of the entire chip} \times 1000$$

To determine “non-specific” nucleic acid hybridization, 75^{th}-percentile values were calculated from the individual averages of each plant cDNA (for a total of 30 different cDNAs). The overall 75-percentile value for S1, S2, and S3 was 48.68, and for L1 and L2 was 40.94.

Statistical analyses. To assess the correlation of intensity value for each cDNA between individual sets of neurons (i.e., S1 vs. S2) or between two neuronal subtypes (i.e., small DRG vs. large DRG), scatter plots were used and the linear relationships were measured. The coefficient of determination (R²) was calculated and indicated the variability of intensity values in one group vs. the other.

To statistically determine whether the intensity values measured from microarray quantitation were true signals, each intensity was compared, via a one-sample t-test, to the 75^{th}-percentile value of the 30 plant cDNAs that were present on each chip (representing nonspecific nucleic acid hybridization). Values not significantly different from the 75-percentile value are presented in FIG. 3 and FIG. 4 and so noted. To determine which cDNAs are statistically significant in their differential gene expression between large and small neurons, the intensity for each cDNA from neuronal sets for large neurons (L1 and L2) and small neurons (S1, S2, and S3) were grouped together and intensity values were averaged for each corresponding cDNA. A two-sample t-test for one-tailed hypotheses was used to detect a gene expression difference between small neurons and large neurons.

### Example 2

### Algorithms to Produce Gene or Protein Expression Profiles

Each cell or tumor type in any given state or age has a unique gene expression pattern that distinguishes it from other tissues or cells. Using profile extraction algorithms, the gene expression profiles from many different cell types may be extracted to create a profile database. Thus, in the broadest sense, unknown samples can then be identified by comparing its profile against such a database.

To create such a database, tissue or cell samples may be divided into classifying groups (i.e., tumor vs. normal; endothelial vs. muscle, etc.). This can be done either manually or if the groups are unknown, by using a clustering algorithm such as k-means. The gene expression data is transformed into a log-ratio value, and the genes with weak differential values are filtered from the data. The gene expression profiles are then extracted using the MaxCor or Mean Log Ratio algorithms of the present invention.

For an unknown sample, it may be necessary to transform the gene expression data of the sample prior to scoring against the expression profiles. The type of data transformation may depend on the profile extraction algorithm used (i.e., MaxCor or Mean Log Ratio). The sample expression data is then scored against the profile database. A high score indicates that the unknown sample contains or is related to the sample from which the profile was derived. However, the most accurate scoring function will depend on the profile extraction algorithm used to extract the gene expression data.

Preparation of data for profile extraction. First, a reference gene expression vector is constructed where A, B, . . . Z denote the groups of samples (e.g., tumor tissue or smooth muscle cell) that will be differentiated and a, b, . . . z denote the number of samples within each group, respectively. As an example, the notation A₂₁ represents the expression intensity from the 2nd gene in sample 1 of group A. If each sample was hybridized to a DNA chip with size n genes, then the following matrices represent expression data from all of the groups A, B, . . . Z, respectively.

$$[ \begin{matrix}{A}_{11} & {A}_{12} & ⋯ & {A}_{1 a} \\ {A}_{21} & {A}_{22} & ⋯ & {A}_{2 a} \\ ⋮ & ⋯ & ⋰ & ⋮ \\ {A}_{n1} & {A}_{n2} & ⋯ & {A}_{n a}\end{matrix} ] [ \begin{matrix}{B}_{11} & {B}_{12} & ⋯ & {B}_{1 b} \\ {B}_{21} & {B}_{22} & ⋯ & {B}_{2 b} \\ ⋮ & ⋯ & ⋰ & ⋮ \\ {B}_{n1} & {B}_{n2} & ⋯ & {B}_{n b}\end{matrix} ] ⋯ [ \begin{matrix}{Z}_{11} & {Z}_{12} & ⋯ & {Z}_{1 z} \\ {Z}_{21} & {Z}_{22} & ⋯ & {Z}_{2 z} \\ ⋮ & ⋯ & ⋰ & ⋮ \\ {Z}_{n1} & {Z}_{n2} & ⋯ & {Z}_{n z}\end{matrix} ]$$

The geometric mean expression value is calculated for each gene in each matrix. Thus, A_{1(geomean)} is the geometric mean of set (A₁₁ A₁₂ . . . A₁ₐ) where A₁ denotes gene 1 in group A.

$$[ \begin{matrix}{A}_{1 ( geomean )} \\ {A}_{2 ( geomean )} \\ ⋮ \\ {A}_{n ( geomean )}\end{matrix} ] [ \begin{matrix}{B}_{1 ( geomean )} \\ {B}_{2 ( geomean )} \\ ⋮ \\ {B}_{n ( geomean )}\end{matrix} ] ⋯ [ \begin{matrix}{Z}_{1 ( geomean )} \\ {Z}_{2 ( geomean )} \\ ⋮ \\ {Z}_{n ( geomean )}\end{matrix} ]$$

The reference gene expression vector is simply the geometric mean of those vectors:

$$[ \begin{matrix}{\overline{X}}_{1} \\ {\overline{X}}_{2} \\ ⋮ \\ {\overline{X}}_{n} \end{matrix} ] where {\overline{X}}_{1} is the geometric mean of \{ {A}_{1 ( geomean )} {B}_{1 ( geomean )} ⋯ {Z}_{1 ( geomean )} \}$$

The original data set is then transformed by taking the log of the ratio relative to the reference gene expression value for each gene creating the matrices {A′ B′ . . . Z′} where A′₁₁=ln(A₁₁/\overline{X}₁) and Z′_{nz}=ln(Z_{nz}/\overline{X}ₙ). The values now represent the fold increase or decrease over the average for each gene.

$$[ \begin{matrix}{A}_{11}^{′} & {A}_{12}^{′} & ⋯ & {A}_{1 a}^{′} \\ {A}_{21}^{′} & {A}_{22}^{′} & ⋯ & {A}_{2 a}^{′} \\ ⋮ & ⋯ & ⋰ & ⋮ \\ {A}_{n1}^{′} & {A}_{n2}^{′} & ⋯ & {A}_{n a}^{′}\end{matrix} ] [ \begin{matrix}{B}_{11}^{′} & {B}_{12}^{′} & ⋯ & {B}_{1 b}^{′} \\ {B}_{21}^{′} & {B}_{22}^{′} & ⋯ & {B}_{2 b}^{′} \\ ⋮ & ⋯ & ⋰ & ⋮ \\ {B}_{n1}^{′} & {B}_{n2}^{′} & ⋯ & {B}_{n b}^{′}\end{matrix} ] ⋯ [ \begin{matrix}{Z}_{11}^{′} & {Z}_{12}^{′} & ⋯ & {Z}_{1 z}^{′} \\ {Z}_{21}^{′} & {Z}_{22}^{′} & ⋯ & {Z}_{2 z}^{′} \\ ⋮ & ⋯ & ⋰ & ⋮ \\ {Z}_{n1}^{′} & {Z}_{n2}^{′} & ⋯ & {Z}_{n z}^{′}\end{matrix} ]$$

The genes with a weak differentiation power are removed from the matrix. The Kruskal-Wallis rank test was used to rank the genes with the highest differentiation power for separating the groups, A, B, . . . Z. A low p-value from the rank test indicates a high differentiation power. A p-value of 0.0025 was used as the cut-off value.

Finally, for each resulting matrix {A″ B″ . . . Z″}, apply a profile extraction algorithm to create a profile representing each group.

Profile extraction using the MaxCor algorithm. The MaxCor algorithm is applied to each group {A″ B″ . . . Z″}0 separately. For each pair of columns in the matrix, the genes coordinately expressed in high, average, or low levels over the mean (defined below) are given a value (1, 0, or −1, respectively), producing a weight vector representing the pair. Thus, for matrix A″,

$$( \frac{a ( a - 1 )}{2} ) ,$$

pairwise calculations are performed to produce a weight vector representing the matrix pair. A final average weight vector which will be the profile for group A, is computed by averaging each weight vector calculated for matrix A″. The profile contains the same number of genes as A″ and its values should be within [−1 to 1]. These values, −1 and 1, represent the genes consistently expressed in low or high levels, respectively, relative to the mean of all groups. The MaxCor algorithm is applied to each group individually to produce a profile for each group.

Value assignment for coordinately expressed genes. For a pair of columns (c1 and c2), the values are normalized to create c1′ and c2′. Thus, c1ᵢ becomes

$$( \frac{c {1}_{i} - \overline{c} 1}{{S}_{c1}} )$$

where \overline{c}1 is the mean of column c1 and S_{c1} is the standard deviation. For each gene pair in c1′ and c2′, the normalized values are stored as vector p12 and then the p12 values are sorted from lowest to highest. A cutoff value is established, such as 0.5, and all genes with a greater normalized value than the cutoff value are collected in p12. The Pearson correlation coefficient is calculated for this set of genes using the values in column c1 and c2. The cutoff value is then continually increased until the correlation coefficient is greater than a set value, such as 0.8. When this is complete, the set of genes meeting this criteria is assigned a value of 1 if both gene values in c1′ and c2′ are positive and −1 if both gene values are negative. For all other genes in c1′ and c2′, a zero value is assigned. The resulting vector is a weight vector which represents the pair.

Sample scoring using the MaxCor algorithm. Before scoring a new sample, the genes in the sample S with weak differentiation values are removed so that the rows remaining are the same as those in the profile vectors, thus creating sample vector S″. The score is the sum of the normalized values for each gene in S″ and its weight in the profile vector. For example, the score between sample vector S″ and profile vector A^{s} is

$${\sum }_{i = 1 - n} {S}_{i}^{′′} {A}_{i}^{s} \cdot$$

The normalized score is (score−mean of randomized score)/(standard deviation of randomized score), where the randomized score is the score between S″ and the profile vector which has its gene positions randomized. Typically, 100 randomized scores are generated to calculate the mean and the standard deviation.

Profile extraction using the Mean Log Ratio approach. This algorithm is also applied to each group or matrix {A″ B″ . . . Z″} individually. For each matrix, the profile vector is the row mean of the matrix. Thus, the profile vectors for groups {A″ B″ . . . Z″} are:

$$[ \begin{matrix}{\overline{A}}_{1}^{′′} \\ {\overline{A}}_{2}^{′′} \\ ⋮ \\ {\overline{A}}_{n}^{′′}\end{matrix} ] [ \begin{matrix}{\overline{B}}_{1}^{′′} \\ {\overline{B}}_{2}^{′′} \\ ⋮ \\ {\overline{B}}_{n}^{′′}\end{matrix} ] ⋯ [ \begin{matrix}{\overline{Z}}_{1}^{′′} \\ {\overline{Z}}_{2}^{′′} \\ ⋮ \\ {\overline{Z}}_{n}^{′′}\end{matrix} ] where {\overline{A}}_{1}^{′′} is the mean of \{ {A}_{11}^{′′} , {A}_{12}^{′′} , ⋯ {A}_{1 a}^{′′} \} \cdot$$

Sample scoring using the Mean Log Ratio expression profiles. Prior to scoring a new sample, the gene expression vector of the sample is transformed by taking the log ratio relative to the reference gene expression vector for each gene. For example, the transformation of the sample S is:

$$S = [ \begin{matrix}{S}_{1} \\ {S}_{2} \\ ⋮ \\ {S}_{n}\end{matrix} ] which leads to {S}^{′} = [ \begin{matrix}{S}_{1}^{′} \\ {S}_{2}^{′} \\ ⋮ \\ {S}_{n}^{′}\end{matrix} ] , where {S}_{1}^{′} = ln ( {S}_{1} \text{/} {\overline{X}}_{1} ) .$$

The genes with weak differentiation values are removed so the rows remaining are the same as those in the profile vectors, thus creating sample vector S″. The score against each profile is then calculated by taking the Euclidean distance between S″0 and the profile vector. The normalized score is (score−mean of randomized score)/(standard deviation of randomized score), where the randomized score is the Euclidean distance between S″ and the profile vector which has randomized gene positions. Typically, 100 randomized scores are generated to calculate the mean and the standard deviation.

### Example 3

### Gene Expression Profiles for Human Primary Cells

Gene expression profiles were collected from a set of human primary cells via DNA microarray technology. These gene expression profiles can then be used to classify unknown cell or tissue samples.

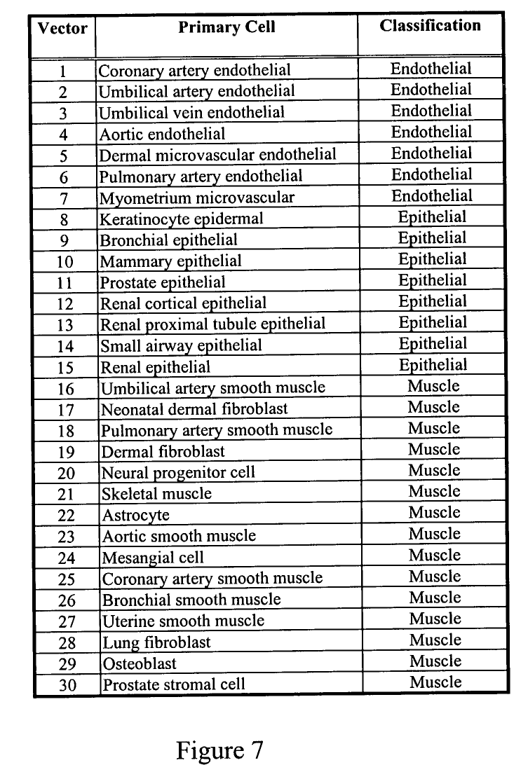

Thirty human primary cell samples were purchased from Clonetics Corporation (San Diego, Calif.). These primary cells were classified into the following categories: endothelial, epithelial, and muscle and also categorized based on the origin of tissue (FIG. 7). Total RNA was extracted, amplified, and labeled with Cy5-dCTP as described in Example 1. The resultant labeled cDNAs were hybridized to microarray chips, which contain 7286 DNA molecules representing 3643 unique genes each spotted twice. Each labeled cDNA probe was separated into two aliquots and each aliquot was hybridized to an identical microarray chip. Following a wash, the cDNA chips were scanned and the intensity of the spots was recorded and converted into a numerical value. To normalize the data, the spot intensities of each chip were divided by the intensity value of the 75th percentile of the chip, then these values were multiplied by 100. For each primary cell, a final gene intensity vector is produced by averaging four intensity values for each gene (2 spots per chip times 2 chips). The controls, low quality samples, and missing data values were removed, and 3940 genes were used for the final analysis.

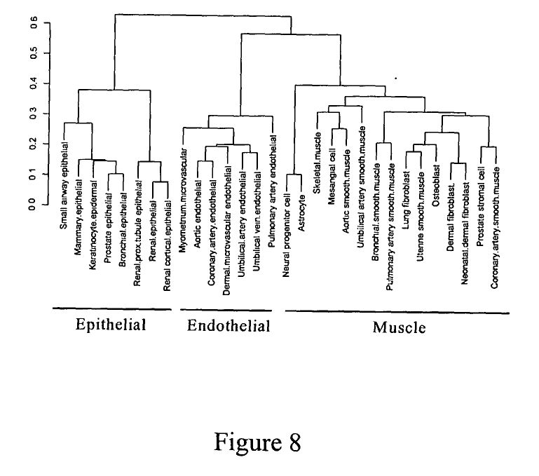

Clustering analysis of the gene expression vectors of the primary cell samples confirmed that these samples could be classified into three groups: endothelial, epithelial, and muscle cell (FIG. 8). A reference vector was generated, and the intensities were converted into a log ratio. A gene was filtered from the matrix if the p-value from the Kruskal-Wallis rank test was greater than 0.0025.

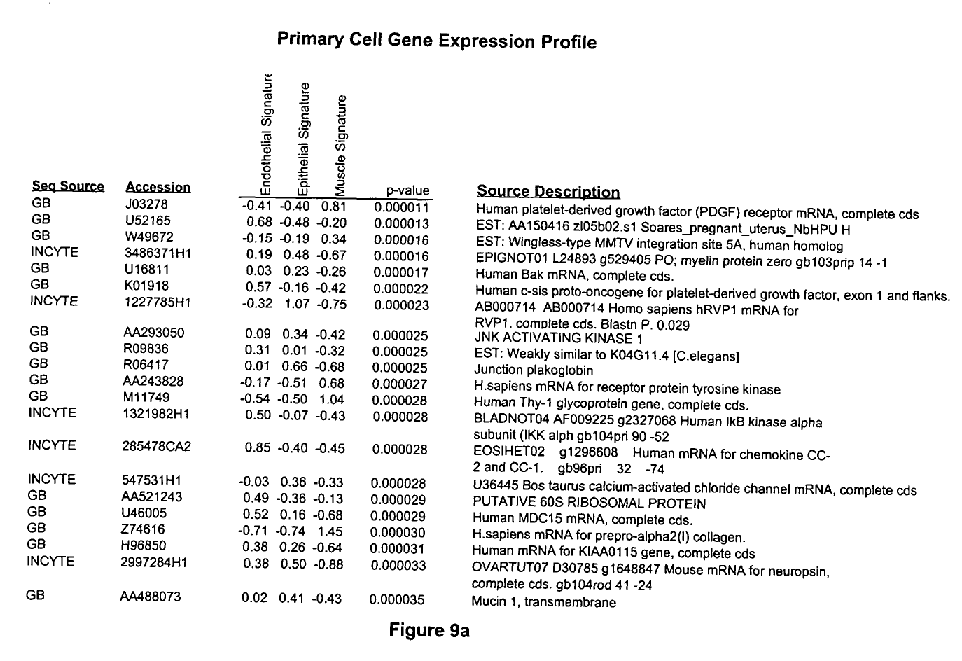

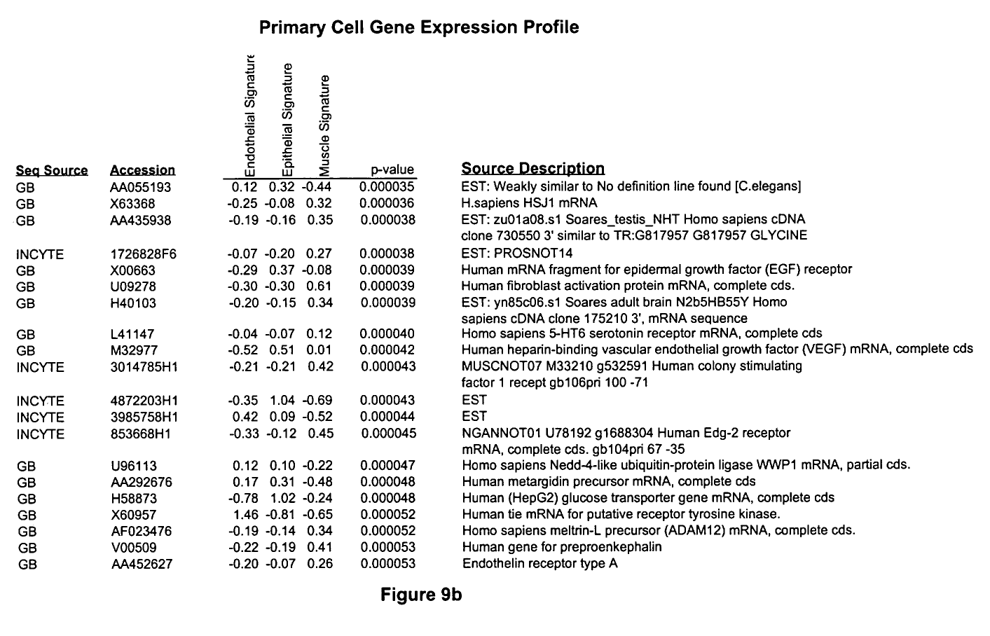

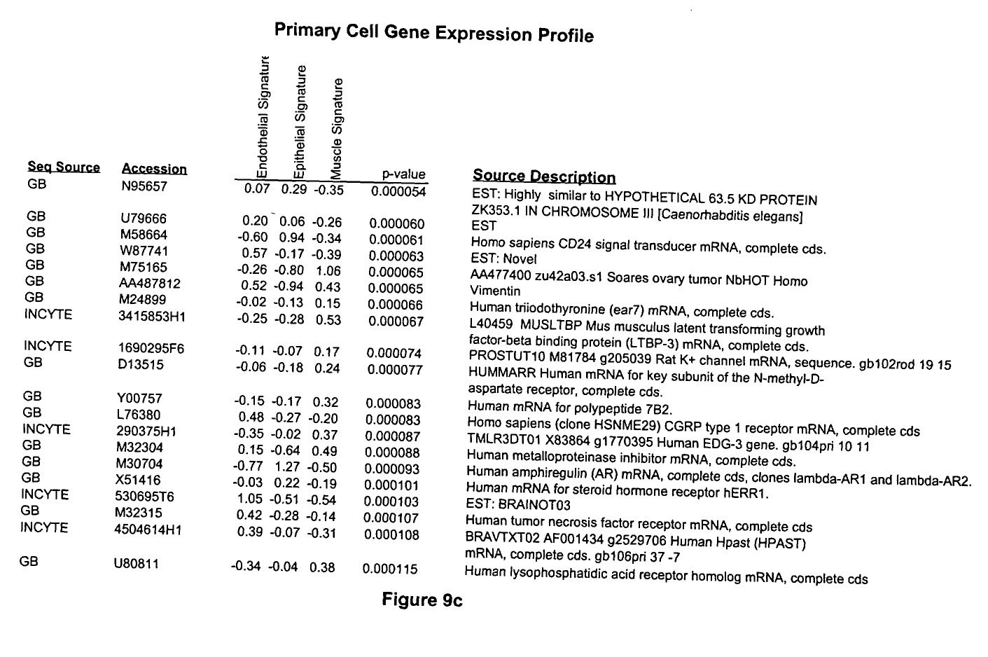

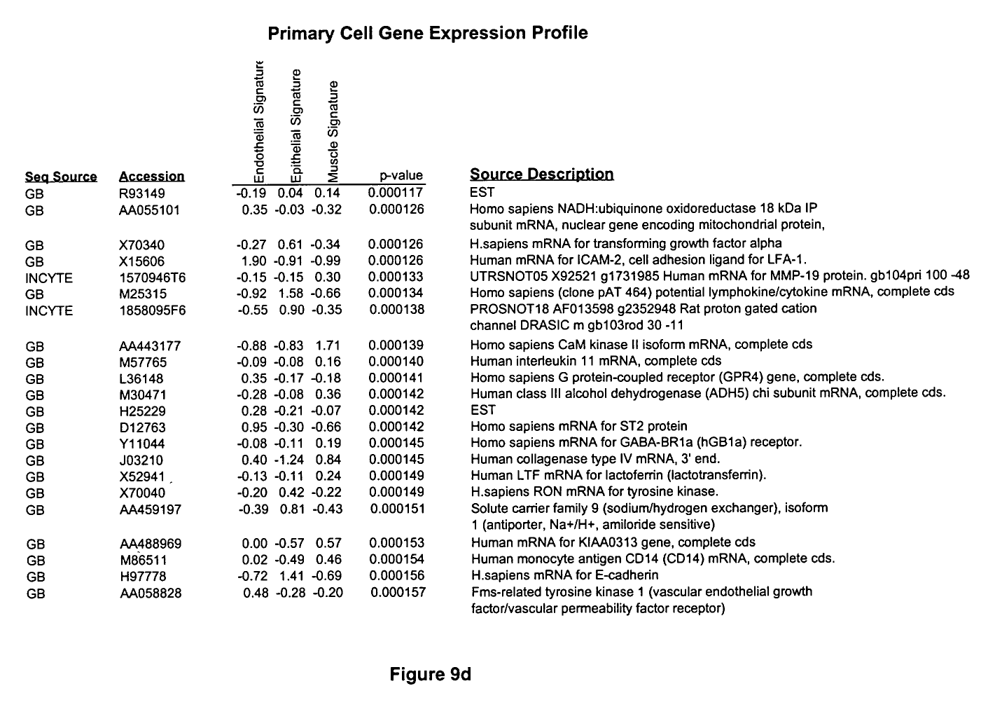

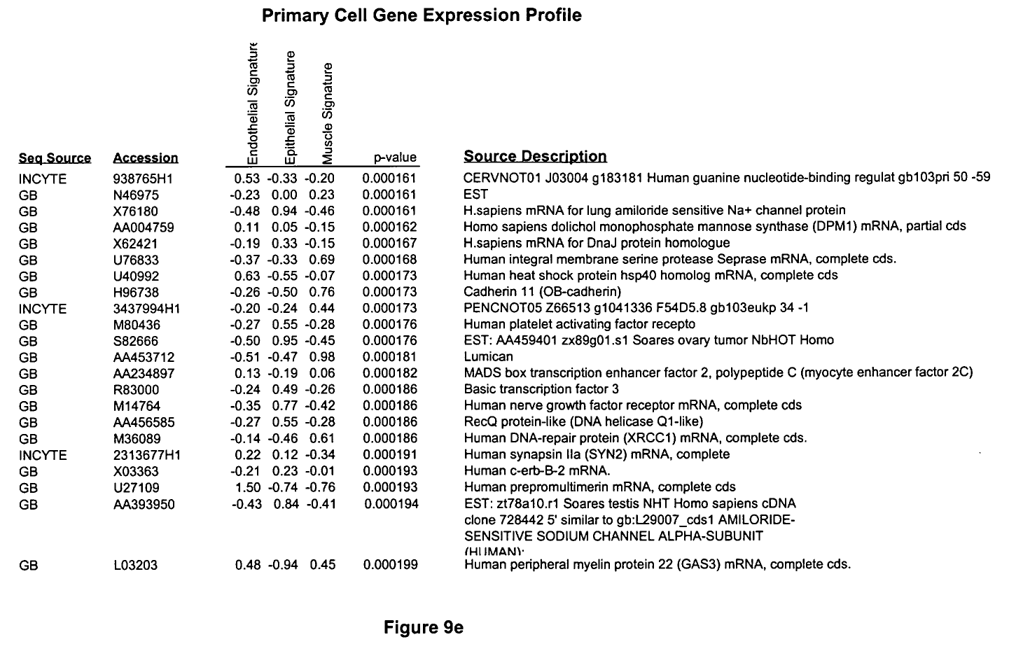

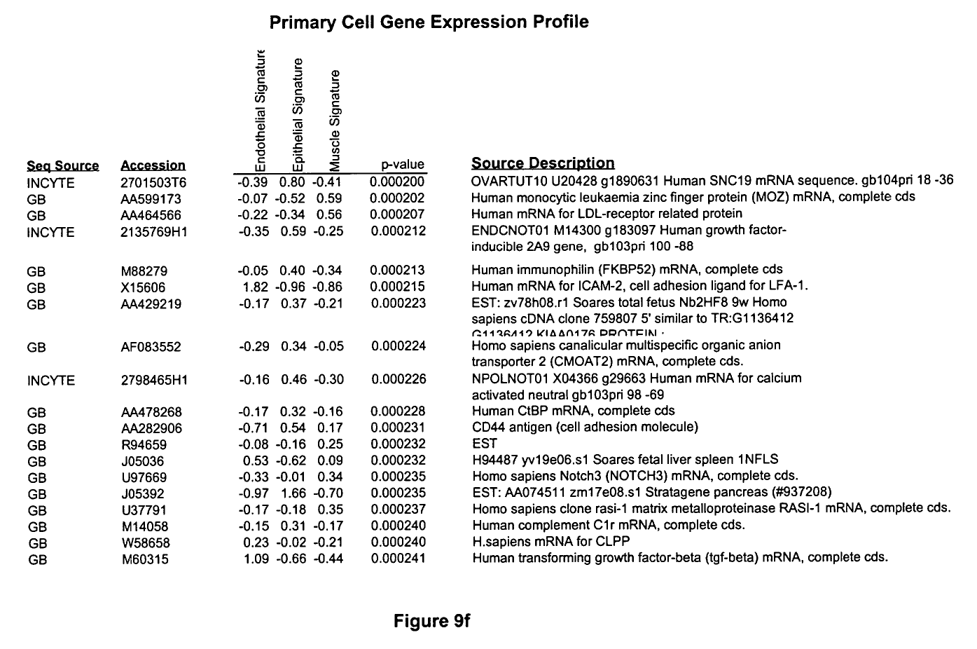

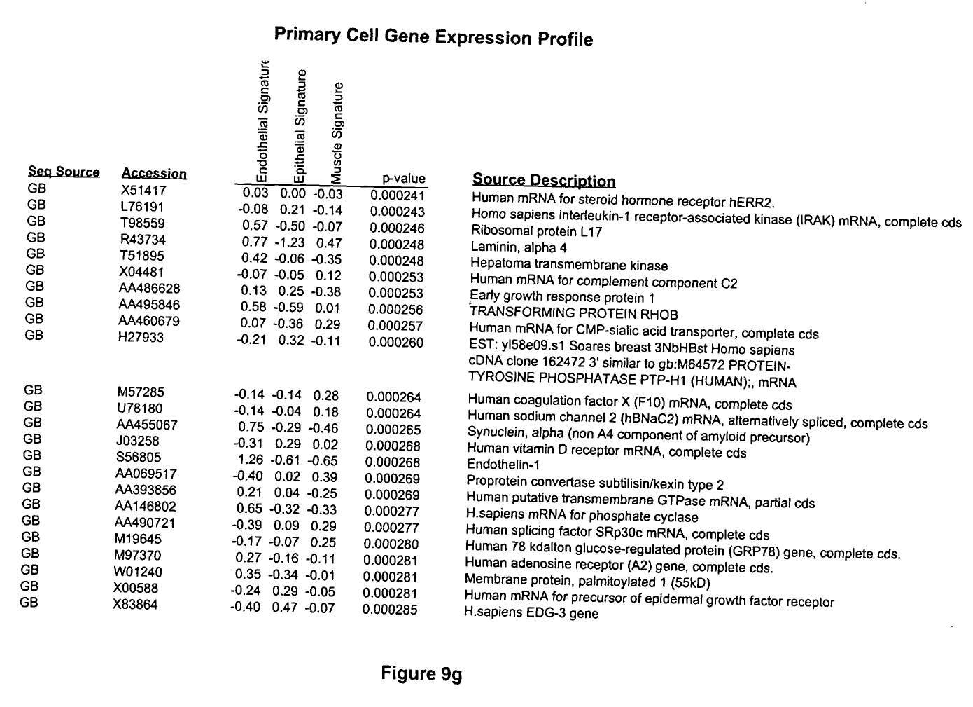

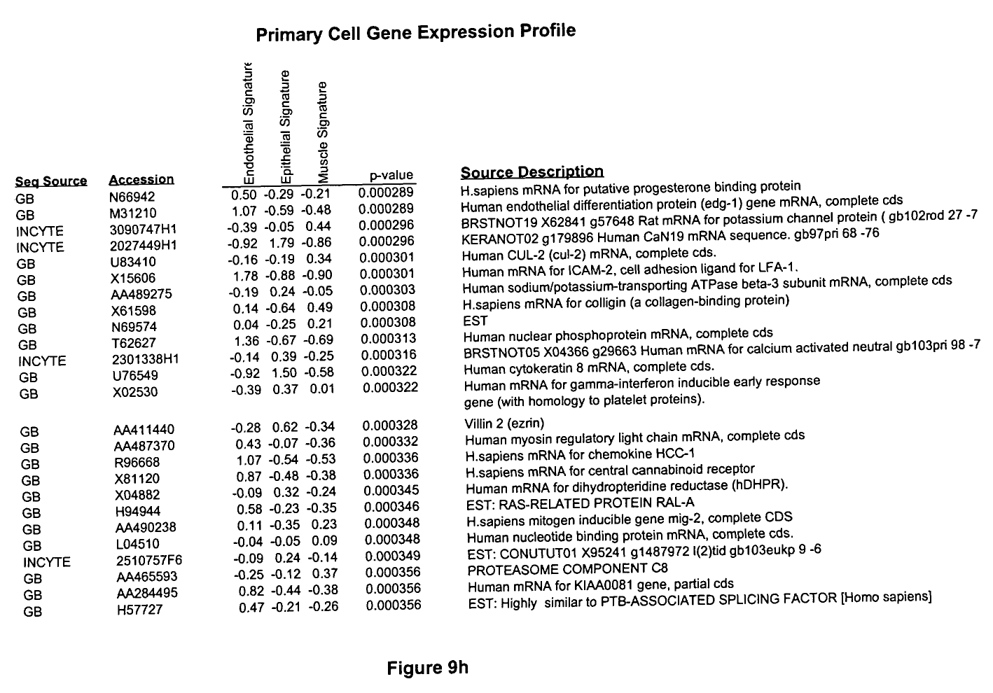

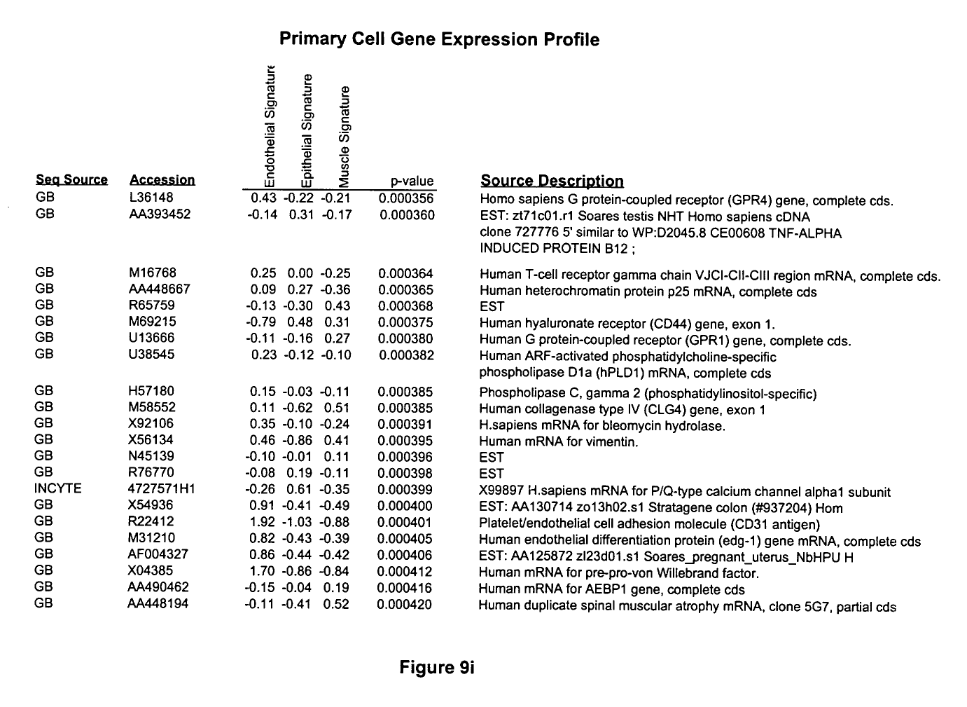

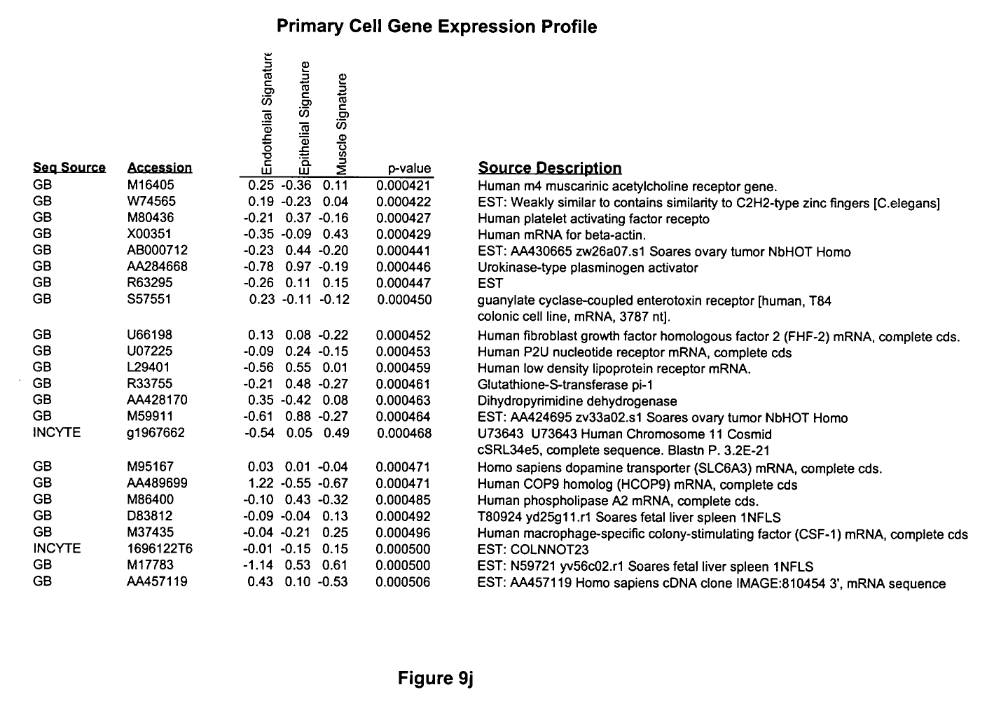

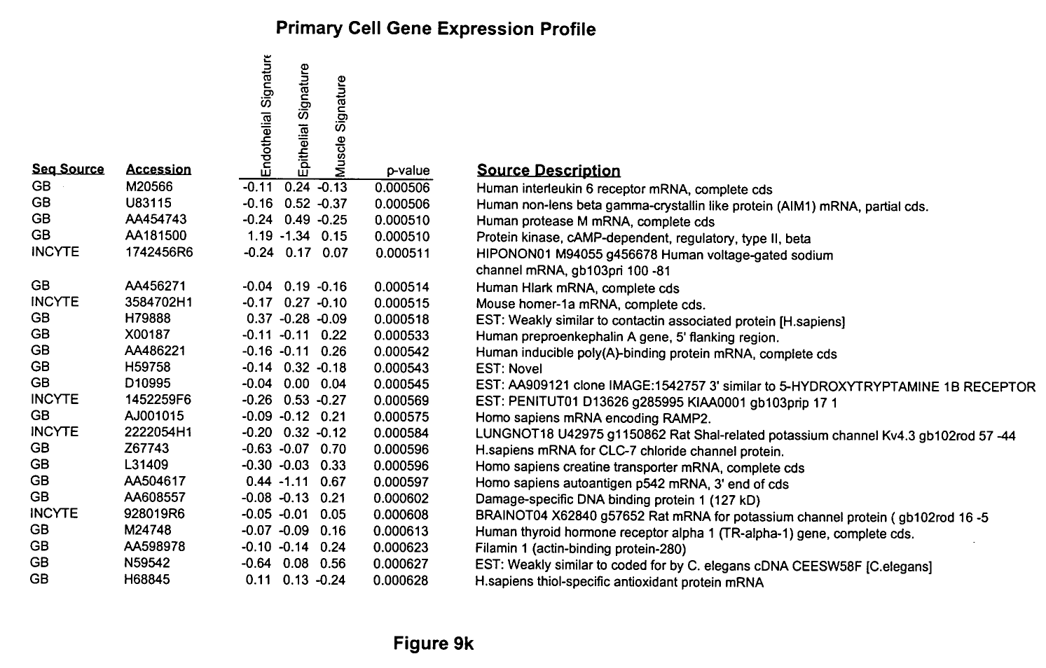

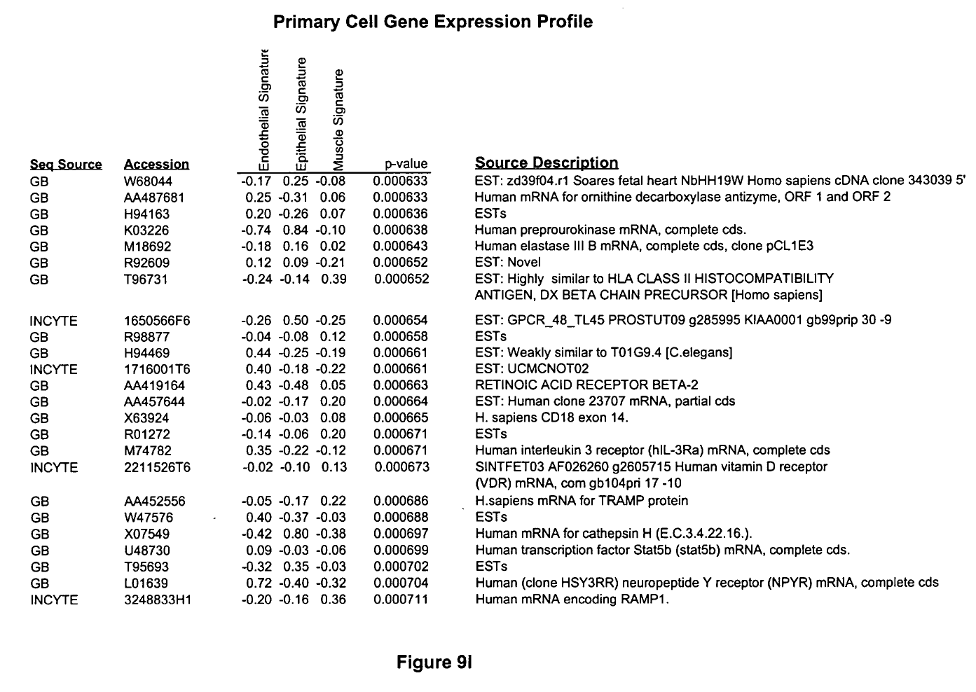

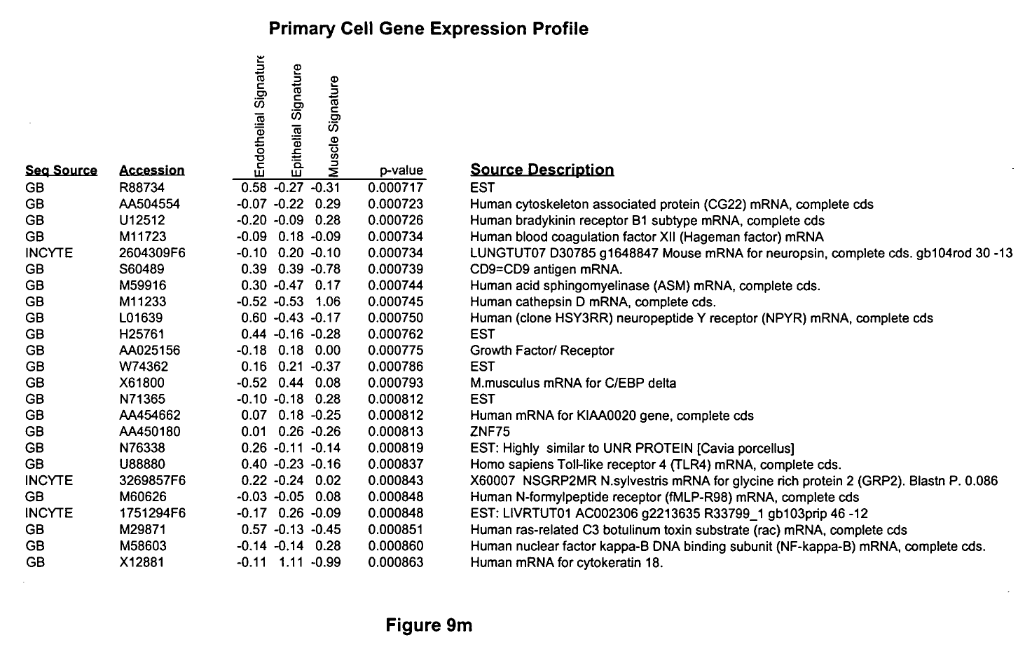

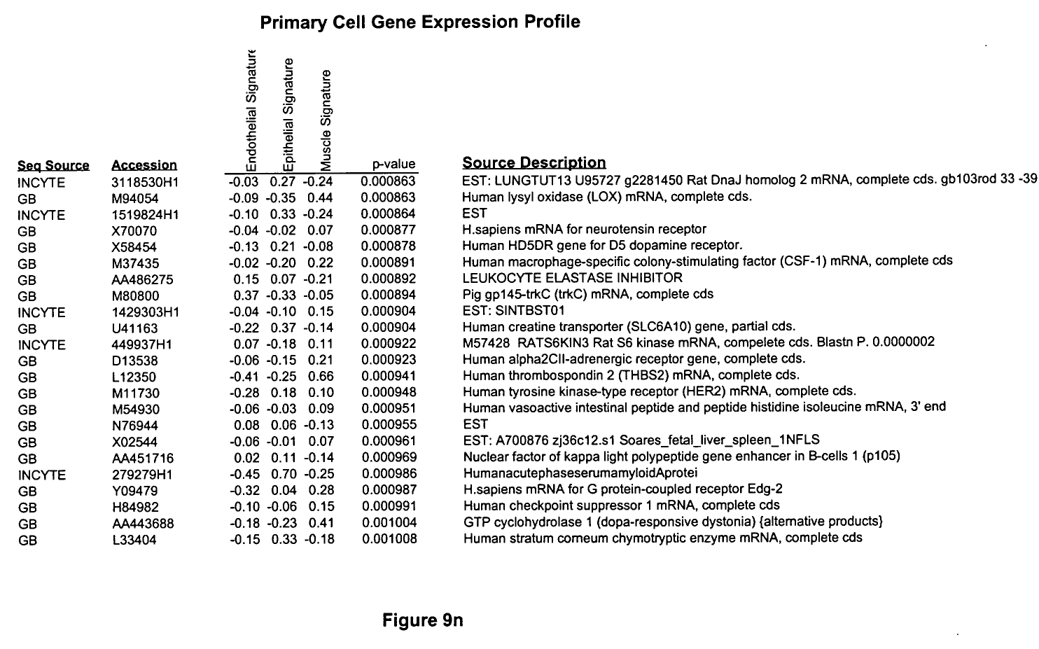

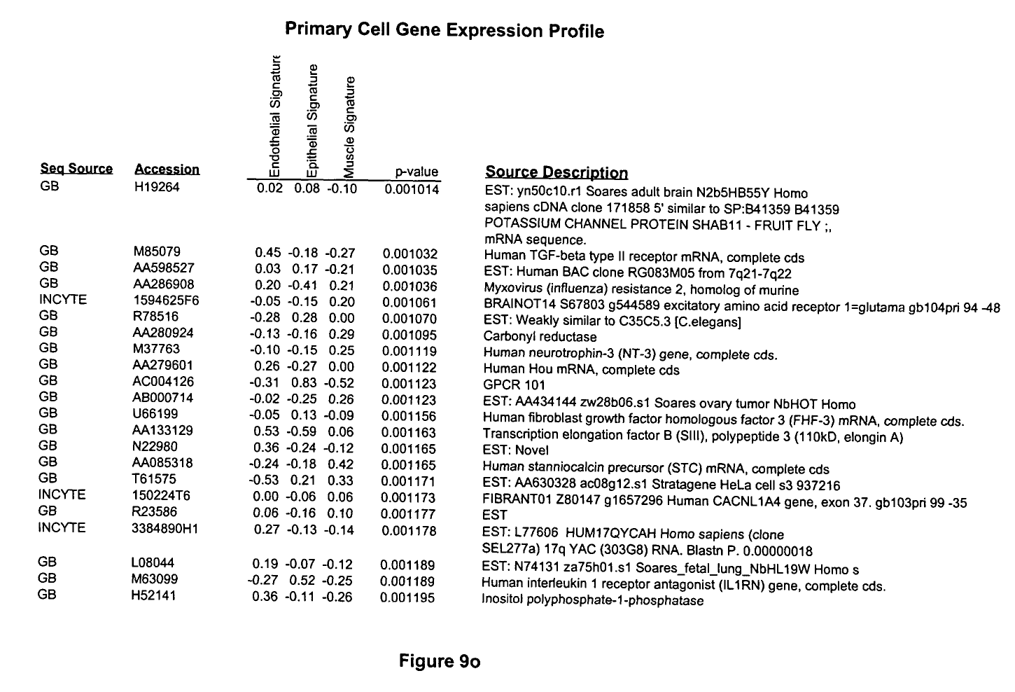

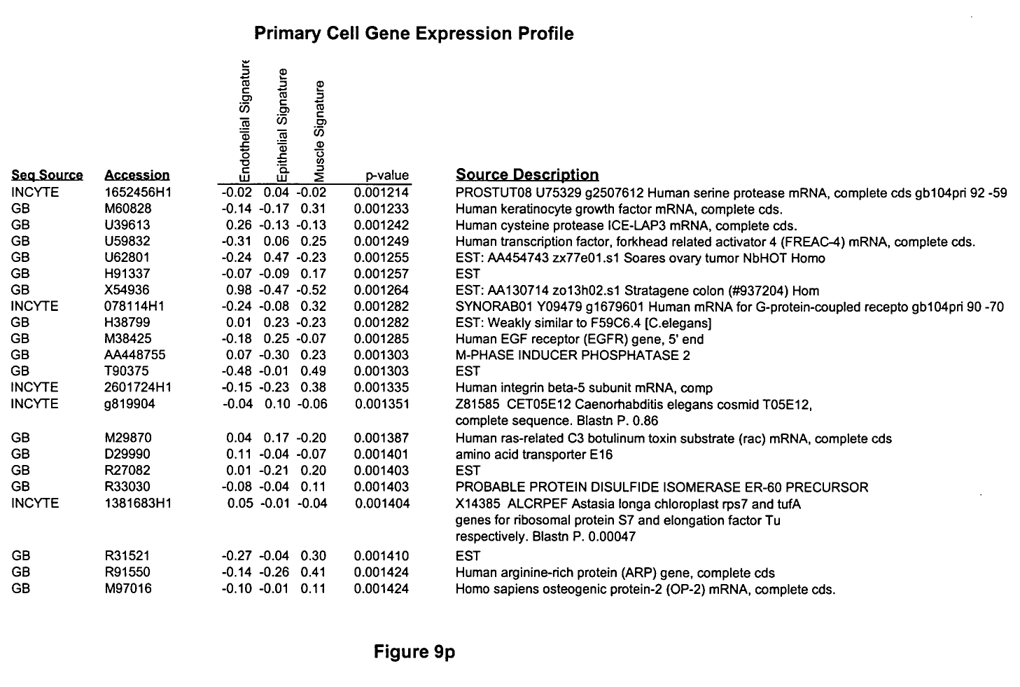

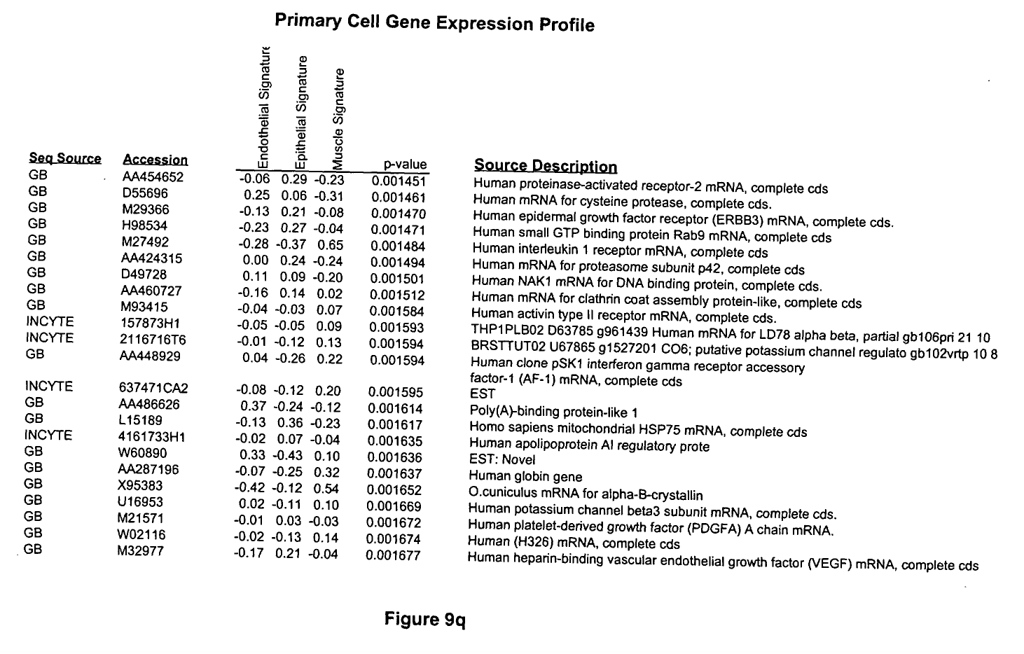

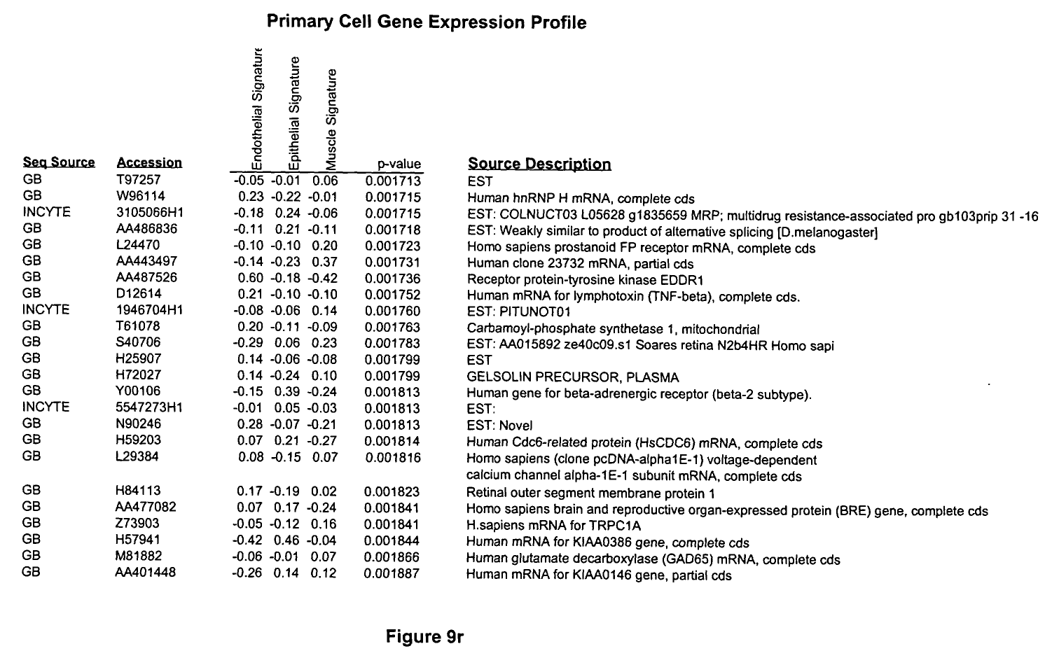

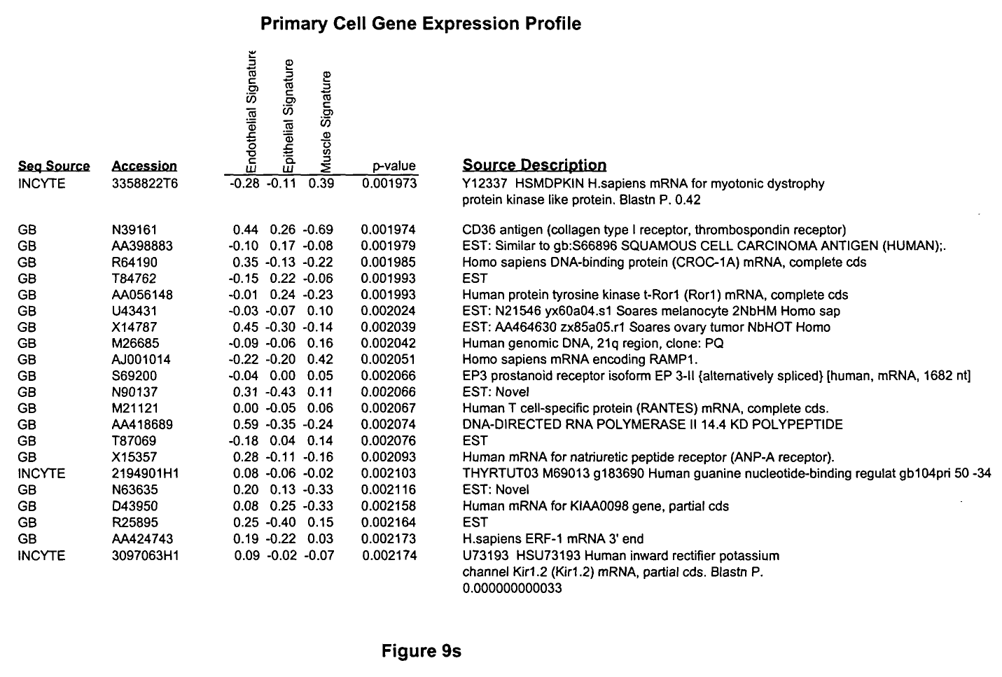

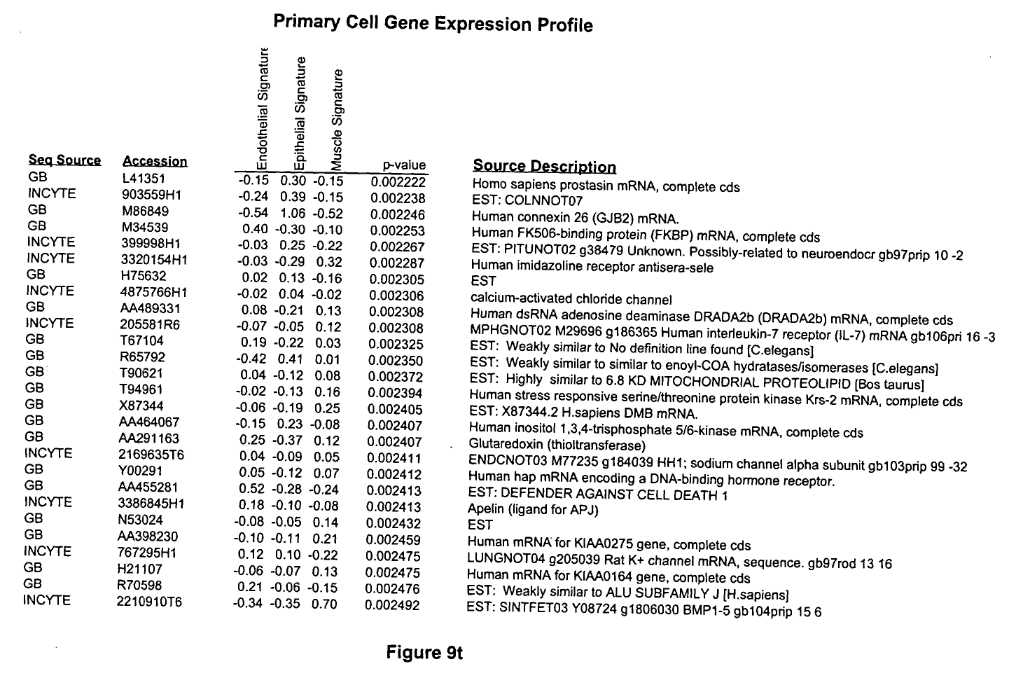

The resultant transformed matrix, composed of 459 genes from the 30 primary cell types, was then used for profile extraction using the Mean Log Ratio algorithm as described (FIG. 9). Four expression profiles were generated, primary, endothelial, epithelial, and muscle (FIGS. 9, 10, 11, and 12). The primary profile represents 186 genes that may be used to classify primary cells. The endothelial profile represents 55 genes that may be used to classify endothelial cells. The epithelial profile represents 52 genes that may be used to classify epithelial cells. Finally, the muscle profile represents 40 genes that may be used to classify muscle cells. The sequence source (Seq. Source) is the gene database (GB: GenBank; and INCYTE: Incyte Genomes) that the sequence was selected from and the Seq ID is the accession number of the particular gene sequence. The endothelial, epithelial, and muscle profile values are the numeric representation of the specific profile. The p-value is based on the Kruskal-Wallis rank test in which smaller p-values represents clones with higher discriminate power for classifying samples. The source description identifies the particular gene.

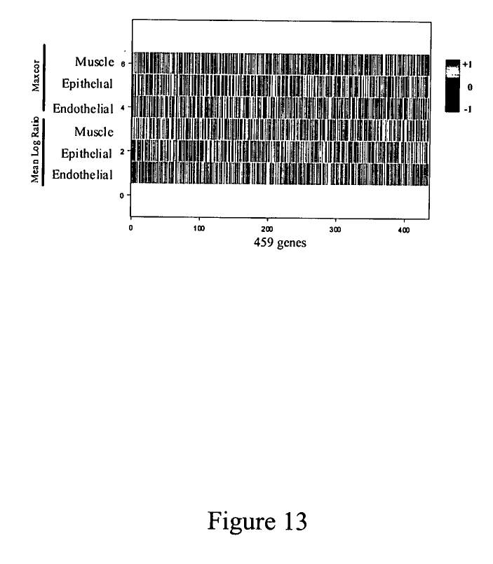

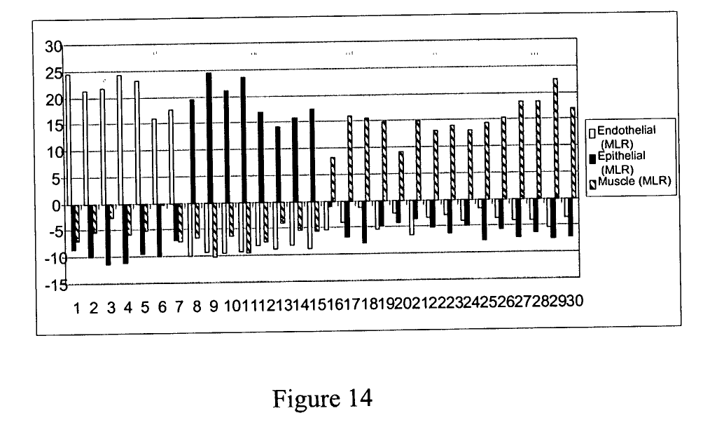

These expression profiles are also shown graphically by assigning colors to the numeric values obtained (FIG. 13). The expression profiles were then used to classify the 30 primary cells by taking each transformed primary cell gene expression vector and scoring it against the three expression profiles separately using the Mean Log Ratio scoring algorithm. The results demonstrated that the endothelial, epithelial, and muscle cell types scored high against their own expression profiles but low against the other two expression profiles (FIG. 14).

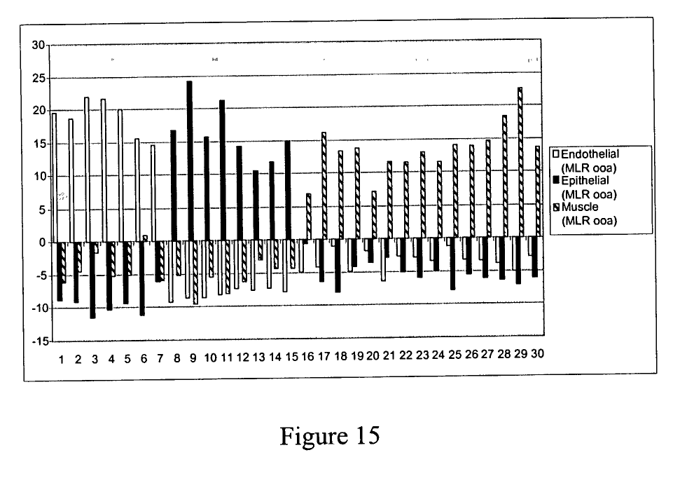

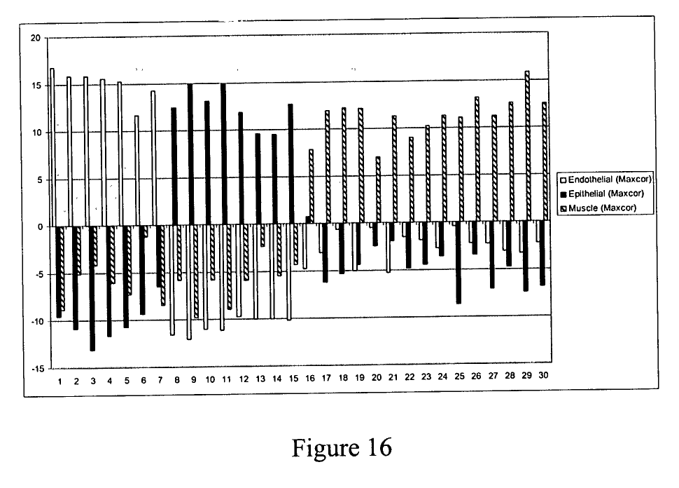

In additional experiments, a different primary cell sample was removed from the profile generation step and then scored against the resultant profile. The results from this analysis were similar to that in FIG. 5 indicating that the expression profiles can be used to score against independent samples (FIG. 15).

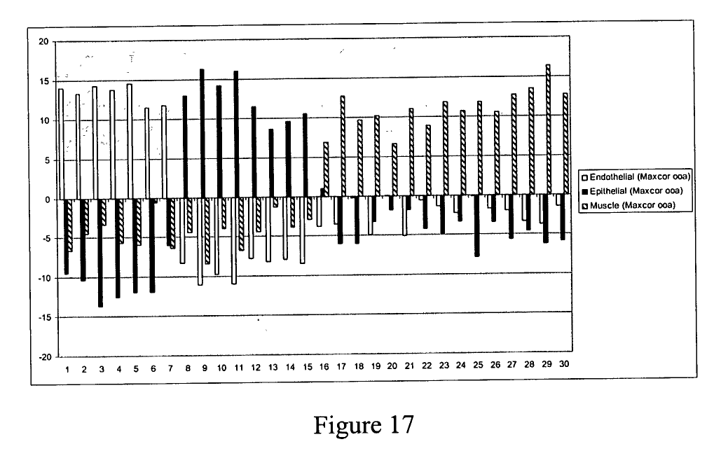

The analysis was repeated using the MaxCor algorithm as described. The self-validation results are shown in FIG. 16 and the omit one analysis result in FIG. 17. The results are essentially the same as that from the Mean Log Ratio analysis.

FIG. 9 shows a gene expression profile for primary cells. Specifically, a primary cell gene expression profile may comprise one or more of the following nucleic acid sequences: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 1; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO: 117; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186. Accordingly, these sequences may be used to identify a primary cell gene expression profile, which then may be used to classify unknown cell or tissue samples.

A primary cell gene expression profile may additionally comprise one or more of the following nucleic acid sequences: SEQ ID NO: 188; SEQ ID NO: 193; SEQ ID NO: 216; SEQ ID NO: 224; SEQ ID NO: 230; SEQ ID NO: 248; SEQ ID NO: 249; SEQ ID NO: 250; SEQ ID NO: 253; SEQ ID NO: 271; SEQ ID NO: 281; SEQ ID NO: 324; SEQ ID NO: 337; SEQ ID NO: 346; SEQ ID NO: 388; SEQ ID NO: 403; SEQ ID NO: 410; SEQ ID NO: 415; SEQ ID NO: 421; SEQ ID NO: 422; SEQ ID NO: 425; SEQ ID NO: 427; SEQ ID NO: 428; SEQ ID NO: 432; SEQ ID NO: 433; SEQ ID NO: 437; SEQ ID NO: 440; SEQ ID NO: 443; SEQ ID NO: 444; SEQ ID NO: 447; SEQ ID NO: 449; SEQ ID NO: 451; SEQ ID NO: 452; SEQ ID NO: 455; SEQ ID NO: 457; SEQ ID NO: 460; SEQ ID NO: 462; SEQ ID NO: 465; SEQ ID NO: 466; SEQ ID NO: 476; SEQ ID NO: 477; SEQ ID NO: 482; SEQ ID NO: 484; SEQ ID NO: 490; SEQ ID NO: 492; SEQ ID NO: 493; SEQ ID NO: 495; SEQ ID NO: 498; SEQ ID NO: 499; SEQ ID NO: 502; SEQ ID NO: 504; SEQ ID NO: 505; SEQ ID NO: 514; SEQ ID NO: 515; SEQ ID NO: 518; SEQ ID NO: 524; SEQ ID NO: 528; SEQ ID NO: 530; SEQ ID NO: 531; SEQ ID NO: 532; SEQ ID NO: 536; SEQ ID NO: 539; SEQ ID NO: 541; SEQ ID NO: 545; SEQ ID NO: 551; SEQ ID NO: 563; SEQ ID NO: 565; SEQ ID NO: 567; SEQ ID NO: 573; SEQ ID NO: 577; SEQ ID NO: 580; SEQ ID NO: 582; SEQ ID NO: 585; SEQ ID NO: 588; SEQ ID NO: 590; SEQ ID NO: 592; SEQ ID NO: 594; SEQ ID NO: 595; SEQ ID NO: 598; SEQ ID NO: 599; SEQ ID NO: 601; SEQ ID NO: 605; SEQ ID NO: 607; SEQ ID NO: 608; SEQ ID NO: 613; SEQ ID NO: 623; SEQ ID NO: 625; SEQ ID NO: 626; SEQ ID NO: 631; SEQ ID NO: 650; SEQ ID NO: 652; SEQ ID NO: 654; SEQ ID NO: 657; SEQ ID NO: 661; SEQ ID NO: 665; SEQ ID NO: 671; SEQ ID NO: 672; SEQ ID NO: 673; SEQ ID NO: 674; SEQ ID NO: 675; SEQ ID NO: 676; SEQ ID NO: 677; SEQ ID NO: 678; SEQ ID NO: 680; SEQ ID NO: 681; SEQ ID NO: 684; SEQ ID NO: 685; SEQ ID NO: 686; SEQ ID NO: 687; SEQ ID NO: 688; SEQ ID NO: 689; SEQ ID NO: 690; SEQ ID NO: 691; SEQ ID NO: 692; SEQ ID NO: 694; SEQ ID NO: 695; SEQ ID NO: 696; SEQ ID NO: 697; SEQ ID NO: 698; SEQ ID NO: 699; SEQ ID NO: 700; SEQ ID NO: 701; SEQ ID NO: 702; SEQ ID NO: 704; SEQ ID NO: 705; SEQ ID NO: 706; SEQ ID NO: 707; SEQ ID NO: 708; SEQ ID NO: 709; SEQ ID NO: 710; SEQ ID NO: 711; SEQ ID NO: 712; SEQ ID NO: 713; SEQ ID NO: 714; SEQ ID NO: 715; SEQ ID NO: 716; SEQ ID NO: 717; SEQ ID NO: 718; SEQ ID NO: 719; SEQ ID NO: 720; SEQ ID NO: 721; SEQ ID NO: 722; SEQ ID NO: 723; SEQ ID NO: 724; SEQ ID NO: 725; SEQ ID NO: 726; SEQ ID NO: 727; SEQ ID NO: 728; SEQ ID NO: 729; SEQ ID NO: 730; SEQ ID NO: 731; SEQ ID NO: 732; SEQ ID NO: 733; SEQ ID NO: 734; SEQ ID NO: 735; SEQ ID NO: 736; SEQ ID NO: 737; SEQ ID NO: 738; SEQ ID NO: 739; SEQ ID NO: 740; SEQ ID NO: 741; SEQ ID NO: 742; SEQ ID NO: 743; SEQ ID NO: 744; SEQ ID NO: 745; SEQ ID NO: 746; SEQ ID NO: 747; SEQ ID NO: 748; SEQ ID NO: 749; SEQ ID NO: 750; SEQ ID NO: 751; SEQ ID NO: 752; SEQ ID NO: 753; SEQ ID NO: 754; SEQ ID NO: 755; SEQ ID NO: 756; SEQ ID NO: 758; SEQ ID NO: 759; SEQ ID NO: 760; SEQ ID NO: 761; SEQ ID NO: 762; SEQ ID NO: 763; SEQ ID NO: 764; SEQ ID NO: 765; SEQ ID NO: 766; SEQ ID NO: 767; SEQ ID NO: 768; SEQ ID NO: 769; SEQ ID NO: 770; SEQ ID NO: 771; SEQ ID NO: 772; SEQ ID NO: 773; SEQ ID NO: 774; SEQ ID NO: 775; SEQ ID NO: 776; SEQ ID NO: 777; SEQ ID NO: 778; SEQ ID NO: 779; SEQ ID NO: 780; SEQ ID NO: 781; SEQ ID NO: 782; SEQ ID NO: 783; SEQ ID NO: 784; SEQ ID NO: 785; SEQ ID NO: 786; SEQ ID NO: 787; SEQ ID NO: 788; SEQ ID NO: 789; SEQ ID NO: 790; SEQ ID NO: 791; SEQ ID NO: 792; SEQ ID NO: 793; SEQ ID NO: 794; SEQ ID NO: 795; SEQ ID NO: 796; SEQ ID NO: 797; SEQ ID NO: 798; SEQ ID NO: 799; SEQ ID NO: 800; SEQ ID NO: 801; SEQ ID NO: 802; and SEQ ID NO: 803.

As the example shows, primary cell gene expression profile may also comprise, for instance, the nucleic acid sequences having the following accession numbers: INCYTE 2997284H1; INCYTE 1726828F6; INCYTE 1690295F6; INCYTE 530695T6; INCYTE 2313677H1; INCYTE 2510757F6; INCYTE 1696122T6; GB M20566; INCYTE 1742456R6; INCYTE 3584702H1; INCYTE 2222054H1; INCYTE 928019R6; INCYTE 1716001T6; INCYTE 2211526T6; INCYTE 2604309F6; INCYTE 3269857F6; INCYTE 1751294F6; INCYTE 3118530H1; INCYTE 1519824H1; INCYTE 1429303H1; INCYTE 449937H1; INCYTE 150224T6; INCYTE 1652456H1; INCYTE 2116716T6; INCYTE 637471CA2; INCYTE 3105066H1; INCYTE 1946704H1; INCYTE 5547273H1; INCYTE 2194901H1; INCYTE 3097063H1; INCYTE 399998H1; INCYTE 3320154H1; GB X87344; INCYTE 2169635T6; and INCYTE 767295H1.

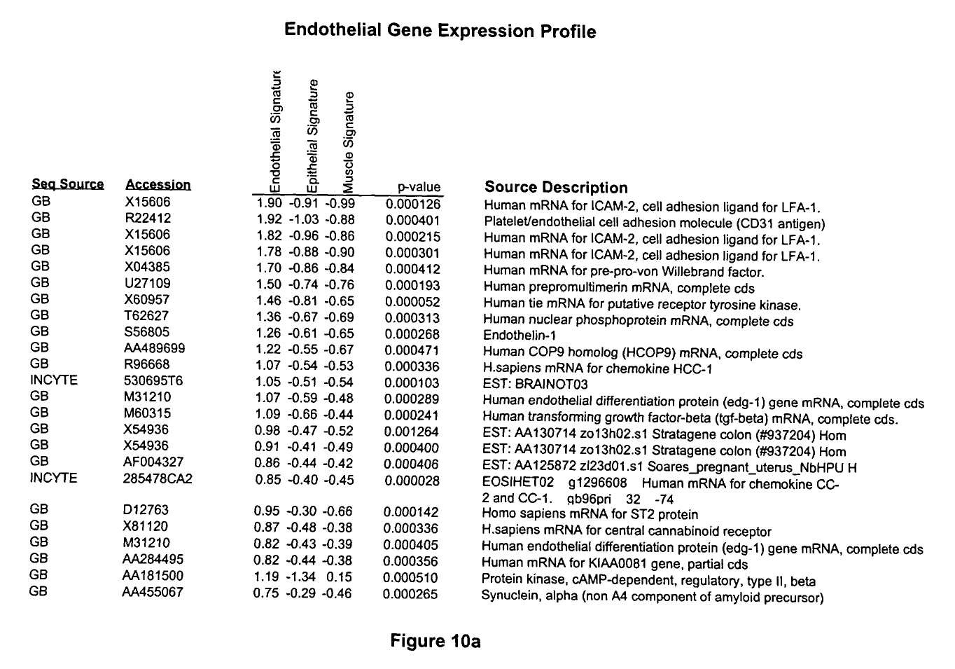

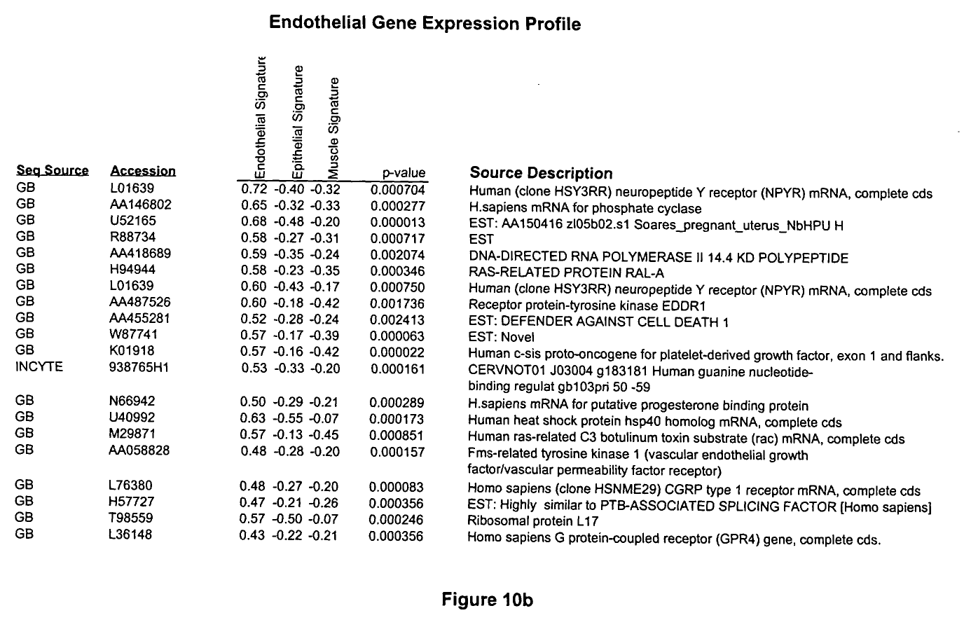

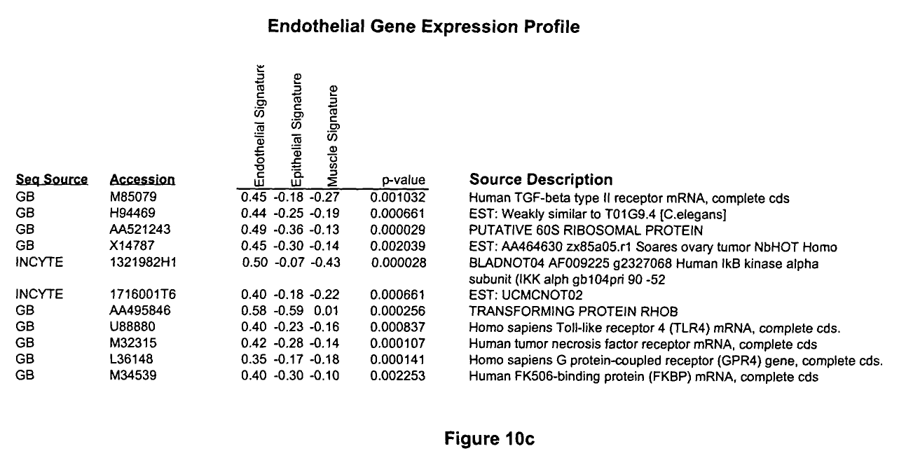

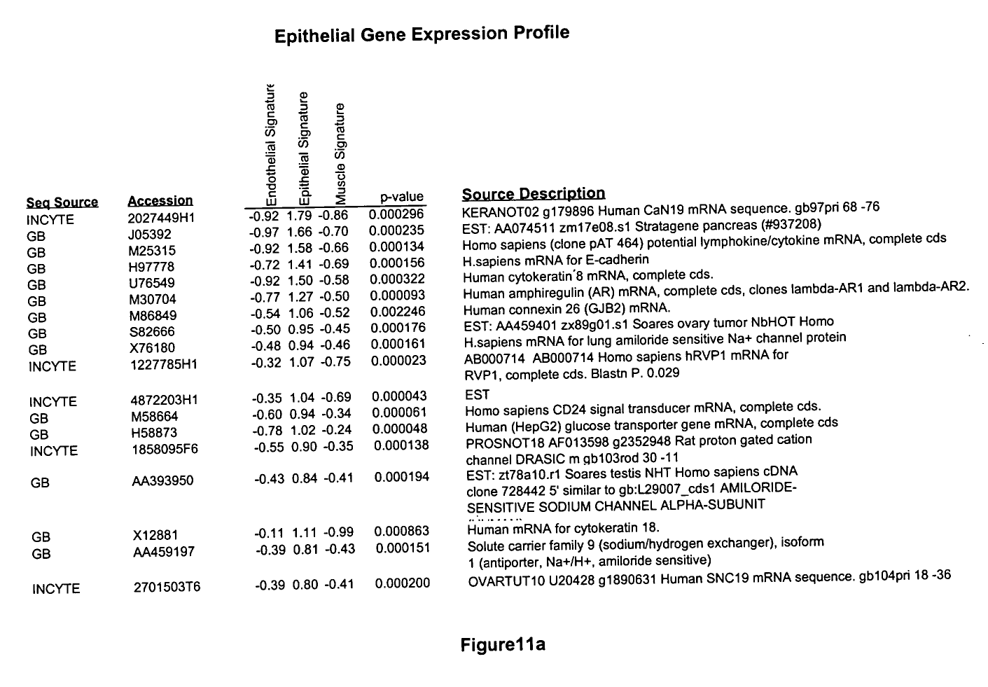

FIG. 10 displays the genes that comprise an endothelial gene expression profile. Specifically, an endothelial gene expression profile may comprise one or more nucleic acid sequences including, but not limited to, SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 48; SEQ ID NO: 63; SEQ ID NO: 70; SEQ ID NO: 82; SEQ ID NO: 94; and SEQ ID NO: 144. Accordingly, these sequences may be used to identify an endothelial gene expression profile, which then may be used to classify unknown cell or tissue samples.

An endothelial gene expression profile may additionally comprise one or more nucleic acid sequences including, but not limited to, SEQ ID NO: 427; SEQ ID NO: 460; SEQ ID NO: 484; SEQ ID NO: 565; SEQ ID NO: 580; SEQ ID NO: 590; SEQ ID NO: 670; SEQ ID NO: 672; SEQ ID NO: 673; SEQ ID NO: 674; SEQ ID NO: 675; SEQ ID NO: 676; SEQ ID NO: 677; SEQ ID NO: 678; SEQ ID NO: 680; SEQ ID NO: 723; SEQ ID NO: 741; and SEQ ID NO: 754.

As the example shows, an endothelial gene expression profile may also comprise, for example, the nucleic acid sequences having the following accession numbers: INCYTE 530695T6 and INCYTE 1716001T6.

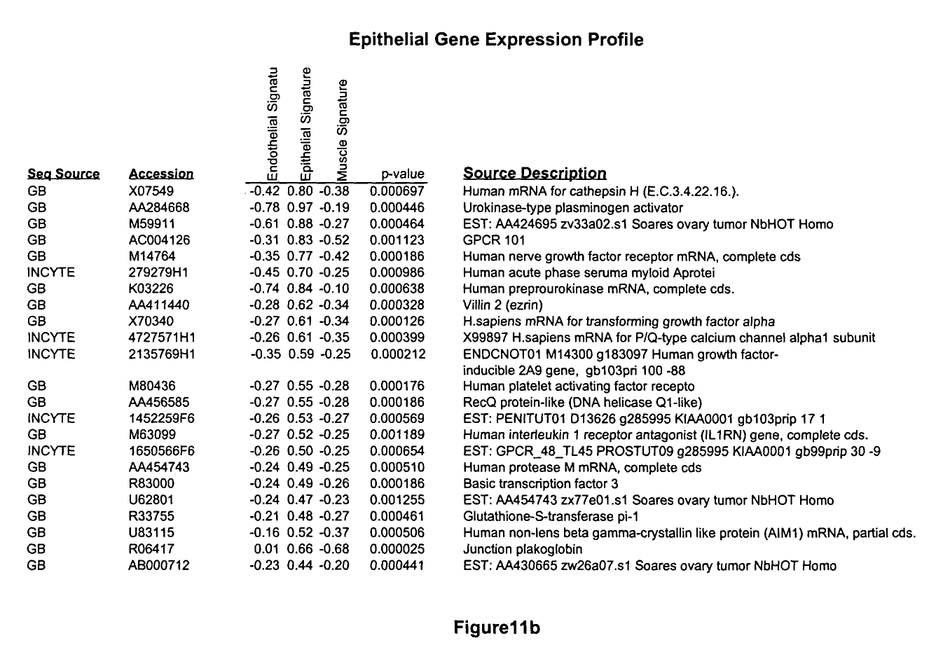

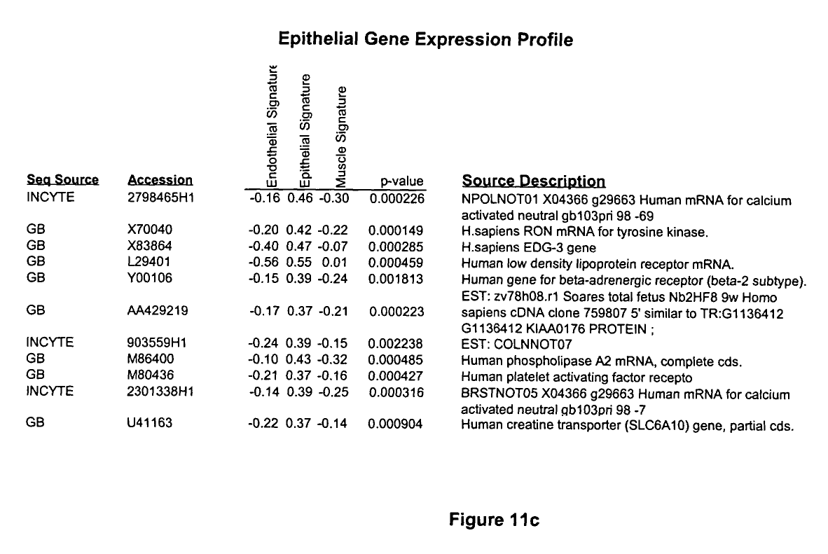

The gene expression profile depicted in FIG. 11 may be used to identify epithelial cells. Specifically, an epithelial gene expression profile may comprise one or more nucleic acid sequences including, but not limited to, SEQ ID NO: 47; SEQ ID NO: 60; SEQ ID NO: 67; SEQ ID NO: 73; SEQ ID NO: 75; SEQ ID NO: 76; SEQ ID NO: 77; SEQ ID NO: 78; SEQ ID NO: 80; SEQ ID NO: 96; SEQ ID NO: 98; SEQ ID NO: 99; SEQ ID NO: 111; SEQ ID NO: 112; SEQ ID NO: 117; SEQ ID NO: 123; SEQ ID NO: 127; SEQ ID NO: 131; SEQ ID NO: 150; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; SEQ ID NO: 186.

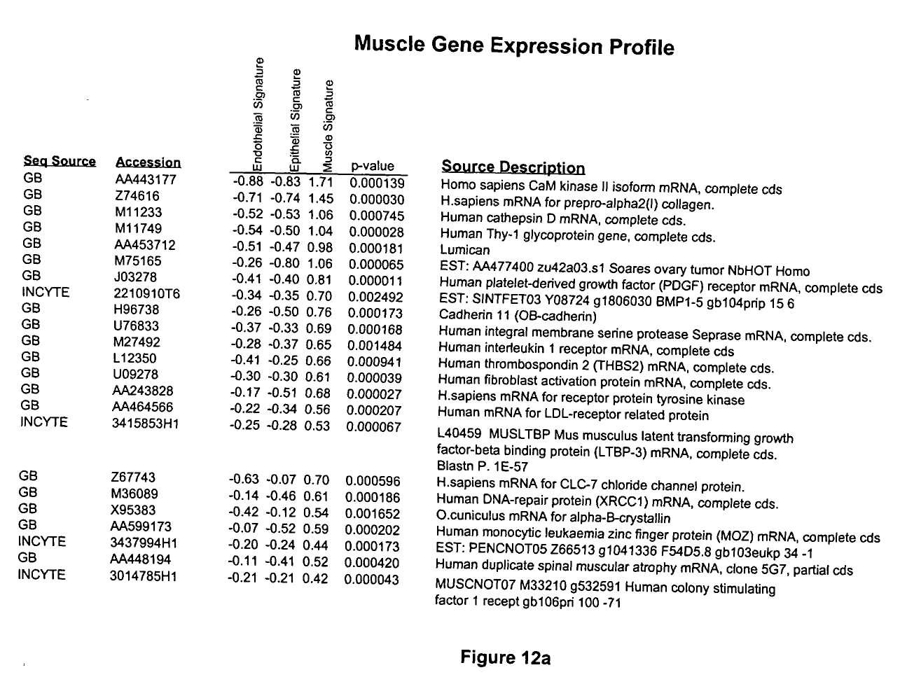

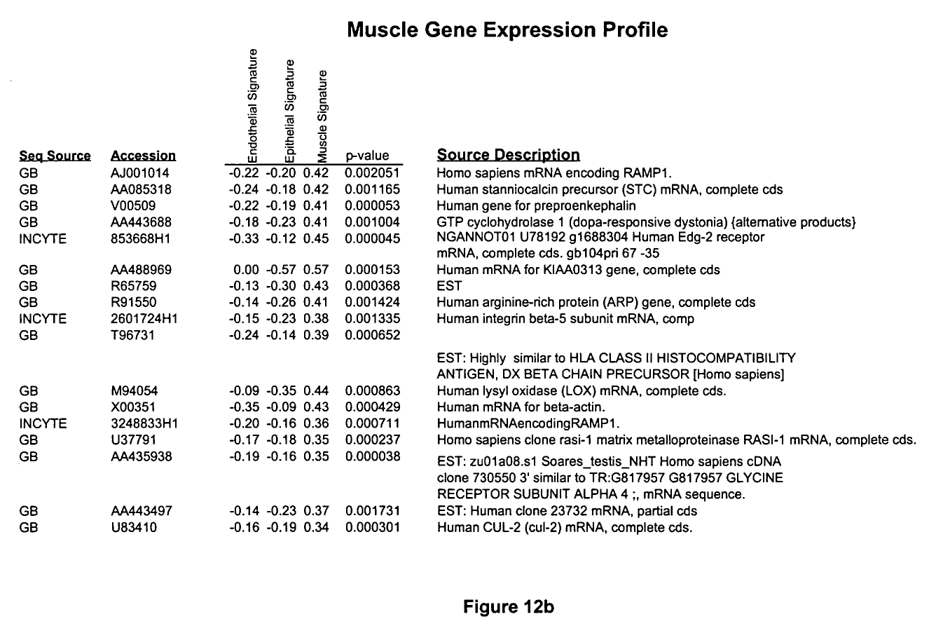

FIG. 12 shows the gene expression profile generated from muscle cells. In one embodiment, a muscle cell gene expression profile may comprise one or more nucleic acid sequences including, but not limited to, SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 54; SEQ ID NO: 55; and SEQ ID NO: 69. Accordingly, these sequences may be used to identify a muscle gene expression profile, which then may be used to classify unknown cell or tissue samples.

A muscle gene expression profile may additionally comprise one or more nucleic acid sequences including, but not limited to, SEQ ID NO: 188; SEQ ID NO: 193; SEQ ID NO: 216; SEQ ID NO: 250; SEQ ID NO: 499; SEQ ID NO: 504; SEQ ID NO: 563; SEQ ID NO: 652; SEQ ID NO: 681; SEQ ID NO: 682; SEQ ID NO: 683; SEQ ID NO: 684; SEQ ID NO: 685; SEQ ID NO: 686; SEQ ID NO: 687; SEQ ID NO: 688; SEQ ID NO: 689; SEQ ID NO: 690; and SEQ ID NO: 691.

### Example 4

### Gene Expression Profiles for Epithelial Cell Subtypes

Gene expression profiles that define a particular type of epithelial cell were generated using the methodologies, microarrays and algorithms of the present invention. Epithelial cell lines were used to generate the cell type specific gene expression profiles. The epithelial cell lines used in this example were derived from various tissues including keratinocyte epithelium, mammary epithelium, bronchial epithelium, prostate epithelium, renal cortical epithelium, renal proximal tubule epithelium, small airway epithelium, and renal epithelium.

Complementary DNA made from each of the eight cell lines was used to probe the microarray. Briefly, and as described in the previous examples, total RNA was extracted, amplified, and labeled. The resultant labeled cDNAs were hybridized to microarray chips. Following one or more washing steps, the microarrays were scanned and the intensity of the spots was recorded and converted into a numerical value and normalized. Next, the alogrithms of the present invention were applied to extract a gene expression profile that defined the subtype of epithelial cell.

The microarrays used in this example comprised the following nucleic acid sequences: SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; SEQ ID NO: 211; SEQ ID NO: 150; SEQ ID NO: 27; SEQ ID NO: 169; SEQ ID NO: 212; SEQ ID NO: 213; SEQ ID NO: 131; SEQ ID NO: 214; SEQ ID NO: 215; SEQ ID NO: 216; SEQ ID NO: 217; SEQ ID NO: 218; SEQ ID NO: 138; SEQ ID NO: 219; SEQ ID NO: 220; SEQ ID NO: 221; SEQ ID NO: 222; SEQ ID NO: 223; SEQ ID NO: 224; SEQ ID NO: 225; SEQ ID NO: 226; SEQ ID NO: 227; SEQ ID NO: 228; SEQ ID NO: 229; SEQ ID NO: 230; SEQ ID NO: 231; SEQ ID NO: 232; SEQ ID NO: 78; SEQ ID NO: 233; SEQ ID NO: 234; SEQ ID NO: 235; SEQ ID NO: 236; SEQ ID NO: 237; SEQ ID NO: 238; SEQ ID NO: 239; SEQ ID NO: 240; SEQ ID NO: 241; SEQ ID NO: 242; SEQ ID NO: 243; SEQ ID NO: 64; SEQ ID NO: 244; SEQ ID NO: 245; SEQ ID NO: 246; SEQ ID NO: 247; SEQ ID NO: 248; SEQ ID NO: 249; SEQ ID NO: 250; SEQ ID NO: 251; SEQ ID NO: 252; SEQ ID NO: 253; SEQ ID NO: 254; SEQ ID NO: 37; SEQ ID NO: 106; SEQ ID NO: 255; SEQ ID NO: 123; SEQ ID NO: 256; SEQ ID NO: 257; SEQ ID NO: 258; SEQ ID NO: 259; SEQ ID NO: 260; SEQ ID NO: 261; SEQ ID NO: 262; SEQ ID NO: 263; SEQ ID NO: 264; SEQ ID NO: 265; SEQ ID NO: 266; SEQ ID NO: 267; SEQ ID NO: 268; SEQ ID NO: 269; SEQ ID NO: 57; SEQ ID NO: 70; SEQ ID NO: 270; SEQ ID NO: 271; SEQ ID NO: 272; SEQ ID NO: 273; SEQ ID NO: 274; SEQ ID NO: 275; SEQ ID NO: 276; SEQ ID NO: 277; SEQ ID NO: 278; SEQ ID NO: 279; SEQ ID NO: 104; SEQ ID NO: 280; SEQ ID NO: 281; SEQ ID NO: 282; SEQ ID NO: 283; SEQ ID NO: 284; SEQ ID NO: 285; SEQ ID NO: 286; SEQ ID NO: 287; SEQ ID NO: 288; SEQ ID NO: 160; SEQ ID NO: 289; SEQ ID NO: 290; SEQ ID NO: 291; SEQ ID NO: 293; SEQ ID NO: 294; SEQ ID NO: 295; SEQ ID NO: 296; SEQ ID NO: 297; SEQ ID NO: 49; SEQ ID NO: 298; SEQ ID NO: 299; SEQ ID NO: 300; SEQ ID NO: 301; SEQ ID NO: 302; SEQ ID NO: 303; SEQ ID NO: 304; SEQ ID NO: 305; SEQ ID NO: 306; SEQ ID NO: 307; SEQ ID NO: 308; SEQ ID NO: 183; SEQ ID NO: 309; SEQ ID NO: 310; SEQ ID NO: 311; SEQ ID NO: 312; SEQ ID NO: 313; SEQ ID NO: 314; SEQ ID NO: 315; SEQ ID NO: 316; SEQ ID NO: 310; SEQ ID NO: 317; SEQ ID NO: 174; SEQ ID NO: 318; SEQ ID NO: 320; SEQ ID NO: 173; SEQ ID NO: 321; SEQ ID NO: 322; SEQ ID NO: 323; SEQ ID NO: 324; SEQ ID NO: 325; SEQ ID NO: 326; SEQ ID NO: 158; SEQ ID NO: 327; SEQ ID NO: 328; SEQ ID NO: 165; SEQ ID NO: 166; SEQ ID NO: 329

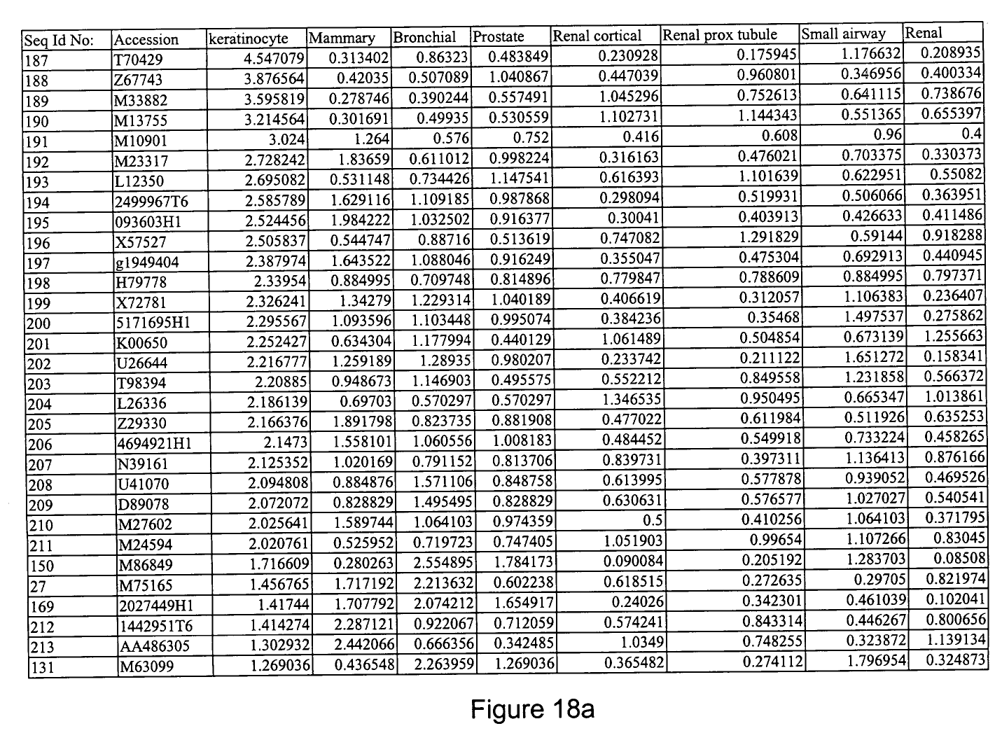

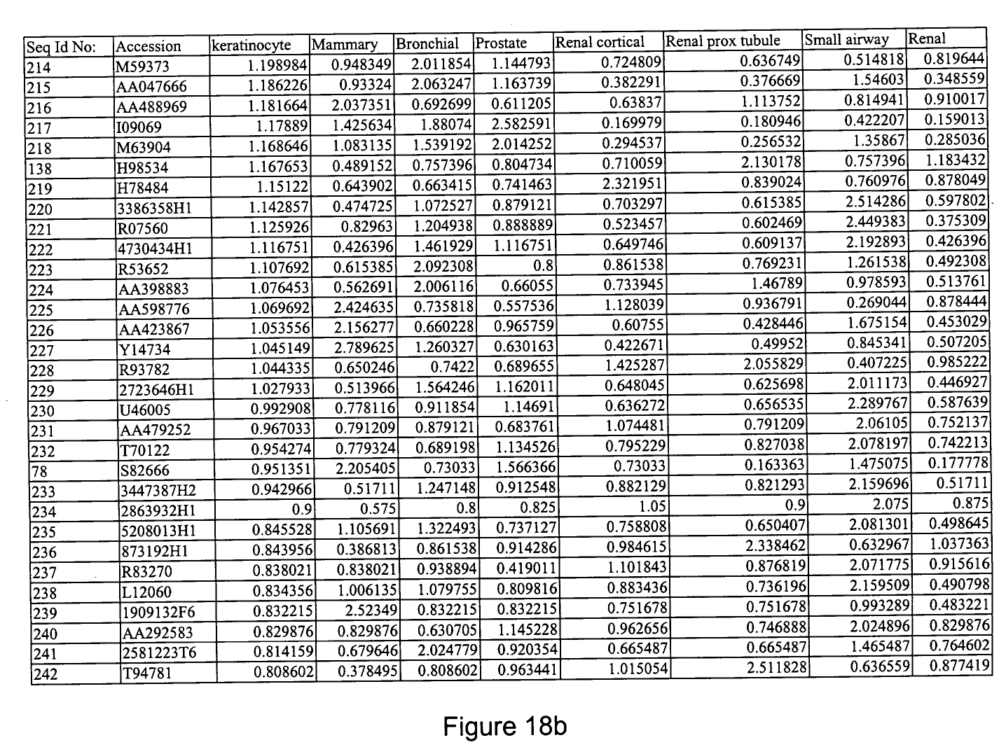

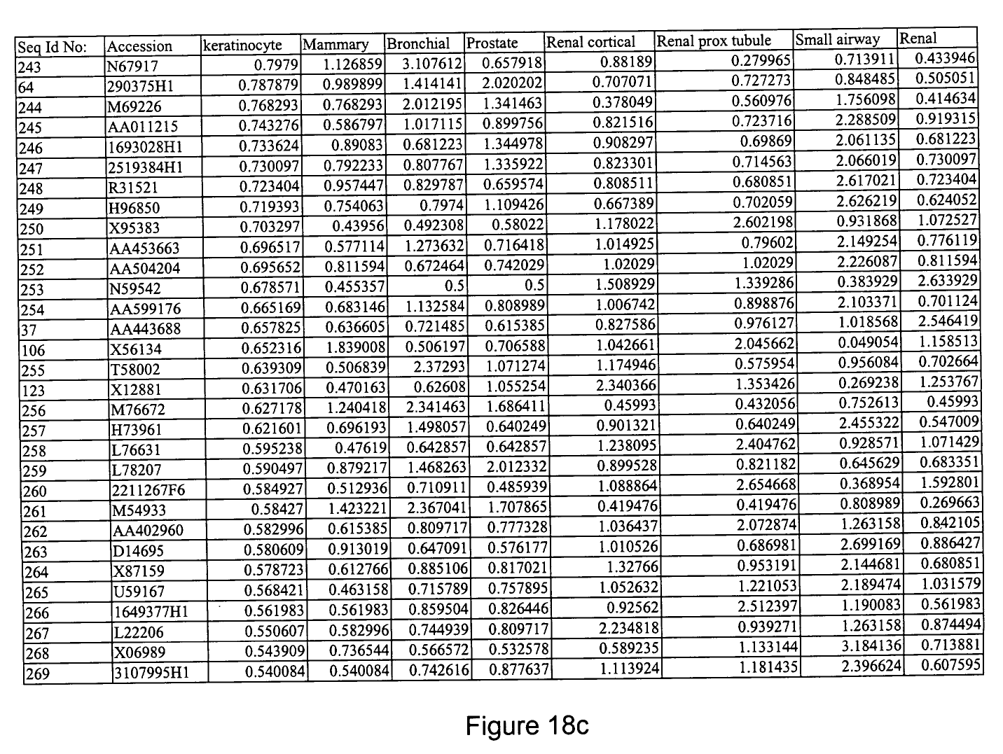

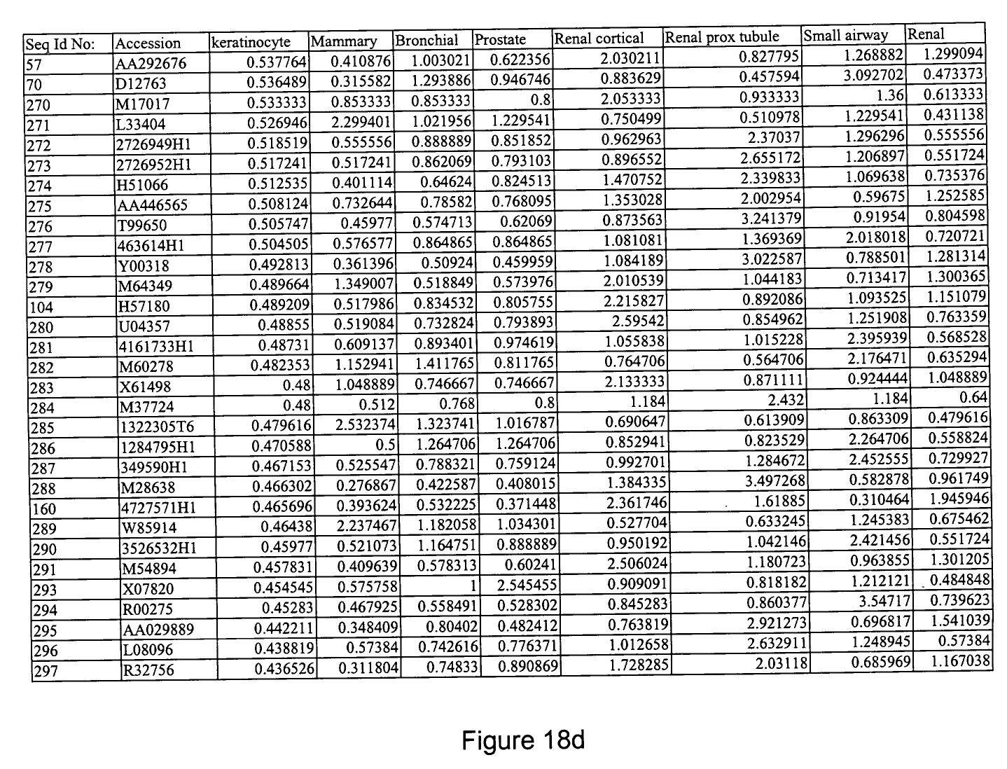

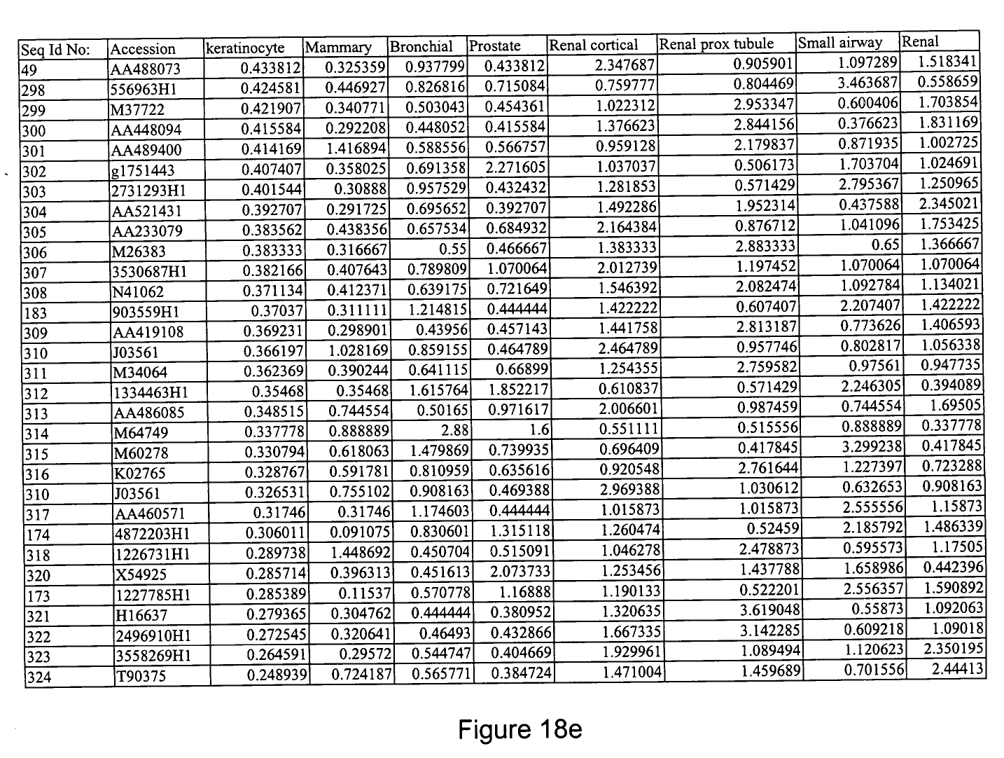

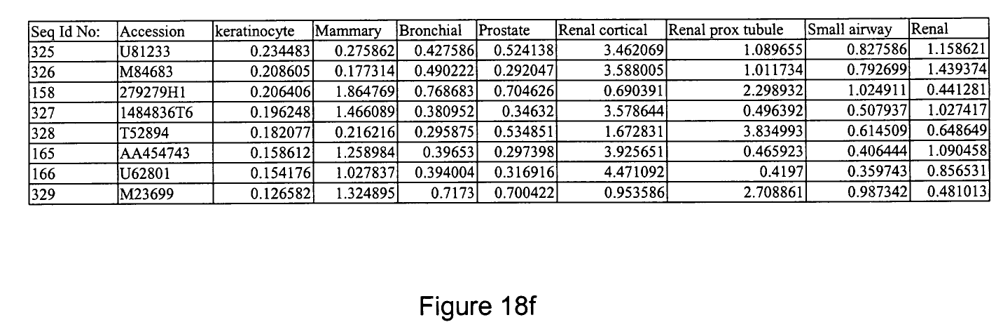

FIG. 18 shows the results from all eight of the hybridizations. The cutoff value was set for expression values over 2.0, i.e., two-fold induction over baseline. This particular portrayal of the data shows the relative expression values sorted for keratinocyte epithelial cells. Several genes, specifically, nucleic acid sequences SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; and SEQ ID NO: 211, show a relative expression value over 2.0, which is the cut-off in the context of the algorithm. These genes represent signature genes, i.e., a gene expression profile of keratinocyte epithelial cells, which may be used to identify and classify unkown samples.

With regard to the other columns, it is possible to sort the data and identify genes representing gene expression profiles of a particular cell type. For example, and referring to FIG. 18, sorting the data based on relative expression values and using the value of 2.0 as a cutoff in the context of the algorithm, the following genes represent a mammary epithelial cells gene expression profile: SEQ ID NO: 212; SEQ ID NO: 213; SEQ ID NO: 216; SEQ ID NO: 225; SEQ ID NO: 226; SEQ ID NO: 227; SEQ ID NO: 78; SEQ ID NO: 239; SEQ ID NO: 271; SEQ ID NO: 285; and SEQ ID NO: 289.

Similarly, and referring to FIG. 18, sorting the data based on relative expression values and using the value of 2.0 as a cutoff in the context of the algorithm, the following genes represent a bronchial epithelial cells gene expression profile: SEQ ID NO: 150; SEQ ID NO: 27; SEQ ID NO: 169; SEQ ID NO: 131; SEQ ID NO: 214; SEQ ID NO: 215; SEQ ID NO: 223; SEQ ID NO: 224; SEQ ID NO: 241; SEQ ID NO: 243; SEQ ID NO: 244; SEQ ID NO: 255; SEQ ID NO: 256; SEQ ID NO: 261; and SEQ ID NO: 314.

Referring to FIG. 18, sorting the data based on relative expression values and using the value of 2.0 as a cutoff in the context of the algorithm, the following genes represent a prostate epithelial cells gene expression profile: SEQ ID NO: 217; SEQ ID NO: 218; SEQ ID NO: 64; SEQ ID NO: 259; SEQ ID NO: 293; SEQ ID NO: 302; and SEQ ID NO: 320.

Likewise, referring to FIG. 18, sorting the data based on relative expression values and using the value of 2.0 as a cutoff in the context of the algorithm, the following genes represent a renal cortical epithelial cells gene expression profile: SEQ ID NO: 219; SEQ ID NO: 123; SEQ ID NO: 267; SEQ ID NO: 57; SEQ ID NO: 270; SEQ ID NO: 279; SEQ ID NO: 104; SEQ ID NO: 28; SEQ ID NO: 283; SEQ ID NO: 160; SEQ ID NO: 291; SEQ ID NO: 300; SEQ ID NO: 305; SEQ ID NO: 307; SEQ ID NO: 310; SEQ ID NO: 313; SEQ ID NO: 310; SEQ ID NO: 325; SEQ ID NO: 326; SEQ ID NO: 327; SEQ ID NO: 165; and SEQ ID NO: 166.

Referring to FIG. 18, sorting the data based on relative expression values and using the value of 2.0 as a cutoff in the context of the algorithm, the following genes represent a renal proximal tubule epithelial cells gene expression profile: SEQ ID NO: 106; SEQ ID NO: 138; SEQ ID NO: 158; SEQ ID NO: 228; SEQ ID NO: 236; SEQ ID NO: 242; SEQ ID NO: 250; SEQ ID NO: 258; SEQ ID NO: 260; SEQ ID NO: 262; SEQ ID NO: 266; SEQ ID NO: 272; SEQ ID NO: 273; SEQ ID NO: 274; SEQ ID NO: 275; SEQ ID NO: 276; SEQ ID NO: 278; SEQ ID NO: 284; SEQ ID NO: 288; SEQ ID NO: 295; SEQ ID NO: 296; SEQ ID NO: 297; SEQ ID NO: 299; SEQ ID NO: 300; SEQ ID NO: 301; SEQ ID NO: 306; SEQ ID NO: 308; SEQ ID NO: 309; SEQ ID NO: 311; SEQ ID NO: 316; SEQ ID NO: 318; SEQ ID NO: 321; SEQ ID NO: 322; SEQ ID NO: 328; and SEQ ID NO: 329.

Moreoever, and referring to FIG. 18, sorting the data based on relative expression values and using the value of 2.0 as a cutoff in the context of the algorithm, the following genes represent a small airway epithelial cells gene expression profile: SEQ ID NO: 173; SEQ ID NO: 174; SEQ ID NO: 183; SEQ ID NO: 220; SEQ ID NO: 221; SEQ ID NO: 222; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 237; SEQ ID NO: 238; SEQ ID NO: 240; SEQ ID NO: 245; SEQ ID NO: 246; SEQ ID NO: 247; SEQ ID NO: 248; SEQ ID NO: 249; SEQ ID NO: 251; SEQ ID NO: 252; SEQ ID NO: 254; SEQ ID NO: 257; SEQ ID NO: 263; SEQ ID NO: 264; SEQ ID NO: 265; SEQ ID NO: 268; SEQ ID NO: 269; SEQ ID NO: 270; SEQ ID NO: 277; SEQ ID NO: 281; SEQ ID NO: 282; SEQ ID NO: 286; SEQ ID NO: 287; SEQ ID NO: 290; SEQ ID NO: 294; SEQ ID NO: 298; SEQ ID NO: 303; SEQ ID NO: 312; SEQ ID NO: 315; SEQ ID NO: 317; and SEQ ID NO: 319.

Still further, and referring to FIG. 18, sorting the data based on relative expression values and using the value of 2.0 as a cutoff in the context of the algorithm, the following genes represent a renal epithelial cells gene expression profile: SEQ ID NO: 37; SEQ ID NO: 253; SEQ ID NO: 304; SEQ ID NO: 323; and SEQ ID NO: 324.

### Example 5

### Rat Toxicology Reference Database

To assess the toxicity of known compounds on gene and/or protein expression, a rat expression database is constructed. The database consists of gene expression profiles and protein expression profiles, as well as serum chemistry, hematology measurements, histopathology, and general clinical observations, from 100 different compounds at two doses and at two timepoints per dose. The compounds contain at least 10 different mechanisms of liver and kidney toxicity.

Sprague-Dawley rats are treated with compound via intraperitoneal administration. Dose groups include a low dose and a high dose for a 24-hour exposure and a low dose and a high dose for a 72-hour exposure. Three animals are treated per dose group as well as two control animal per timepoint. Following treatment, tissue are collected for gene expression and/or protein expression analysis including liver, kidney, white blood cells, lung, heart, intestine, testes, and spleen. Other toxicological evaluations include serum chemistry, hematology, organ weights, animal weights, and clinical observations.

Dose selection is based on literature reports with low dose defined as the lowest historical dose that elicited an endpoint and high dose is defined as the dose reported to result in a significant number of animals exhibiting characteristic toxicity.

The toxic effects of these compounds on gene expression and protein expression are analyzed using a toxicity microarray. For each compound, 15 rats are treated with the compound and tissue samples from each rat are collected and analyzed. The expression patterns in liver, kidney, heart, brain, intestine, testes, spleen, and white blood cells are analyzed following treatment with a toxic compounds. To generate the target nucleic acids, RNA or protein is isolated from each tissue sample and prepared for microarray hybridization as described above. Genes and/or proteins demonstrating alterations in expression level are selected for inclusion on the rat toxicity microarray. In addition, approximately 600 genes and/or protein-capture agents derived therefrom identified as toxicologically relevant based on review of the scientific literature are also be included on the microarray. In total, about 4,000 cDNAs or protein-capture agents reflecting the genes and/or proteins susceptible to the toxicity of these compounds.

Data reflecting the gene expression profiles of each tissue and toxin is placed in the database including an annotation describing dosage and clinical observations The database provides information describing mechanisms of action as well as previously reported alterations of gene expression observed following administration of these compounds. The database is also used in the drug discovery process by providing information which permits the elimination of potentially toxic compounds.

### Example 6

### Expression Profiles as a Diagnostic for Disease

The microarray technology may also be used to identify a particular disease (e.g., cancer), and provide a patient diagnosis. Initially, reference genes and/or proteins are generated for both normal and cancer cell types. Isolated cell types are derived by a number of methods known in the art (e.g., FACS sorting, magnoferric solutions, magnetic beads in combination with cell-specific antibodies). Cells from tissues are isolated by tissue staining with a cell-specific antibody, followed by laser capture microscopy or electrostatic methods. RNA is isolated from the cells and then probes are created for the generation of microarrays using the methods described above. Similarly, protein may be isolated from the cells and used to probe a microarray comprising protein-capture agnets using the methods described above.

Data from the microarrays for each cell type is then placed in a database along with an annotation describing cell type and location. Using cluster analysis and algorithms, gene and/or protein expression profiles for each cell type are determined.

For a diagnosis of Hodgkin lymphoma or non-Hodgkin lymphoma, biological samples are collected from patients and RNA or protein is isolated from the samples, as described above. The cDNA or protein is then hybridized to microarrays containing genes or protein-capture agents representing normal, Hodgkin lymphoma, and non-Hodgkin lymphoma samples. Based on the gene expression profiles and/or protein expression profiles, patients are diagnosed with either Hodgkin lymphoma or non-Hodgkin lymphoma.

The expression data from these patient samples is then added to the database. In addition, clinical information regarding the patient and treatment course as well as clinical outcome are also included in the database; thus, providing expression profiles for disease, disease stage, and outcome.

Microarray technology is also used to identify a course of treatment and as a drug discovery method. Normal and tumorogenic cells are treated with a known cancer drug (e.g., tamoxifen) or a novel pharmacological agent. As described above, RNA or protein is isolated and then hybridized to a microarray containing normal and cancer cell genes or protein-capture agents. A comparison of the expression levels following treatment provides an expression profile of the particular drug indicating which genes or proteins are activated or deactivated by the drug. This information is also added to the database. The database thus contains information describing the gene expression profiles and/or protein expression profiles of normal and cancer cells, gene expression profiles and/or protein expression profiles of patient samples, gene expression profiles and/or protein expression profiles of patients undergoing treatment, and gene expression profiles and/or protein expression profiles of in vitro cell studies. This information is used to diagnose and classify a disease, select and monitor a treatment course, and identify a prognostic indicator.

Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

We claim:

1. An endothelial cell gene expression profile comprising one or more nucleic acid sequences substantially homologous to a nucleic acid sequence or complementary sequence thereof selected from the group selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 48; SEQ ID NO: 63; SEQ ID NO: 70; SEQ ID NO: 82; SEQ ID NO: 94; and SEQ ID NO: 144.

2. A muscle cell gene expression profile comprising one or more nucleic acid sequences substantially homologous to a nucleic acid sequence or complementary sequence thereof selected from the group selected from the group consisting of SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 39; SEQ ID NO: 40; SEQ ID NO: 41; SEQ ID NO: 42; SEQ ID NO: 54; SEQ ID NO: 55; and SEQ ID NO: 69.

3. A primary cell gene expression profile comprising one or more nucleic acid sequences substantially homologous to a nucleic acid sequence or complementary sequence thereof selected from the group selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186.

4. An epithelial cell gene expression profile comprising one or more nucleic acid sequences substantially homologous to a nucleic acid sequence or complementary sequence thereof selected from the group selected from the group consisting of SEQ ID NO: 47; SEQ ID NO: 60; SEQ ID NO: 67; SEQ ID NO: 73; SEQ ID NO: 75; SEQ ID NO: 76; SEQ ID NO: 77; SEQ ID NO: 78; SEQ ID NO: 80; SEQ ID NO: 96; SEQ ID NO: 98; SEQ ID NO: 99; SEQ ID NO: 111; SEQ ID NO: 112; SEQ ID NO: 123; SEQ ID NO: 127; SEQ ID NO: 131; SEQ ID NO: 150; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186.

5. A keratinocyte epithelial cell gene expression profile comprising one or more nucleic acid sequences substantially homologous to a nucleic acid sequence or complementary sequence thereof selected from the group selected from the group consisting of SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; and SEQ ID NO: 211.

6. A mammary epithelial cell gene expression profile comprising one or more nucleic acid sequences substantially homologous to a nucleic acid sequence or complementary sequence thereof selected from the group selected from the group consisting of SEQ ID NO: 78; SEQ ID NO: 212; SEQ ID NO: 213; SEQ ID NO: 216; SEQ ID NO: 225; SEQ ID NO: 226; SEQ ID NO: 227; SEQ ID NO: 239; SEQ ID NO: 271; SEQ ID NO: 285; and SEQ ID NO: 289.

7. A bronchial epithelial cell gene expression profile comprising one or more nucleic acid sequences substantially homologous to a nucleic acid sequence or complementary sequence thereof selected from the group selected from the group consisting of SEQ ID NO: 27; SEQ ID NO: 131; SEQ ID NO: 150; SEQ ID NO: 169; SEQ ID NO: 214; SEQ ID NO: 215; SEQ ID NO: 223; SEQ ID NO: 224; SEQ ID NO: 241; SEQ ID NO: 243; SEQ ID NO: 244; SEQ ID NO: 255; SEQ ID NO: 256; SEQ ID NO: 261; and SEQ ID NO: 314.

8. A prostate epithelial cell gene expression profile comprising one or more nucleic acid sequences substantially homologous to a nucleic acid sequence or complementary sequence thereof selected from the group selected from the group consisting of SEQ ID NO: 64; SEQ ID NO: 217; SEQ ID NO: 218; SEQ ID NO: 259; SEQ ID NO: 293; SEQ ID NO: 302; and SEQ ID NO: 320.

9. A renal cortical epithelial cell gene expression profile comprising one or more nucleic acid sequences substantially homologous to a nucleic acid sequence or complementary sequence thereof selected from the group selected from the group consisting of SEQ ID NO: 49; SEQ ID NO: 57; SEQ ID NO: 104; SEQ ID NO: 123; SEQ ID NO: 160; SEQ ID NO: 165; SEQ ID NO: 166; SEQ ID NO: 219; SEQ ID NO: 267; SEQ ID NO: 270; SEQ ID NO: 279; SEQ ID NO: 280; SEQ ID NO: 283; SEQ ID NO: 291; SEQ ID NO: 305; SEQ ID NO: 307; SEQ ID NO: 310; SEQ ID NO: 313; SEQ ID NO: 325; SEQ ID NO: 326; and SEQ ID NO: 327.

10. A renal proximal tubule epithelial cell gene expression profile comprising one or more nucleic acid sequences substantially homologous to a nucleic acid sequence or complementary sequence thereof selected from the group selected from the group consisting of SEQ ID NO: 106; SEQ ID NO: 138; SEQ ID NO: 158; SEQ ID NO: 228; SEQ ID NO: 236; SEQ ID NO: 242; SEQ ID NO: 250; SEQ ID NO: 258; SEQ ID NO: 260; SEQ ID NO: 262; SEQ ID NO: 266; SEQ ID NO: 272; SEQ ID NO: 273; SEQ ID NO: 274; SEQ ID NO: 275; SEQ ID NO: 276; SEQ ID NO: 278; SEQ ID NO: 284; SEQ ID NO: 288; SEQ ID NO: 295; SEQ ID NO: 296; SEQ ID NO: 297; SEQ ID NO: 299; SEQ ID NO: 300; SEQ ID NO: 301; SEQ ID NO: 306; SEQ ID NO: 308; SEQ ID NO: 309; SEQ ID NO: 311; SEQ ID NO: 316; SEQ ID NO: 318; SEQ ID NO: 321; SEQ ID NO: 322; SEQ ID NO: 328; and SEQ ID NO: 329.

11. A small airway epithelial cell gene expression profile comprising one or more nucleic acid sequences substantially homologous to a nucleic acid sequence or complementary sequence thereof selected from the group selected from the group consisting of SEQ ID NO: 173; SEQ ID NO: 174; SEQ ID NO: 183; SEQ ID NO: 220; SEQ ID NO: 221; SEQ ID NO: 222; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 237; SEQ ID NO: 238; SEQ ID NO: 240; SEQ ID NO: 245; SEQ ID NO: 246; SEQ ID NO: 247; SEQ ID NO: 248; SEQ ID NO: 249; SEQ ID NO: 251; SEQ ID NO: 252; SEQ ID NO: 254; SEQ ID NO: 257; SEQ ID NO: 263; SEQ ID NO: 264; SEQ ID NO: 265; SEQ ID NO: 268; SEQ ID NO: 269; SEQ ID NO: 270; SEQ ID NO: 277; SEQ ID NO: 281; SEQ ID NO: 282; SEQ ID NO: 286; SEQ ID NO: 287; SEQ ID NO: 290; SEQ ID NO: 294; SEQ ID NO: 298; SEQ ID NO: 303; SEQ ID NO: 312; SEQ ID NO: 315; SEQ ID NO: 317; and SEQ ID NO: 319.

12. A renal epithelial cell gene expression profile comprising one or more nucleic acid sequences substantially homologous to a nucleic acid sequence or complementary sequence thereof selected from the group selected from the group consisting of SEQ ID NO: 37; SEQ ID NO: 253; SEQ ID NO: 304; SEQ ID NO: 323; and SEQ ID NO: 324.

13. A gene expression profile comprising one or more genes, wherein said gene expression profile is generated from a cell type selected from the group consisting of coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesangial cells, coronary artery smooth muscle, bronchial smooth muscle, uterine smooth muscle, lung fibroblast, osteoblasts, and prostate stromal cells.

14. A microarray comprising an endothelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 48; SEQ ID NO: 63; SEQ ID NO: 70; SEQ ID NO: 82; SEQ ID NO: 94; and SEQ ID NO: 144.

15. A microarray comprising muscle cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 39; SEQ ID NO: 40; SEQ ID NO: 41; SEQ ID NO: 42; SEQ ID NO: 54; SEQ ID NO: 55; and SEQ ID NO: 69.

16. A microarray comprising a primary cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 1; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 46; SEQ ID NO: 47; SEQ ID NO: 48; SEQ ID NO: 49; SEQ ID NO: 50; SEQ ID NO: 51; SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; SEQ ID NO: 76; SEQ ID NO: 77; SEQ ID NO: 78; SEQ ID NO: 79; SEQ ID NO: 80; SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; SEQ ID NO: 102; SEQ ID NO: 103; SEQ ID NO: 104; SEQ ID NO: 105; SEQ ID NO: 106; SEQ ID NO: 107; SEQ ID NO: 108; SEQ ID NO: 109; SEQ ID NO: 110; SEQ ID NO: 111; SEQ ID NO: 112; SEQ ID NO: 113; SEQ ID NO: 114; SEQ ID NO: 115; SEQ ID NO: 116; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 127; SEQ ID NO: 128; SEQ ID NO: 129; SEQ ID NO: 130; SEQ ID NO: 131; SEQ ID NO: 132; SEQ ID NO: 133; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; SEQ ID NO: 148; SEQ ID NO: 149; SEQ ID NO: 150; SEQ ID NO: 151; SEQ ID NO: 152; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186.

17. A microarray comprising an epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 47; SEQ ID NO: 60; SEQ ID NO: 67; SEQ ID NO: 73; SEQ ID NO: 75; SEQ ID NO: 76; SEQ ID NO: 77; SEQ ID NO: 78; SEQ ID NO: 80; SEQ ID NO: 96; SEQ ID NO: 98; SEQ ID NO: 99; SEQ ID NO: 111; SEQ ID NO: 112; SEQ ID NO: 123; SEQ ID NO: 127; SEQ ID NO: 131; SEQ ID NO: 150; SEQ ID NO: 153; SEQ ID NO: 154; SEQ ID NO: 155; SEQ ID NO: 156; SEQ ID NO: 157; SEQ ID NO: 158; SEQ ID NO: 159; SEQ ID NO: 160; SEQ ID NO: 161; SEQ ID NO: 162; SEQ ID NO: 163; SEQ ID NO: 164; SEQ ID NO: 165; SEQ ID NO: 166; SEQ ID NO: 167; SEQ ID NO: 168; SEQ ID NO: 169; SEQ ID NO: 170; SEQ ID NO: 171; SEQ ID NO: 172; SEQ ID NO: 173; SEQ ID NO: 174; SEQ ID NO: 175; SEQ ID NO: 176; SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; and SEQ ID NO: 186.

18. A microarray comprising a keratinocyte epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; and SEQ ID NO: 211.

19. A microarray comprising a mammary epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 78; SEQ ID NO: 212; SEQ ID NO: 213; SEQ ID NO: 216; SEQ ID NO: 225; SEQ ID NO: 226; SEQ ID NO: 227; SEQ ID NO: 239; SEQ ID NO: 271; SEQ ID NO: 285; and SEQ ID NO: 289.

20. A microarray comprising a bronchial epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 27; SEQ ID NO: 131; SEQ ID NO: 150; SEQ ID NO: 169; SEQ ID NO: 214; SEQ ID NO: 215; SEQ ID NO: 223; SEQ ID NO: 224; SEQ ID NO: 241; SEQ ID NO: 243; SEQ ID NO: 244; SEQ ID NO: 255; SEQ ID NO: 256; SEQ ID NO: 261; and SEQ ID NO: 314.

21. A microarray comprising a prostate epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 64; SEQ ID NO: 217; SEQ ID NO: 218; SEQ ID NO: 259; SEQ ID NO: 293; SEQ ID NO: 302; and SEQ ID NO: 320.

22. A microarray comprising a renal cortical epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 49; SEQ ID NO: 57; SEQ ID NO: 104; SEQ ID NO: 123; SEQ ID NO: 160; SEQ ID NO: 165; SEQ ID NO: 166; SEQ ID NO: 219; SEQ ID NO: 267; SEQ ID NO: 270; SEQ ID NO: 279; SEQ ID NO: 280; SEQ ID NO: 283; SEQ ID NO: 291; SEQ ID NO: 305; SEQ ID NO: 307; SEQ ID NO: 310; SEQ ID NO: 313; SEQ ID NO: 325; SEQ ID NO: 326; and SEQ ID NO: 327.

23. A microarray comprising renal proximal tubule epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 106; SEQ ID NO: 138; SEQ ID NO: 158; SEQ ID NO: 228; SEQ ID NO: 236; SEQ ID NO: 242; SEQ ID NO: 250; SEQ ID NO: 258; SEQ ID NO: 260; SEQ ID NO: 262; SEQ ID NO: 266; SEQ ID NO: 272; SEQ ID NO: 273; SEQ ID NO: 274; SEQ ID NO: 275; SEQ ID NO: 276; SEQ ID NO: 278; SEQ ID NO: 284; SEQ ID NO: 288; SEQ ID NO: 295; SEQ ID NO: 296; SEQ ID NO: 297; SEQ ID NO: 299; SEQ ID NO: 300; SEQ ID NO: 301; SEQ ID NO: 306; SEQ ID NO: 308; SEQ ID NO: 309; SEQ ID NO: 311; SEQ ID NO: 316; SEQ ID NO: 318; SEQ ID NO: 321; SEQ ID NO: 322; SEQ ID NO: 328; and SEQ ID NO: 329.

24. A microarray comprising a small airway epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 173; SEQ ID NO: 174; SEQ ID NO: 183; SEQ ID NO: 220; SEQ ID NO: 221; SEQ ID NO: 222; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 237; SEQ ID NO: 238; SEQ ID NO: 240; SEQ ID NO: 245; SEQ ID NO: 246; SEQ ID NO: 247; SEQ ID NO: 248; SEQ ID NO: 249; SEQ ID NO: 251; SEQ ID NO: 252; SEQ ID NO: 254; SEQ ID NO: 257; SEQ ID NO: 263; SEQ ID NO: 264; SEQ ID NO: 265; SEQ ID NO: 268; SEQ ID NO: 269; SEQ ID NO: 270; SEQ ID NO: 277; SEQ ID NO: 281; SEQ ID NO: 282; SEQ ID NO: 286; SEQ ID NO: 287; SEQ ID NO: 290; SEQ ID NO: 294; SEQ ID NO: 298; SEQ ID NO: 303; SEQ ID NO: 312; SEQ ID NO: 315; SEQ ID NO: 317; and SEQ ID NO: 319.

25. A microarray comprising a renal epithelial cell gene expression profile comprising one or more nucleic acid sequences substantially homologous to a nucleic acid sequence or complementary sequence thereof, or portions of said nucleic acid sequence or complementary sequence thereof, selected from the group consisting of SEQ ID NO: 37; SEQ ID NO: 253; SEQ ID NO: 304; SEQ ID NO: 323; and SEQ ID NO: 324.

26. A microarray comprising one or more nucleic acid sequences substantially homologous to a nucleic acid sequence or complementary sequence thereof, or portions of said nucleic acid sequence or complementary sequence thereof, selected from the group consisting of SEQ ID NO: 27; SEQ ID NO: 37; SEQ ID NO: 49; SEQ ID NO: 57; SEQ ID NO: 64; SEQ ID NO: 70; SEQ ID NO: 78; SEQ ID NO: 104; SEQ ID NO: 106; SEQ ID NO: 123; SEQ ID NO: 131; SEQ ID NO: 138; SEQ ID NO: 150; SEQ ID NO: 158; SEQ ID NO: 160; SEQ ID NO: 165; SEQ ID NO: 166; SEQ ID NO: 169; SEQ ID NO: 173; SEQ ID NO: 174; SEQ ID NO: 183; SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; SEQ ID NO: 195; SEQ ID NO: 196; SEQ ID NO: 197; SEQ ID NO: 198; SEQ ID NO: 199; SEQ ID NO: 200; SEQ ID NO: 201; SEQ ID NO: 202; SEQ ID NO: 203; SEQ ID NO: 204; SEQ ID NO: 205; SEQ ID NO: 206; SEQ ID NO: 207; SEQ ID NO: 208; SEQ ID NO: 209; SEQ ID NO: 210; SEQ ID NO: 211; SEQ ID NO: 212; SEQ ID NO: 213; SEQ ID NO: 214; SEQ ID NO: 215; SEQ ID NO: 216; SEQ ID NO: 217; SEQ ID NO: 218; SEQ ID NO: 219; SEQ ID NO: 220; SEQ ID NO: 221; SEQ ID NO: 222; SEQ ID NO: 223; SEQ ID NO: 224; SEQ ID NO: 225; SEQ ID NO: 226; SEQ ID NO: 227; SEQ ID NO: 228; SEQ ID NO: 229; SEQ ID NO: 230; SEQ ID NO: 231; SEQ ID NO: 232; SEQ ID NO: 233; SEQ ID NO: 234; SEQ ID NO: 235; SEQ ID NO: 236; SEQ ID NO: 237; SEQ ID NO: 238; SEQ ID NO: 239; SEQ ID NO: 240; SEQ ID NO: 241; SEQ ID NO: 242; SEQ ID NO: 243; SEQ ID NO: 244; SEQ ID NO: 245; SEQ ID NO: 246; SEQ ID NO: 247; SEQ ID NO: 248; SEQ ID NO: 249; SEQ ID NO: 250; SEQ ID NO: 251; SEQ ID NO: 252; SEQ ID NO: 253; SEQ ID NO: 254; SEQ ID NO: 255; SEQ ID NO: 256; SEQ ID NO: 257; SEQ ID NO: 258; SEQ ID NO: 259; SEQ ID NO: 260; SEQ ID NO: 261; SEQ ID NO: 262; SEQ ID NO: 263; SEQ ID NO: 264; SEQ ID NO: 265; SEQ ID NO: 266; SEQ ID NO: 267; SEQ ID NO: 268; SEQ ID NO: 269; SEQ ID NO: 270; SEQ ID NO: 271; SEQ ID NO: 272; SEQ ID NO: 273; SEQ ID NO: 274; SEQ ID NO: 275; SEQ ID NO: 276; SEQ ID NO: 277; SEQ ID NO: 278; SEQ ID NO: 279; SEQ ID NO: 280; SEQ ID NO: 281; SEQ ID NO: 282; SEQ ID NO: 283; SEQ ID NO: 284; SEQ ID NO: 285; SEQ ID NO: 286; SEQ ID NO: 287; SEQ ID NO: 288; SEQ ID NO: 289; SEQ ID NO: 290; SEQ ID NO: 291; SEQ ID NO: 293; SEQ ID NO: 294; SEQ ID NO: 295; SEQ ID NO: 296; SEQ ID NO: 297; SEQ ID NO: 298; SEQ ID NO: 299; SEQ ID NO: 300; SEQ ID NO: 301; SEQ ID NO: 302; SEQ ID NO: 303; SEQ ID NO: 304; SEQ ID NO: 305; SEQ ID NO: 306; SEQ ID NO: 307; SEQ ID NO: 308; SEQ ID NO: 309; SEQ ID NO: 310; SEQ ID NO: 311; SEQ ID NO: 312; SEQ ID NO: 313; SEQ ID NO: 314; SEQ ID NO: 315; SEQ ID NO: 316; SEQ ID NO: 317; SEQ ID NO: 318; SEQ ID NO: 320; SEQ ID NO: 321; SEQ ID NO: 322; SEQ ID NO: 323; SEQ ID NO: 324; SEQ ID NO: 325; SEQ ID NO: 326; SEQ ID NO: 327; SEQ ID NO: 328; and SEQ ID NO: 329.

27. A microarray comprising a gene expression profile comprising one or more genes or oligonucleotide probes obtained therefrom, wherein said gene expression profile is generated from a cell type selected from the group comprising coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesangial cells, coronary artery smooth muscle, bronchial smooth muscle, uterine smooth muscle, lung fibroblast, osteoblasts, and prostate stromal cells.

28. A method of determining the level of RNA expression for a sample comprising the steps of:
determining the level of RNA expression for an RNA sample, wherein said RNA sample is amplified, fluorescently labeled, and hybridized to a microarray containing a plurality of nucleic acid sequences, and wherein said microarray is scanned for fluorescence;
normalizing said expression level using an algorithm; and
scoring said RNA sample against a gene expression profile database.

29. The method of claim 28, wherein said RNA sample is obtained from a patient.

30. The method of claim 29, wherein said RNA sample is selected from the group consisting of blood, urine, amniotic fluid, plasma, semen, bone marrow, and tissue biopsy.

31. The method of claim 28, wherein said algorithm is the MaxCor algorithm.

32. The method of claim 28, wherein said algorithm is the Mean Log Ratio algorithm.

33. A method for constructing a gene expression profile comprising the steps of:
hybridizing prepared RNA samples to at least one microarray containing a plurality of nucleic acid sequences representing human genes;
obtaining an expression level for each of said plurality of nucleic acid sequences representing human genes on each of said at least one microarrays; and
normalizing said expression level for each of said plurality of nucleic acid sequences representing human genes on each of said at least one microarrays to control standards.

34. The method of claim 33 further comprising the steps of:
applying an algorithm to each of said normalized gene expression levels;
performing a correlation analysis for all of said normalized gene expression microarrays within a group of samples;
establishing a gene expression profile; and
validating the gene expression profile.

35. The method of claim 34, wherein said algorithm is the MaxCor algorithm.

36. The method of claim 35, wherein applying said MaxCor algorithm to each of said normalized gene expression levels assigns a numeric value to each gene represented on said at least one microarray based upon expression level.

37. The method of claim 36, wherein said numeric value is a number between the range of (−1, +1).

38. The method of claim 37, wherein a negative value of said numeric value represents a gene with relatively lower expression.

39. The method of clam 37, wherein a zero value of said numeric value represents no relative gene expression difference.

40. The method of claim 37, wherein a positive value of said numeric value represents a gene with relatively higher expression.

41. The method of claim 36, wherein said numeric value is a number between the range of (−2, +2).

42. The method of claim 41, wherein a negative value of said numeric value represents a gene with relatively lower expression.

43. The method of clam 41, wherein a zero value of said numeric value represents no relative gene expression difference.

44. The method of claim 41, wherein a positive value of said numeric value represents a gene with relatively higher expression.

45. The method of claim 34, wherein said algorithm is the Mean Log Ratio algorithm.

46. The method of claim 45, wherein applying said Mean Log Ratio algorithm to each of said gene expression microarrays assigns a numeric value to each gene contained on said microarray based upon expression level.

47. The method of claim 46, wherein said numeric value is between the range of (−1, +1).

48. The method of claim 47, wherein a negative value of said numeric value represents a gene with relatively lower expression.

49. The method of claim 47, wherein a zero value of said numeric value represents no relative gene expression difference.

50. The method of claim 47, wherein a positive value of said numeric value represents a gene with relatively higher expression.

51. The method of claim 46, wherein said numeric value is a number between the range of (−2, +2).

52. The method of claim 51, wherein a negative value of said numeric value represents a gene with relatively lower expression.

53. The method of clam 51, wherein a zero value of said numeric value represents no relative gene expression difference.

54. The method of claim 51, wherein a positive value of said numeric value represents a gene with relatively higher expression.

55. A method, in a computer system, for constructing and analyzing a gene expression profile comprising the steps of:
inputting gene expression data for each of a plurality of genes;
normalizing expression data by transforming said data into log ratio values;
filtering weak differential values;
applying an algorithm to each of said normalized gene expression values;
performing a classification analysis for all of said normalized gene expression values;
establishing a gene expression profile; and
validating the gene expression profile.

56. The method of claim 55, wherein said algorithm is the MaxCor algorithm.

57. The method of claim 55, wherein said algorithm is the Mean Log Ratio algorithm.

58. A computer program for constructing and analyzing a gene expression profile comprising:
computer code that receives as input gene expression data for a plurality of genes;
computer code that normalizes expression data by transforming said data into log ratio values;
computer code that applies an algorithm to each of said normalized gene expression values;
computer code that performs a correlation analysis for all of said normalized gene expression values;
computer code that establishes and validates the gene expression profile; and
computer readable medium that stores computer code.

59. The computer program of claim 58, wherein said algorithm is the MaxCor algorithm.

60. The computer program of claim 58, wherein said algorithm is the Mean Log Ratio algorithm.

61. A method for determining the phenotype of a cell comprising the steps of
applying an algorithm to extract a gene expression profile from gene expression data generated from said cell; and
matching said gene expression profile to a gene expression profile generated from a cell of known phenotype.

62. The method of claim 61, wherein said algorithm is the MaxCor algorithm.

63. The method of claim 61, wherein said algorithm is the Mean Log Ratio algorithm.

64. The method of claim 61, wherein said applying step comprises setting a cutoff value for expression relative to normalized values, wherein said cutoff value is at least about two-fold induction above the normalized values.

65. The method of claim 61, wherein said matching step is performed using a database comprising one or more gene expression profiles generated from cells of known phenotype.

66. A method for distinguishing cell types comprising the step of matching a gene expression profile generated from a biological sample using an algorithm to a known gene expression profile of a specific cell type.

67. The method of claim 66, wherein said algorithm is the MaxCor algorithm.

68. The method of claim 66, wherein said algorithm is the Mean Log Ratio algorithm.

69. The method of claim 66, wherein said specific cell type is selected from the group consisting of coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesangial cells, coronary artery smooth muscle, bronchial smooth muscle, uterine smooth muscle, lung fibroblast, osteoblasts, and prostate stromal cells.

70. A microarray comprising one or more protein-capture agents that specifically bind to all or a portion of one or more of the proteins encoded by the genes comprising the gene expression profile of claim 1.

71. A microarray comprising one or more protein-capture agents that specifically bind to all or a portion of one or more of the proteins encoded by the genes comprising the gene expression profile of claim 2.

72. A microarray comprising one or more protein-capture agents that specifically bind to all or a portion of one or more of the proteins encoded by the genes comprising the gene expression profile of claim 3.

73. A microarray comprising one or more protein-capture agents that specifically bind to all or a portion of one or more of the proteins encoded by the genes comprising the gene expression profile of claim 4

74. A microarray comprising one or more protein-capture agents that specifically bind to all or a portion of one or more of the proteins encoded by the genes comprising the gene expression profile of claim 5.

75. A microarray comprising one or more protein-capture agents that specifically bind to all or a portion of one or more of the proteins encoded by the genes comprising the gene expression profile of claim 6.

76. A microarray comprising one or more protein-capture agents that specifically bind to all or a portion of one or more of the proteins encoded by the genes comprising the gene expression profile of claim 7.

77. A microarray comprising one or more protein-capture agents that specifically bind to all or a portion of one or more of the proteins encoded by the genes comprising the gene expression profile of claim 8.

78. A microarray comprising one or more protein-capture agents that specifically bind to all or a portion of one or more of the proteins encoded by the genes comprising the gene expression profile of claim 9.

79. A microarray comprising one or more protein-capture agents that specifically bind to all or a portion of one or more of the proteins encoded by the genes comprising the gene expression profile of claim 10.

80. A microarray comprising one or more protein-capture agents that specifically bind to all or a portion of one or more of the proteins encoded by the genes comprising the gene expression profile of claim 11.

81. A microarray comprising one or more protein-capture agents that specifically bind to all or a portion of one or more of the proteins encoded by the genes comprising the gene expression profile of claim 12.

82. A method for determining the phenotype of a cell comprising the steps of
applying an algorithm to extract a protein expression profile from protein expression data generated from said cell; and
matching said protein expression profile to a protein expression profile generated from a cell of known phenotype.

83. The method of claim 82, wherein said algorithm is the MaxCor algorithm.

84. The method of claim 82, wherein said algorithm is the Mean Log Ratio algorithm.

85. The method of claim 82, wherein said applying step comprises setting a cutoff value for expression relative to normalized values, wherein said cutoff value is at least about two-fold induction above the normalized values.

86. The method of claim 82, wherein said matching step is performed using a database comprising one or more protein expression profiles generated from cells of known phenotype.

87. A method for distinguishing cell types comprising the step of matching a protein expression profile generated from a biological sample using an algorithm to a known protein expression profile of a specific cell type.

88. The method of claim 87, wherein said algorithm is the MaxCor algorithm.

89. The method of claim 87, wherein said algorithm is the Mean Log Ratio algorithm.

90. The method of claim 87, wherein said specific cell type is selected from the group consisting of coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesangial cells, coronary artery smooth muscle, bronchial smooth muscle, uterine smooth muscle, lung fibroblast, osteoblasts, and prostate stromal cells.
